(19) United States
(12) Patent Application Publication
(10) Pub. No.: US 2020/0323898 A1
(43) Pub. Date: Oct. 15, 2020

(54) COMPOSITIONS AND METHODS FOR TREATING ACUTE MYELOID LEUKEMIA

(71) Applicants: MEMORIAL SLOAN KETTERING CANCER CENTER, New York, NY (US); COLD SPRING HARBOR LABORATORY, Cold Spring Harbor, NY (US)

(72) Inventors: Scott Lowe, New York, NY (US); Lingbo Zhang, Cold Spring Harbor, NY (US); Chichao Chen, New York, NY (US)

(21) Appl. No.: 16/767,280

(22) PCT Filed: Nov. 27, 2018
(86) PCT No.: PCT/US2018/062676

§ 371 Date: May 27, 2020

Related U.S. Application Data
(60) Provisional application No. 62/591,652, filed on Nov. 28, 2017.

(51) Int. Cl. A61K 31/7105; A61K 9/00; G01N 33/68; C12Q 1/48; A61P 35/02

(52) U.S. Cl. CPC A61K 31/7105; A61K 9/0053; A61K 9/0014; A61K 9/0043; A61K 9/0019; G01N 2800/52; A61K 9/0051; G01N 33/6803; C12Q 1/485; A61P 35/02; G01N 2333/91215; A61K 9/0021

(57) ABSTRACT

The present disclosure relates generally to methods for ameliorating or treating acute myeloid leukemia (AML). In particular, the present technology relates to administering a therapeutically effective amount of one or more compositions that inhibit the vitamin B6 pathway to a subject diagnosed with, or at risk for AML.

### CROSS-REFERENCE TO RELATED APPLICATIONS

This application claims the benefit of and priority to U.S. Provisional Patent Application No. 62/591,652, filed Nov. 28, 2017, the entire contents of which are incorporated herein by reference.

### STATEMENT OF GOVERNMENT SUPPORT

This invention was made with government support under CA190261, awarded by the National Institutes of Health. The government has certain rights in the invention.

### TECHNICAL FIELD

The present technology relates generally to compositions and methods for ameliorating or treating acute myeloid leukemia. In particular, the present technology relates to administering a therapeutically effective amount of one or more compositions that inhibit the vitamin B6 pathway to a subject diagnosed with, or at risk for acute myeloid leukemia.

### BACKGROUND

The following description of the background of the present technology is provided simply as an aid in understanding the present technology and is not admitted to describe or constitute prior art to the present technology.

Metabolic reprogramming is an emerging hallmark of cancer, allowing cancer cells to fulfill the metabolic and energetic requirements to undergo abnormal proliferation (M. G. Vander Heiden, Nat Rev Drug Discov 10:671-684 (2011); R. A. Cairns et al., Nat Rev Cancer 11:85-95 (2011); J. R. Cantor & D. M. Sabatini, Cancer Discov 2:881-898 (2012); N. N. Pavlova & C. B. Thompson, Cell Metab 23:27-47 (2016)). However, since normal proliferative cells in adult tissues share some common metabolic features with cancer cells, many of metabolic events are not selectively required by cancer cells. Further, limited insights into the molecular mechanisms of metabolic reprogramming and the absence of druggable targets precludes the use of therapeutic strategies that selectively target this cellular process to treat cancer.

Acute myeloid leukemia (AML) has the worst 5-year-survival rate of all leukemias. Despite therapeutic improvement in recent decades, AML remains a clinically challenging disease.

### SUMMARY OF THE PRESENT TECHNOLOGY

In one aspect, the present disclosure provides a method for treating or preventing AML in a subject in need thereof, comprising administering to the subject a therapeutically effective amount of at least one inhibitor of Vitamin B6 signaling pathway, wherein the at least one inhibitor is selected from the group consisting of isoniazid, aftin-4, 2-difluoromethylornithine (DFMO), gingkotoxin, aminooxyacetic acid, and myriocin.

In another aspect, the present disclosure provides a method for treating or preventing AML in a subject in need thereof, comprising administering to the subject a therapeutically effective amount of at least one sgRNA or shRNA that targets one or more genes selected from the group consisting of PDXK, PNPO, AZIN1, ODC1, GOT2, ALAS1, SPTLC1, and SPTLC2. In some embodiments, the at least one sgRNA or shRNA comprises a nucleic acid sequence selected from the group consisting of:

(SEQ ID NO: 1)
5′ TGGCTACGTGGGTAACAGAG 3′ (Pdxk sg-1),
(SEQ ID NO: 2)
5′ ATCCAGAGCCATGTTGTCCG 3′ (Pdxk sg-2),
(SEQ ID NO: 3)
5′ GTGCAGTTTTCAAACCACAC 3′ (Pdxk sg-3),
(SEQ ID NO: 4)
5′ GCTTGGGGTGCCTGCAGAGA 3′ (Odc1-a106),
(SEQ ID NO: 5)
5′ TGCTGTTGACAGTGAGCGCCAAGGTGAACGATGTCAATAATAGTG
AAGCCACAGATGTATTATTGACATCGTTCACCTTGATGCCTACTGCCT
CGGA 3′ (Pdxk.307),
(SEQ ID NO: 6)
TGCTGTTGACAGTGAGCGCCAGGTTCAATGTGAGGTTACATAGTGAAG
CCACAGATGTATGTAACCTCACATTGAACCTGATGCCTACTGCCTCGG
A 3′ (Pdxk.307),
(SEQ ID NO: 7)
5′ ACGCCCAGGATGGGATCTGG 3′ (Got2.a41),
(SEQ ID NO: 8)
5′ AAAGAATACCTGCCCATTGG 3′ (Got2.a99),
(SEQ ID NO: 9)
5′ GACTGGAGCCTTAAGGGTCG 3′ (Got2.a140),
(SEQ ID NO: 10)
5′ ATACAGAGCCACGTCATCCG 3′ (hPdxk-aa15),
(SEQ ID NO: 11)
5′ CGGCTACGTGGGCAACCGGG 3′ (hPdxk-aa22),
(SEQ ID NO: 12)
5′ GCCTACCGTACACCAGCCTG 3′ (hPdxk-aa105),
(SEQ ID NO: 13)
5′ GTCCCCAGTGCCCACAAAGA 3′ (hPDXK-aa230),
(SEQ ID NO: 14)
5′ AATGGCTTTAGTGCAAGAAT 3′ (Azin1-a100),
(SEQ ID NO: 15)
5′ GAACTACTCCGTTGGCCTGT 3′ (Azin1-a14),
(SEQ ID NO: 16)
5′ GCCAAGATCTCAAGCACGGC 3′ (Azin1-a76),
(SEQ ID NO: 17)
5′ ATATTGACGTCATTGGTGTG 3′ (Odc1-a194),
(SEQ ID NO: 18)
5′ AGGCAGCAGCGTCTTCCGCA 3′ (ALAS1 sg-1),
(SEQ ID NO: 19)
5′ CACCGTTTTAAAAACTCGGT 3′ (ALAS1 sg-2),
(SEQ ID NO: 20)
5′ CTCGGGATAAGAATGGGCAT 3′ (ALAS1 sg-3),
(SEQ ID NO: 21)
5′ TGCGTAAAAGGGAGTGACGC 3′ (Odc1-a62),
(SEQ ID NO: 22)
5′ GCTGGCCAACCCTCGAGTTA 3′ (SPTLC1 sg-1),
(SEQ ID NO: 23)
5′ GATGGTGCAGGCGCTGTACG 3′ (SPTLC1 sg-2),
(SEQ ID NO: 24)
5′ TCAACTACAACATCGTGTCC 3′ (SPTLC1 sg-3),
(SEQ ID NO: 25)
5′ GCTCCAGGCACACTACAGAT 3′ (SPTLC2 sg-1),
(SEQ ID NO: 26)
5′ GAACGGCTGCGTCAAGAACG 3′ (SPTLC2 sg-2),
(SEQ ID NO: 27)
5′ AATCTCGAAGATATCCAAAG 3′ (SPTLC2 sg-3),
(SEQ ID NO: 28)
5′ GGTGTGTGGTTTCCCCAGGT 3′ (hGOT2.a162),
(SEQ ID NO: 29)
5′ GATGGGTGTGTGGTTTCCCC 3′ (hGOT2.a163),
(SEQ ID NO: 30)
5′ GGACGCGGGTCCACTCCCGT 3′ (hGOT2.a218),
(SEQ ID NO: 31)
5′ TGGACCCGCGTCCGGAACAG 3′ (hGOT2.a224),
(SEQ ID NO: 39)
5′ ACGATGAACATGTTAGACAT 3′ (hAZIN1-a233),
(SEQ ID NO: 40)
5′ CTATGTTTATGAACATACCC 3′ (hAZIN1-a33),
(SEQ ID NO: 41)
5′ TATCTGCTTGATATTGGCGG 3′ (hODC1-a235),
(SEQ ID NO: 42)
5′ CAACGCTGGGTTGATTACGC 3′ (hODC1-a254),
(SEQ ID NO: 982)
5′ GGAGGTCCTGGGGAACGTAC 3′ (Pdxk sg-4),
(SEQ ID NO: 983)
5′ CATGGCAGCGAAGAGGTCCC 3′ (Pdxk sg-5),
(SEQ ID NO: 984)
5′ AGCTGTCTTCGTGGGCACCG 3′ (Pdxk sg-6),
(SEQ ID NO: 985)
5′ TGTAACCTCACATTGAACCTGA 3′,
(SEQ ID NO: 986)
5′ TTATTGACATCGTTCACCTTGA 3′,
(SEQ ID NO: 987)
5′ CATGCGCAAGAGTTACCGCG 3′ (hPNPO-a42:);
(SEQ ID NO: 988)
5′ ATGACCGGATAGTCTTTCGG 3′ (hPNPO-a232);
(SEQ ID NO: 989)
5′ GAGTTACCGCGGGGACCGAG 3′ (hPNPO-a45);
and
(SEQ ID NO: 990)
5′ TTCTGTGATCCCTGATCGGG 3′ (hPNPO-a181).

Additionally or alternatively, in some embodiments of the methods of the present technology, the subject displays elevated expression levels of PDXK protein in leukemic cells prior to treatment. In certain embodiments, treatment with the at least one sgRNA, shRNA, or inhibitor of Vitamin B6 pathway results in a decrease in PDXK and/or PLP levels in the subject compared to that observed prior to treatment.

In some embodiments of the methods of the present technology, the subject has been diagnosed as having AML. Signs or symptoms of AML may comprise one or more of leukemic cell proliferation, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite.

In any of the above embodiments of the methods disclosed herein, the subject may harbor one or more point mutations in NRAS, DNMT3A, FLT3, KIT, IDH1, IDH2, CEBPA and NPM1 and/or one or more gene fusions selected from the group consisting of CBFB-MYH11, DEK-NUP214, MLL-MLLT3, PML-RARA, RBM15-MKL1, RPN1-EVI1 and RUNX1-RUNX1T1. In certain embodiments, the subject is human.

Additionally or alternatively, in some embodiments of the methods of the present technology, the at least one sgRNA, shRNA, or inhibitor of Vitamin B6 pathway is administered orally, topically, intranasally, systemically, intravenously, subcutaneously, intraperitoneally, intradermally, intraocularly, iontophoretically, transmucosally, or intramuscularly. In some embodiments, the at least one sgRNA, shRNA, or inhibitor of Vitamin B6 pathway is administered daily for 6 weeks or more. In other embodiments, the at least one sgRNA, shRNA, or inhibitor of Vitamin B6 pathway is administered daily for 12 weeks or more.

Additionally or alternatively, in some embodiments, the methods further comprise separately, sequentially or simultaneously administering one or more additional therapeutic agents to the subject. Examples of additional therapeutic agents include cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

In one aspect, the present disclosure provides a method for inhibiting leukemic cell proliferation in a subject in need thereof, comprising administering to the subject a therapeutically effective amount of at least one inhibitor of Vitamin B6 pathway, wherein the at least one inhibitor is selected from the group consisting of isoniazid, aftin-4, DFMO, gingkotoxin, aminooxyacetic acid, and myriocin, and wherein the subject suffers from a disease or condition characterized by elevated expression levels and/or increased activity of PDXK.

In another aspect, the present disclosure provides a method for inhibiting leukemic cell proliferation in a subject in need thereof, comprising administering to the subject a therapeutically effective amount of at least one sgRNA or shRNA that targets one or more genes selected from the group consisting of PDXK, PNPO, AZIN1, ODC1, GOT2, ALAS1, SPTLC1, and SPTLC2, wherein the subject suffers from a disease or condition characterized by elevated expression levels and/or increased activity of PDXK.

In one aspect, the present disclosure provides a method for monitoring the therapeutic efficacy of a dosage of an inhibitor of Vitamin B6 pathway in a subject diagnosed with AML comprising: (a) detecting PDXK protein levels or intracellular PLP levels in a test sample obtained from the subject after the subject has been administered the dosage of the inhibitor of Vitamin B6 pathway, wherein the inhibitor of Vitamin B6 pathway is isoniazid, aftin-4, DFMO, gingkotoxin, aminooxyacetic acid, or myriocin; and (b) determining that the dosage of the inhibitor of Vitamin B6 pathway is effective when the PDXK protein levels or intracellular PLP levels in the test sample are reduced compared to that observed in a control sample obtained from the subject prior to administration of the inhibitor of Vitamin B6 pathway. The test sample may be tissues, cells or biological fluids (blood, plasma, saliva, urine, serum etc.) present within a subject. In certain embodiments, the method further comprises detecting intracellular levels of PLP in the subject.

Also disclosed herein are methods for monitoring the therapeutic efficacy of an inhibitory RNA that targets a gene selected from the group consisting of PDXK, PNPO, AZIN1, ODC1, GOT2, ALAS1, SPTLC1, and SPTLC2 in a subject diagnosed with AML comprising: (a) detecting PDXK protein levels or intracellular PLP levels in a test sample obtained from the subject after the subject has been administered the inhibitory RNA; and (b) determining that the inhibitory RNA is effective when the PDXK protein levels or intracellular PLP levels in the test sample are reduced compared to that observed in a control sample obtained from the subject prior to administration of the inhibitory RNA. The inhibitory RNA may be a shRNA or a sgRNA. The test sample may be tissues, cells or biological fluids (blood, plasma, saliva, urine, serum etc.) present within a subject. In certain embodiments, the method further comprises detecting intracellular levels of PLP in the subject.

In any of the above embodiments of the methods disclosed herein, the intracellular PLP levels are detected via high-performance liquid chromatography-mass spectrometry.

### BRIEF DESCRIPTION OF THE DRAWINGS

FIG. 1A shows a schematic diagram of the number of metabolic genes enriched in AML cells.

FIG. 1B shows the ratio of the number of reads on day 9 over day 1 corresponding to each sgRNA. Nras(G12D)/MLL-AF9 leukemic cells were infected with viral sgRNA pool. Genomic DNAs were extracted from day 1 and day 9 of cultured cells. Integrated sgRNAs were PCR amplified and sequenced.

FIG. 1C shows a schematic diagram representing the dependency of each indicated cell line on the listed genes from previously published reports (See Wang, T. et al., Cell 168:890-903 (2017); McDonald, E. R. et al., Cell 170:577-592 (2017)). Each row represents a gene, BCL2 and PDXK were labeled with red color. Each column represents a cell line with red color indicating AML cell lines and black color indicating non-AML cell lines. ‘NS’ represented no statistical difference, ‘*’ represented differences with P values of <0.05, ‘**’ represented differences with P values of <0.01, and ‘***’ represented differences with P values of <0.005 of Wilcox test.

FIG. 1D shows the percentage of GFP⁺ sgRNA infected cells in the culture system that were quantified at day 2, day 7, day 13, day 18, and day 24 of cell culture. iMEF cells were infected with viruses encoding the indicated sgRNAs. The average and standard deviation (STDEV) of the relative GFP⁺ percentage are shown.

FIG. 1E shows the expression levels of PDXK protein in sorted GFP⁺ and GFP⁻ cells. iMEF cells were infected with viruses encoding the indicated sgRNAs. Western blot analysis was performed to measure the expression levels of PDXK protein.

FIG. 1F shows the percentage of GFP⁺ sgRNA infected cells in the culture system that were quantified at day 2, day 4, day 6, day 8, day 10, day 12, and day 14 of cell culture. Nras(G12D)/MLL-AF9 leukemic cells were infected with viruses encoding the indicated sgRNAs. The average and standard deviation (STDEV) of the relative GFP⁺ percentage are shown.

FIG. 1G shows the expression levels of PDXK protein in sorted GFP⁺ and GFP⁻ cells. Nras(G12D)/MLL-AF9 leukemic cells were infected with viruses encoding the indicated sgRNAs. Western blot analysis was performed to measure the expression levels of PDXK protein.

FIG. 1H shows five indicated leukemic cell lines that were infected with viruses encoding the indicated shRNAs. The percentage of GFP⁺ shRNA infected cells in the cell culture system were quantified from day 1 to day 14 of cell culture. The average and STDEV of the relative GFP⁺ percentage are shown.

FIG. 1I shows the percentage of the five indicated leukemic cell lines that were cultured with the indicated concentrations of isoniazid at day 6 of cell culture.

FIG. 2A shows the levels of pyridoxal phosphate (PLP) in Nras(G12D)/MLL-AF9 leukemic cells that were infected with viruses encoding the indicated sgRNAs. The abundance of PLP was measured by high-performance liquid chromatography-mass spectrometry (HPLC-MS) and normalized to the level of Rosa26 sgRNA group (negative control). The average and STDEV are shown. Western blot analysis was performed to measure the expression levels of PDXK protein. “***” represents p<0.001 oft-test.

FIG. 2B shows the levels of PLP in Nras(G12D)/MLL-AF9 leukemic cells that were infected with viruses encoding the indicated shRNAs. The abundance of PLP was measured by high-performance liquid chromatography-mass spectrometry (HPLC-MS) and normalized to the level of Ren.713 shRNA group (negative control). The average and STDEV are shown. Western blot analysis was performed to measure the expression levels of PDXK protein. “*” represents p<0.05 oft-test, and “**” represents p<0.01 oft-test.

FIG. 2C shows the levels of PLP in Molm13 leukemic cells that were infected with viruses encoding the indicated sgRNAs. The abundance of PLP was measured by high-performance liquid chromatography-mass spectrometry (HPLC-MS) and normalized to the level of Rosa26 sgRNA group (negative control). The average and STDEV are shown. Western blot analysis was performed to measure the expression levels of PDXK protein. “***” represents p<0.001 oft-test.

FIG. 2D shows the levels of PLP in iMEF cells that were infected with viruses encoding the indicated sgRNAs. The abundance of PLP was measured by high-performance liquid chromatography-mass spectrometry (HPLC-MS) and normalized to the level of Rosa26 sgRNA group (negative control). The average and STDEV are shown. Western blot analysis was performed to measure the expression levels of PDXK protein. “***” represents p<0.001 oft-test.

FIG. 2E shows the levels of PLP in Nras(G12D)/MLL-AF9 leukemic cells that were cultured with the indicated concentrations of isoniazid at day 5 of cell culture. The abundance of PLP was measured by high-performance liquid chromatography-mass spectrometry (HPLC-MS) and normalized to the level [³D]-labeled PLP (internal control). The average and STDEV are shown. Western blot analysis was performed to measure the expression levels of PDXK protein.

FIG. 2F shows the expression levels of PDXK protein by western blot analysis in Molm13 leukemic cells that were infected with viruses encoding control vector, human wild type PDXK or mutant (D235A) PDXK and the hygromycin resistance gene. After hygromycin selection, Molm13 leukemic cells were infected with viruses encoding the indicated sgRNAs targeting Rosa26 or human PDXK.

FIG. 2G shows the percentage of GFP⁺ sgRNA infected cells in the culture system that were quantified at day 4, day 10, day 16, day 22, and day 30 of cell culture. Molm13 leukemic cells were infected with viruses encoding control vector, human wild type PDXK or mutant (D235A) PDXK and the hygromycin resistance gene. After hygromycin selection, Molm13 leukemic cells were infected with viruses encoding the indicated sgRNAs targeting Rosa26 or human PDXK. The average and STDEV of relative GFP⁺ percentages are shown.

FIG. 2H shows the levels of PLP in Molm13 leukemic cells that were infected with viruses encoding the indicated sgRNAs. Molm13 leukemic cells were infected with viruses encoding control vector, human wild type PDXK or mutant (D235A) PDXK and the hygromycin resistance gene. After hygromycin selection, Molm13 leukemic cells were infected with viruses encoding the indicated sgRNAs targeting Rosa26 or human PDXK. The abundance of PLP was measured by high-performance liquid chromatography-mass spectrometry (HPLC-MS) and normalized to the level of Rosa26 sgRNA group (negative control). The average and STDEV are shown.

FIG. 2I shows the number of Nras(G12D)/MLL-AF9 leukemic cells that were cultured in the absence or presence of 1 μg/ml of vitamin B6 pyridoxine over day 1 to day 4 of cell culture. The average and STDEV are shown.

FIG. 2J shows the number of iMEF cells that were cultured in the absence or presence of 1 μg/ml of vitamin B6 pyridoxine over day 1 to day 4 of cell culture. The average and STDEV are shown.

FIG. 3A shows luciferase levels observed in sub-lethally irradiated mice that were transplanted with Nras(G12D)/MLL-AF9 cells infected with viruses encoding shRNAs targeting Renilla luciferase (n=12) or PDXK (Pdxk.307, n=13; and Pdxk.3259, n=11). Doxycycline (Dox) was supplemented in the mouse diet 5 days after transplantation. Luciferase signals were monitored at day 5 and day 14 post transplant.

FIG. 3B shows the quantification of luciferase intensities from FIG. 3A. “NS” represents no statistical significance oft-test, “*” represents p<0.05 oft-test, “**” represents p<0.01 of t-test., and “***” represents p<0.001 oft-test.

FIG. 3C shows shows the expression levels of PDXK protein on day 1 to day 4 of cell culture. Nras(G12D)/MLL-AF9 leukemic cells were infected with viruses encoding the indicated shRNAs. Western blot analysis was performed to measure the expression levels of PDXK protein.

FIG. 3D shows the survival curves of the animals from FIG. 3A. “***” represents p<0.001 of Log-rank (Mantel-cox) test.

FIG. 3E shows luciferase levels observed in sub-lethally irradiated mice that were transplanted with Nras(G12D)/MLL-AF9 cells. A daily dose of control PBS (n=12) or 90 mg/kg isoniazid (n=15) was intraperitoneally injected into the animals and luciferase signals were detected at day 4 and day 10 post transplant.

FIG. 3F shows the quantification of luciferase intensities from FIG. 3E. “NS” represents no statistical significance oft-test, and “*” represents p<0.05 oft-test.

FIG. 3G shows the survival curves of the animals shown in FIG. 3E. “***” represents p<0.001 of Log-rank (Mantel-cox) test.

FIG. 4A shows the relative abundance of the indicated metabolites in control leukemic cells or leukemic cells with PDXK depletion. The abundance of the indicated metabolites was measured by high-performance liquid chromatography-mass spectrometry (HPLC-MS).

FIG. 4B shows the expression levels of PLP dependent genes in mouse Nras(G12D)/MLL-AF9 leukemic cells and human Molm13 leukemic cells.

FIG. 4C shows the percentage of GFP⁺ Nras(G12D)/MLL-AF9 leukemic cells or iMEF cells that were infected with viruses encoding the indicated sgRNAs at day 10 of cell culture. The log 2 ratios of GFP⁺ percentage at day 24 over day 1 corresponding to each indicated sgRNA are shown.

FIG. 4D shows the fold of cell proliferation of Nras(G12D)/MLL-AF9 leukemic cells that were cultured in the absence or presence of the indicated concentrations of isoniazid and putrescine on day 4 of cell culture. The average and SEM of fold of cell proliferation are shown. “*” represents p<0.05 of one-way ANOVA test, “**” represents p<0.01 of one-way ANOVA test, “***” represents p<0.001 of one-way ANOVA test.

FIG. 4E shows the fold of cell proliferation of Nras(G12D)/MLL-AF9 leukemic cells that were cultured in the absence or presence of the indicated concentrations of isoniazid and uridine on day 4 of cell culture. The average and SEM of fold of cell proliferation are shown. “*” represents p<0.05 of one-way ANOVA test, “**” represents p<0.01 of one-way ANOVA test, “***” represents p<0.001 of one-way ANOVA test.

FIG. 4F shows the fold of cell proliferation of Nras(G12D)/MLL-AF9 leukemic cells that were cultured in the absence or presence of the indicated concentrations of isoniazid and asparagine on day 4 of cell culture. The average and SEM of fold of cell proliferation are shown. “*” represents p<0.05 of one-way ANOVA test, “**” represents p<0.01 of one-way ANOVA test, “***” represents p<0.001 of one-way ANOVA test.

FIG. 4G shows a schematic diagram of the ornithine—putrescine/nucleotides and oxaloacetate—nucleotides/aspartate/asparagine metabolic pathways.

FIG. 5A shows the gene expression profiles of leukemic cells from 285 AML patients classified into 16 groups based on molecular signatures and CD34⁺ HSPCs from 3 healthy individuals (downloaded from P. J. Valk et al., N Engl J Med 350:1617-1628 (2004)). For 2752 genes encoding metabolic enzymes and transporters, root mean square deviation (RMSD) was calculated (J. Hu et al., Nat Biotechnol 31:522-529 (2013)) between leukemic cells and HSPCs and among leukemic cells from different subtypes of AML.

FIG. 5B shows the average of RMSD among different subtypes of AMLs and the average of RMSD between AML and HSPCs.

FIG. 5C shows a schematic diagram of the CRISPR/Cas functional genomic screening procedure described herein. AML cells were infected with viral sgRNA pools and genomic DNA was extracted from cultured cells at day 1 and day 9. sgRNA inserts were PCR amplified for deep sequencing.

FIG. 5D shows a schematic diagram of the CRISPR/Cas functional genomic screening results. Potential drug targets are genes whose inhibition results in blocking leukemic cell proliferation.

FIG. 6A shows a schematic diagram representing the dependency of each indicated cell line on PLP-dependent genes from a previously published report (See Rauscher, B. et al., Nucleic Acids Res 45:D679-D686 (2017)). Each row represents a gene, and each column represents a cell line with red color indicating AML cell lines and black color indicating non-AML cell lines. Log fold change indicated by red and green color intensity is shown.

FIG. 6B shows a schematic diagram representing the dependency of each indicated cell line on the listed genes with unsupervised clustering from a previously published report (See Rauscher, B. et al., Nucleic Acids Res 45:D679-D686 (2017)). Each row represents a gene, and each column represents a cell line with red color indicating AML cell lines and black color indicating non-AML cell lines. “NS” represents no statistical significant difference, and “***” represents p<0.005 of Wilcoxon test.

FIG. 6C shows a schematic diagram representing the dependency of each indicated cell line on the listed genes with unsupervised clustering from a previously published report (See Rauscher, B. et al., Nucleic Acids Res 45:D679-D686 (2017)). Each row represents a gene, and each column represents a cell line with red color indicating AML cell lines and black color indicating non-AML cell lines. “NS” represents no statistical significant difference, and “***” represents p<0.005 of Wilcoxon test.

FIG. 6D shows the percentage of GFP⁺ sgRNA infected cells in the culture system that were quantified at day 2, day 7, day 15, day 22, and day 25 of cell culture. 3T3 cells were infected with viruses encoding the indicated sgRNAs.

FIG. 6E shows the percentage of GFP⁺ sgRNA infected cells in the culture system that were quantified at day 2, day 6, day 10, day 14, and day 26 of cell culture. Human sarcoma cells were infected with viruses encoding the indicated sgRNAs.

FIG. 6F shows the expression levels of PDXK protein by western blot analysis in human sarcoma cells that were infected with viruses encoding the indicated sgRNAs.

FIG. 7A shows the expression levels of PDXK protein in sorted GFP⁺ and GFP⁻ cells. Human Molm13 cells were infected with viruses encoding the indicated sgRNAs. Western blot analysis was performed to measure the expression levels of PDXK protein.

FIG. 7B shows the percentage of GFP⁺ sgRNA infected cells in the culture system that were quantified at day 2, day 8, day 14, day 22, and day 30 of cell culture. Human Molm13 cells were infected with viruses encoding the indicated sgRNAs. The average and STDEV of the relative GFP⁺ percentage are shown.

FIG. 7C shows the percentage of GFP⁺ sgRNA infected cells in the culture system that were quantified at day 2, day 9, day 14, day 23, and day 30 of cell culture. Human ML2 cells were infected with viruses encoding the indicated sgRNAs. The average and STDEV of the relative GFP⁺ percentage are shown.

FIG. 7D shows the percentage of GFP⁺ sgRNA infected cells in the culture system that were quantified at day 2, day 9, day 14, day 23, and day 30 of cell culture. Human SEMK2 cells were infected with viruses encoding the indicated sgRNAs. The average and STDEV of the relative GFP⁺ percentage are shown.

FIG. 7E shows the percentage of GFP⁺ sgRNA infected cells in the culture system that were quantified at day 2, day 8, day 14, day 23, and day 29 of cell culture. Human Thp1 leukemic cells were infected with viruses encoding the indicated sgRNAs. The average and STDEV of the relative GFP⁺ percentage are shown.

FIG. 7F shows the percentage of GFP⁺ sgRNA infected cells in the culture system that were quantified at day 2, day 8, day 14, day 23, and day 30 of cell culture. Human K562 leukemic cells were infected with viruses encoding the indicated sgRNAs. The average and STDEV of the relative GFP⁺ percentage are shown.

FIG. 7G shows the percentage of the seven indicated leukemic cell lines that were cultured with the indicated concentrations of isoniazid at day 6 of cell culture.

FIG. 8A shows the expression levels of PDXK in AML cells and CD34⁺ HSPCs.

FIG. 8B shows the expression levels of PDXK in each subtype of AML cells and in CD34⁺ HSPCs.

FIG. 8C shows the expression levels of PDXK protein in mouse bone marrow HSPCs that were infected with viruses encoding the indicated shRNAs.

FIG. 8D shows the GFP⁺ percentages of mouse bone marrow HSPCs that were infected with viruses encoding the indicated shRNAs. The percentage of GFP⁺ infected cells in the culture system were quantified at day 2, day 4, day 6, and day 8 of cell culture. The average and STDEV of the relative GFP⁺ percentage are shown.

FIG. 8E shows the GFP⁺ percentages of mouse Nras(G12D)/MLL-AF9 leukemic cells that were infected with viruses encoding the indicated shRNAs. The percentage of GFP⁺ infected cells in the culture system were quantified at day 2, day 4, day 6, and day 8 of cell culture. The average and STDEV of the relative GFP⁺ percentage are shown.

FIG. 8F shows the percentage of Nras(G12D)/MLL-AF9 leukemic cells or bone marrow HSPCs that were cultured with the indicated concentrations of isoniazid at day 6 of cell culture.

FIG. 9A shows Nras(G12D)/MLL-AF9 leukemic cells that were infected with viruses encoding the indicated sgRNAs. GFP⁺ cells in the culture system were sorted for May-Grunwald Giemsa (MGG) staining.

FIG. 9B shows Nras(G12D)/MLL-AF9 leukemic cells that were infected with viruses encoding the indicated sgRNAs. GFP⁺ cells in the culture system on day 4, day 6, and day 8 of cell culture were gated for c-Kit expression analysis.

FIG. 9C shows Nras(G12D)/MLL-AF9 leukemic cells that were infected with viruses encoding the indicated sgRNAs. GFP⁺ and GFP⁻ cells in the culture system on day 9 of cell culture were gated for cell cycle analysis. The average and STDEV are shown. “*” represents p<0.05 oft-test.

FIG. 9D shows Nras(G12D)/MLL-AF9 leukemic cells that were infected with viruses encoding the indicated sgRNAs. GFP⁺ cells in the culture system on day 12 of cell culture were gated for cell cycle analysis. The average and STDEV are shown. “***” represents p<0.001 oft-test.

FIG. 9E shows human Molm13 leukemic cells that were infected with viruses encoding the indicated sgRNAs. GFP⁺ cells in the culture system on day 17 of cell culture were gated for cell cycle analysis. The average and STDEV are shown. “**” represents p<0.01 oft-test.

FIG. 9F shows Nras(G12D)/MLL-AF9 leukemic cells that were infected with viruses encoding the indicated sgRNAs. GFP⁺ and GFP⁻ cells in the culture system on day 9 of cell culture were gated for Annexin V and 7-AAD apoptosis analysis. The average and STDEV are shown. “**” represents p<0.01 oft-test, and “***” represents p<0.001 oft-test.

FIG. 10A shows a volcano of fold change and p value comparing 6 PDXK genetic depletion samples and 15 PDXK wild type samples. Nras(G12D)/MLL-AF9 leukemic cells were infected with viruses encoding control sgRNAs or sgRNAs targeting PDXK, followed by RNA-Seq based gene expression analysis.

FIG. 10B shows the expression level of PDXK in a principal component analysis (PCA) of the gene expression analysis performed in FIG. 10A.

FIG. 10C shows pathways identified by KEGG pathway and gene ontology (GO) analysis that are downregulated upon PDXK depletion in mouse or human AML cells.

FIG. 10D shows pathways identified by a gene set enrichment analysis (GSEA) that are downregulated upon PDXK depletion.

FIG. 11A shows a schematic diagram of PLP and isoniazid as well as the PLP-isoniazid product. PLP detection in cells was confirmed by mass and retention time relative to the pure standard (chromatograms are for C8H10NO6P, extracted with a 20 ppm window). The peak detected in Molm13 cell extracts was also confirmed by the spiked addition of the pure standard.

FIG. 11B shows the levels of PLP in Nras(G12D)/MLL-AF9 leukemic cells that were cultured with the indicated concentrations of isoniazid. Cells were processed with or without PBS washing to remove residual isoniazid in cell culture before HPLC-MS analysis. The abundance of PLP was measured by HPLC-MS and normalized to the level in the absence of isoniazid with PBS washing. The average and STDEV are shown.

FIG. 11C shows the levels of [³D]-labeled PLP in Nras(G12D)/MLL-AF9 leukemic cells that were cultured with the indicated concentrations of isoniazid. Cells were processed with or without PBS washing to remove residual isoniazid in cell culture before HPLC-MS analysis. Abundance levels of [³D]-labeled PLP (internal control) were measured by HPLC-MS and normalized to the level in the absence of isoniazid with PBS washing. The average and STDEV are shown. Without one PBS wash, all PLP and [³D]-labeled PLP reacted with residual isoniazid in cell culture, therefore all PLP measurements upon isoniazid treatment were performed after one PBS wash.

FIG. 11D shows the levels of PLP in Molm13 cells that were cultured with the indicated concentrations of isoniazid. Cells were processed with or without PBS washing to remove residual isoniazid in cell culture before HPLC-MS analysis. The abundance of PLP was measured by HPLC-MS and normalized to the level in the absence of isoniazid with PBS washing. The average and STDEV are shown.

FIG. 11E shows the levels of [³D]-labeled PLP in Molm13 cells that were cultured with the indicated concentrations of isoniazid. Cells were processed with or without PBS washing to remove residual isoniazid in cell culture before HPLC-MS analysis. Abundance levels of [³D]-labeled PLP (internal control) were measured by HPLC-MS and normalized to the level in the absence of isoniazid with PBS washing. The average and STDEV are shown.

FIG. 11F shows the expression levels of PDXK protein by western blot analysis in Molm13 cells that were cultured with the indicated concentrations of isoniazid.

FIG. 11G shows the levels of the PLP-isoniazid complex in Molm13 cells that were cultured with the indicated concentrations of isoniazid. Cells were processed with or without PBS washing to remove residual isoniazid in cell culture before HPLC-MS analysis. Abundance levels of PLP-isoniazid complex were measured by HPLC-MS and normalized to the level in the presence of 1.6 mM isoniazid without PBS washing. The average and STDEV are shown.

FIG. 11H shows the levels of the [³D]-labeled PLP-isoniazid complex in Molm13 cells that were cultured with the indicated concentrations of isoniazid. Cells were processed with or without PBS washing to remove residual isoniazid in cell culture before HPLC-MS analysis. Abundance levels of [³D]-labeled PLP-isoniazid complex (internal control) were measured by HPLC-MS and normalized to the level in the presence of 1.6 mM isoniazid without PBS washing. The average and STDEV are shown.

FIG. 11I shows the levels of the pyridoxal in Molm13 cells that were cultured with the indicated concentrations of isoniazid. Cells were processed with or without PBS washing to remove residual isoniazid in cell culture before HPLC-MS analysis. Abundance levels of pyridoxal were measured by HPLC-MS and normalized to the level in the absence of isoniazid with PBS washing. The average and STDEV are shown.

FIG. 11J shows the levels of the [³D]-pyridoxal in Molm13 cells that were cultured with the indicated concentrations of isoniazid. Cells were processed with or without PBS washing to remove residual isoniazid in cell culture before HPLC-MS analysis. Abundance levels of [³D]-pyridoxal (internal control) were measured by HPLC-MS and normalized to the level in the absence of isoniazid with PBS washing. The average and STDEV are shown.

FIG. 11K shows the levels of the pyridoxine in Molm13 cells that were cultured with the indicated concentrations of isoniazid. Cells were processed with or without PBS washing to remove residual isoniazid in cell culture before HPLC-MS analysis. Abundance levels of pyridoxine were measured by HPLC-MS and normalized to the level in the absence of isoniazid with PBS washing. The average and STDEV are shown.

FIG. 11L shows the levels of the [³D]-pyridoxine in Molm13 cells that were cultured with the indicated concentrations of isoniazid. Cells were processed with or without PBS washing to remove residual isoniazid in cell culture before HPLC-MS analysis. Abundance levels of [³D]-pyridoxine (internal control) were measured by HPLC-MS and normalized to the level in the absence of isoniazid with PBS washing. The average and STDEV are shown.

FIG. 12A shows the percentage of GFP⁺ sgRNA infected cells in the culture system that were quantified at day 4, day 10, day 16, day 22, and day 30 of cell culture. Human Molm13 leukemic cells were infected with viruses encoding mouse or human PDXK, and the hygromycin resistance gene. After hygromycin selection, leukemic cells were infected with viruses encoding the indicated sgRNAs targeting Rosa26, Cr8, or human PDXK. sgRNAs a15 and a22 target exon regions of PDXK, and sgRNA a105 targets intron-exon junction of PDXK. The average and STDEV of relative GFP⁺ percentages are shown.

FIG. 12B shows the expression levels of PDXK protein by western blot analysis in human Molm13 leukemic cells that were infected with viruses encoding mouse or human PDXK, and the hygromycon resistance gene. After hygromycin selection, leukemic cells were infected with viruses encoding the indicated sgRNAs targeting Rosa26, Cr8, or human PDXK.

FIG. 12C shows the percentage of GFP⁺ sgRNA infected cells in the culture system that were quantified at day 4, day 10, day 16, day 22, and day 30 of cell culture. Human Molm13 leukemic cells were infected with viruses encoding mouse wild type PDXK or human PDXK with an a232-a235 deletion, and the hygromycin resistance gene. After hygromycin selection, leukemic cells were infected with viruses encoding the indicated sgRNAs targeting Rosa26 or human PDXK. The average and STDEV of relative GFP⁺ percentages are shown.

FIG. 12D shows the expression levels of PDXK protein by western blot analysis in human Molm13 leukemic cells that were infected with viruses encoding mouse wild type PDXK or human PDXK with an a232-a235 deletion, and the hygromycin resistance gene. After hygromycin selection, leukemic cells were infected with viruses encoding the indicated sgRNAs targeting Rosa26 or human PDXK.

FIG. 12E shows the levels of PLP in human Molm13 leukemic cells that were infected with viruses encoding the indicated sgRNAs. Molm13 leukemic cells were infected with viruses encoding mouse wild type PDXK or human PDXK with an a232-a235 deletion, and the hygromycin resistance gene. After hygromycin selection, Molm13 leukemic cells were infected with viruses encoding the indicated sgRNAs targeting Rosa26 or human PDXK. The abundance of PLP was measured by high-performance liquid chromatography-mass spectrometry (HPLC-MS) and normalized to the level of Rosa26 sgRNA group (negative control). The average and STDEV are shown.

FIG. 13A shows the percentages of GFP⁺ bone marrow cells observed in sub-lethally irradiated mice that were transplanted with Nras(G12D)/MLL-AF9 cells infected with viruses encoding shRNAs targeting Renilla luciferase or PDXK. Doxycycline (Dox) was supplemented in the mouse diet 5 days after transplantation.

FIG. 13B shows shows the levels of PLP in plasma and barrow marrow cells isolated from sub-lethally irradiated mice that were transplanted with Nras(G12D)/MLL-AF9 cells infected with viruses encoding shRNAs targeting Renilla luciferase or PDXK. A daily dose of control PBS or isoniazid (90 mg/kg) was intraperitoneally injected into the animals and PLP levels were detected at day 12 post transplant. The abundance of PLP was measured by HPLC-MS and normalized to the level of the PBS injection group. The average and STDEV are shown. “*” represents p<0.05 oft-test. and “**” represents p<0.01 oft-test.

FIG. 14A shows the abundance ratio of the indicated metabolites isolated on day 12 of cell culture from Molm13 leukemic cells with or without depletion of PDXK. The abundance of the indicated metabolites was measured by HPLC-MS. “NS” represents no statistical significant difference, “*” represents p<0.05, “**” represents p<0.01, “***” represents p<0.005 oft-test.

FIG. 14B shows the abundance ratio of the indicated metabolites isolated on day 5 of cell culture from Nras(G12D)/MLL-AF9 leukemic cells with or without depletion of PDXK. The abundance of the indicated metabolites was measured by HPLC-MS. “NS” represents no statistical significant difference, “*” represents p<0.05, “**” represents p<0.01, “***” represents p<0.005 oft-test.

FIG. 14C shows the abundance ratio of the indicated metabolites isolated on day 5 of cell culture from Nras(G12D)/MLL-AF9 leukemic cells with or without depletion of PDXK. The abundance of the indicated metabolites was measured by HPLC-MS. “NS” represents no statistical significant difference, “*” represents p<0.05, “**” represents p<0.01, “***” represents p<0.005 oft-test.

FIG. 15A shows the percentage of GFP⁺ sgRNA infected cells in the culture system that were quantified at day 2, day 6, day 10, and day 14 of cell culture. Nras(G12D)/MLL-AF9 leukemic cells were infected with viruses encoding the indicated sgRNAs. The average and STDEV of the relative GFP⁺ percentage are shown.

FIG. 15B shows the percentage of GFP⁺ sgRNA infected cells in the culture system that were quantified at day 2, day 6, day 10, and day 14 of cell culture. iMEF cells were infected with viruses encoding the indicated sgRNAs. The average and STDEV of the relative GFP⁺ percentage are shown.

FIG. 15C shows the percentage of GFP⁺ sgRNA infected cells in the culture system that were quantified at day 2, day 6, day 10, and day 14 of cell culture. Nras(G12D)/MLL-AF9 leukemic cells were infected with viruses encoding the indicated sgRNAs. The average and STDEV of the relative GFP⁺ percentage are shown.

FIG. 15D shows the percentage of GFP⁺ sgRNA infected cells in the culture system that were quantified at day 2, day 8, day 14, day 20, and day 26 of cell culture. Molm13 leukemic cells were infected with viruses encoding Rosa26 or GOT2 sgRNAs. The average and STDEV of the relative GFP⁺ percentage are shown.

FIG. 15E shows the percentage of GFP⁺ sgRNA infected cells in the culture system that were quantified at day 2, day 8, day 14, day 20, and day 26 of cell culture. Molm13 leukemic cells were infected with viruses encoding Rosa26 or AZIN1 sgRNAs. The average and STDEV of the relative GFP⁺ percentage are shown.

FIG. 15F shows the percentage of GFP⁺ sgRNA infected cells in the culture system that were quantified at day 2, day 8, day 14, day 20, and day 26 of cell culture. Molm13 leukemic cells were infected with viruses encoding Rosa26 or ODC1 sgRNAs. The average and STDEV of the relative GFP⁺ percentage are shown.

FIG. 16A shows a schematic diagram illustrating the role of pyridoxine-5′-phosphate oxidase (PNPO) in producing PLP using other phosphorylated vitamin B6 species (PNP and PMP).

FIG. 16B shows that when cultured with regular medium containing PN and PL, human Molm13 leukemic cells are partially sensitive to PNPO knockout, while PDXK knockout Molm13 cells exhibited full suppression of tumor growth.

FIG. 16C shows the levels of PLP in Molm13 leukemic cells that were infected with viruses encoding the indicated sgRNAs. The abundance of PLP was measured by HPLC-MS. The average and STDEV are shown. Western blot analysis shows the expression levels of PDXK protein.

FIG. 16D shows that when Molm13 cells are cultured with medium containing only PN (no PM and trace amount of PL), the cells are more sensitive to PNPO knockout as they now depend more on the PNPO pathway to make intracellular PLP.

FIG. 16E shows that when Molm13 cells are cultured with medium containing excess PL, the cellular growth is not dependent on PNPL anymore.

FIG. 17A shows the chemical structure of ginkgotoxin.

FIG. 17B demonstrates that ginkgotoxin can inhibit PDXK and reduce intracellular levels of PLP in human Molm13 leukemic cells.

FIG. 17C shows that ginkgotoxin can suppress the growth of the five indicated leukemic cell lines.

FIG. 17D shows luciferase levels observed in sub-lethally irradiated mice that were transplanted with Nras(G12D)/MLL-AF9 cells infected with viruses encoding shRNA targeting Renilla luciferase. Luciferase signals were monitored at day 11 and day 14 post transplant.

FIG. 17E shows the quantification of luciferase intensities from FIG. 17D.

FIG. 17F shows the survival curves of the animals shown in FIG. 17D.

FIG. 18A shows the chemical structure of aftin-4.

FIG. 18B demonstrates that aftin-4 can nhibit PDXK and reduce intracellular levels of PLP in human Molm13 leukemic cells.

FIG. 18C shows that aftin-4 can suppress the growth of the six indicated leukemic cell lines.

FIG. 19A shows a schematic diagram of significant changes in metabolite signature upon PDXK suppression in mouse AML cells. A decrease in mucleotide precursors was observed for uridine-5′-diphosphoglucuronic acid (UDP), adenine, orotic acid, and N-carcarmoyl-DL-aspartic acid.

FIG. 19B shows a schematic diagram of significant changes in metabolite signature upon PDXK knockout in mouse embryonic fibroblast cells. In contrast, the embryonic fibroblasts were more insensitive and did not show significant changes in nucleotide precursors.

FIG. 19C shows that upon PDXK knockout, human AML cells exhibit a decreased level of metabolites involved in nucleotide synthesis, including TMP, 2′-deoxycytidine, AMP, ADP, GDP, and uridine.

FIG. 20A shows the correlation between metabolite abundance ratios of Pdxk.307/Ren.713 and Pdxk.3259/Ren.713 in leukemic cells. Nras(G12D)/MLL-AF9 leukemic cells were infected with viruses encoding the indicated shRNAs. 5 days after doxycycline induction, leukemic cells were collected for HPLC-MS analysis.

FIG. 20B shows the abundance ratios of the various indicated metabolites in leukemic cells. Nras(G12D)/MLL-AF9 leukemic cells were either infected with viruses encoding the indicated shRNAs or cultured with the indicated compounds. 5 days after doxycycline induction or culture with compounds, leukemic cells were collected for HPLC-MS analysis.

FIG. 20C shows the percentage of GFP⁺ Nras(G12D)/MLL-AF9 leukemic cells that were infected with viruses encoding the indicated GOT2 sgRNAs at day 2 through day 14 of cell culture. The average and STDEV of the relative GFP⁺ percentage are shown.

FIG. 20D shows the expression levels of pyridoxal phosphate dependent transaminase GOT2 protein in Nras(G12D)/MLL-AF9 leukemic cells that were infected with viruses encoding the indicated sgRNAs at day 3 of cell culture.

FIG. 21A shows the expression levels of PDXK protein in human Molm13 (MLL-AF9/FLT3-ITD) leukemic cells that were infected with viruses encoding the indicated sgRNAs at day 3 of cell culture.

FIG. 21B shows the GFP⁺ percentages of human Molm13 (MLL-AF9/FLT3-ITD) leukemic cells that were infected with viruses encoding the indicated sgRNAs. GFP⁺ percentages were monitored over day 2 through day 30 of cell culture. Average and STDEV are shown.

FIG. 21C shows the GFP⁺ percentages of human Thp1 (MLL-AF9/Nras) leukemic cells that were infected with viruses encoding the indicated sgRNAs. GFP⁺ percentages were monitored over day 2 through day 30 of cell culture. Average and STDEV are shown.

FIG. 21D shows the GFP⁺ percentages of human Mv4-11 (MLL-AF4) leukemic cells that were infected with viruses encoding the indicated sgRNAs. GFP⁺ percentages were monitored over day 2 through day 30 of cell culture. Average and STDEV are shown.

FIG. 21E shows the GFP⁺ percentages of human Ml-2 (MLL-AF6/Kras) leukemic cells that were infected with viruses encoding the indicated sgRNAs. GFP⁺ percentages were monitored over day 2 through day 30 of cell culture. Average and STDEV are shown.

FIG. 21F shows the GFP⁺ percentages of human K562 leukemic cells that were infected with viruses encoding the indicated sgRNAs. GFP⁺ percentages were monitored over day 2 through day 30 of cell culture. Average and STDEV are shown.

FIG. 22A shows the GFP⁺ percentages of human Molm13 (MLL-AF9/FLT3-ITD) leukemic cells that were infected with viruses encoding the indicated sgRNAs targeting Rosa26, Cr8, or human PDXK together with viruses encoding MSCV vector or mouse PDXK cDNA or human PDXK cDNA. GFP⁺ percentages were monitored during culture. Error bar represents STDEV of 4 repeats.

FIG. 22B shows the expression levels of PDXK protein in human Molm13 (MLL-AF9/FLT3-ITD) leukemic cells that were infected with viruses encoding the indicated sgRNAs targeting Rosa26, Cr8, or human PDXK together with viruses encoding MSCV vector or mouse PDXK cDNA or human PDXK cDNA at day 3 of cell culture.

FIG. 23 shows that PDXK is required for the proliferation of hematopoietic malignant cells. Genome-wide cancer dependency results were downloaded as previously described in E. R. McDonald et al., Cell 170:577-592.e510 (2017). The average and STDEV of sensitivity score corresponding to each cancer type were calculated by combining sensitivity scores of multiple cell lines that belong to the same cancer type. The average and STDEV of sensitivity score corresponding to each cancer type are shown. Sensitivity scores corresponding to AML, acute lymphoblastic leukemia (ALL), and lymphoma are labeled with red color.

FIG. 24A shows the dependency of indicated cell lines on the listed genes that were downloaded from T. Wang et al., Cell 168:890-903.e815 (2017).

FIG. 24B shows the expression levels of PDXK protein in RagMEF cells that were infected with viruses encoding the indicated sgRNAs at day 3 of culture.

FIG. 24C shows the GFP⁺ percentages of RagMEF cells that were infected with viruses encoding the indicated sgRNAs. The percentage of GFP⁺ infected cells in the culture system were quantified from day 2 to day 24. The average and standard deviation (STDEV) of the relative GFP⁺ percentage are shown.

FIG. 24D shows the GFP⁺ percentages of 3T3 cells that were infected with viruses encoding the indicated sgRNAs. The percentage of GFP⁺ infected cells in the culture system were quantified from day 2 to day 24. The average and standard deviation (STDEV) of the relative GFP⁺ percentage are shown.

FIG. 25A shows the relative cell proliferation of human leukemic cells that were cultured with the indicated concentrations of Aftin-4 at day 6 of cell culture. The average and STDEV of relative cell proliferation are shown.

FIG. 25B shows the relative cell proliferation of human leukemic cells that were cultured with the indicated concentrations of gingkotoxin (Gkt) at day 6 of cell culture. The average and STDEV of relative cell proliferation are shown.

FIG. 25C shows the relative cell proliferation of human leukemic cells that were cultured with the indicated concentrations of isoniazid at day 6 of cell culture. The average and STDEV of relative cell proliferation are shown.

FIG. 26A shows the relative cell proliferation of Nras(G12D)/MLL-AF9 leukemic cells or bone marrow lineage negative HSPCs that were cultured with the indicated concentrations of gingkotoxin at day 6 of cell culture. The average and STDEV of relative cell proliferation of 3 replicates are shown.

FIG. 26B shows the relative cell proliferation of Nras(G12D)/MLL-AF9 leukemic cells or bone marrow lineage negative HSPCs that were cultured with the indicated concentrations of isoniazid at day 6 of cell culture. The average and STDEV of relative cell proliferation of 3 replicates are shown.

FIG. 27A shows Nras(G12D)/MLL-AF9 leukemic cells that were infected with viruses encoding the indicated sgRNAs. GFP⁺ and GFP⁻ cells were gated for cell cycle analysis.

FIG. 27B shows the percentage of S-phase leukemic cells that were infected with viruses encoding the indicated sgRNAs. GFP⁺ and GFP⁻ cells were gated for cell cycle analysis. The average and STDEV of 3 replicates are shown.

FIG. 28A shows Nras(G12D)/MLL-AF9 leukemic cells that were infected with viruses encoding the indicated sgRNAs. GFP⁺ and GFP⁻ cells were gated for Annexin V and 7-AAD apoptosis analysis.

FIG. 28B shows the percentage of apoptotic cells that were infected with viruses encoding the indicated sgRNAs. GFP⁺ and GFP⁻ cells were gated for Annexin V and 7-AAD apoptosis analysis. The average and STDEV of 3 replicates are shown.

FIG. 29A shows the redox status of Nras(G12D)/MLL-AF9 leukemic cells that were treated with tert-butyl hydroperoxide.

FIG. 29B shows Nras(G12D)/MLL-AF9 leukemic cells that were infected with viruses encoding the indicated sgRNAs. GFP⁺ cells were gated for redox status analysis.

FIG. 30A shows Nras(G12D)/MLL-AF9 leukemic cells that were cultured in the absence or presence of 1 μg/ml vitamin B6 pyridoxine. Flow cytometry analysis was performed to monitor cell cycle progression.

FIG. 30B shows the percentage of S-phase leukemic cells that were cultured in the absence or presence of 1 μg/ml vitamin B6 pyridoxine. Flow cytometry analysis was performed to monitor cell cycle progression. The average and STDEV of six replicates are shown.

FIG. 31A shows the correlation between metabolite abundance ratios of Pdxk.307/Ren.713 and Pdxk.3259/Ren.713 in leukemic cells. Nras(G12D)/MLL-AF9 leukemic cells were either infected with viruses encoding the indicated shRNAs or cultured with the indicated compounds. 5 days after doxycycline induction or culture with compounds, leukemic cells were collected for HPLC-MS analysis.

FIG. 31B shows the correlation between metabolite abundance ratios of Isoniazid/DMSO and Pdxk.307/Ren.713 in leukemic cells.

FIG. 31C shows the correlation between metabolite abundance ratios of Isoniazid/DMSO and Pdxk.3259/Ren.713 in leukemic cells.

FIG. 32A shows the relative GFP⁺ percentages of Molm13 leukemic cells that were infected with the indicated sgRNAs. GFP⁺ percentages were monitored during culture. Error bar represents STDEV.

FIG. 32B shows the relative GFP⁺ percentages of Thp1 leukemic cells that were infected with the indicated sgRNAs. GFP⁺ percentages were monitored during culture. Error bar represents STDEV.

FIG. 33A shows the percentage of Nras(G12D)/MLL-AF9 leukemic cells that were cultured with the various indicated concentrations of myriocin at day 3 of cell culture.

FIG. 33B shows the percentage of human HL60 cells that were cultured with the various indicated concentrations of myriocin at day 4 of cell culture.

FIG. 34A shows the percentage of viable Nras(G12D)/MLL-AF9 leukemic cells that were cultured with the various indicated concentrations of AOA at day 3 of cell culture.

FIG. 34B shows the percentage of viable human Molm13 leukemic cells that were cultured with the various indicated concentrations of AOA at day 3 of cell culture.

FIG. 34C shows the percentage of viable human Thp1 leukemic cells that were cultured with the various indicated concentrations of AOA at day 3 of cell culture.

FIG. 34D shows the percentage of viable human m12 leukemic cells that were cultured with the various indicated concentrations of AOA at day 3 of cell culture.

FIG. 34E shows the percentage of viable human Nomol leukemic cells that were cultured with the various indicated concentrations of AOA at day 3 of cell culture.

FIG. 34F shows the percentage of viable human Semk2 leukemic cells that were cultured with the various indicated concentrations of AOA at day 3 of cell culture.

FIG. 34G shows the percentage of viable human Kasumi leukemic cells that were cultured with the various indicated concentrations of AOA at day 3 of cell culture.

FIG. 34H shows quantification of luciferase intensities observed in sub-lethally irradiated mice that were transplanted with Nras(G12D)/MLL-AF9 cells. A daily dose of control PBS or AOA was administered to the animals and luciferase signals were detected at day 4, and day 10 post transplant. “NS” represents no statistical significance oft-test, and “*” represents p<0.05 oft-test.

FIG. 34I shows the survival curves of the animals shown in FIG. 25H. “*” represents p<0.05 oft-test.

FIG. 35 shows a list of human metabolic genes that are enriched in AML cells. Gene expression profiling performed on leukemic cells and CD34⁺ HSPCs were downloaded from P. J. Valk et al., N Engl J Med 350:1617-1628 (2004).

FIG. 36 shows a list of mouse metabolic genes that are enriched in AML cells. Gene expression profiling performed on leukemic cells and CD34⁺ HSPCs were downloaded from P. J. Valk et al., N Engl J Med 350:1617-1628 (2004).

FIG. 37 shows that the CRISPR/Cas functional genomic screening disclosed herein identified metabolic vulnerabilities in AML. Nras(G12D)/MLL-AF9 leukemic cells were infected with viral CRISPR/Cas sgRNA pool. Genomic DNA was extracted in from day 1 and day 9 cultured cells. sgRNA inserts were PCR amplified for deep sequencing. sgRNA inserts (SEQ ID NOs: 43-981) and Log 2 reads ratios corresponding to each sgRNA (day 9/day 1) are listed.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-S.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of AML. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “individual”, “patient”, or “subject” can be an individual organism, a vertebrate, a mammal, or a human. In some embodiments, the individual, patient or subject is a human.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample. As used herein, preventing AML, includes preventing or delaying the initiation of symptoms of AML. As used herein, prevention of AML also includes preventing a recurrence of one or more signs or symptoms of AML.

As used herein, the term “sample” means biological sample material derived from living cells of a subject. Biological samples may include tissues, cells, protein or membrane extracts of cells, and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof (e.g, ameliorating or treating AML).

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Inhibitors of Vitamin B6 Pathway

In one aspect, the present disclosure provides inhibitory RNAs (e.g., sgRNAs, antisense RNAs or shRNAs) that inhibit the vitamin B6 pathway by targeting at least one gene selected from the group consisting of PDXK, PNPO, AZIN1, ODC1, GOT2, ALAS1, SPTLC1, and SPTLC2. The mammalian nucleic acid sequences of PDXK, PNPO, AZIN1, ODC1, GOT2, ALAS1, SPTLC1, and SPTLC2 are known in the art (e.g., NCBI Gene IDs: 8566, 55163, 51582, 4953, 2806, 211, 10558, and 9517). The inhibitory nucleic acids of the present technology may comprise a nucleic acid molecule which is complementary to a portion of a PDXK, PNPO, AZIN1, ODC1, GOT2, ALAS1, SPTLC1, and SPTLC2 nucleic acid sequence. In some embodiments, the inhibitory RNAs (e.g., sgRNAs, antisense RNAs or shRNAs) target at least one exon and/or intron of PDXK, PNPO, AZIN1, ODC1, GOT2, ALAS1, SPTLC1, or SPTLC2.

The present disclosure also provides an antisense nucleic acid comprising a nucleic acid sequence that is complementary to and specifically hybridizes with a portion of a PDXK, PNPO, AZIN1, ODC1, GOT2, ALAS1, SPTLC1, or SPTLC2 mRNA. The antisense nucleic acid may be antisense RNA, or antisense DNA. Antisense nucleic acids based on the known nucleic acid sequences of PDXK, PNPO, AZIN1, ODC1, GOT2, ALAS1, SPTLC1, or SPTLC2 can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire coding strand of PDXK, PNPO, AZIN1, ODC1, GOT2, ALAS1, SPTLC1, or SPTLC2, or to a portion thereof, e.g., all or part of the protein coding region (or open reading frame). In some embodiments, the antisense nucleic acid is an oligonucleotide which is complementary to only a portion of the coding region of PDXK, PNPO, AZIN1, ODC1, GOT2, ALAS1, SPTLC1, or SPTLC2 mRNA. In certain embodiments, an antisense nucleic acid molecule can be complementary to a noncoding region of the PDXK, PNPO, AZIN1, ODC1, GOT2, ALAS1, SPTLC1, or SPTLC2 coding strand. In some embodiments, the noncoding region refers to the 5′ and 3′ untranslated regions that flank the coding region and are not translated into amino acids. For example, the antisense oligonucleotide can be complementary to the region surrounding the translation start site of PDXK, PNPO, AZIN1, ODC1, GOT2, ALAS1, SPTLC1, or SPTLC2. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-metnylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual 13-units, the strands run parallel to each other (Gaultier et al., Nucleic Acids. Res. 15:6625-6641(1987)). The antisense nucleic acid molecule can also comprise a 2′-O-methylribonucleotide (Inoue et al., Nucleic Acids Res. 15:6131-6148 (1987)) or a chimeric RNA-DNA analogue (Inoue et al., FEBS Lett 215:327-330 (1987)).

The present disclosure also provides a short hairpin RNA (shRNA) or small interfering RNA (siRNA) comprising a nucleic acid sequence that is complementary to and specifically hybridizes with a portion of a PDXK, PNPO, AZIN1, ODC1, GOT2, ALAS1, SPTLC1, or SPTLC2 mRNA, thereby reducing or inhibiting gene expression. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hydridize to a mRNA of a target gene. Following intracellular delivery, the siRNA or shRNA molecule associates with an RNA-induced silencing complex (RISC), which then binds and degrades a complementary target mRNA (such as PDXK, PNPO, AZIN1, ODC1, GOT2, ALAS1, SPTLC1, or SPTLC2 mRNA).

The present disclosure also provides a synthetic guide RNA (sgRNA) comprising a nucleic acid sequence that is complementary to and specifically hybridizes with a portion of a PDXK, PNPO, AZIN1, ODC1, GOT2, ALAS1, SPTLC1, or SPTLC2 nucleic acid sequence. Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of a PDXK, PNPO, AZIN1, ODC1, GOT2, ALAS1, SPTLC1, or SPTLC2 nucleic acid sequence, and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465.

In some embodiments, a synthetic guide RNA is a single RNA represented as comprising the following elements: 5′-X1-X2-Y-Z-3′

where X1 and X2 represent the crRNA segment, where X1 is the targeting sequence that binds to a portion of a PDXK, PNPO, AZIN1, ODC1, GOT2, ALAS1, SPTLC1, or SPTLC2 nucleic acid sequence, X2 is a stem sequence the hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of a PDXK, PNPO, AZIN1, ODC1, GOT2, ALAS1, SPTLC1, or SPTLC2 nucleic acid sequence, X2 is a stem sequence the hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator:

(a)
(SEQ ID NO: 991)
NNNNNNNNNNNNNNNNNNNNgtttttgtactctcaagatttaGAAAtaaa
tcttgcagaagctacaaagataaggcttcatgccgaaatcaacaccctgt
cattttatggcagggtgttttcgttatttaaTTTTTT;
(b)
(SEQ ID NO: 992)
NNNNNNNNNNNNNNNNNNNNgtttttgtactctcaGAAAtgcagaagcta
caaagataaggcttcatgccgaaatcaacaccctgtcattttatggcagg
gtgttttcgttatttaaTTTTTT;
(c)
(SEQ ID NO: 993)
NNNNNNNNNNNNNNNNNNNNgtttttgtactctcaGAAAtgcagaagcta
caaagataaggcttcatgccgaaatcaacaccctgtcattttatggcagg
gtgtTTTTTT;
(d)
(SEQ ID NO: 994)
NNNNNNNNNNNNNNNNNNNNgttttagagctaGAAAtagcaagttaaaat
aaggctagtccgttatcaacttgaaaaagtggcaccgagtcggtgcTTTT
TT;
(e)
(SEQ ID NO: 995)
NNNNNNNNNNNNNNNNNNNNgttttagagctaGAAATAGcaagttaaaat
aaggctagtccgttatcaacttgaaaaagtgTTTTTTT;
and
(f)
(SEQ ID NO: 996)
NNNNNNNNNNNNNNNNNNNNgttttagagctagAAATAGcaagttaaaat
aaggctagtccgttatcaTTTTTTTT.

Selection of suitable oligonucleotides for use in as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

Examples of suitable inhibitory RNAs (e.g., sgRNAs, antisense RNAs or shRNAs) include those with sequences comprising 5′ TGGCTACGTGGGTAACAGAG 3′ (Pdxk sg-1) (SEQ ID NO: 1), 5′ ATCCAGAGCCATGTTGTCCG 3′ (Pdxk sg-2) (SEQ ID NO: 2), 5′ GTGCAGTTTTCAAACCACAC 3′ (Pdxk sg-3) (SEQ ID NO: 3), 5′ GCTTGGGGTGCCTGCAGAGA 3′ (Odc1-a106) (SEQ ID NO: 4), 5′ TGCTGTTGACAGTGAGCGCCAAGGTGAACGATGTCAATAATAGTGAAGCCACAG ATGTATTATTGACATCGTTCACCTTGATGCCTACTGCCTCGGA 3′ (Pdxk.307) (SEQ ID NO: 5), 5′ TGCTGTTGACAGTGAGCGCCAGGTTCAATGTGAGGTTACATAGTGAAGCCACAG ATGTATGTAACCTCACATTGAACCTGATGCCTACTGCCTCGGA 3′ (Pdxk.3259) (SEQ ID NO: 6), 5′ ACGCCCAGGATGGGATCTGG 3′ (Got2.a41) (SEQ ID NO: 7), 5′ AAAGAATACCTGCCCATTGG 3′ (Got2.a99) (SEQ ID NO: 8), 5′ GACTGGAGCCTTAAGGGTCG 3′ (Got2.a140) (SEQ ID NO: 9), 5′ ATACAGAGCCACGTCATCCG 3′ (hPdxk-aa15) (SEQ ID NO: 10), 5′ CGGCTACGTGGGCAACCGGG 3′ (hPdxk-aa22) (SEQ ID NO: 11), 5′ GCCTACCGTACACCAGCCTG 3′ (hPdxk-aa105) (SEQ ID NO: 12), 5′ GTCCCCAGTGCCCACAAAGA 3′ (hPDXK-aa230) (SEQ ID NO: 13), 5′ AATGGCTTTAGTGCAAGAAT 3′ (Azin1-a100) (SEQ ID NO: 14), 5′ GAACTACTCCGTTGGCCTGT 3′ (Azin1-a14) (SEQ ID NO: 15), 5′ GCCAAGATCTCAAGCACGGC 3′ (Azin1-a76) (SEQ ID NO: 16), 5′ ATATTGACGTCATTGGTGTG 3′ (Odc1-a194) (SEQ ID NO: 17), 5′ AGGCAGCAGCGTCTTCCGCA 3′ (ALAS1 sg-1) (SEQ ID NO: 18), 5′ CACCGTTTTAAAAACTCGGT 3′(ALAS1 sg-2) (SEQ ID NO: 19), 5′ CTCGGGATAAGAATGGGCAT 3′ (ALAS1 sg-3) (SEQ ID NO: 20), 5′ TGCGTAAAAGGGAGTGACGC 3′ (Odc1-a62) (SEQ ID NO: 21), 5′ GCTGGCCAACCCTCGAGTTA 3′ (SPTLC1 sg-1) (SEQ ID NO: 22), 5′ GATGGTGCAGGCGCTGTACG 3′ (SPTLC1 sg-2) (SEQ ID NO: 23), 5′ TCAACTACAACATCGTGTCC 3′ (SPTLC1 sg-3) (SEQ ID NO: 24), 5′ GCTCCAGGCACACTACAGAT 3′ (SPTLC2 sg-1) (SEQ ID NO: 25), 5′ GAACGGCTGCGTCAAGAACG 3′ (SPTLC2 sg-2) (SEQ ID NO: 26), 5′ AATCTCGAAGATATCCAAAG 3′ (SPTLC2 sg-3) (SEQ ID NO: 27), 5′ GGTGTGTGGTTTCCCCAGGT 3′ (hGOT2.a162) (SEQ ID NO: 28), 5′ GATGGGTGTGTGGTTTCCCC 3′ (hGOT2.a163) (SEQ ID NO: 29), 5′ GGACGCGGGTCCACTCCCGT 3′(hGOT2.a218) (SEQ ID NO: 30), 5′ TGGACCCGCGTCCGGAACAG 3′ (hGOT2.a224) (SEQ ID NO: 31), 5′ ACGATGAACATGTTAGACAT 3′ (hAZIN1-a233) (SEQ ID NO: 39), 5′ CTATGTTTATGAACATACCC 3′ (hAZIN1-a33) (SEQ ID NO: 40), 5′ TATCTGCTTGATATTGGCGG 3′ (hODC1-a235) (SEQ ID NO: 41), 5′ CAACGCTGGGTTGATTACGC 3′ (hODC1-a254) (SEQ ID NO: 42), 5′ GGAGGTCCTGGGGAACGTAC 3′ (Pdxk sg-4) (SEQ ID NO: 982), 5′ CATGGCAGCGAAGAGGTCCC 3′ (Pdxk sg-5) (SEQ ID NO: 983), 5′ AGCTGTCTTCGTGGGCACCG 3′ (Pdxk sg-6) (SEQ ID NO: 984), 5′ TGTAACCTCACATTGAACCTGA 3′ (SEQ ID NO: 985), 5′ TTATTGACATCGTTCACCTTGA 3′ (SEQ ID NO: 986), 5′ CATGCGCAAGAGTTACCGCG 3′ (hPNPO-a42:) (SEQ ID NO: 987); 5′ ATGACCGGATAGTCTTTCGG 3′ (hPNPO-a232) (SEQ ID NO: 988); 5′ GAGTTACCGCGGGGACCGAG 3′ (hPNPO-a45) (SEQ ID NO: 989); and 5′ TTCTGTGATCCCTGATCGGG 3′ (hPNPO-a181) (SEQ ID NO: 990) or any complementary sequences thereof.

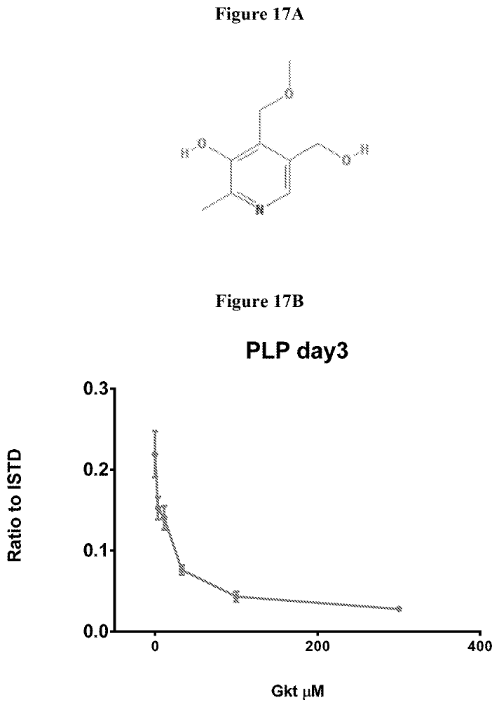

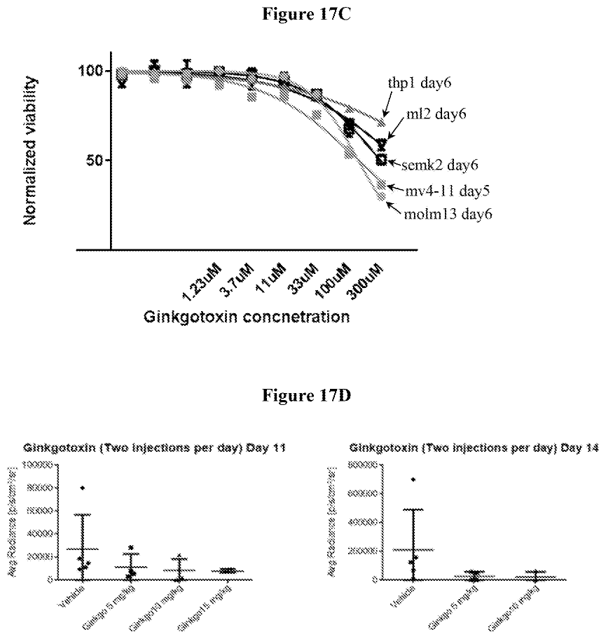

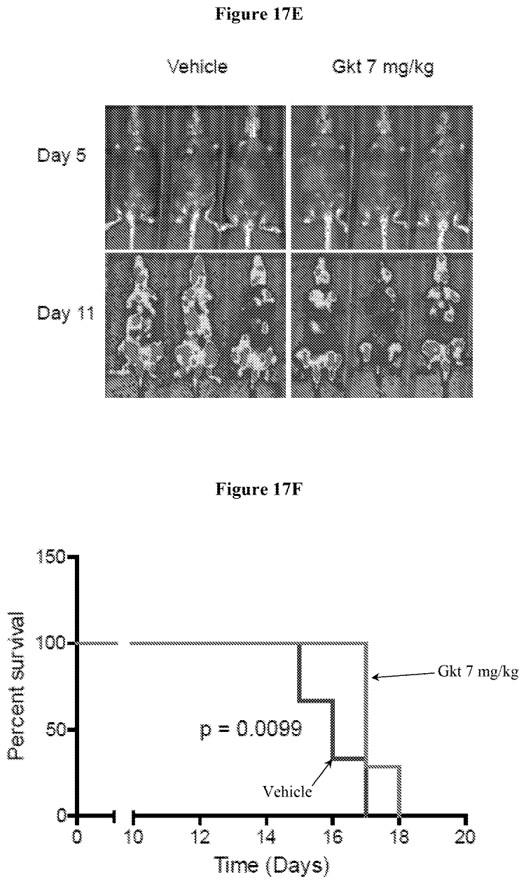

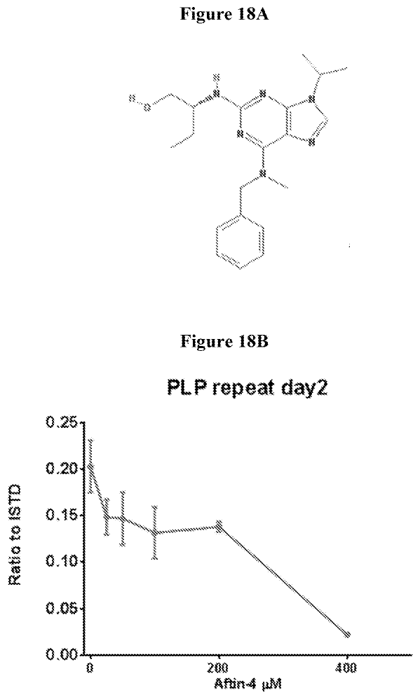

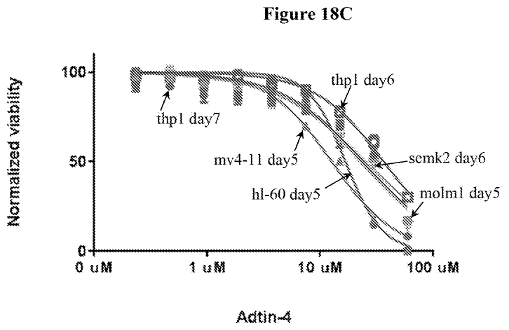

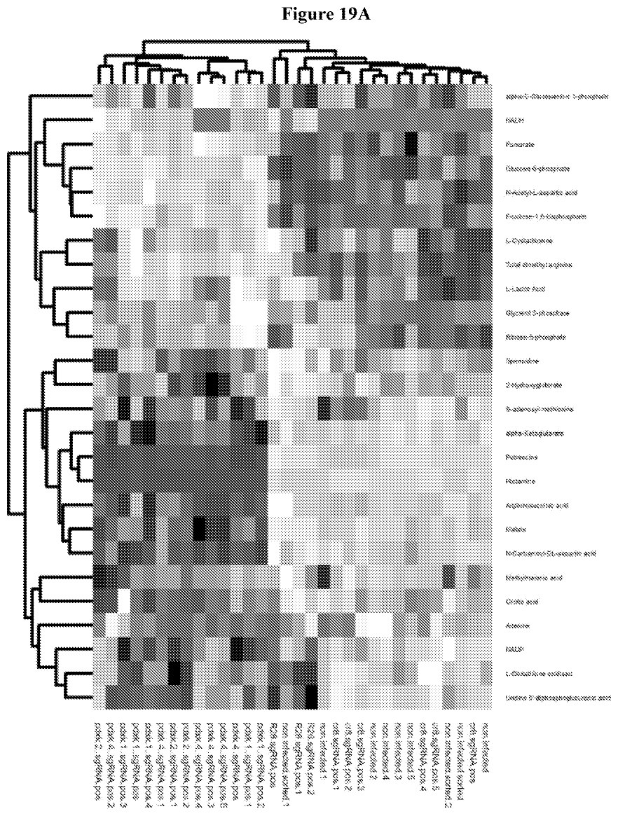

The present disclosure also provides pharmacological inhibitors of the vitamin B6 pathway including, but not limited to, isoniazid, aftin-4 (see FIG. 18A), gingkotoxin (see FIG. 17A), DFMO, aminooxyacetic acid (AOA), and myriocin.

### Therapeutic Methods

The following discussion is presented by way of example only, and is not intended to be limiting.

One aspect of the present technology includes methods of treating a disease or condition characterized by elevated expression levels and/or increased activity of PDXK. Additionally or alternatively, in some embodiments, the present technology includes methods of treating AML. In one aspect, the present disclosure provides a method for inhibiting leukemic cell proliferation in a subject in need thereof, comprising administering to the subject a therapeutically effective amount of at least one inhibitor of Vitamin B6 pathway, wherein the at least one inhibitor is selected from the group consisting of isoniazid, aftin-4, DFMO, gingkotoxin, aminooxyacetic acid, and myriocin, and wherein the subject suffers from a disease or condition characterized by elevated expression levels and/or increased activity of PDXK. In another aspect, the present disclosure provides a method for inhibiting leukemic cell proliferation in a subject in need thereof, comprising administering to the subject a therapeutically effective amount of at least one inhibitory RNA that targets one or more genes selected from the group consisting of PDXK, PNPO, AZIN1, ODC1, GOT2, ALAS1, SPTLC1, and SPTLC2, wherein the subject suffers from a disease or condition characterized by elevated expression levels and/or increased activity of PDXK

In some embodiments, the subject is diagnosed as having, suspected as having, or at risk of having a disease or condition characterized by elevated expression levels and/or increased activity of PDXK. Additionally or alternatively, in some embodiments, the subject is diagnosed as having AML.

In therapeutic applications, compositions or medicaments comprising a vitamin B6 pathway inhibitor disclosed herein are administered to a subject suspected of, or already suffering from such a disease or condition (such as, a subject diagnosed with a disease or condition characterized by elevated expression levels and/or increased activity of PDXK and/or a subject diagnosed with AML), in an amount sufficient to cure, or at least partially arrest, the symptoms of the disease, including its complications and intermediate pathological phenotypes in development of the disease.

Subjects suffering from a disease or condition characterized by elevated expression levels and/or increased activity of PDXK and/or a subject diagnosed with AML can be identified by any or a combination of diagnostic or prognostic assays known in the art. For example, typical symptoms of AML include, but are not limited to, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite.

In some embodiments, the subject may exhibit one or more point mutations in NRAS, DNMT3A, FLT3, KIT, IDH1, IDH2, CEBPA, and NPM1 and/or one or more chromosomal alterations (e.g., an inversion, translocation, or gene fusion) such as CBFB-MYH11, DEK-NUP214, MLL-MLLT3, PML-RARA, RBM15-MKL1, RPN1-EVI1 and RUNX1-RUNX1T1, and are detectable using techniques known in the art. See Naoe & Kiyoi, Int J Hematol. 97(2):165-74 (2013); Shih et al., Nat Rev Cancer. 12(9):599-612 (2012).

In some embodiments, subjects with a disease or condition characterized by elevated expression levels and/or increased activity of PDXK, and/or subjects suffering from AML that are treated with the vitamin B6 pathway inhibitor will show amelioration or elimination of one or more of the following symptoms: enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite.

In certain embodiments, subjects with a disease or condition characterized by elevated expression levels and/or increased activity of PDXK, and/or subjects suffering from AML that are treated with the vitamin B6 pathway inhibitor will show reduced leukemic cell proliferation and/or increased survival compared to untreated AML subjects. In certain embodiments, subjects with a disease or condition characterized by elevated expression levels and/or increased activity of PDXK, and/or subjects suffering from AML that are treated with the vitamin B6 pathway inhibitor will show reduced PDXK and PLP expression levels compared to untreated AML subjects.

In one aspect, the present disclosure provides a method for monitoring the therapeutic efficacy of a dosage of an inhibitor of Vitamin B6 pathway in a subject diagnosed with AML comprising: (a) detecting PDXK protein levels or intracellular PLP levels in a test sample obtained from the subject after the subject has been administered the dosage of the inhibitor of Vitamin B6 pathway, wherein the inhibitor of Vitamin B6 pathway is isoniazid, aftin-4, DFMO, gingkotoxin, aminooxyacetic acid, or myriocin; and (b) determining that the dosage of the inhibitor of Vitamin B6 pathway is effective when the PDXK protein levels or intracellular PLP levels in the test sample are reduced compared to that observed in a control sample obtained from the subject prior to administration of the inhibitor of Vitamin B6 pathway. In some embodiments, the intracellular PLP levels are detected via high-performance liquid chromatography-mass spectrometry. The test sample may be tissues, cells or biological fluids (blood, plasma, saliva, urine, serum etc.) present within a subject. Alternatively, PLP intracellular levels may be used to determine efficacy of the inhibitor of Vitamin B6 pathway in the subject. Accordingly, in certain embodiments, the method further comprises detecting intracellular levels of PLP in the subject, wherein a decrease in PLP intracellular levels relative to those observed in the subject prior to treatment is indicative of the therapeutic efficacy of the dosage of the inhibitor of Vitamin B6 pathway.

Also disclosed herein are methods for monitoring the therapeutic efficacy of an inhibitory RNA that targets a gene selected from the group consisting of PDXK, PNPO, AZIN1, ODC1, GOT2, ALAS1, SPTLC1, and SPTLC2 in a subject diagnosed with AML comprising: (a) detecting PDXK protein levels or intracellular PLP levels in a test sample obtained from the subject after the subject has been administered the inhibitory RNA; and (b) determining that the inhibitory RNA is effective when the PDXK protein levels or intracellular PLP levels in the test sample are reduced compared to that observed in a control sample obtained from the subject prior to administration of the inhibitory RNA. The inhibitory RNA may be a shRNA or a sgRNA. The test sample may be tissues, cells or biological fluids (blood, plasma, saliva, urine, serum etc.) present within a subject. In certain embodiments, the method further comprises detecting intracellular levels of PLP in the subject, wherein a decrease in PLP intracellular levels relative to those observed in the subject prior to treatment is indicative of the therapeutic efficacy of the inhibitory RNA. In some embodiments, the intracellular PLP levels are detected via high-performance liquid chromatography-mass spectrometry.

### Prophylactic Methods

In one aspect, the present technology provides a method for preventing or delaying the onset of a disease or condition characterized by elevated expression levels and/or increased activity of PDXK. Additionally or alternatively, in some aspects, the present technology provides a method for preventing or delaying the onset AML.

Subjects at risk or susceptible to a disease or condition characterized by elevated expression levels and/or increased activity of PDXK, and/or subjects at risk or susceptible to AML include those that exhibit one or more point mutations in NRAS, DNMT3A, FLT3, KIT, IDH1, IDH2, CEBPA, and NPM1 and/or one or more chromosomal alterations (e.g., an inversion, translocation, or gene fusion) such as CBFB-MYH11, DEK-NUP214, MLL-MLLT3, PML-RARA, RBM15-MKL1, RPN1-EVI1 and RUNX1-RUNX1T1. Such subjects can be identified by, e.g., any or a combination of diagnostic or prognostic assays known in the art.

In prophylactic applications, pharmaceutical compositions or medicaments comprising a vitamin B6 pathway inhibitor disclosed herein are administered to a subject susceptible to, or otherwise at risk of a disease or condition characterized by elevated expression levels and/or increased activity of PDXK, and/or a subject susceptible to, or otherwise at risk of AML, in an amount sufficient to eliminate or reduce the risk, or delay the onset of the disease, including biochemical, histologic and/or behavioral symptoms of the disease, its complications and intermediate pathological phenotypes presenting during development of the disease. Administration of a prophylactic vitamin B6 pathway inhibitor can occur prior to the manifestation of symptoms characteristic of the disease or disorder, such that the disease or disorder is prevented or, alternatively, delayed in its progression.

In some embodiments, treatment with the vitamin B6 pathway inhibitor will prevent or delay the onset of one or more of the following symptoms: leukemic cell proliferation, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite. In certain embodiments, (a) subjects with a disease or condition characterized by elevated expression levels and/or increased activity of PDXK, and/or (b) subjects with AML that are treated with the vitamin B6 pathway inhibitor will show PDXK and/or PLP expression levels that resemble those observed in healthy control subjects.

For therapeutic and/or prophylactic applications, a composition comprising a vitamin B6 pathway inhibitor disclosed herein, is administered to the subject. In some embodiments, the vitamin B6 pathway inhibitor is administered one, two, three, four, or five times per day. In some embodiments, the vitamin B6 pathway inhibitor is administered more than five times per day. Additionally or alternatively, in some embodiments, the vitamin B6 pathway inhibitor is administered every day, every other day, every third day, every fourth day, every fifth day, or every sixth day. In some embodiments, the vitamin B6 pathway inhibitor is administered weekly, bi-weekly, tri-weekly, or monthly. In some embodiments, the vitamin B6 pathway inhibitor is administered for a period of one, two, three, four, or five weeks. In some embodiments, the vitamin B6 pathway inhibitor is administered for six weeks or more. In some embodiments, the vitamin B6 pathway inhibitor is administered for twelve weeks or more. In some embodiments, the vitamin B6 pathway inhibitor is administered for a period of less than one year. In some embodiments, the vitamin B6 pathway inhibitor is administered for a period of more than one year. In some embodiments, the vitamin B6 pathway inhibitor is administered throughout the subject's life.

In some embodiments of the methods of the present technology, the vitamin B6 pathway inhibitor is administered daily for 1 week or more. In some embodiments of the methods of the present technology, the vitamin B6 pathway inhibitor is administered daily for 2 weeks or more. In some embodiments of the methods of the present technology, the vitamin B6 pathway inhibitor is administered daily for 3 weeks or more. In some embodiments of the methods of the present technology, the vitamin B6 pathway inhibitor is administered daily for 4 weeks or more. In some embodiments of the methods of the present technology, the vitamin B6 pathway inhibitor is administered daily for 6 weeks or more. In some embodiments of the methods of the present technology, the vitamin B6 pathway inhibitor is administered daily for 12 weeks or more. In some embodiments, the vitamin B6 pathway inhibitor is administered daily throughout the subject's life.

### Determination of the Biological Effect of Inhibitors of the Vitamin B6 Pathway

In various embodiments, suitable in vitro or in vivo assays are performed to determine the effect of a specific inhibitor of the vitamin B6 pathway and whether its administration is indicated for treatment. In various embodiments, in vitro assays can be performed with representative animal models, to determine if a given inhibitor of the vitamin B6 pathway exerts the desired effect on reducing or eliminating signs and/or symptoms of AML. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In some embodiments, in vitro or in vivo testing is directed to the biological function of one or more inhibitors of the vitamin B6 pathway.

Animal models of AML may be generated using techniques known in the art. Such models may be used to demonstrate the biological effect of inhibitors of the vitamin B6 pathway in the prevention and treatment of conditions arising from disruption of a particular gene, and for determining what comprises a therapeutically effective amount of the one or more inhibitors of the vitamin B6 pathway disclosed herein in a given context.

### Modes of Administration and Effective Dosages

Any method known to those in the art for contacting a cell, organ or tissue with one or more inhibitors of the vitamin B6 pathway disclosed herein may be employed. Suitable methods include in vitro, ex vivo, or in vivo methods. In vivo methods typically include the administration of one or more inhibitors of the vitamin B6 pathway to a mammal, suitably a human. When used in vivo for therapy, the one or more inhibitors of the vitamin B6 pathway described herein are administered to the subject in effective amounts (i.e., amounts that have desired therapeutic effect). The dose and dosage regimen will depend upon the degree of the disease state of the subject, the characteristics of the particular inhibitor of the vitamin B6 pathway used, e.g., its therapeutic index, and the subject's history.

The effective amount may be determined during pre-clinical trials and clinical trials by methods familiar to physicians and clinicians. An effective amount of one or more inhibitors of the vitamin B6 pathway useful in the methods may be administered to a mammal in need thereof by any of a number of well-known methods for administering pharmaceutical compounds. The inhibitors may be administered systemically or locally.

The one or more inhibitors of the vitamin B6 pathway described herein can be incorporated into pharmaceutical compositions for administration, singly or in combination, to a subject for the treatment or prevention of AML. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more inhibitors of the vitamin B6 pathway disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more inhibitors of the vitamin B6 pathway disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more inhibitors of the vitamin B6 pathway concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more inhibitors of the vitamin B6 pathway may be defined as a concentration of inhibitor at the target tissue of 10′ to 10′ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, one or more inhibitors of the vitamin B6 pathway disclosed herein may be combined with one or more additional therapies for the prevention or treatment of AML. Additional therapeutic agents include, but are not limited to, chemotherapeutic agents, arsenic trioxide (Trisenox), all-trans retinoic acid (ATRA), and stem cell transplants.

In some embodiments, the one or more inhibitors of the vitamin B6 pathway disclosed herein may be separately, sequentially or simultaneously administered with at least one additional therapeutic agent selected from the group consisting of alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent.

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

In certain embodiments, an additional therapeutic agent is administered to a subject in combination with the one or more inhibitors of the vitamin B6 pathway disclosed herein such that a synergistic therapeutic effect is produced. For example, administration of one or more inhibitors of the vitamin B6 pathway with one or more additional therapeutic agents for the prevention or treatment of AML will have greater than additive effects in the prevention or treatment of the disease. For example, lower doses of one or more of the therapeutic agents may be used in treating or preventing AML resulting in increased therapeutic efficacy and decreased side-effects. In some embodiments, the one or more inhibitors of the vitamin B6 pathway disclosed herein are administered in combination with any of the at least one additional therapeutic agents described above, such that a synergistic effect in the prevention or treatment of AML results.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits for the prevention and/or treatment of AML comprising one or more of: isoniazid, aftin-4, DFMO, gingkotoxin, aminooxyacetic acid, myriocin, and/or one or more inhibitory RNAs comprising a nucleic acid sequence of any one of SEQ ID NOs: 1-31, 39-42, or 982-990. Optionally, the above described components of the kits of the present technology are packed in suitable containers and labeled for the prevention and/or treatment of AML.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

The present technology is further illustrated by the following Examples, which should not be construed as limiting in any way. The following Examples demonstrate the preparation, characterization, and use of illustrative compositions of the present technology that inhibit the vitamin B6 pathway.

### Example 1: Experimental Materials and Methods

CRISPR/Cas sgRNA library construction and functional genomic screening. A CRISPR/Cas sgRNA library was designed as previously described in H. Koike-Yusa et al., Nat Biotechnol 32:267-273 (2014). sgRNAs targeting Renilla luciferase were included as negative controls. sgRNAs targeting Bcl2, Mcl1, Myc, Pcna, Rpa1, and Rpa3 were included as controls for positive regulators of leukemic cell proliferation. sgRNA sequences targeting Trp53 were included as controls for negative regulators of leukemic cell proliferation. sgRNA sequences targeting the above control genes and 236 metabolic genes were adapted from H. Koike-Yusa et al., Nat Biotechnol 32:267-273 (2014).

sgRNA inserts were designed as GTGGAAAGGACGAAACACCG (U6 promoter: SEQ ID NO: 32)+20 nt sgRNA+GTTTTAGAGCTAGAAATAGC (tracrRNA; SEQ ID NO: 33)+GGCCCTGGGGGATCTTT (barcode sequence; SEQ ID NO: 34). sgRNA inserts were synthesized by CustomArray, Inc. (Bothell, Wash.) and were amplified with the forward primer: 5′ AAAGATCCCCCAGGGCC 3′ (SEQ ID NO: 35) and the reverse primer: 5′ TTATATATCTTGTGGAAAGGACGAAACACC 3′ (SEQ ID NO: 36) in a first round PCR reaction, and were subsequently amplified with the forward primer: 5′ TAGCCTTATTTTAACTTGCTATTTCTAGCTCTAAAAC 3′ (SEQ ID NO: 37), and the reverse primer: 5′ ATTTCTTGGCTTTATATATCTTGTGGAAAGG 3′ (SEQ ID NO: 38) in a second round PCR reaction. Second round PCR products were then treated with T4 Polynucleotide Kinase followed with a Gibson assembly reaction with a LentiCRISPR v2 vector (Addgene, Watertown Mass.) that had been digested with BsmBI and treated with alkaline phosphatase. Transformation was performed using electrocompetent cells (Lucigen Corp., Middleton, Wis.), and bacterial colonies were collected for plasmid extraction (QIAGEN Plasmid Maxi Kit, Qiagen, Hilden, Germany).

For functional genomic screening, viral sgRNA pools were produced by transfecting HEK293 cells with LentiCRISPR v2 sgRNA libraries with VSVG and Pak2 packaging vectors. Two days after transfection, viral sgRNA pool supernatants were collected and filtered. Nras(G12D)/MLL-AF9 leukemic cells (cultured in RPMI medium supplemented with 10 ng/ml SCF, 2 ng/ml IL-6 and 0.7 ng/ml IL-3) were spin infected with viral sgRNA pool supernatants at 1,500 RPM for 60 minutes (37° C.). Viral infection was performed at a viral titer such that the majority of leukemic cells only contained one sgRNA integration. After spin infection, viral supernatants were substituted with leukemic cell culture medium. Genomic DNA was extracted from leukemic cells that were collected at day 1 and day 9 of cell culture. The integrated sgRNAs were PCR amplified and were pooled for deep sequencing analysis. Deep sequencing reads corresponding to each sgRNA at each day were identified after demultiplexing of the deep sequencing results.

Culture, Infection, and Compound Treatment of Leukemic Cells, iMEF Cells, and 3T3 Cells.

Mouse AML cell lines, human AML cell lines, iMEF cell line and 3T3 cell line were infected with virus encoding Cas9 (Addgene 52962, Watertown Mass.) followed with blastcitidin selection to generate Cas9 cell lines. All viral sgRNA infection experiments were performed on Cas9 cell lines. All viral shRNA infection experiments were performed on cell lines carrying rtTA3. Mouse leukemic cell lines including Nras(G12D)/MLL-AF9, MLL-AF9, Nras(G12D)/IDH, Nras(G12D)/NPM1, and IDH/Flt3 were cultured in RPMI medium supplemented with 10% Fetal Bovine Serum (FBS), L-glutamine, penicillin streptomycin, recombinant mouse SCF, IL-3 and IL-6. Human leukemic cell lines MOLM13, M12, Semk2, THP1, and K562 were cultured in RPMI medium supplemented with 10% FBS, L-glutamine, penicillin, and streptomycin. Vitamin B6 depleted medium (VB6−) was prepared by customized pyridoxin (−) RPMI (Gibco, Gaithersburg, Md.) with 10% dialyzed FBS, L-glutamine, penicillin, and streptomycin. Pyridoxin hydrochloride (Sigma, St Louis Mo.) was added back to VB6(−) medium to make the VB6 (+) medium used for leukemia and MEF culture. Control or gene targeting sgRNAs or shRNAs were delivered by lentivirus. Spin infections were performed at 37° C. and 1500 RPM for 60 minutes. After spin infection, viral supernatants were substituted with leukemic cell culture medium. For compound treatment, isoniazid was added in culture medium at indicated concentrations.

Molecular Cloning.

PDXK shRNAs were cloned into a doxycycline inducible H2 vector (D9891-25G, Sigma-Aldrich, St. Louis, Mo.). Leukemic cells were infected with viruses encoding shRNAs targeting Renilla luciferase or PDXK, followed by either in vitro culture or transplantation. For in vitro culture experiments, doxycycline was supplemented into culture medium, and the percentage of GFP⁺ cells was monitored. For in vivo AML mouse model experiments, the dietary feed of the mice was supplemented with doxycycline, and luciferase expression levels were monitored. For in vivo drug efficacy experiments, leukemic cells were transplanted into sub-lethally irradiated mice. Control PBS or isoniazid (90 mg/kg) was injected 6 hours after transplant and then daily into the transplant containing mice, and luciferase expression levels were monitored. High-performance liquid chromatography-mass spectrometry (HPLC-MS) was performed as previously described in A. M. Intlekofer et al., Nat Chem Biol 13, 494-500 (2017).

Bone marrow hematopoietic stem and progenitor cell isolation, culture, and compound treatment. For isolation of bone marrow cells, the bone marrow from the femur and tibia was flushed into ice cold PBS buffer. After red blood cell lysis with ammonium chloride (STEMCELL 07850, Vancouver Canada), the bone marrow cells were resuspended with PBS and filtered into a round bottom tube with cell strainer cap. The hematopoietic cells lineages were stained with biotin mouse lineage panel (BD Biosciences 559971, San Jose Calif.), then isolated by magnetic beads separation. For c-Kit positive cells, mouse hematopoietic cells were isolated from bone marrow using MACS CD117 isolation system. Purified bone marrow cells were cultured in RPMI medium supplemented with recombinant mouse SCF, IL-3, and IL-6. Control or Pdxk targeting sgRNAs or shRNAs were delivered by lentivirus. Spin infections were performed at 37 degree and 1500 RPM for 60 minutes. After spin infection, viral supernatants were substituted with leukemic cell culture medium. For compound treatment, isoniazid was added in culture at indicated concentrations.

Cell Cycle, Apoptosis, Differentiation, and Redox Experiments.

For competition and cell proliferation experiments, mouse or human leukemic cells were infected with viruses encoding sgRNAs or shRNAs. Spin infections were performed at 37 degree and 1500 RPM for 60 minutes. After spin infection, viral supernatants were substituted with leukemic cell culture medium. GFP+ percentage or cell number was monitored during culture using guava flow cytometer. The absolute cell number was measured by using flow cytometry absolute count standard (Bangs Laboratories Inc., Fishers Ind.). The experimental results were analyzed by guavasoft software.

For cell cycle experiment, Nras(G12D)/MLL-AF9 cells or MOLM13 cells were infected with viruses encoding sgRNAs either targeting Rosa26 or Pdxk. On day 9, 10, 12, 17 of infection, Nras(G12D)/MLL-AF9 cells or MOLM13 cells were stained with EdU Alexa Fluor 647 Flow Cytometry Assay Kit (Invitrogen C10634, Carlsbad Calif.). Briefly, 10 μM Edu was pulsed in culture for 20 mins. Cells were then washed and fixed with Click-iT fixative for 15 min following by Click-iT reaction. The percentages of S-phase cell in population were measured by flow cytometry analysis.

For apoptosis experiment, Nras(G12D)/MLL-AF9 cells were infected with viruses encoding either control Rosa26 sgRNA or Pdxk sgRNAs. On day 9 of infection, cultured cells were stained with Annexin-APC and 7-AAD. GFP+ and GFP− populations were gated for standard flow cytometry analysis.

For differentiation experiment, Nras(G12D)/MLL-AF9 or MOLM13 cells were infected with viruses encoding either control Rosa26 sgRNA or Pdxk sgRNAs. On day 9 of infection, cultured cells were stained with c-Kit or Mac-1 antibodies. GFP+ and GFP− populations were gated for standard flow cytometry analysis to detect c-Kit and Mac-1 expression levels. Cultured cells were also processed with May-Grunwald Giemsa staining.

Leukemia Mouse Models and Xenogen Bioluminescence Imaging.

The mouse model of human AML was produced as previously described with some modification. For doxycycline inducible shRNA mouse models, Nras(G12D)/MLL-AF9 leukemic cells infected with viruses encoding shRNAs either targeting Renilla luciferase or Pdxk. Leukemic cells were collected and resuspended in PBS at a concentration of 5 million/ml. 5-6 weeks old B6-LY5 mice (Charles River Labs, Wilmington Mass.) were sublethally irradiated (450 cGy) and transplanted with 1 million leukemic cells through tail vein injection. 6 days after transplantation, Xenogen bioluminescence imaging was utilized to monitor luciferase signal intensity. Before imaging, all mice were anesthetized with 2% isoflurane, and anesthesia was also maintained during the image acquisition inside Xenogen dark chamber. Briefly, 300 mg/kg luciferin (Gold Biotechnology, Olivette, Mo.) was intraperitoneally injected into mice. 10 minutes after luciferin injection, Xenogen bioluminescence imaging was applied to monitor luciferase signal intensities of the mice. After confirmation of disease establishment, doxycycline was supplemented into mouse diet and water to induce shRNA expression. Xenogen bioluminescence imaging was then utilized every three days to monitor luciferase signal intensities and disease progression.

For isoniazid injection experiment, Nras(G12D)/MLL-AF9 leukemic cells were collected and resuspended in PBS at a concentration of 5 million/ml. 5-6 weeks old B6-LY5 mice (Charles River Labs, Wilmington Mass.) were sublethally irradiated (450 cGy) and transplanted with 1 million leukemic cells through tail vein injection. PBS or 90 mg/kg isoniazid were intraperitoneally injected daily.

The leukemia disease progress was monitored by in vivo bioluminescent imaging using IVIS Lumina image system (Xenogen) every 3 days. Before imaging, all mice were anesthetized with 2% isoflurane, and anesthesia was also maintained during the image acquisition inside Xenogen dark chamber. 300 mg/kg D-Luciferin (Gold Biotechnology Olivette, Mo.) in PBS at 15 mg/ml was intraperitoneally injected ˜12 minutes before image capturing. The bioluminescence was acquired and quantitatively analyzed by the Living Image software.

Western Blot Experiments.

For viral shRNA infection, leukemic cells or bone marrow HSPCs were infected with virus encoding control shRNA or shRNAs targeting PDXK. After doxycycline induction, leukemic cells were collected and washed once with PBS. Cell pellets were counted and lysed with laemmli lysis buffer at room temperature for 4 hours and boiled at 95 degree for 5 minutes. For viral sgRNA infection, leukemic cells and iMEF cells were infected with virus encoding control sgRNA or sgRNAs targeting PDXK. After infection, GFP+ cells were sorted and proteins were extracted using RIPA buffer. For bone marrow cells, cells were collected from moribund mice, and GFP+ cells were sorted and proteins were extracted using RIPA buffer.

Protein samples were run on 4-15% Mini-PROTEAN® TGX™ Gel, transferred to PVDF membranes (Millipore, Burlington Mass.). After blocking with 5% skimmed milk, membranes were incubated with primary antibodies (1:1000) overnight and HRP-conjugated secondary antibody (1:10000) for 1 hour. Primary antibodies include anti-PDXK antibody (Sigma HPA030196, St Louis, Mo.), anti-GFP antibody (Hypromatrix HM2020, Worcester Mass.), and anti-Actin antibody (Sigma A3854, St Louis, Mo.). Images were acquired using FluorChem imaging system or X-ray films and quantified by Image J.

High-Performance Liquid Chromatography-Mass Spectrometry (HPLC-MS).

LC-MS grade solvents were purchased from Thermo Fisher Scientific (Waltham, Mass.), metabolite standards were purchased from Sigma (St. Louis, Mo.) unless stated otherwise. Nras(G12D)/MLL-AF9 cells infected with viruses encoding Pdxk shRNAs were harvested after 5 days of doxycycline induction. Human MOLM13 cells infected with viruses encoding Pdxk sgRNAs were harvested at day 11 post infection. Nras(G12D)/MLL-AF9 cells or MOLM13 cells were treated with indicated concentrations of isoniazid and harvested after 5 days treatment.

Measurement of Vitamin B6 Species.

In each case, 2 million cells were collected by centrifugation at 440×g, media was aspirated and 1004 of ice-cold 10% trichloroacetic acid with or without 100 ng/mL d3-pyridoxal (d3-PL), d3-pyridoxine (d3-PN), d3-pyridoxamine (d3-PM), d3-pyridoxal phosphate (d3-PLP) (IsoSciences LLC, Ambler Pa.) was added for metabolite extraction. For experiments involving isoniazid treatment, cells were washed once with PBS to remove residual drug and minimize post-lysis reactions between isoniazid and PLP, which was confirmed by monitoring the d3-PLP internal standard. After overnight incubation at −80° C., samples were vortexed and transferred to 1.7 mL conical eppendorf tubes and centrifuged at 21,000×g for 20 min at 4° C. The supernatant was transferred to autosampler vials for LC-MS analysis. Vitamin B species separation was performed using an Agilent 1290 infinity pump and XSelect HSS T3 column (150×2.1 mm, 3.5 μm; Waters Corporation, Milford Mass.). Mobile phase A was 0.1% heptaflurobutyric acid (HFBA, Thermo Fisher, Waltham Mass.) and 0.1% formic acid (FA, Thermo Fisher, Waltham Mass.) in water, mobile phase B was 0.1% HFBA and 0.1% FA in acetonitrile. The injection volume was 10 μL and LC gradient conditions were: 0 min: 0% B; 1 min: 0% B; 7 min: 25% B; 8 min: 25% B; 9 min: 100% B; 10.5 min: 100% B, with 4.5 min of re-equilibration time. MS detection was performed using an Agilent 6230 TOF accurate mass spectrometer with Dual JetStream source operating in positive ionization mode. MS parameters were: gas temp 250° C.; gas flow: 13 L/min; nebulizer pressure: 45 psig; sheath gas temp: 225° C.; sheath gas flow: 12 L/min; VCap: 3,500 V; Fragmentor: 175 V; Skimmer: 65 V; Octopole RF: 750 V. Active mass axis correction was performed using a second nebulizer and pyridine (80.049480) and roxithromycin (837.53185). Data was acquired from m/z 50-1700 at 1.5 spectra/sec. Accurate mass (±20 ppm) and retention time was confirmed relative to the pure standards. Data analysis was performed using MassHunter software (Agilent, Santa Clara Calif.).

Metabolite Profiling.

2 million cells were washed once with PBS for the amino acid detection and isoniazid treatment. The supernatant was vacuum dried down (Genevac EZ-2 Elite). Cells were harvested as described above with the exception that the extraction solvent was 80:20 methanol: water containing 1.5 μM ¹³C¹⁵N-labeled amino acid mix (Cambridge Isotope Laboratories) and the 50 ng/mL vitamin B6 internal standards described above. After overnight incubation at −80° C., samples were vortexed well and transferred to 1.7 mL conical eppendorf tubes and centrifuged at 21,000×g for 20 mins at 4° C. Samples were re-suspended in 70 μL of water and divided between hydrophilic interaction liquid chromatography (HILIC) and Heptafluorobutyric acid (HFBA) methods. For HFBA positive mode profiling, 30 μL of re-suspended extract+20 μL MPA were mixed and analyzed as described for B6 species. For HILIC negative mode profiling, 404 of re-suspended extract was diluted with 604 of acetonitrile. HILIC metabolite separation was performed using an Agilent 1290 Infinity pump and ZIC-pHILIC polymeric column (PEEK 150×2.1 mm, 5 μm; Merck Sequent). Mobile phase A was 90:10 water: acetonitrile containing 10 mM ammonium bicarbonate (pH 9.4), mobile phase B was 90:10 acetonitrile: water containing 10 mM ammonium bicarbonate (pH 9.4). The injection volume was 5 μL and LC gradient conditions were: 0 min: 95% B; 1 min: 95% B; 10 min: 50% B; 13 min: 50% B; 14 min: 30% B; 16 min 30% B, with 7 min of re-equilibration time. MS detection was performed using an Agilent 6545 Q-TOF mass spectrometer with Dual JetStream source operating in negative ionization mode. MS parameters were: gas temp: 200° C.; gas flow: 10 L/min; nebulizer pressure: 40 psig; sheath gas temp: 300° C.; sheath gas flow: 12 L/min; VCap: 3,000 V; Fragmentor: 125 V; Skimmer: 45 V; Octopole RF: 750 V. Active reference mass correction was through a second nebulizer using masses with m/z: 119.03632 and 980.016375. Data was acquired from m/z 50-1700 at 1 spectra/sec. Accurate mass (±20 ppm) and reference metabolite standards and MS/MS spectra collected from pooled QC samples were used to confirm the identity of the metabolites. Data analysis was performed within MassHunter software (Agilent, Santa Clara Calif.) and metabolites reported have <30% CV in pooled QC samples injected regularly throughout the analytical batch.

Plasmid Construction.

For sgRNA experiments, the pLKO5.sgRNA.EFS.GFP vector was used (Addgene 57822, Watertown Mass.). For shRNA experiments, RT3GEN vector was used (Fellmann et al., Cell Rep 5:1704-1713 (2013)). For c-kit positive bone marrow cells viral shRNA infection experiment, miR-E based MLS retroviral vector. The full length of mouse or human PDXK cDNA was cloned into MSCV Hygro-PGK-Hygro vector (Clontech 634401, Mountain View Calif.). The deletion and point mutant of PDXK were made by QuikChange Lightning Multi Site-Directed Mutagenesis Kit (Agilent Technologies, Santa Clara Calif.).

### Example 2: Identification of PDXK Function in AML Cell Proliferation

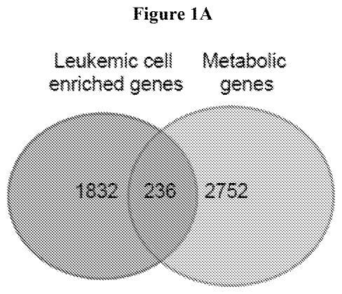

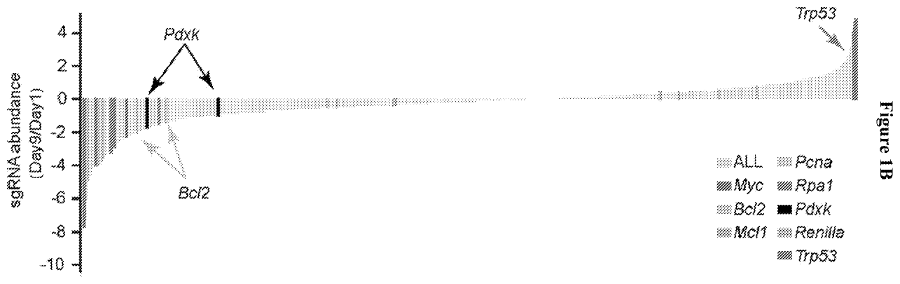

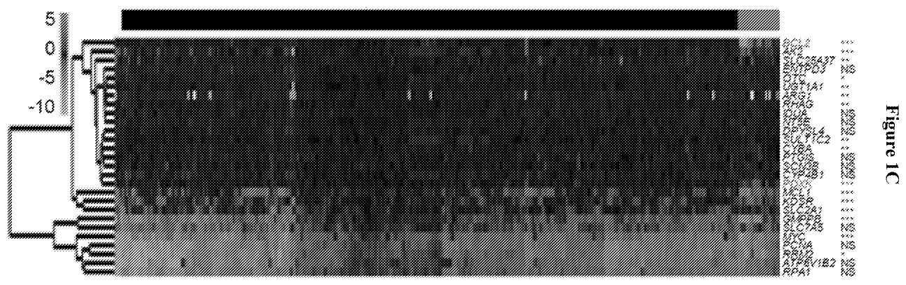

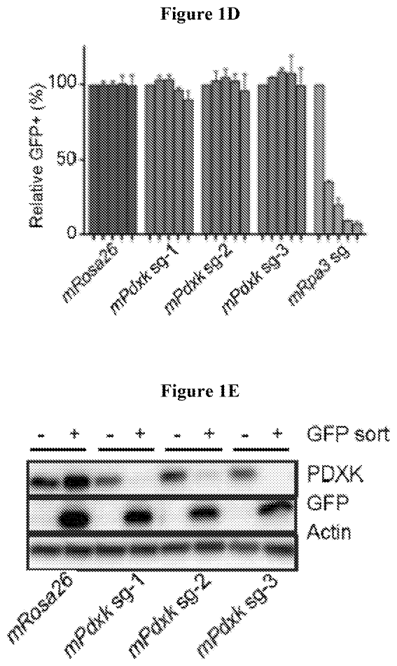

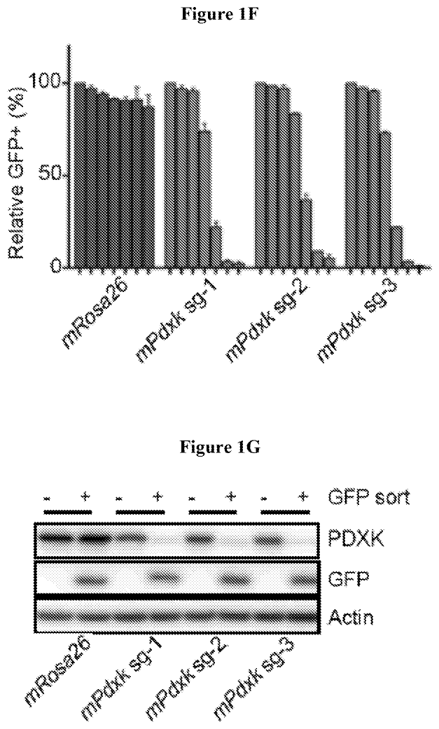

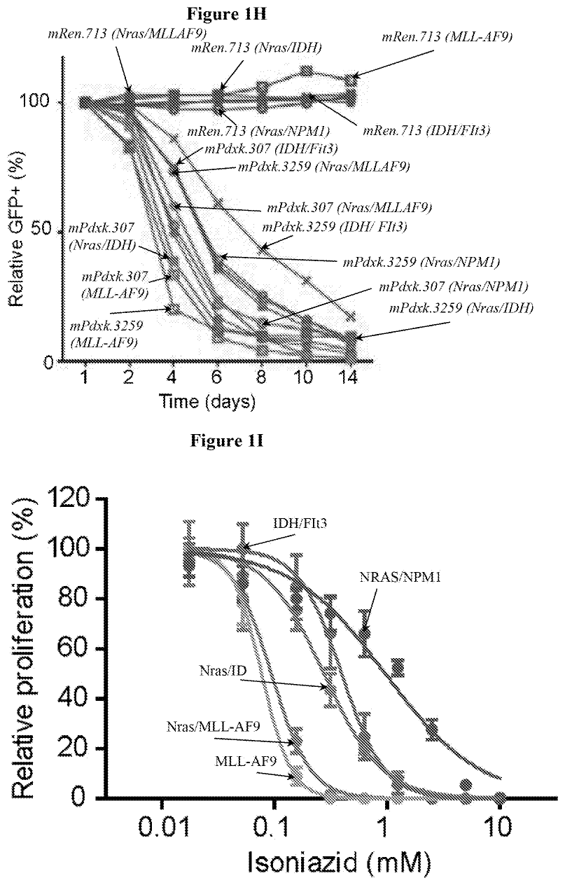

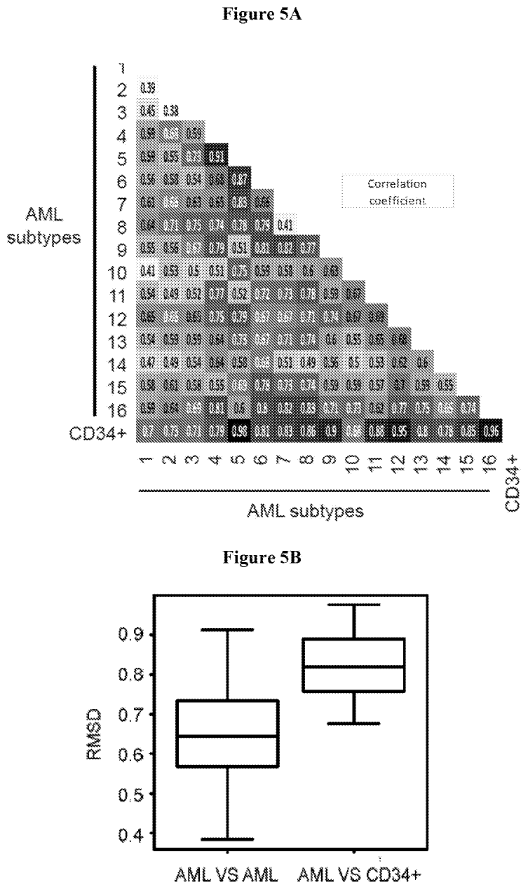

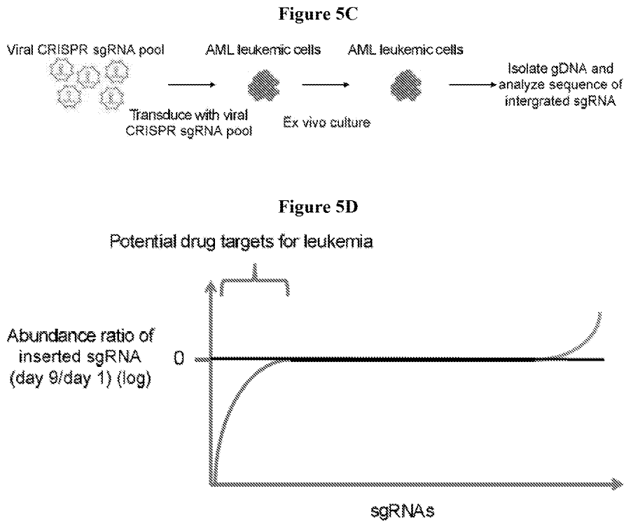

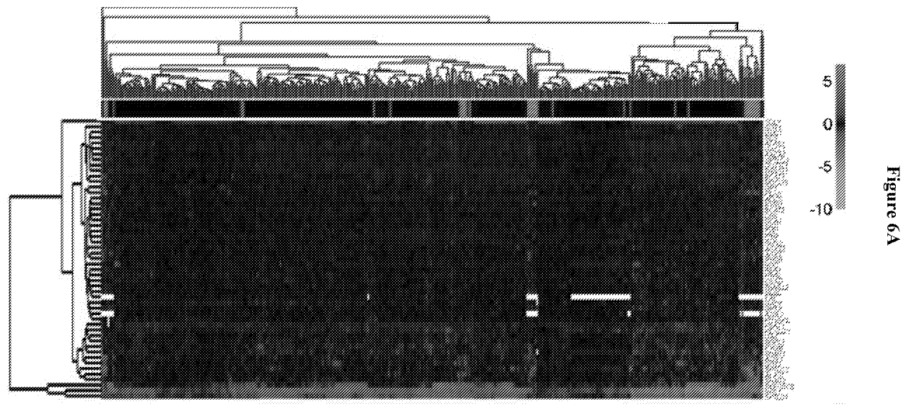

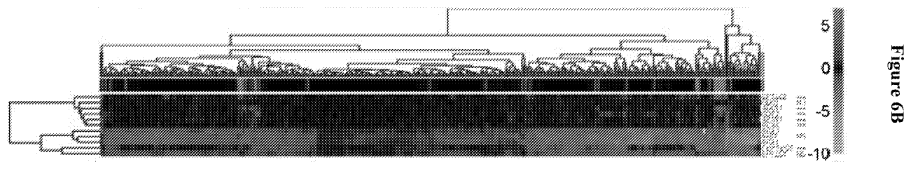

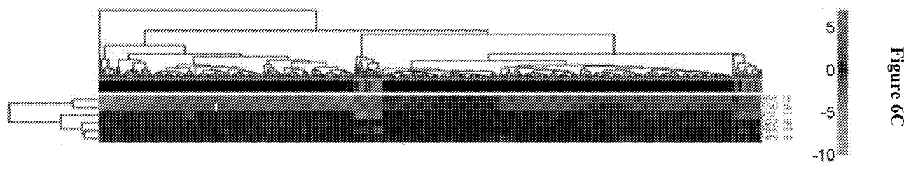

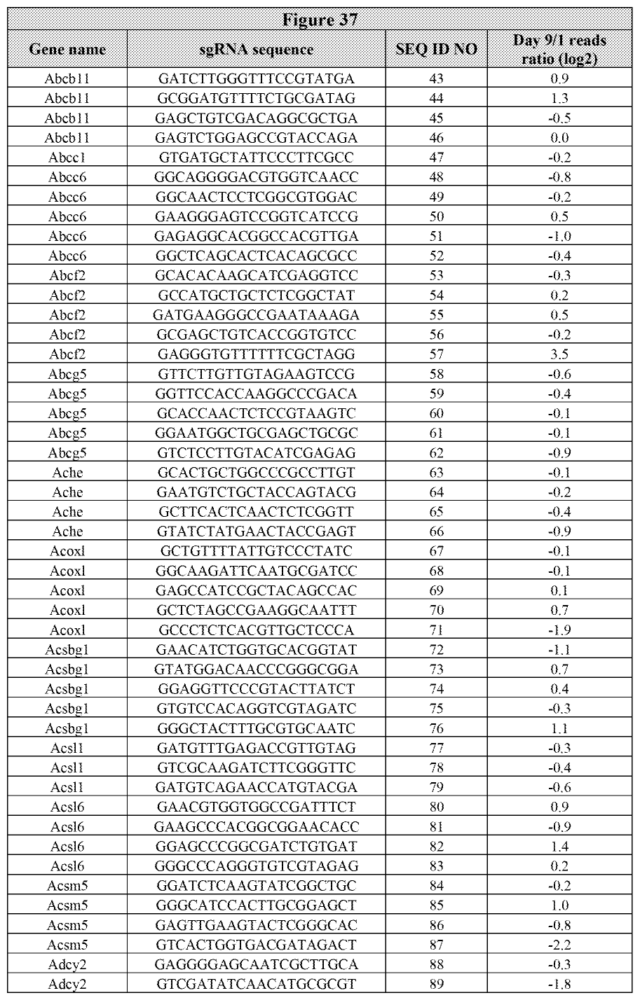

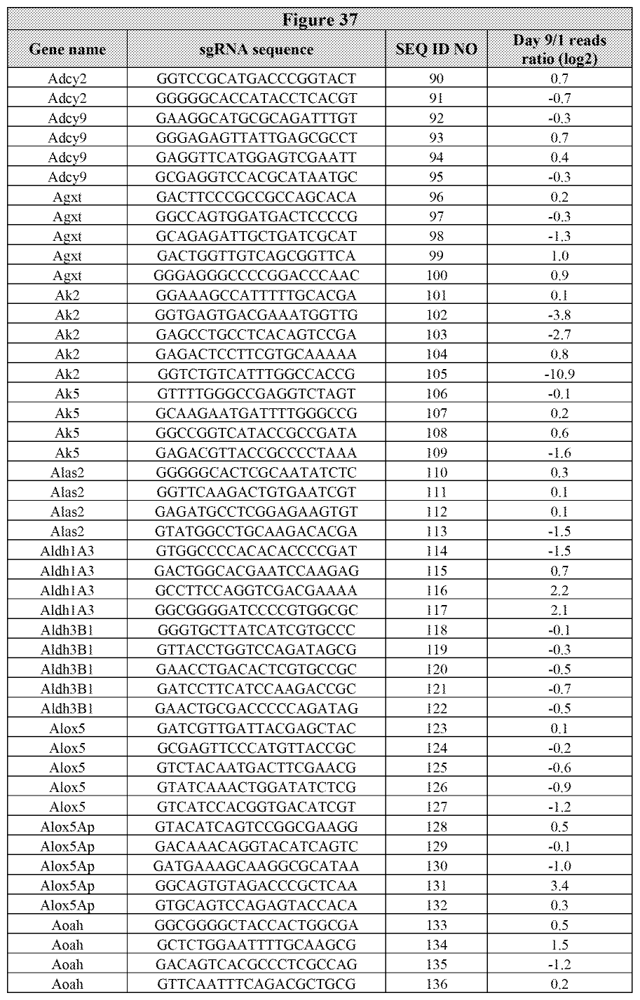

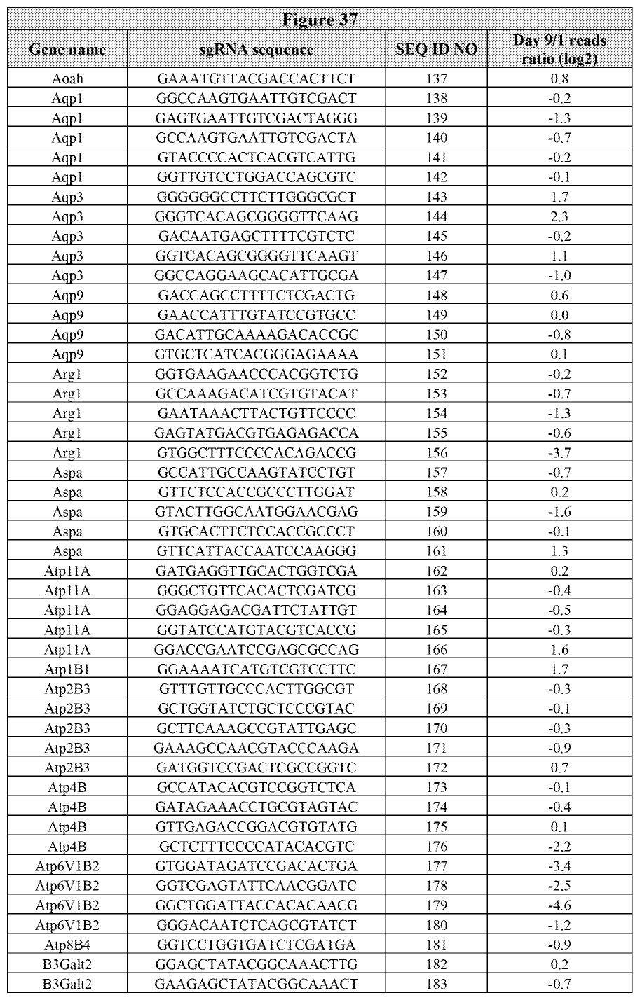

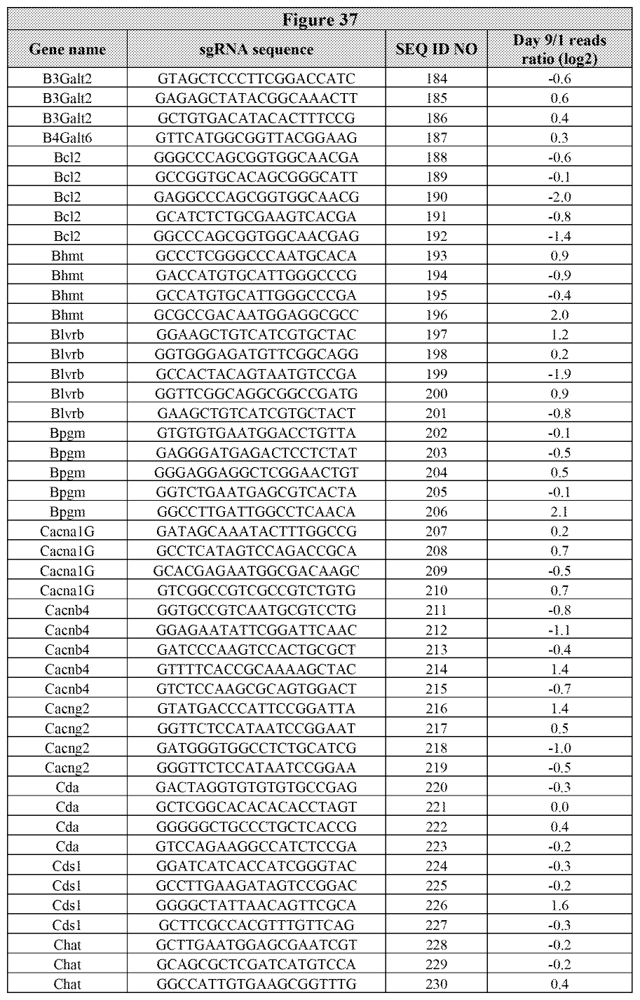

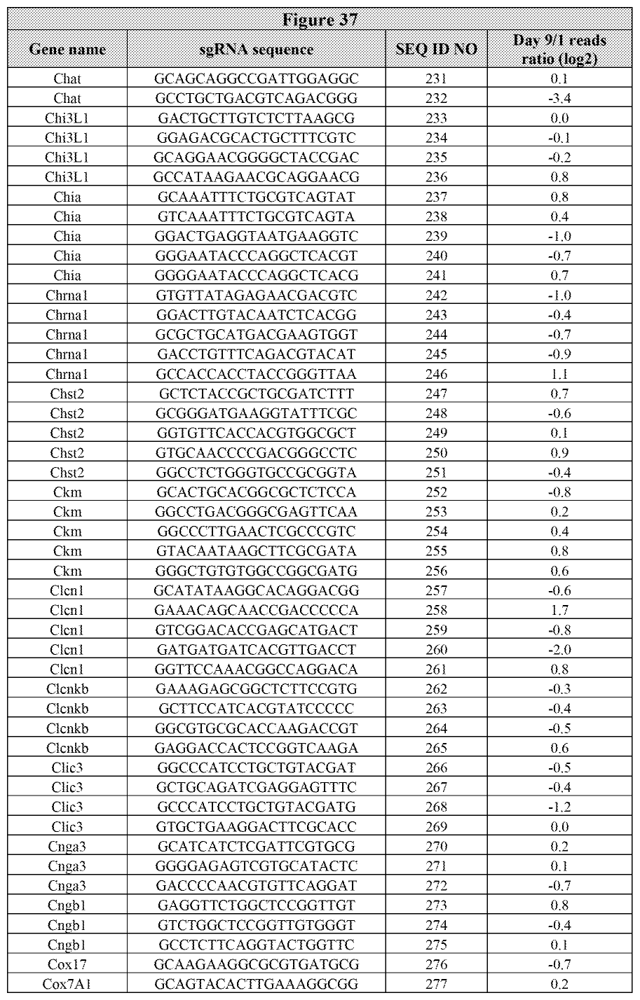

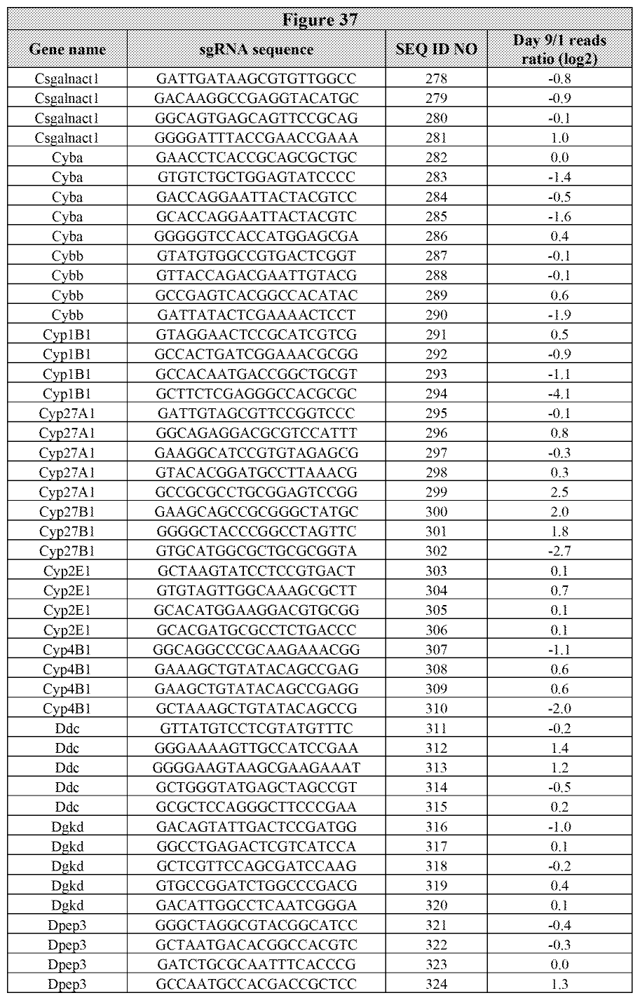

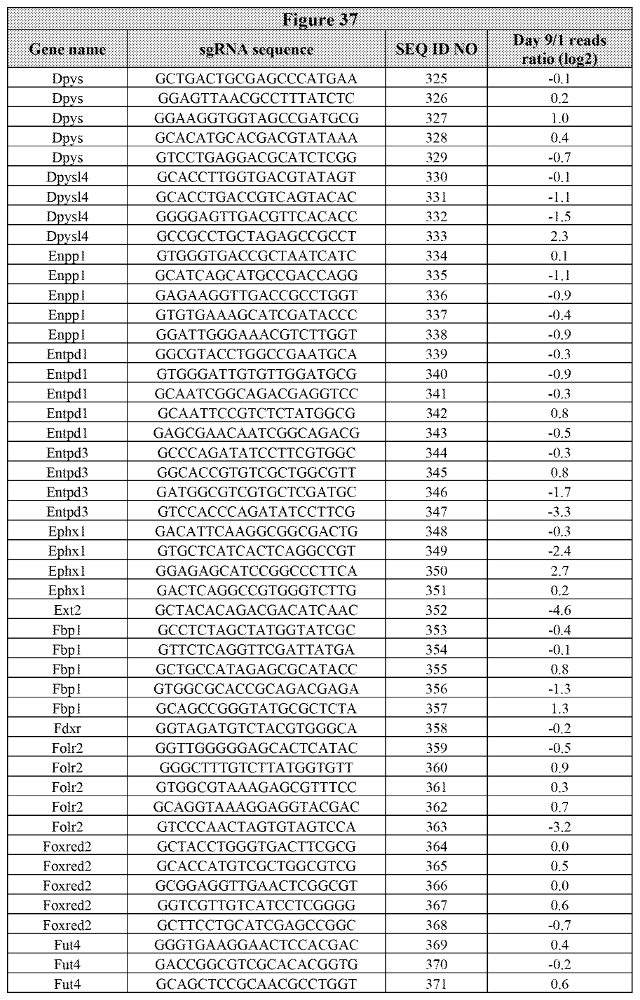

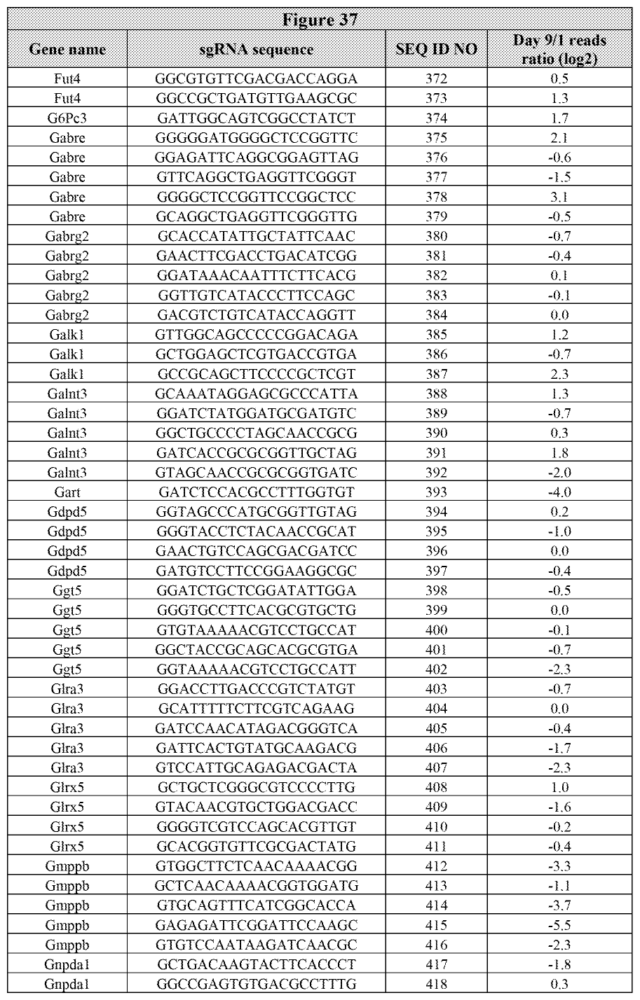

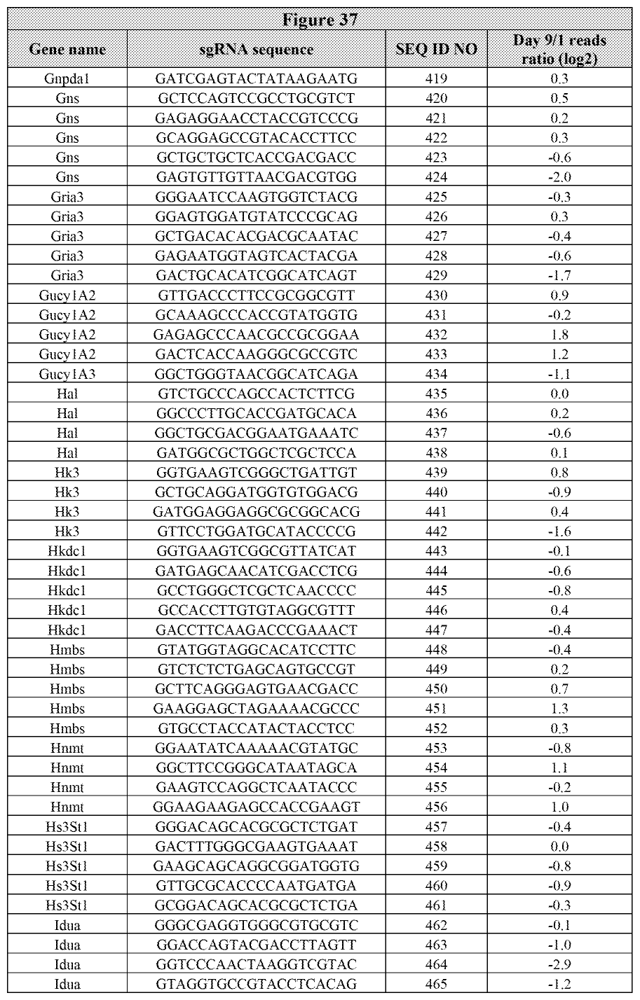

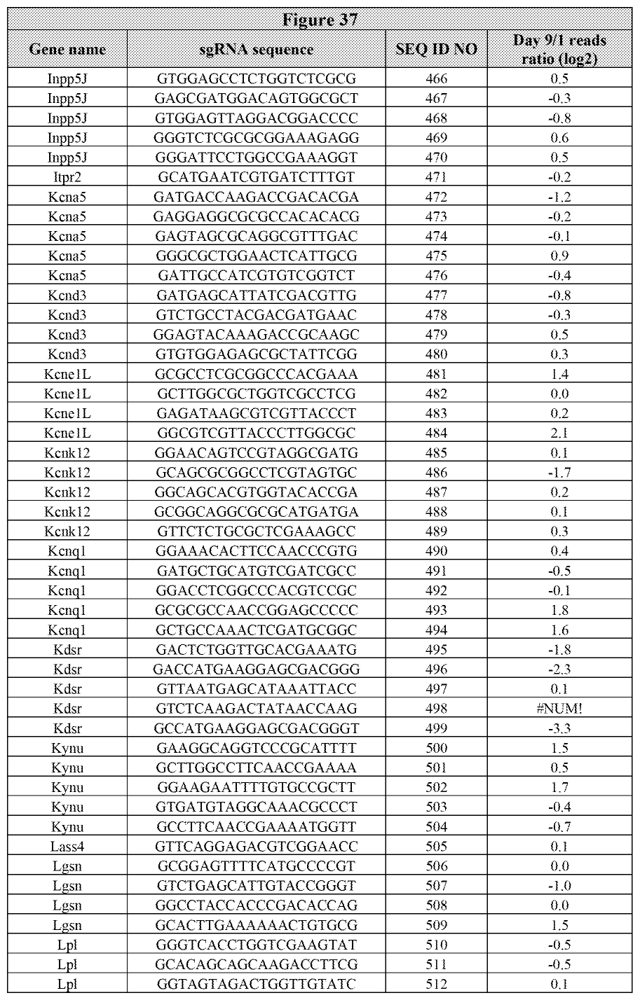

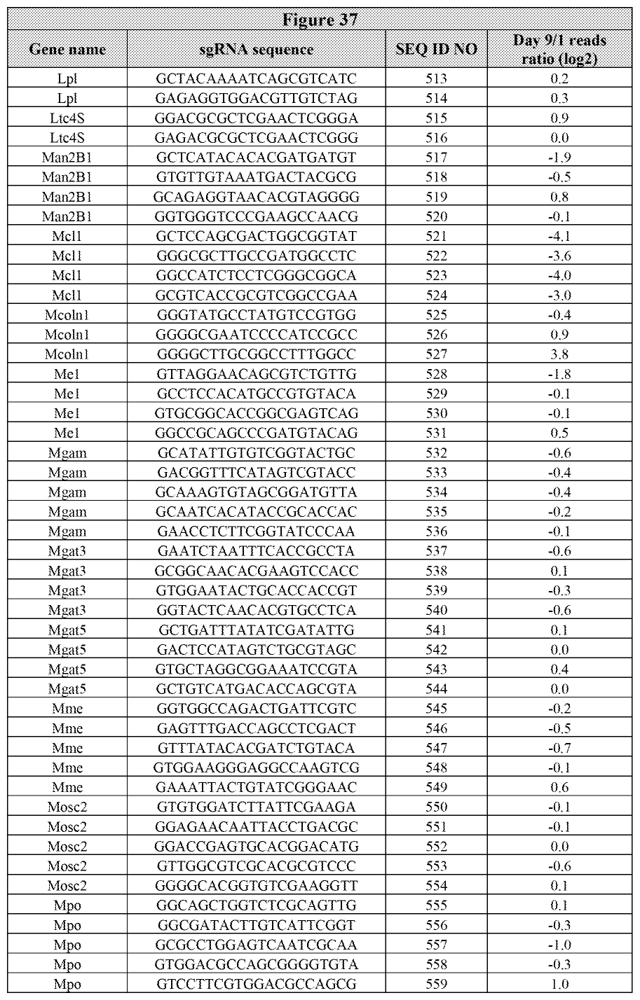

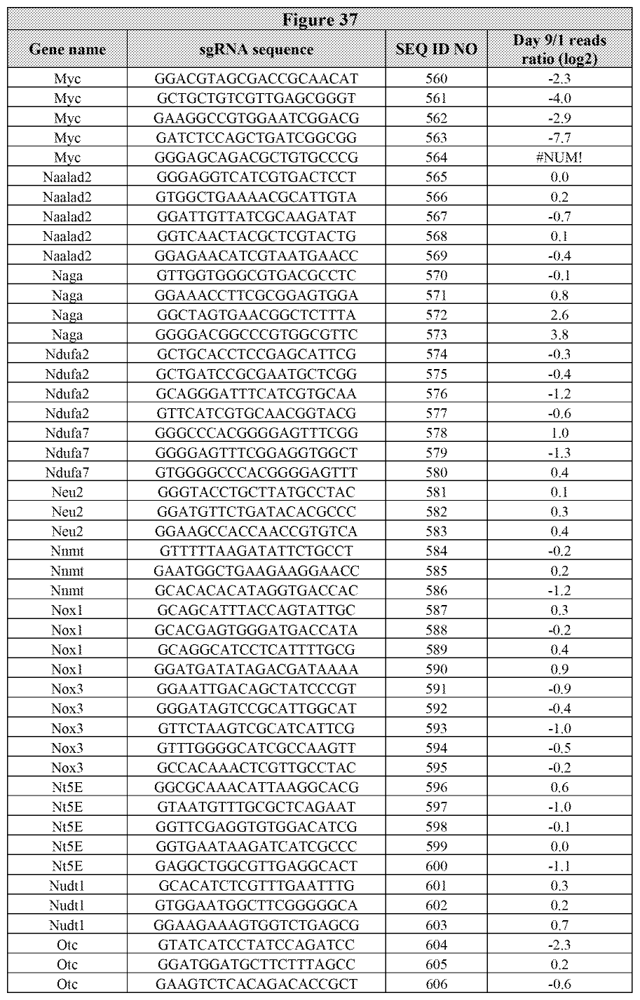

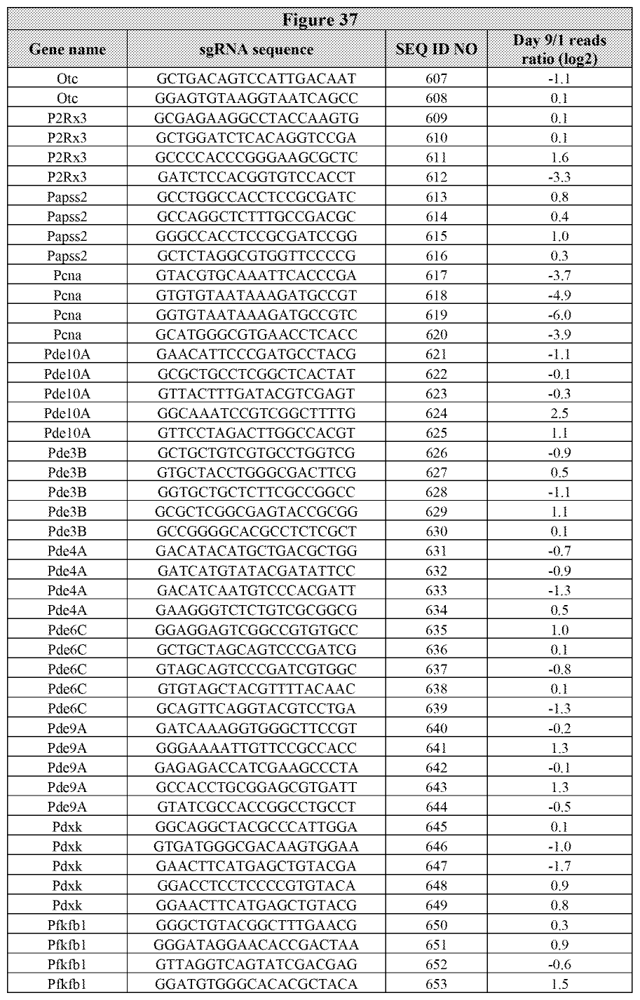

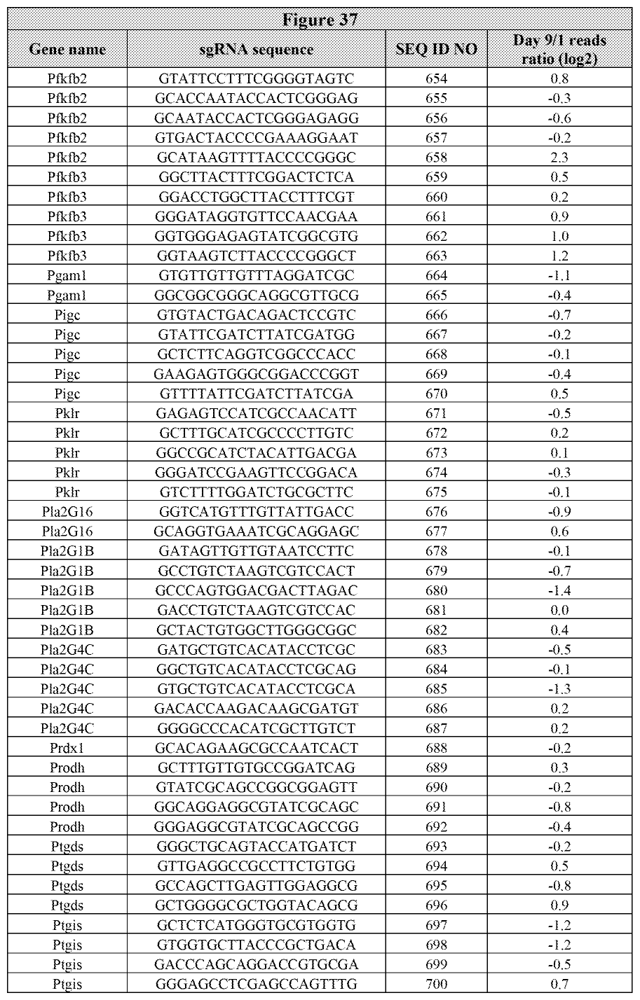

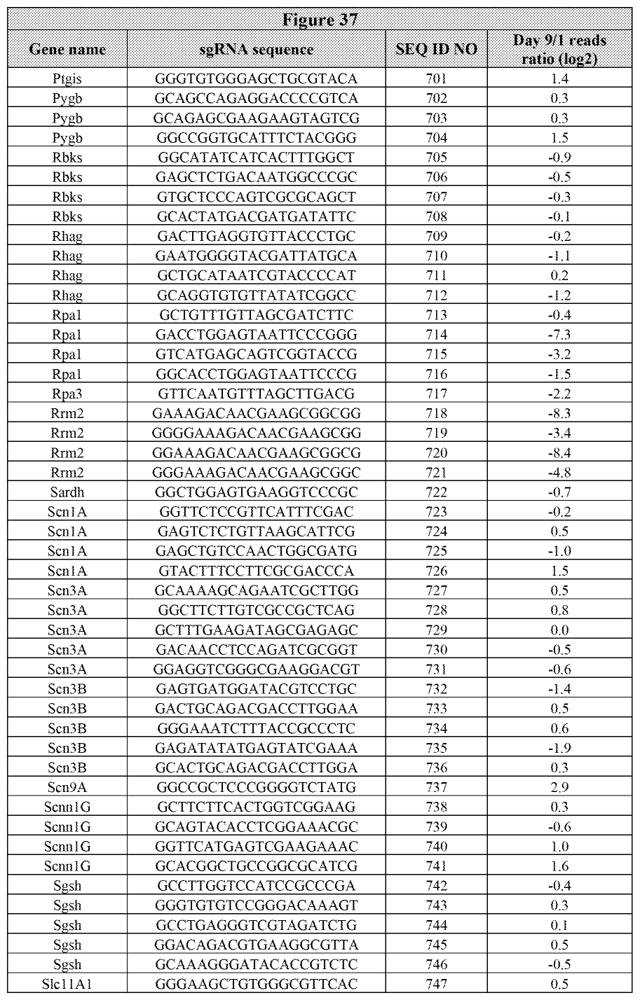

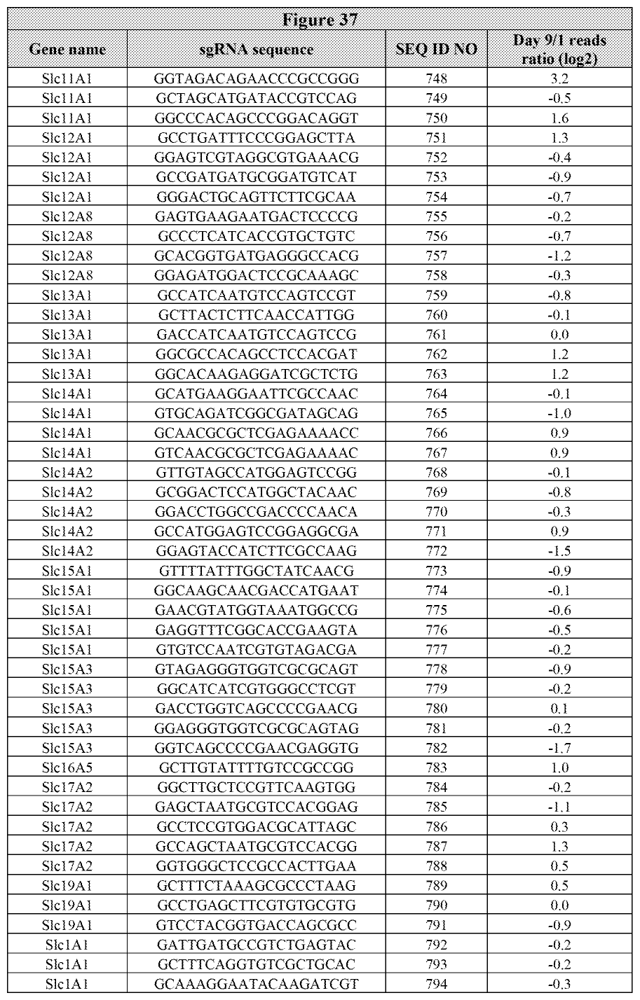

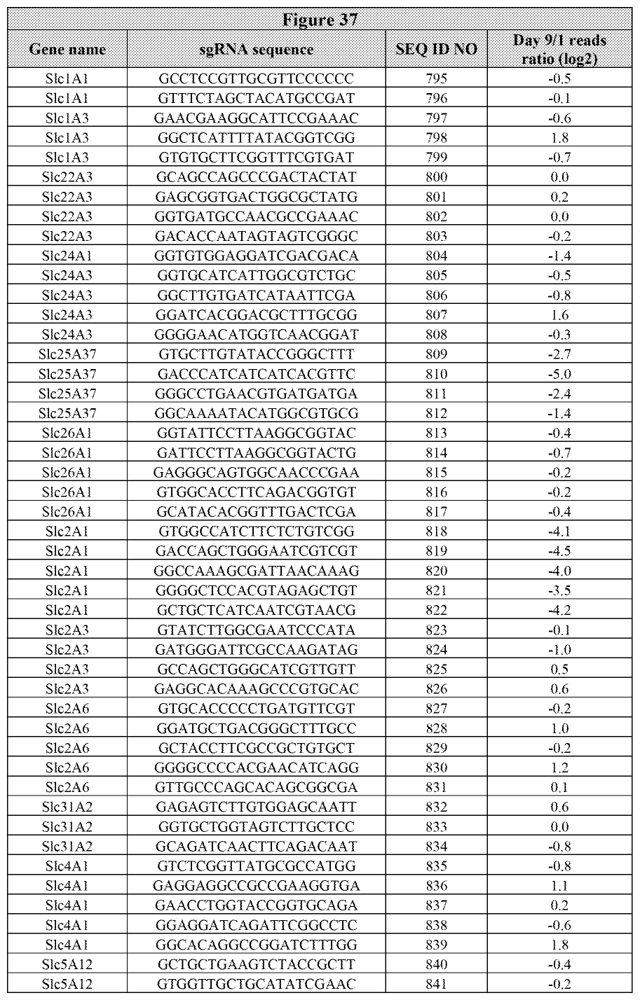

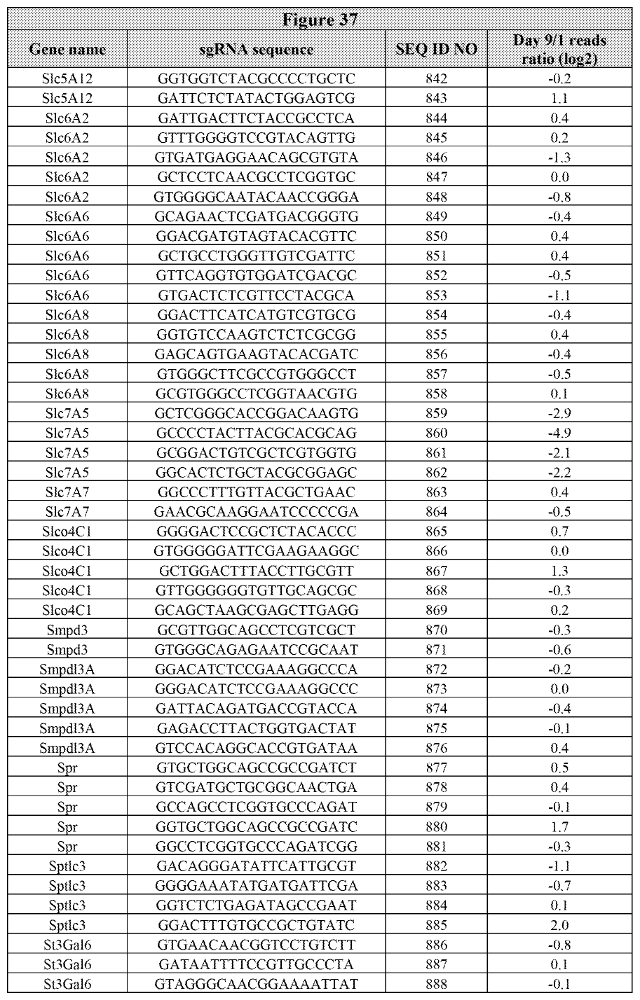

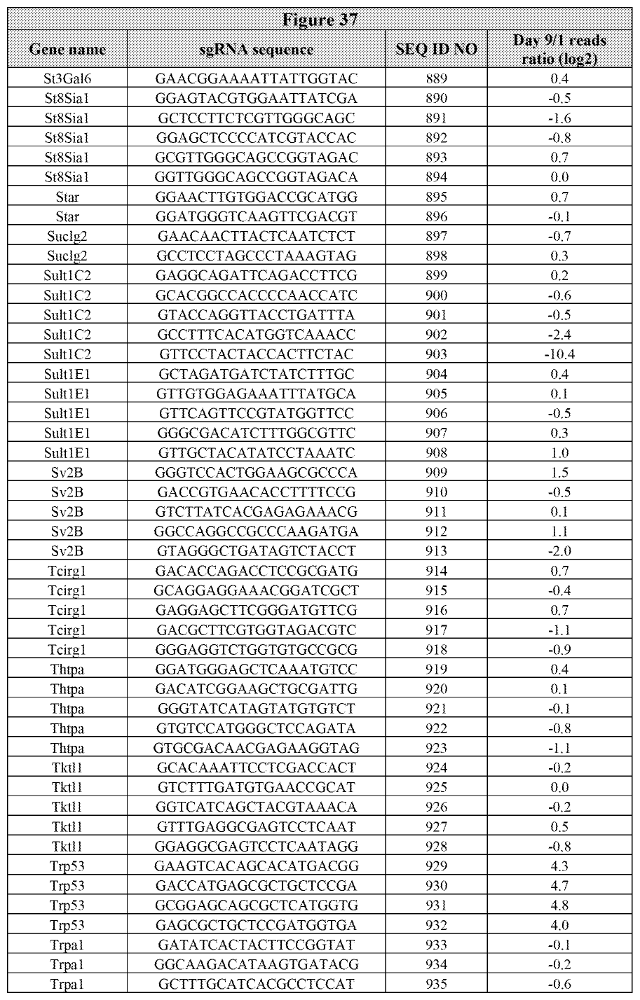

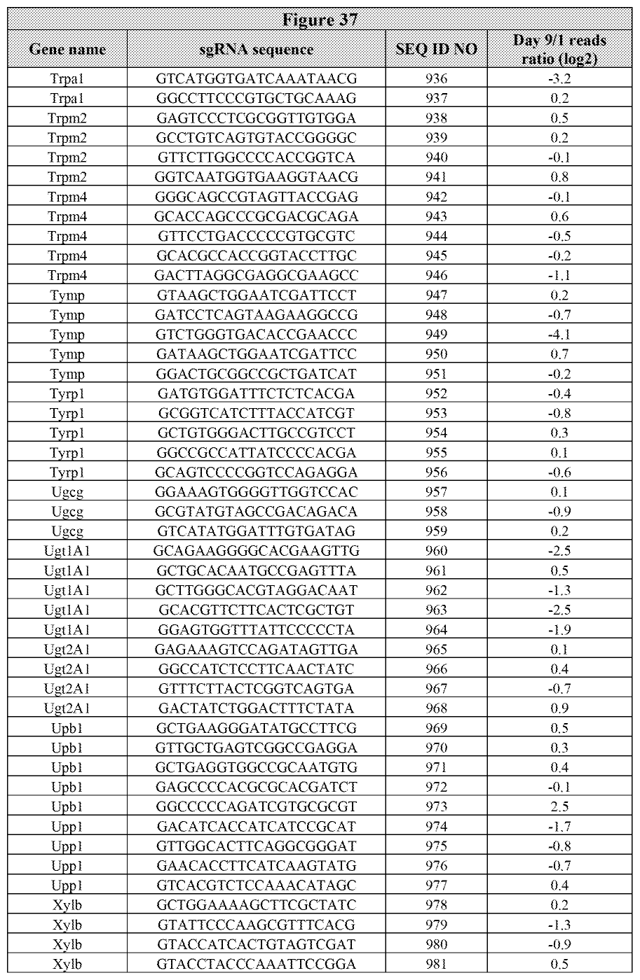

To identify AML specific metabolic vulnerabilities, 2752 genes encoding metabolic enzymes and transporters were analyzed and 236 genes were found to be abundantly expressed in leukemic cells (FIG. 1A, FIGS. 5A-5D, FIG. 35 (human), and FIG. 36 (mouse)). A CRISPR/Cas sgRNA library targeting these 236 genes was constructed and Nras(G12D)/MLL-AF9 leukemic cells were infected with the pooled viral sgRNA library to investigate their role in leukemic cell proliferation. Deep sequencing was performed to measure copy number changes of each sgRNA from day 1 to day 9 of culture (See FIGS. 5A-5D). As shown in FIG. 37, control sgRNAs were properly identified: control sgRNAs targeting genes known to be essential for AML cell proliferation were depleted in the screen (e.g. Myc, Bcl2, Mcl1, Pcna, and Rpa1), whereas sgRNAs that target the tumor suppressor Trp53 were enriched (FIG. 1B). The abundance of neutral sgRNAs (e.g. targeting Renilla luciferase) remained largely unchanged. These observations support the robustness of the screening results.

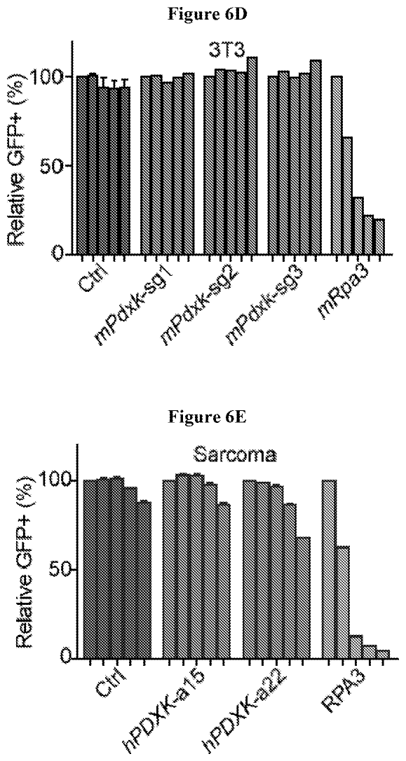

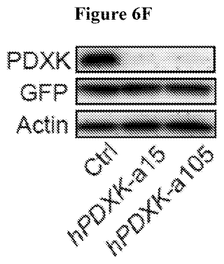

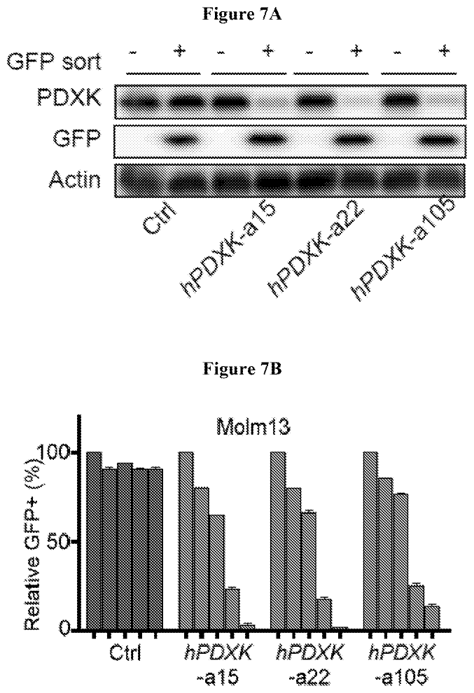

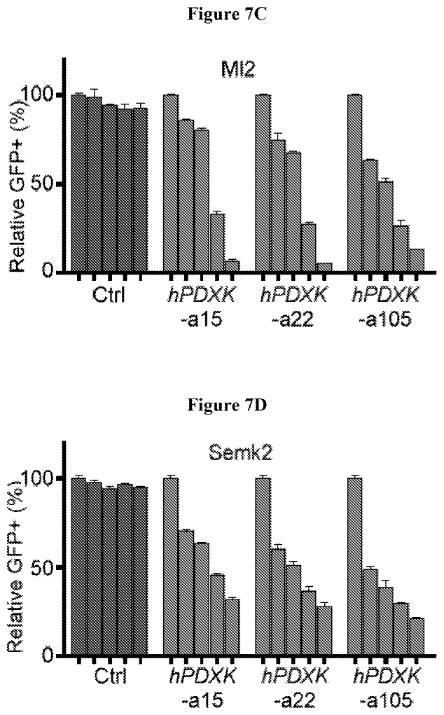

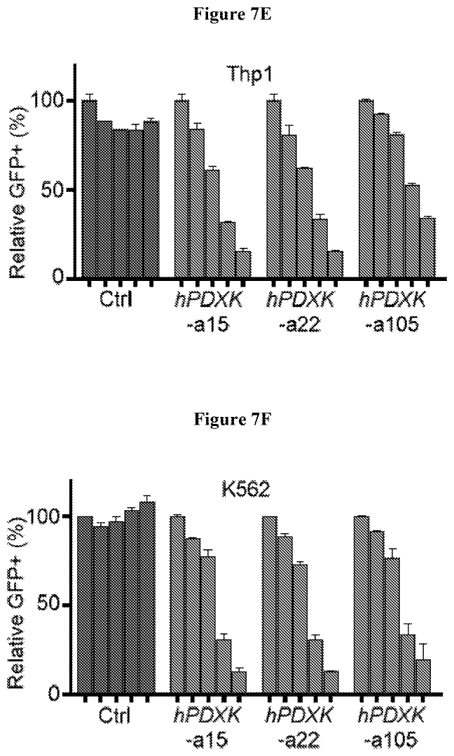

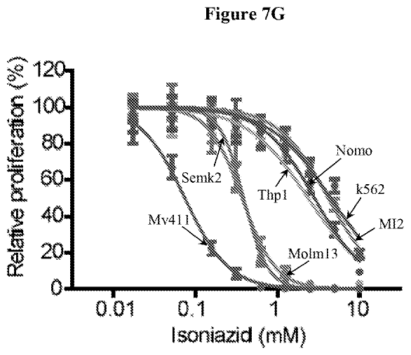

Among the 236 screened metabolic genes, 23 genes were identified with at least two sgRNA hits each with more than 50% copy number depletion (FIG. 1B and FIG. 37). These 23 genes regulate multiple different metabolic processes. For example, RRM2 is involved in nucleotide production, GMPPB is involved in mannose metabolism, SLC25A37 and SLC2A1 are transporters for small molecules, IDUA controls glycan degradation, and SULT1C2 is involved in sulfate metabolism. To prioritize hits from the screen for follow up study, publically available data from a series of genome-wide CRISPR/Cas9 screens in human cancer cells was analyzed (Rauscher et al., Nucleic Acids Res 45:D679-D686 (2017)). Pyridoxal kinase (PDXK), which catalyzes the formation of the bioactive form of vitamin B6, pyridoxal phosphate (PLP), was selected for further functional analysis because sgRNAs targeting PDXK were selectively required for leukemia cell proliferation relative to other normal and cancer cell types (FIGS. 1C to 1G and FIGS. 6A to 6F). Indeed, the requirement for PDXK to support proliferation across a large number of cell lines was similar to BCL-2 (FIG. 1C). To validate these findings, individual sgRNAs and short hairpin RNAs (shRNAs) that target PDXK were generated and competition assays performed in multiple murine and human AML cell lines. Consistent with CRISPR/Cas9 screening results, knockout of Pdxk using individual sgRNAs inhibited proliferation of Nras(G12D)/MLL-AF9 leukemia cells (FIGS. 1F and G). The validity of these findings was reinforced by shRNA-mediated knockdown of Pdxk as an orthogonal approach (FIG. 1H). Although the rate at which PDXK sgRNAs was depleted from AML cells was slower than observed for sgRNAs targeting broadly essential genes such as PCNA and RPA1, their effects were much more specific for leukemia cells. Hence, in contrast to sgRNAs targeting PCNA and RPA1, PDKX sgRNAs showed modest to no anti-proliferative effects in many normal and non-leukemia cancer cells (FIG. 1C to G and FIGS. 6A-6F). Knockdown of Pdxk inhibited the proliferation of 4 additional mouse AML cell lines produced by different oncogenic events (e.g. Nras(G12D)/IDH, IDH/Flt3, Nras(G12D)/NPM1, and MLL-AF9) (FIG. 1H), as well as a panel of human leukemic cell lines (MOLM13, ML2, SEMK2, THP1, and K562) (FIGS. 7A-7F).

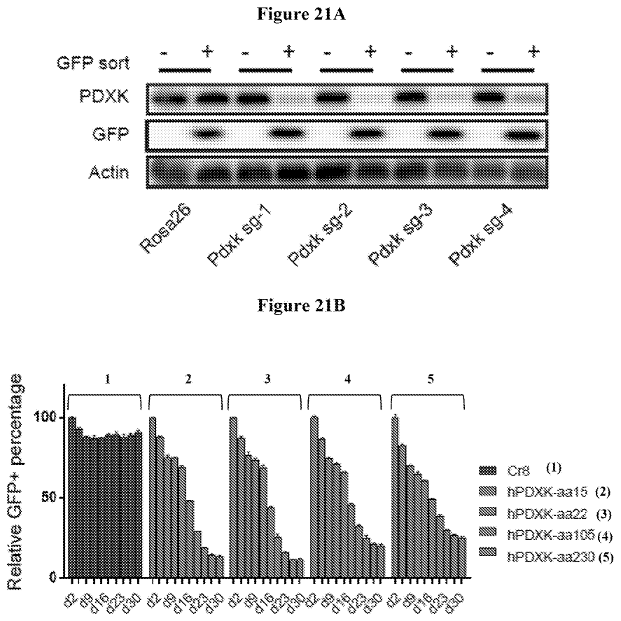

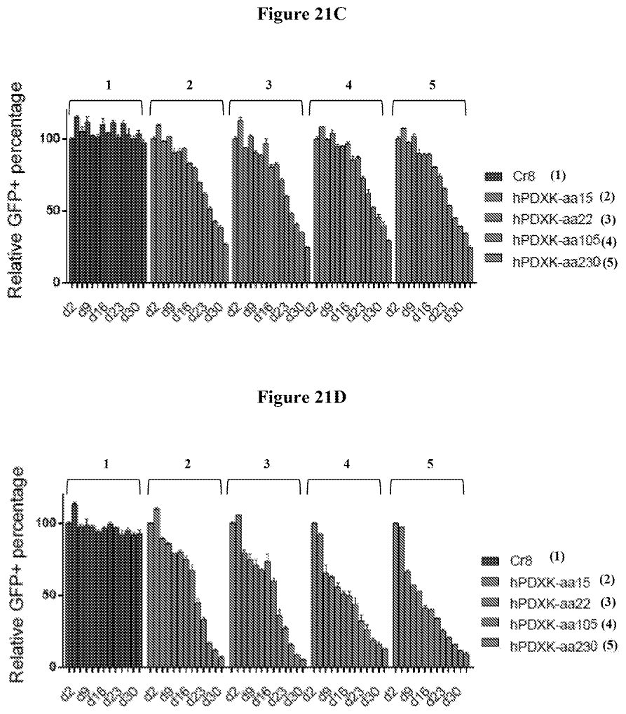

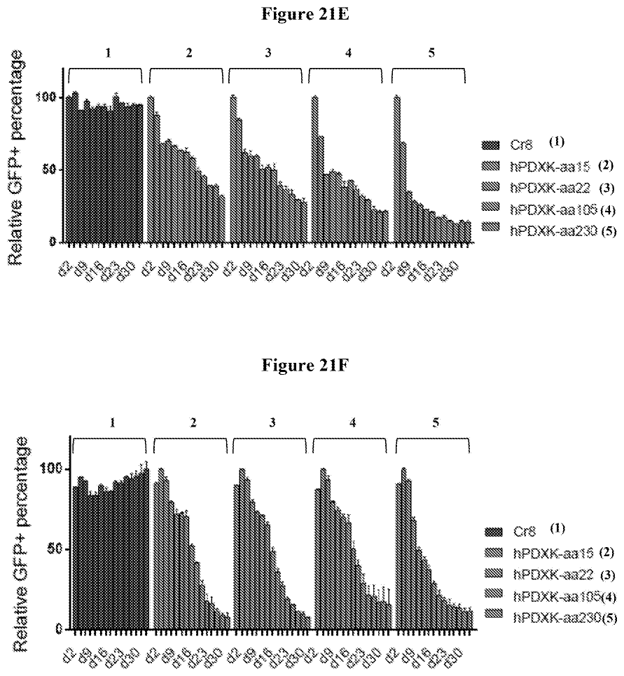

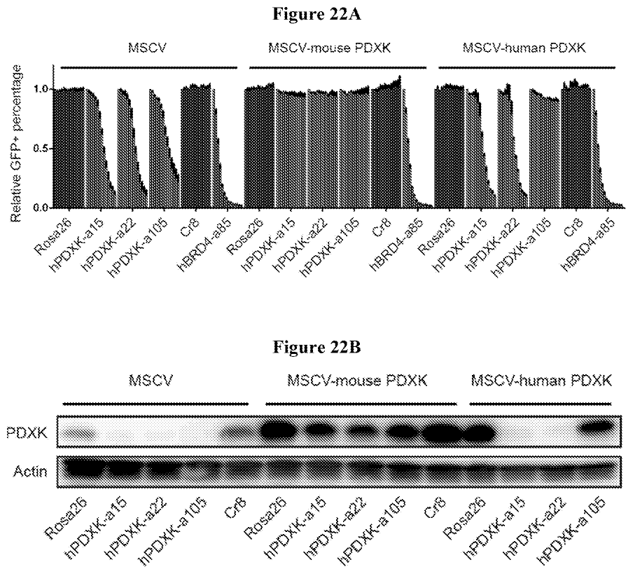

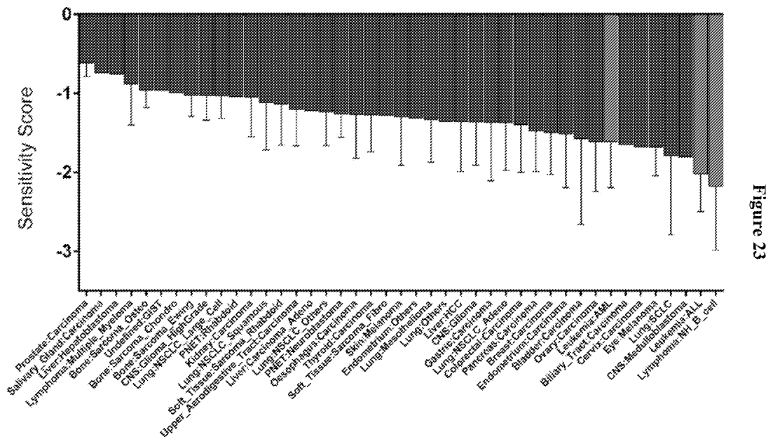

As shown in FIGS. 21A-21F, PDXK is also required for the proliferation of five human AML cell lines that harbor genetic abnormalities, including Kras/MLL-AF6, FLT3-ITD/MLL-AF9, Nras/MLL-AF9, and MLL-AF4. Further, mouse PDXK cDNA completely rescued the decreased leukemic cell proliferation phenotype that was induced by three independent human PDXK sgRNAs, while human PDXK cDNA only specifically rescued the effects of the human PDXK sgRNA that targeted a PDXK intron-exon junction. Human PDXK cDNA failed to rescue the effects of two other sgRNAs that targeted PDXK exons. See FIGS. 22A-22B. These results are consistent with previous reports that identified PDXK as a cancer cell dependency in multiple hematopoietic malignancies, including AML, acute lymphoblastic leukemia (ALL), and lymphoma. See FIG. 23.

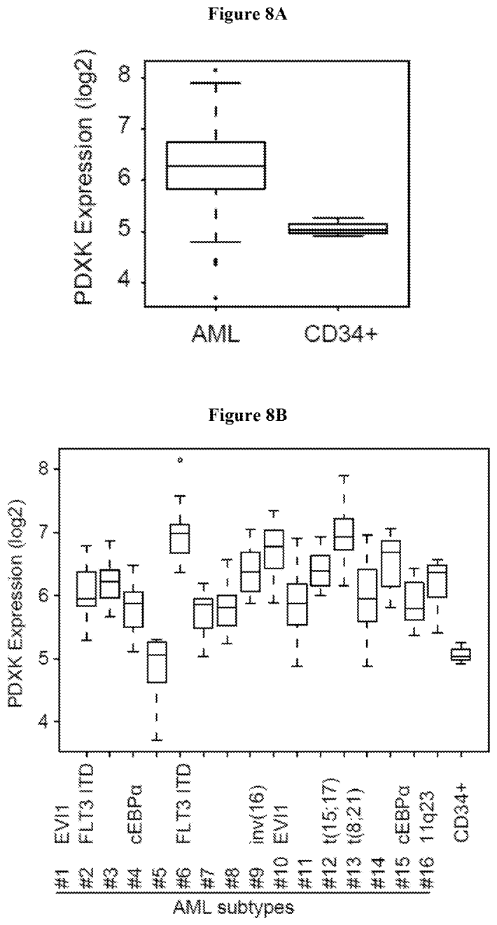

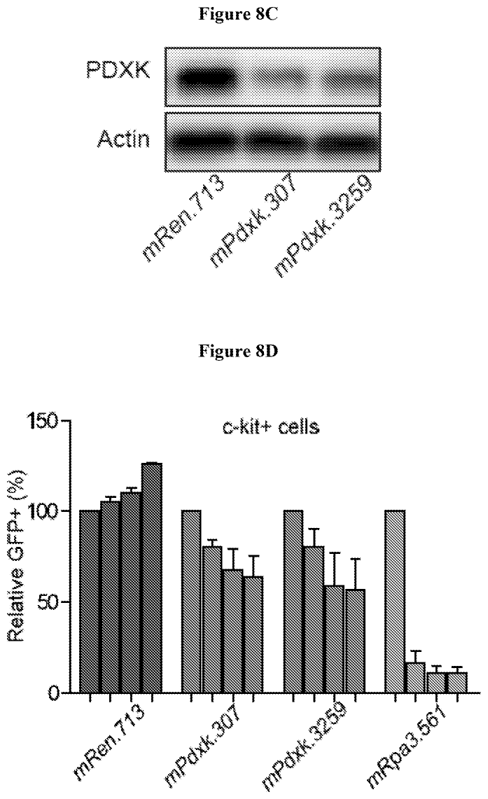

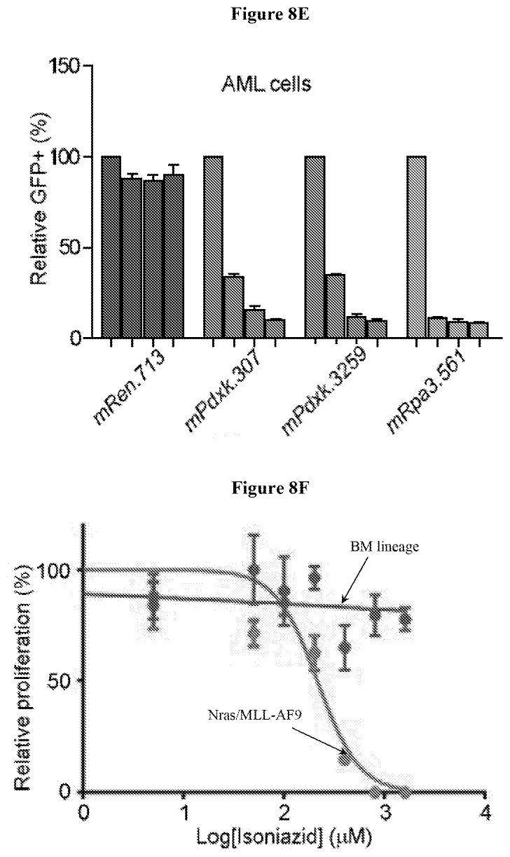

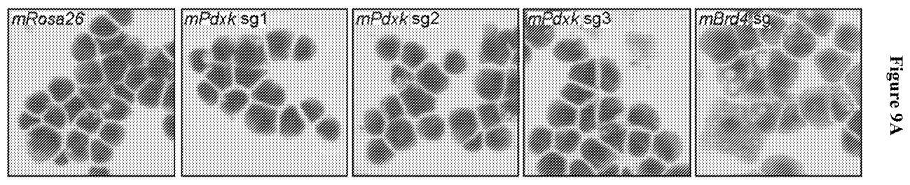

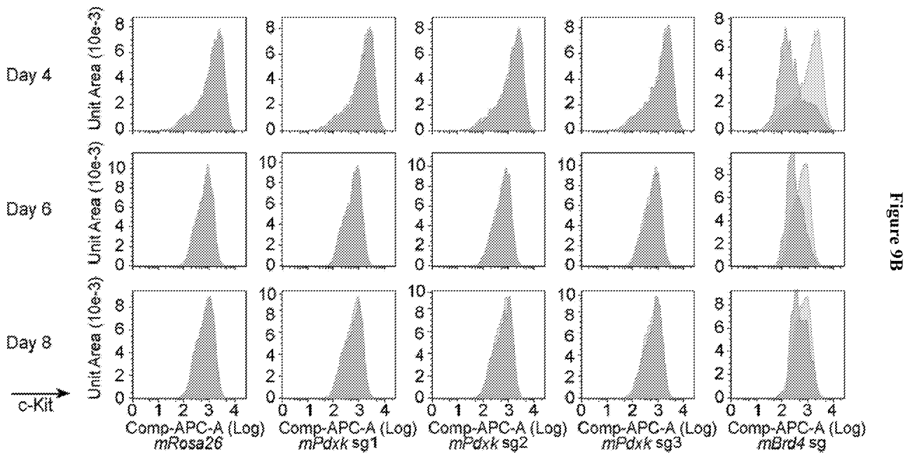

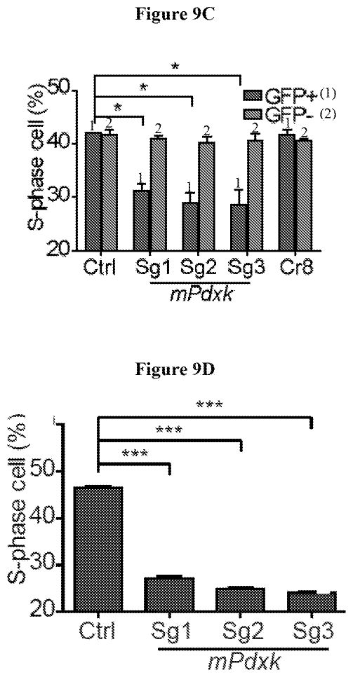

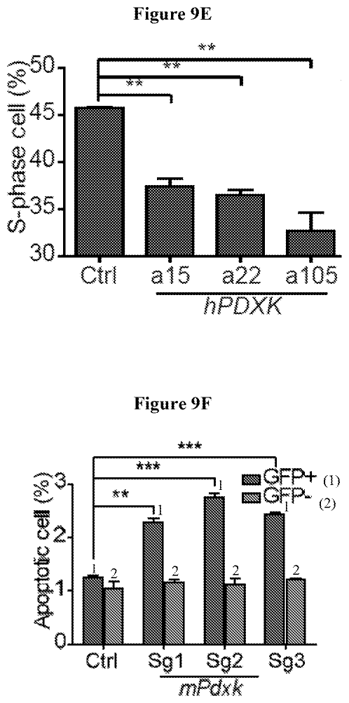

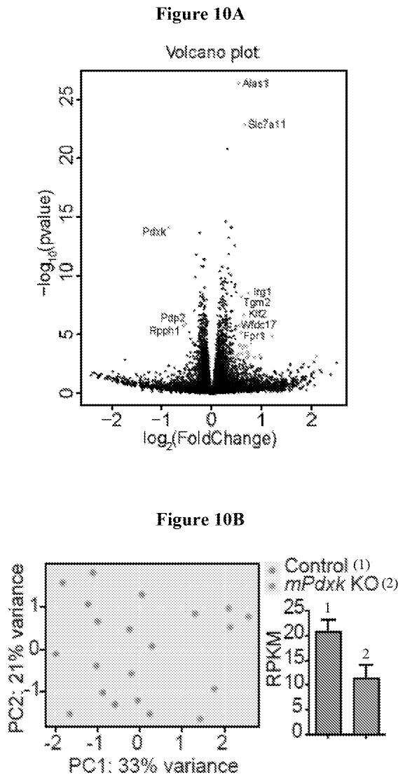

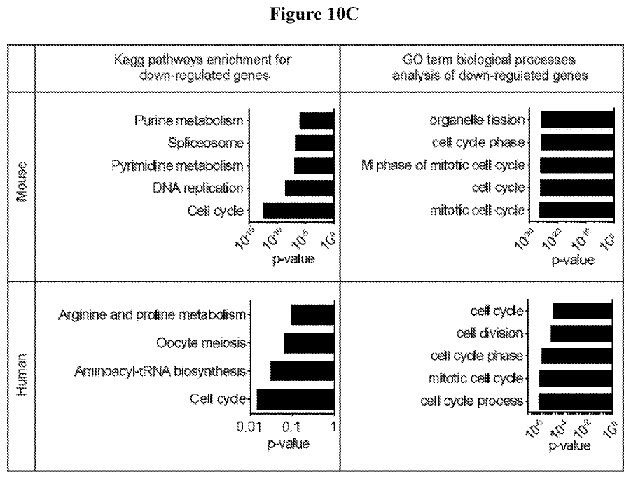

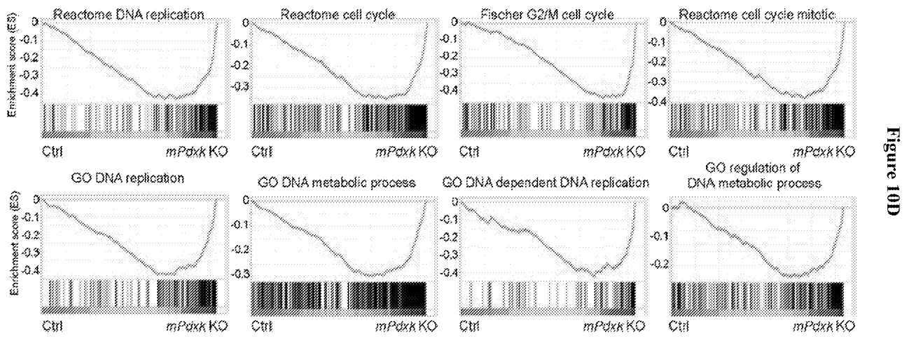

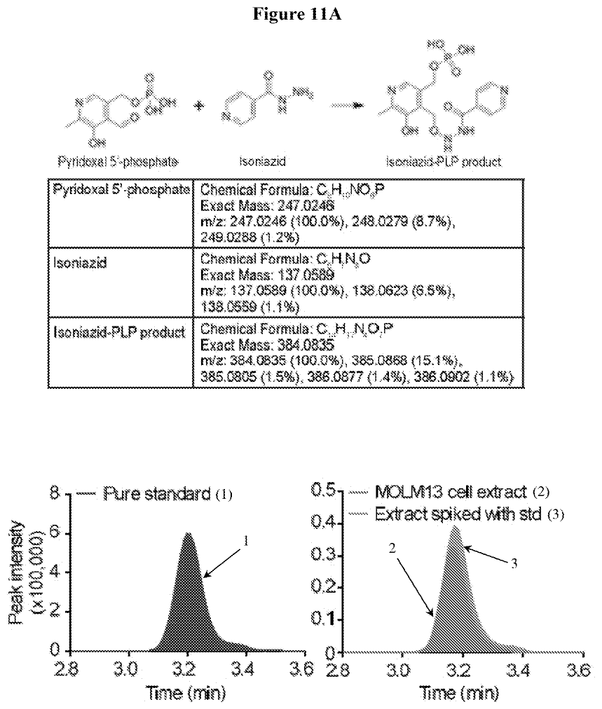

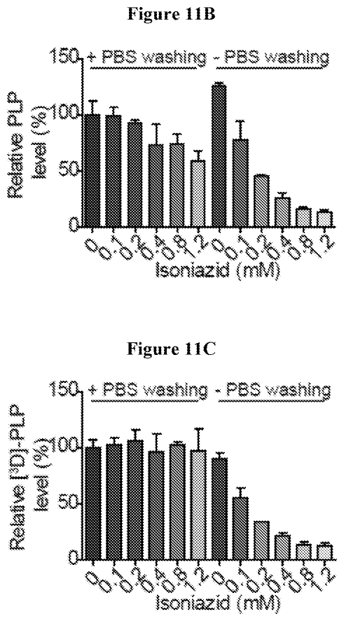

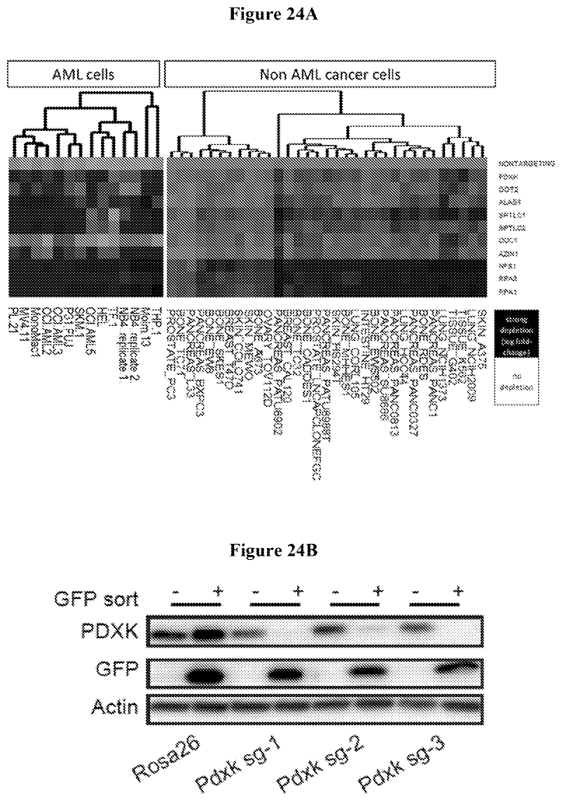

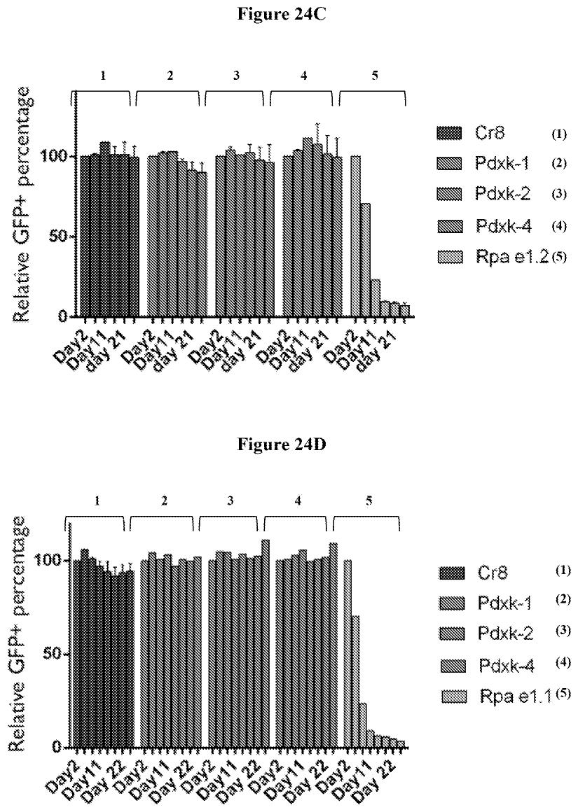

By contrast, Pdxk depletion had no influence on the proliferation of several immortalized murine and human cell lines (immortalized mouse embryonic fibroblasts (iMEFs), 3T3 cells, and sarcoma cells) (FIGS. 1D-E and FIGS. 6D-F). Consistent with the observation that Pdxk is highly expressed in leukemic cells but not normal hematopoietic stem and progenitor cells (HSPCs) (FIGS. 8A and 8B), depletion of Pdxk had only subtle effects on bone marrow HSPCs in in vitro competition assays (FIGS. 8C-8E). In addition, depletion of PDXK had no influence on the proliferation of RagMEF and 3T3 cell lines (FIGS. 24B-24D). Thus, normal HSPCs are not as dependent on the vitamin B6 pathway for the purpose of cell proliferation. Collectively, these results establish PDXK as a metabolic vulnerability in AML. Of note, PDXK depletion did not trigger leukemia cell differentiation but instead reduced cell cycle progression and produced a slight increase in apoptosis (FIGS. 9A-9F). Accordingly, gene set enrichment analysis of RNA-seq data obtained following PDXK sgRNAs revealed a significant downregulation of genes associated with “cell cycle progression”, “DNA replication”, and “nucleotide metabolism” (FIGS. 10A-10D). PDXK is not universally required for cellular proliferation. In comparison to DNA replication protein RPA1 and RPA3, which are broadly required for proliferation, PDXK is selectively required for the proliferation of leukemic cell lines (FIGS. 24A-24D).

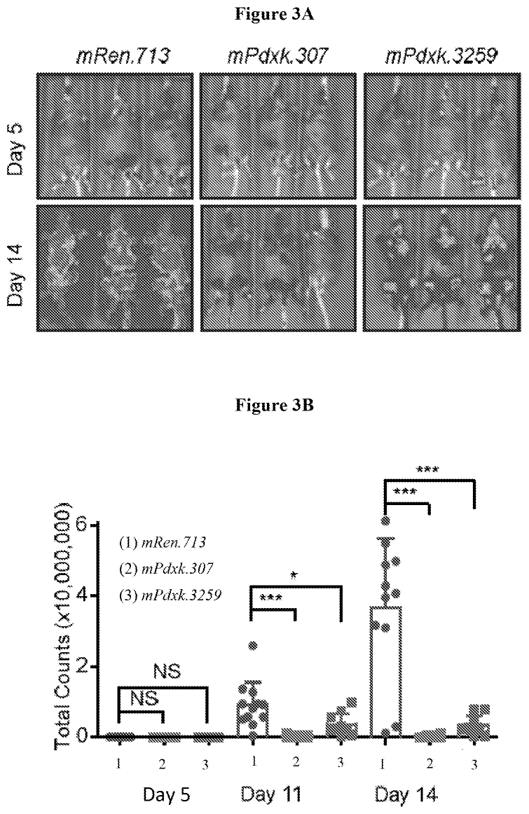

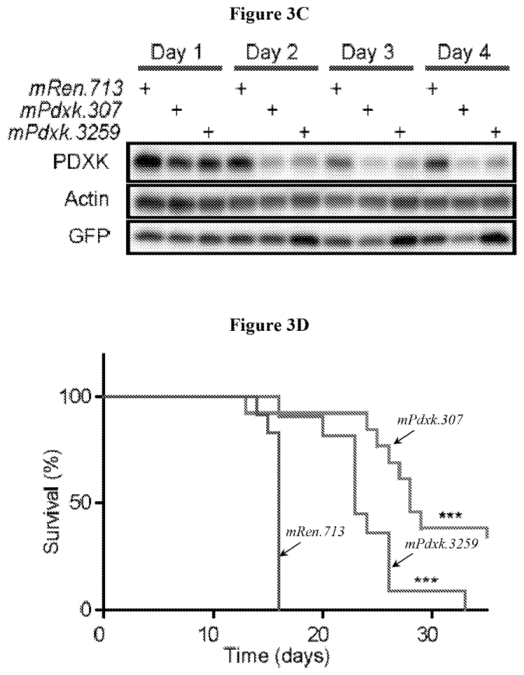

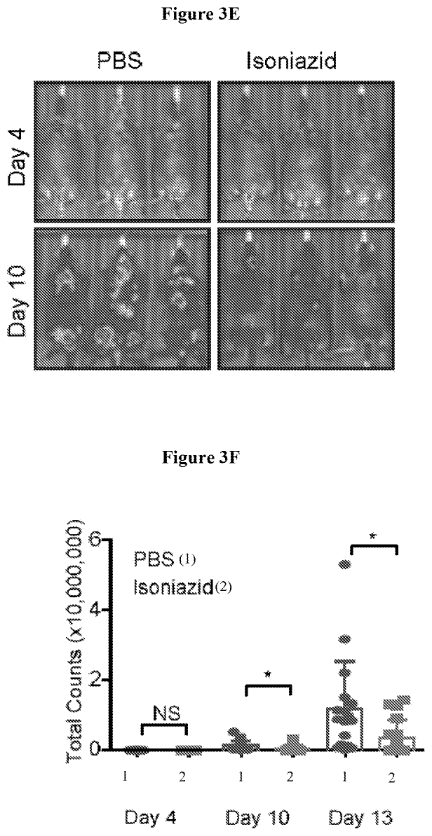

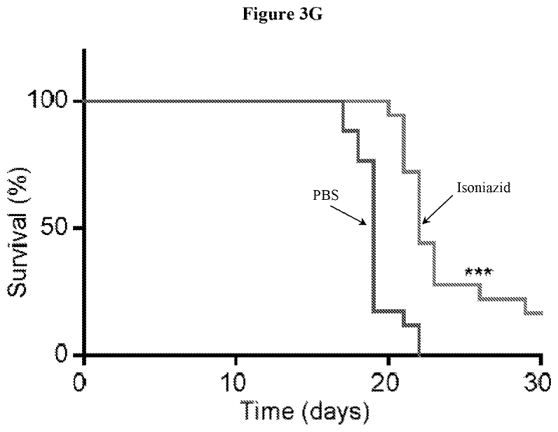

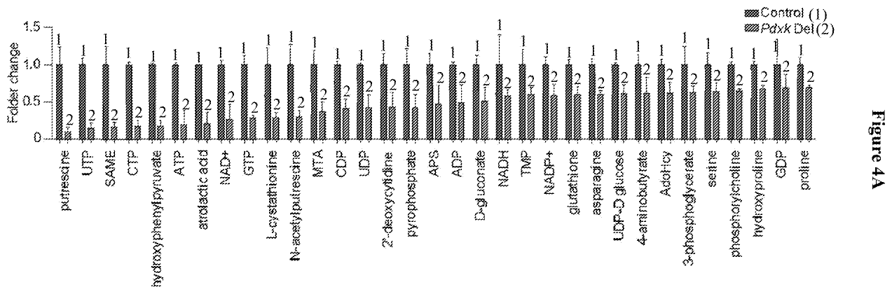

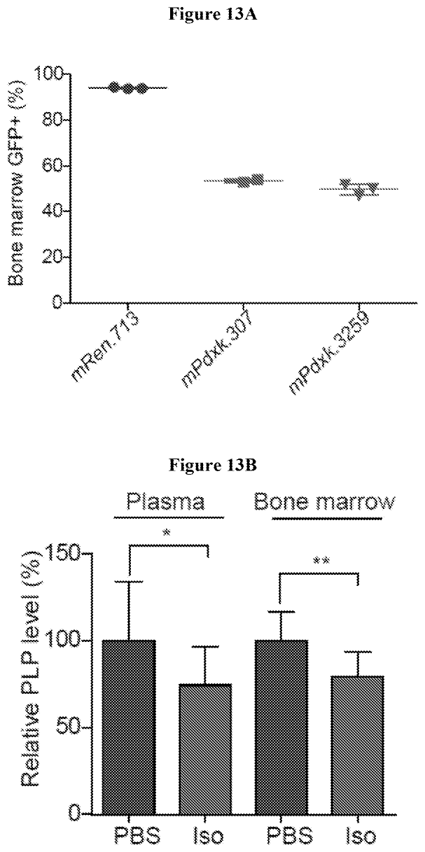

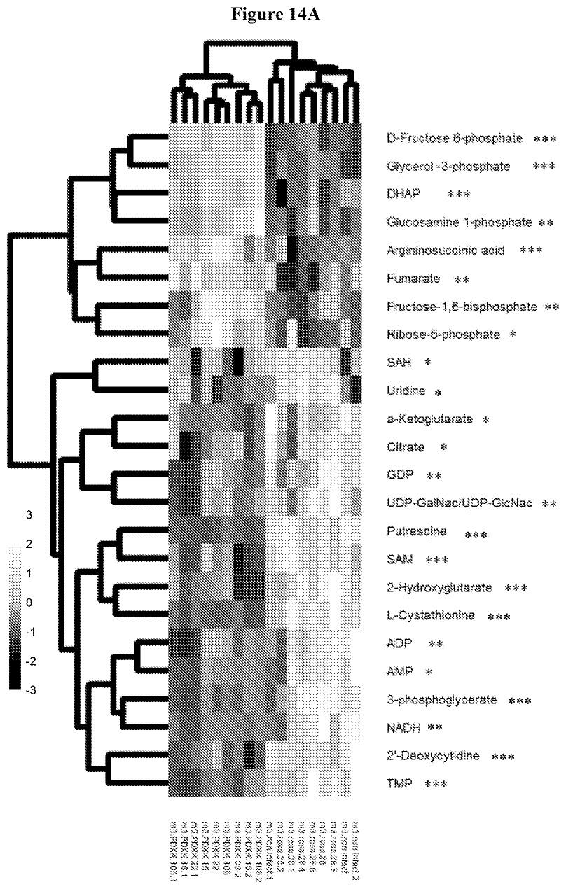

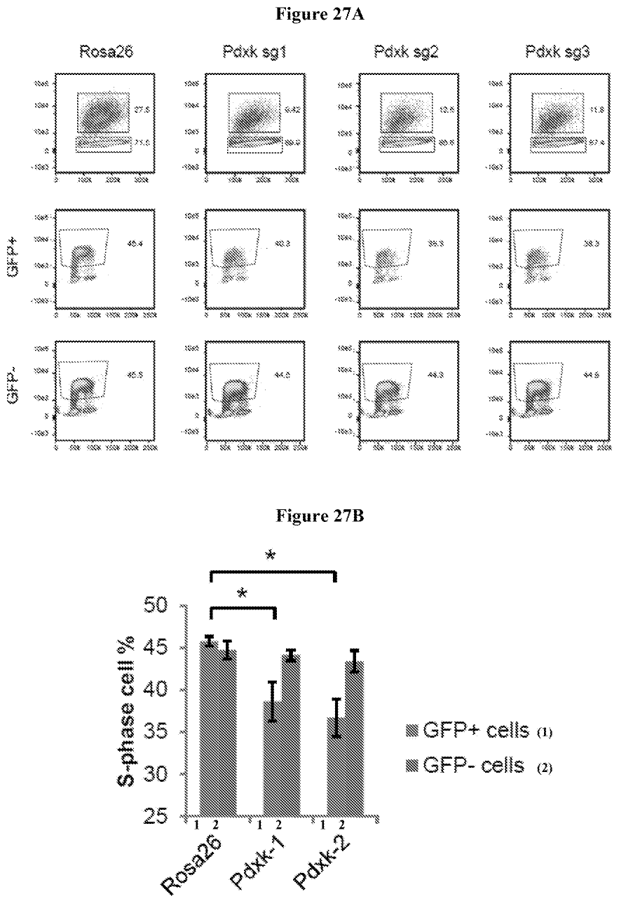

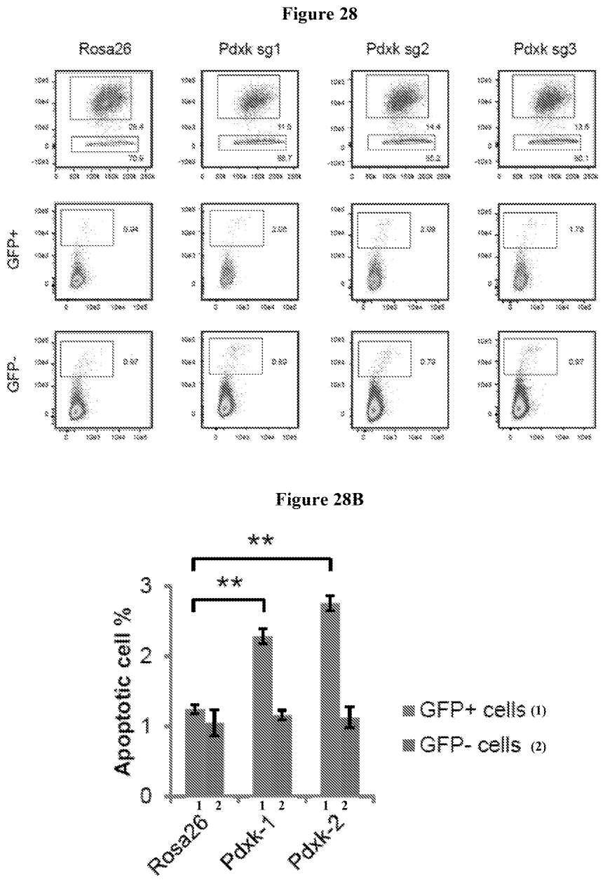

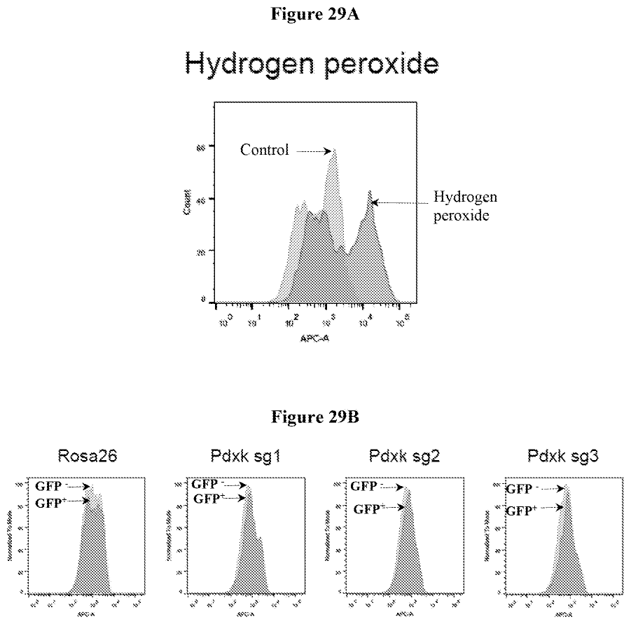

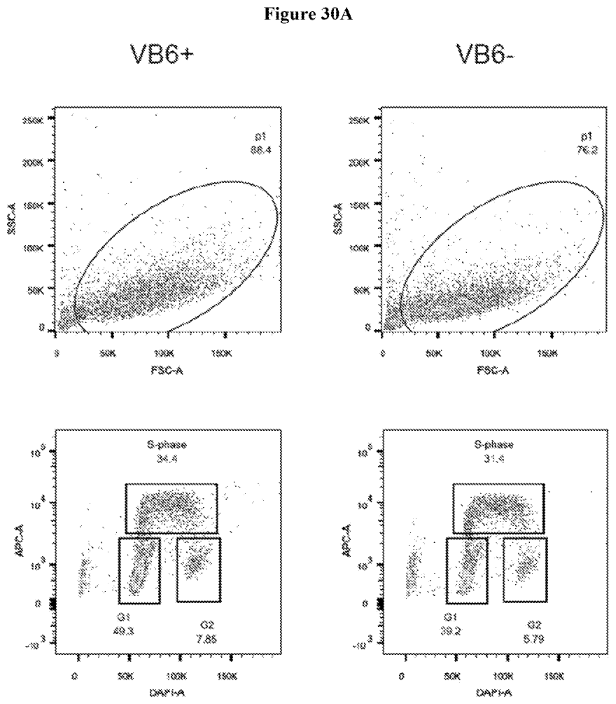

To test the in vivo significance of PDXK for leukemia disease progression, the consequences of suppressing PDXK in established leukemia in vivo was evaluated. A previously generated AML mouse model with a reverse tetracycline transactivator (rtTA) and a tetracycline-responsive element (TRE) promoter, where shRNA expression is induced by doxycycline was used (Zuber et al., Nat Biotech 29:79-83 (2011)). In this mouse model, Nras(G12D) was co-expressed with luciferase to allow for monitoring of disease progression with bioluminescence imaging. These Nras(G12D)/MLL-AF9 leukemic cells were transduced with viruses encoding shRNAs targeting control Renilla luciferase or Pdxk, and were subsequently transplanted into sub-lethally irradiated recipient mice (See e.g., Zuber et al., Genes Dev 23:877-889 (2009)). Induction of Pdxk shRNAs by doxycyline treatment significantly delayed disease progression and extended overall animal survival (FIGS. 3A to 3D). Consistent with the decrease of luciferase intensity (FIG. 3A to 3C), the percentage of GFP+ cells in bone marrow was decreased (FIG. 13A) and the knockdown of PDXK was also reduced in these GFP+ cells (data not shown), demonstrating that leukemia cells escaping PDXK knockdown or lacking shRNA expression are responsible for progressive disease. In contrast, loss of Pdxk function resulted in cell cycle delay and induced apoptosis (FIGS. 27A-27B and FIGS. 28A-28B). Further, FIGS. 29A-29B show that depletion of Pdxk activity had no effect on redox status. Taken together, these results demonstrate that elimination of PDXK activity inhibits cell cycle progression and promotes apoptosis. While not wishing to be bound by theory, it is believed that PDXK inhibitors block leukemic cell growth by inhibiting cell cycle progression and inducing apoptosis.

In addition, depletion of PDXK had no influence on the proliferation of RagMEF and 3T3 cell lines (FIGS. 24B-24D). Thus, normal HSPCs are not as dependent on the vitamin B6 pathway for the purpose of cell proliferation. Collectively, these results establish PDXK as a metabolic vulnerability in AML.

These results demonstrate that PDXK is selectively required for the proliferation of leukemic cells and that inhibitors of PDXK can effectively inhibit leukemic cell proliferation both in vitro and in vivo. Accordingly, the vitamin B6 pathway inhibitors disclosed herein are useful in methods for inhibiting leukemic cell proliferation and treating AML in a subject in need thereof.

### Example 3: PDXK Catalyzes PLP Formation in Leukemic Cells

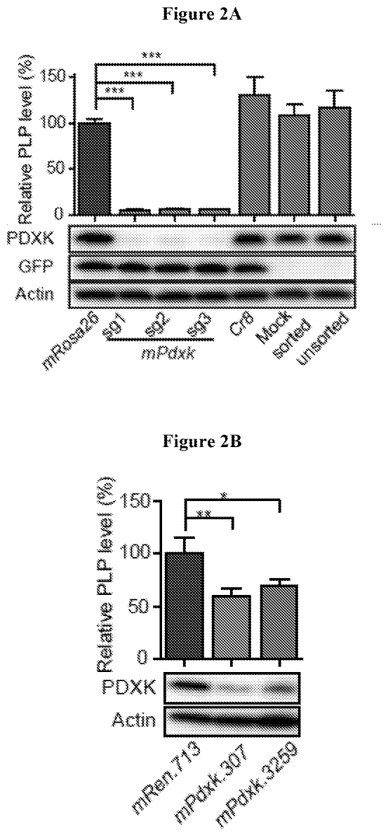

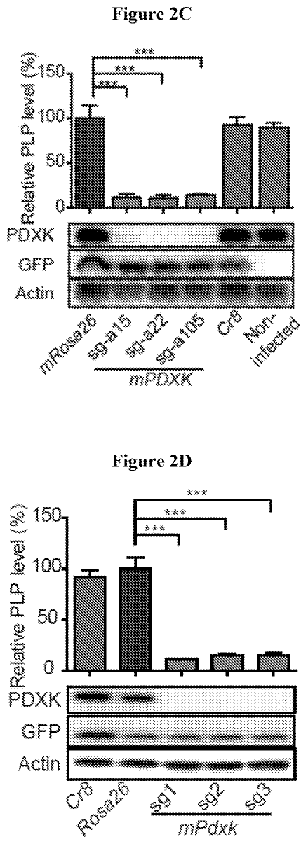

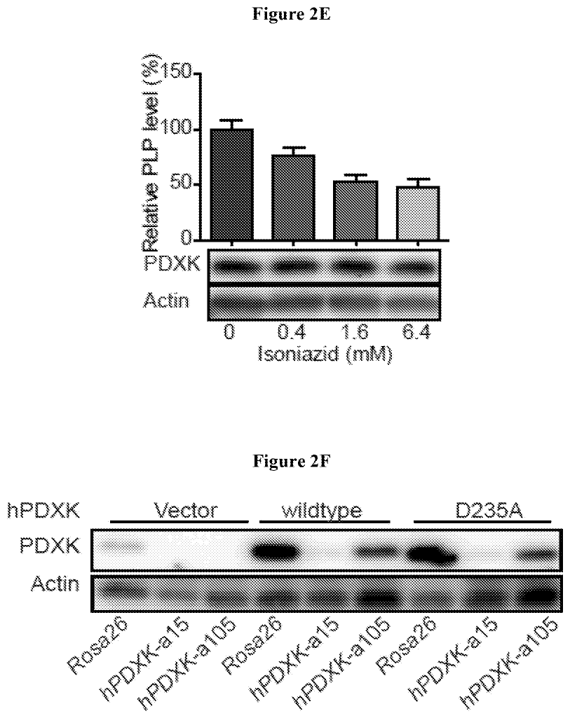

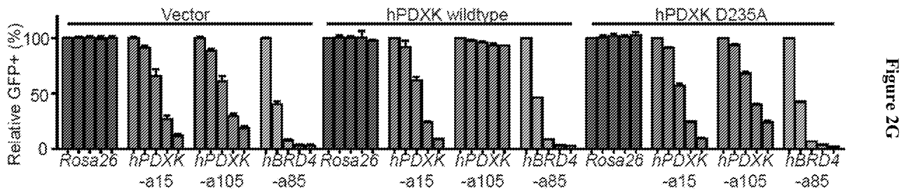

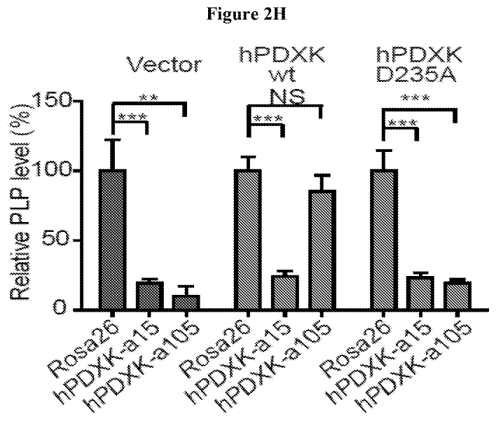

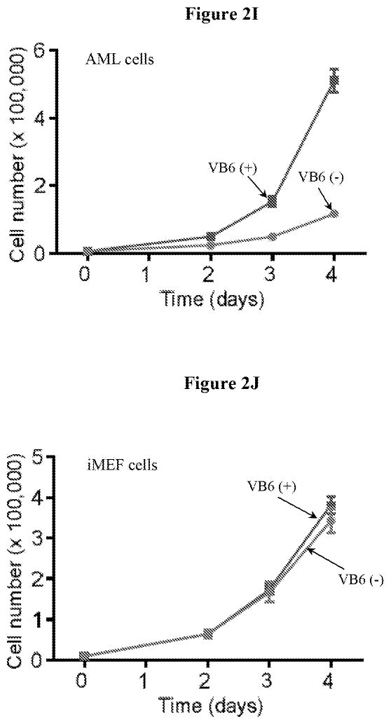

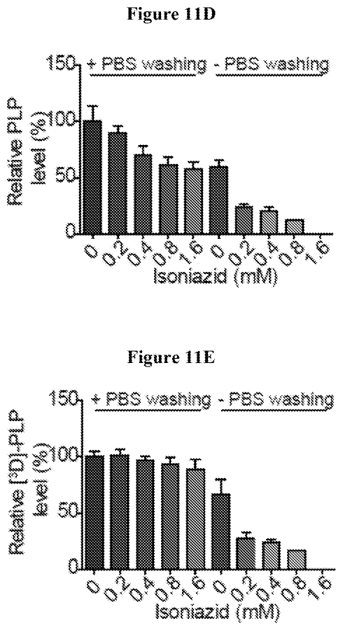

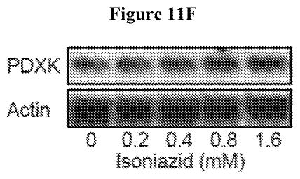

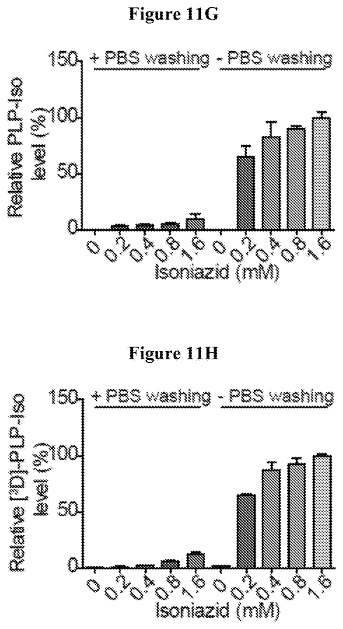

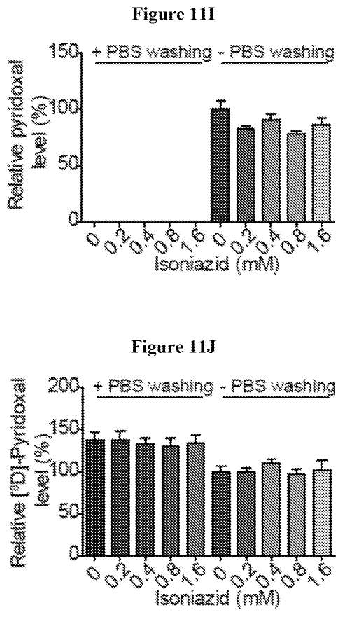

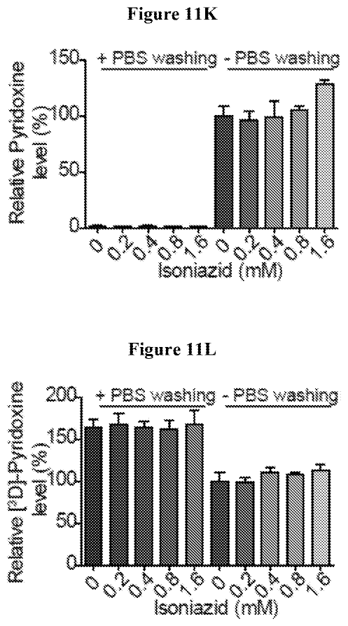

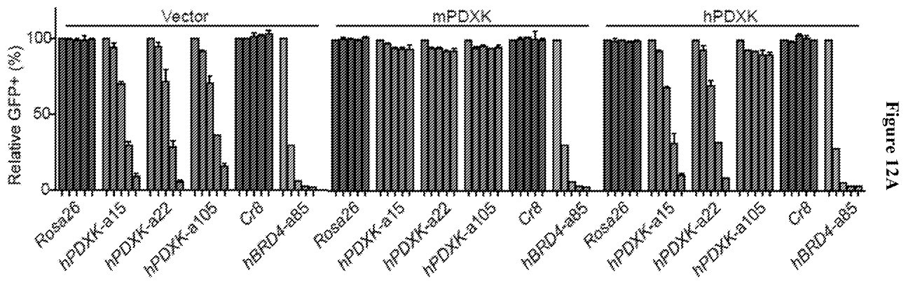

The PDXK product PLP is a cofactor for multiple enzymes involved in amino acid, nucleic acid, and lipid metabolism. Although plasma PLP levels can be readily measured, no reliable assays currently detect intracellular PLP. To determine whether genetic blockage of PDXK signaling affected PLP levels in leukemia cells, a high-performance liquid chromatography-mass spectrometry (HPLC-MS) based method was developed to compare intracellular PLP levels in leukemic cells in the presence or absence of PDXK inhibition (FIGS. 11A-11L). As anticipated, genetic inhibition of PDXK dramatically decreased PLP levels in mouse and human leukemic cells (FIGS. 2A to 2C and FIGS. 11A-11L). iMEFs displayed a similar reduction in PLP levels, despite their limited sensitivity to PDXK disruption (FIG. 2D).

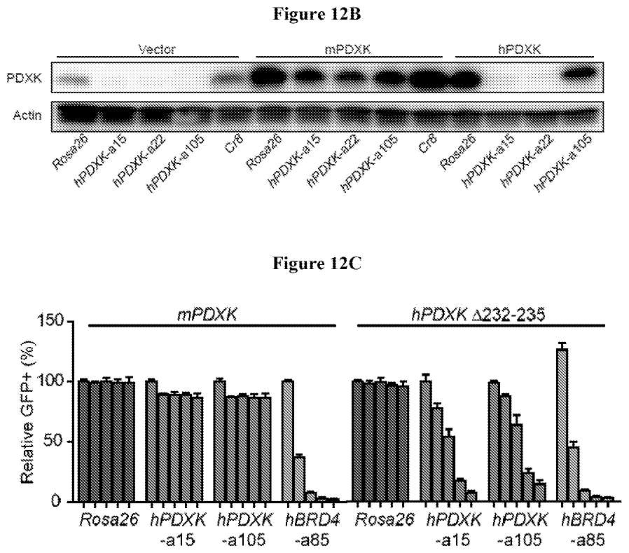

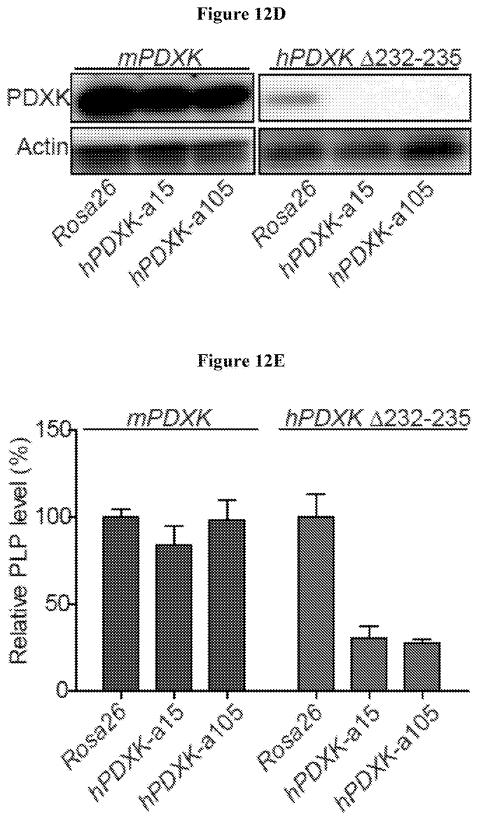

The availability of a pharmacodynamic marker of PDXK inhibition permits correlation between the leukemia selective dependence of PDXK and its role in PLP production and vitamin B6 metabolism. Hence, re-expression of a wild-type PDXK cDNA but not a kinase dead PDXK mutant (D235A) was able to rescue the proliferative defects and PLP depletion produced by a PDXK sgRNA that targets the intron-exon junction of the human gene (FIGS. 12A-12E and FIGS. 2F to 2H). Thus, the kinase function of PDXK is necessary for AML proliferation. Furthermore, depletion of pyridoxine, the absorbable form of vitamin B6, suppressed proliferation of AML cells in culture (FIG. 2I), but had no influence on the proliferation of immortalized fibroblasts (FIG. 2J). Of note, trace levels of PLP was detected in B6-deficient media with dialyzed serum used in these B6-depletion experiments (data not shown). However, these results demonstrate vitamin B6 conversion to PLP by PDXK is preferentially required for leukemic cell proliferation.

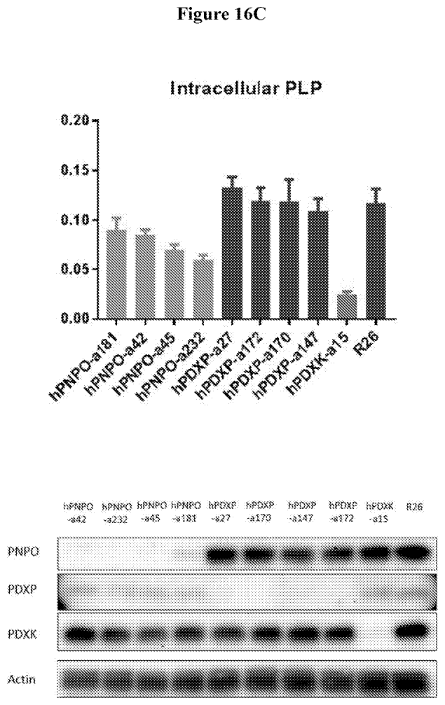

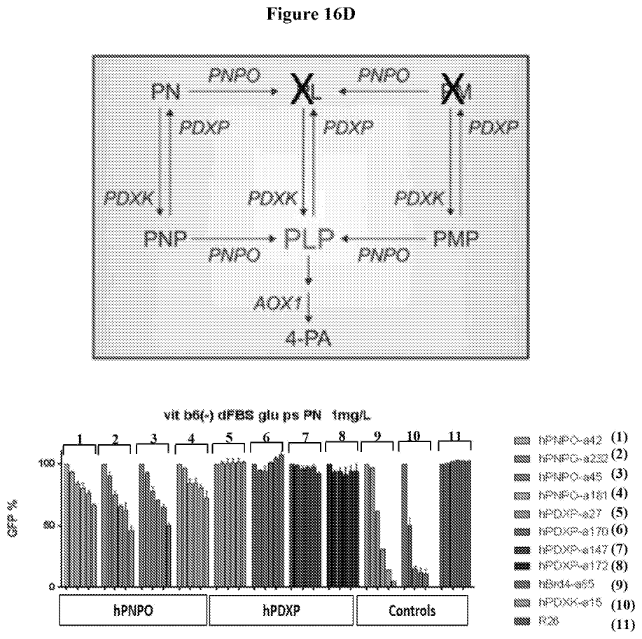

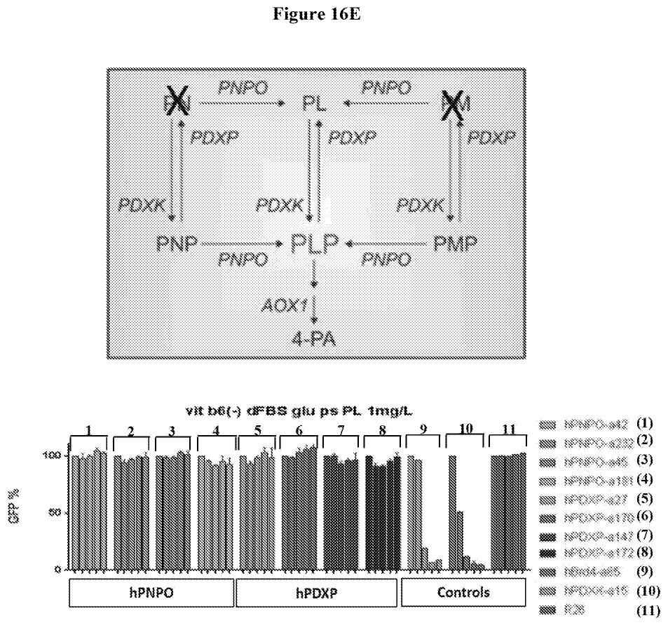

Inhibition of another PLP-production pathway (PNPO) partially inhibited growth inhibitory in AML cells, which demonstrates that cell growth is regulated by intracellular PLP levels. See FIGS. 16A-16E. The sequences of the PNPO sgRNAs are as follows:

hPNPO-a42:
(SEQ ID NO: 987)
5′ CATGCGCAAGAGTTACCGCG 3′;
hPNPO-a232
(SEQ ID NO: 988)
5′ ATGACCGGATAGTCTTTCGG 3′;
hPNPO-a45
(SEQ ID NO: 989)
5′ GAGTTACCGCGGGGACCGAG 3′;
and
hPNPO-a181
(SEQ ID NO: 990)
5′ TTCTGTGATCCCTGATCGGG 3′.

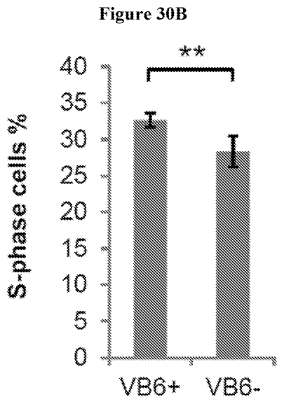

In addition, since unphosphorylated vitamin B6 species (PN, PL, and PM) but not the phosphorylated forms (PNP, PLP, PMP) can cross cell membrane, these results also demonstrated that PDXK is the only enzyme that is both necessary and sufficient to sustain the intracellular level of PLP in AML cells. Consistent with these findings, genome wide-CRISPR screens indicate that, in contrast to PDXK, pyridoxamine phosphate oxidase (PNPO) and pyridoxal phosphatase (PDXP) are not required for AML cell proliferation (FIG. 6C). These findings are also consistent with the results of FIGS. 30A-30B, which demonstrate that depletion of vitamin B6 in culture medium delays cell cycle progression in leukemic cells.

Taken together, these results demonstrate that PDXK catalyzes PLP formation in leukemic cells and that PLP is selectively required for leukemic cell proliferation. Accordingly, the vitamin B6 pathway inhibitors disclosed herein are useful in methods for inhibiting leukemic cell proliferation and treating AML in a subject in need thereof.

### Example 4: Pharmacological Inhibition of PDXK/Vitamin B6 Pathway Reduces AML Cell Proliferation

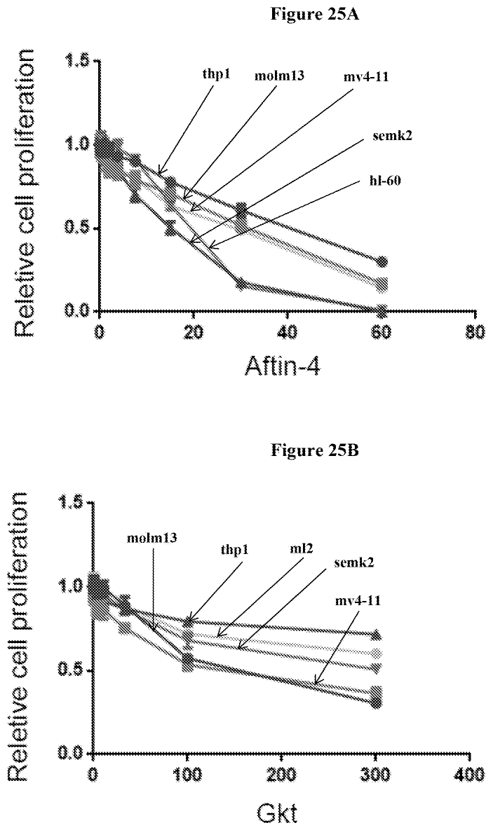

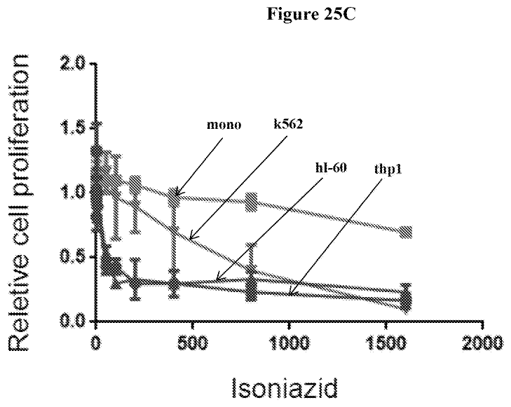

Isoniazid inhibits TB by suppressing the activity of enoyl-acyl carrier protein reductase, thereby blocking mycolic acids and mycobacterial cell wall synthesis. An inadvertent off-target effect of isoniazid results from its ability to bind and sequester PLP, thereby preventing it from regulating its downstream enzymes and producing symptoms of vitamin B6 deficiency in some patients (Laine-Cessac et al., Biochem Pharmacol 54:863-870 (1997)). Indeed, isoniazid reduced intracellular PLP levels, albeit to a lesser extent than the genetic approaches (FIG. 2E). Nevertheless, similar to PDXK depletion, isoniazid blocked proliferation of mouse and human leukemic cells harboring different genetic drivers (FIG. 1I and FIG. 7G) while producing only a subtle reduction in the proliferation of bone marrow HSPCs (FIG. 8F). Therefore, targeting PDXK production of PLP represents a pharmacologically accessible strategy to inhibit leukemic cell proliferation. See also FIGS. 25A-25C.

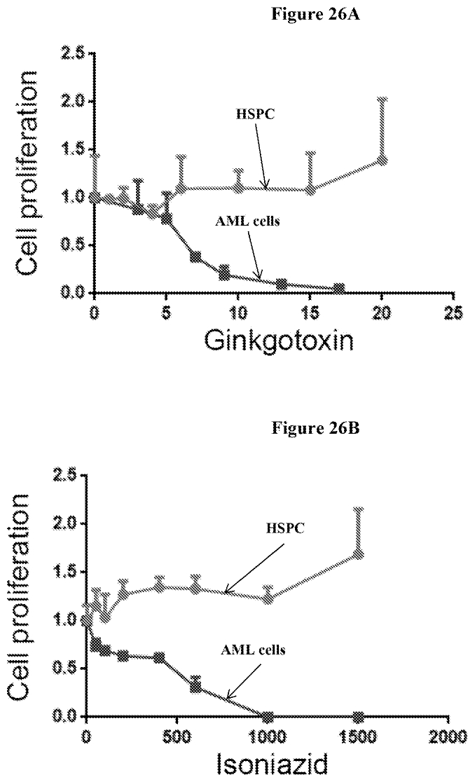

Similar effects on leukemic cell proliferation were observed when AML cells were treated with two other PDXK inhibitors, Aftin-4 and gingkotoxin (see FIGS. 25A-25B). Treatment with gingkotoxin reduced intracellular levels of PLP, suppressed tumor growth of human AML cells in vitro, and slowed down disease progression and improved survival in animals bearing AML cells. See FIGS. 17B-17F. As shown in FIGS. 18B-18C, treatment with aftin-4 also reduced intracellular levels of PLP, and inhibited the growth of human AML cells in vitro. In addition, PDXK inhibitors gingkotoxin and isoniazid had little effect on the proliferation of bone marrow lineage negative HSPCs (FIGS. 26A-26B). Thus, normal HSPCs are not as dependent on the vitamin B6 pathway for the purpose of cell proliferation.

The effects of pharmacological suppression of PLP on leukemic maintenance were assessed. Treatment of mice bearing Nras(G12D)/MLL-AF9 leukemia with isoniazid produced a ˜25% reduction of PLP in plasma and bone marrow cells (FIG. 13B), delayed disease progression and produced a survival advantage (FIGS. 3E-3G). Taken together, these results demonstrate that PDXK regulates leukemia disease progression in vivo.

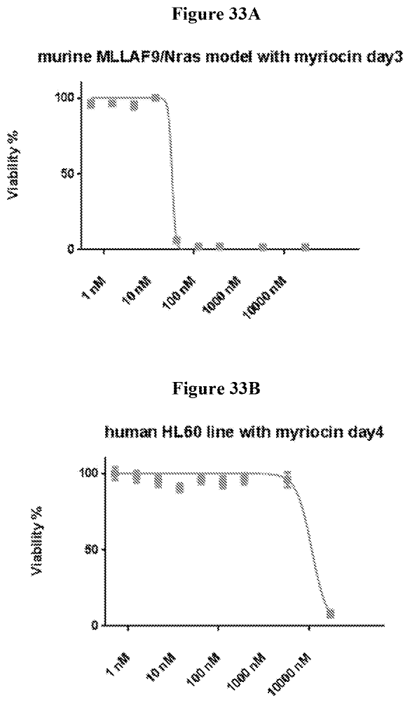

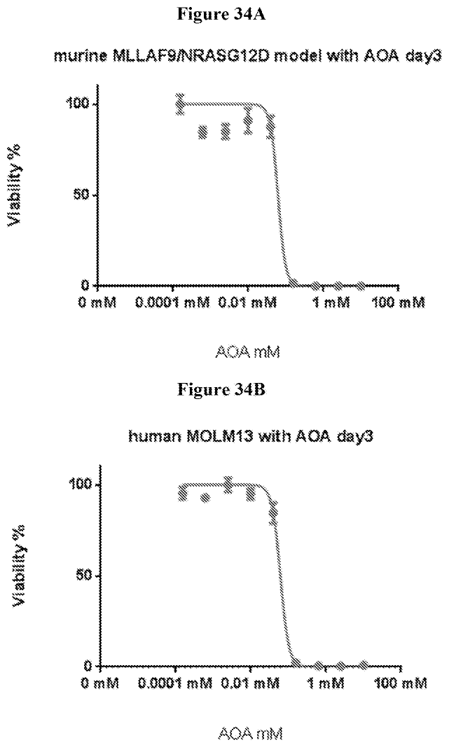

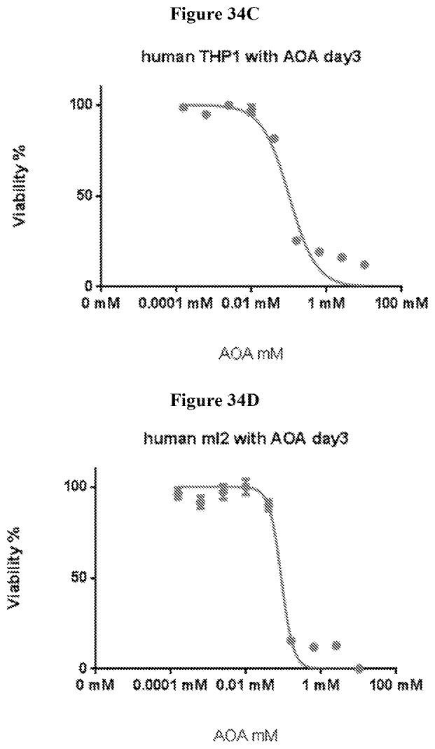

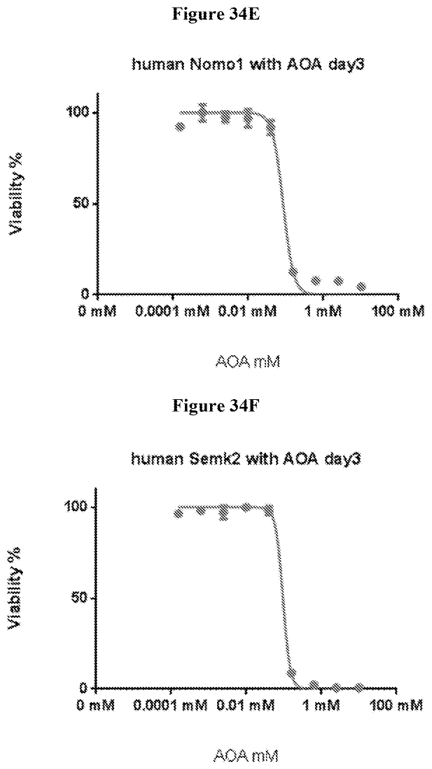

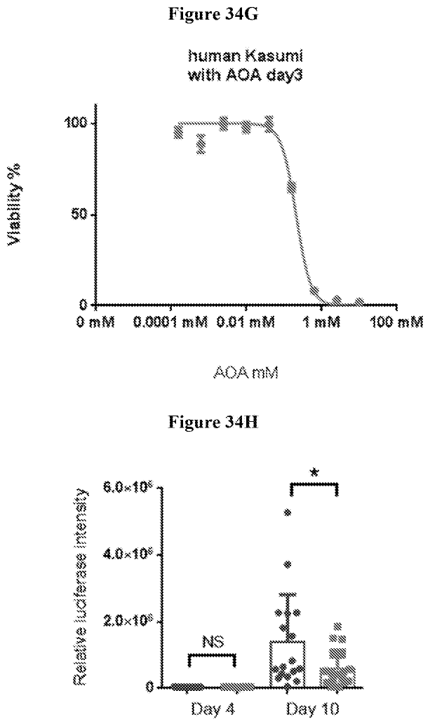

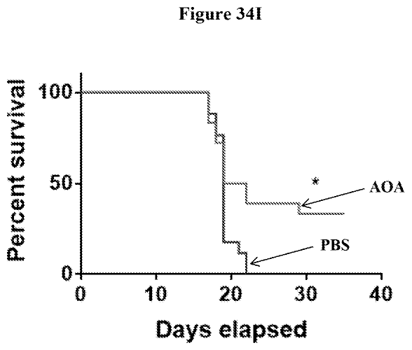

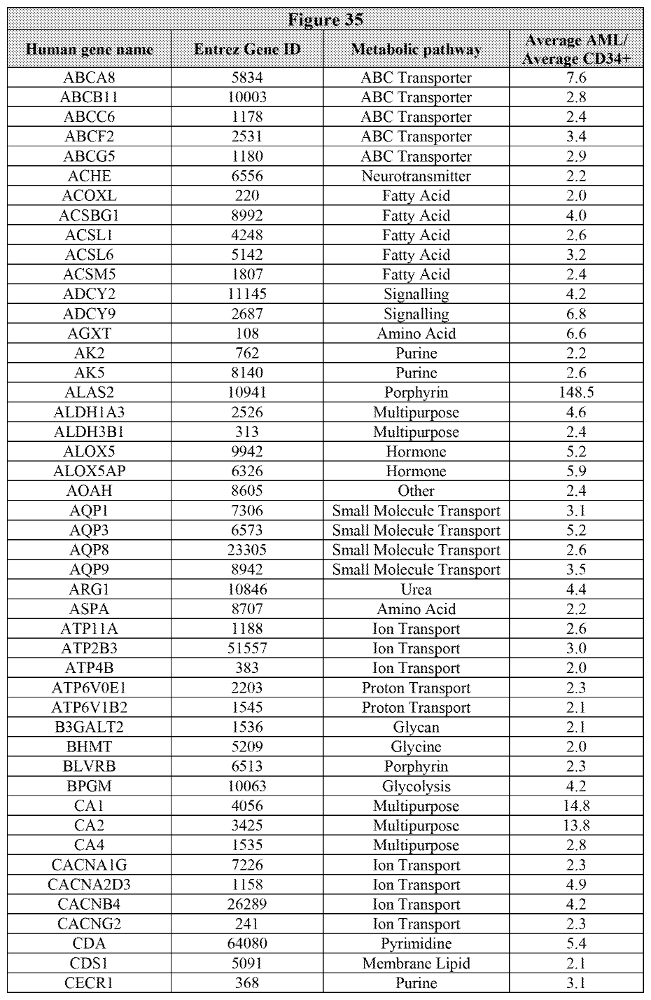

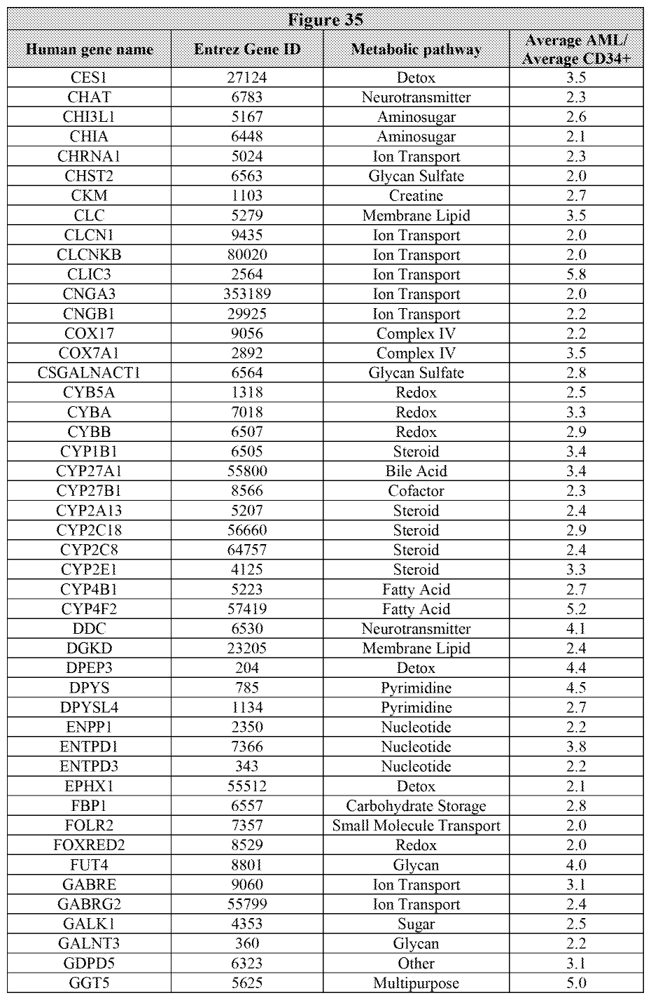

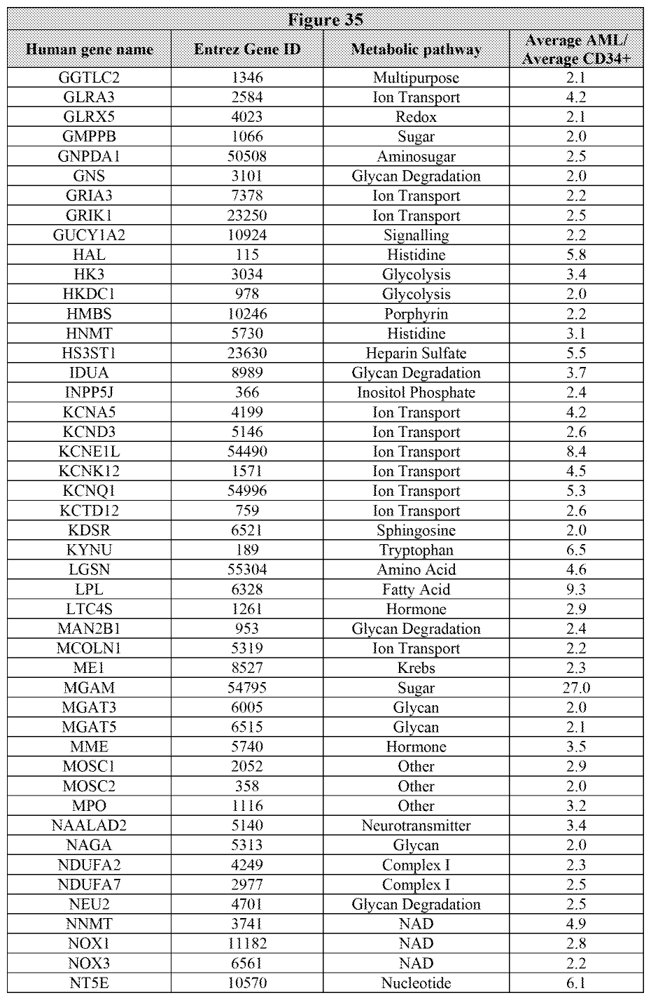

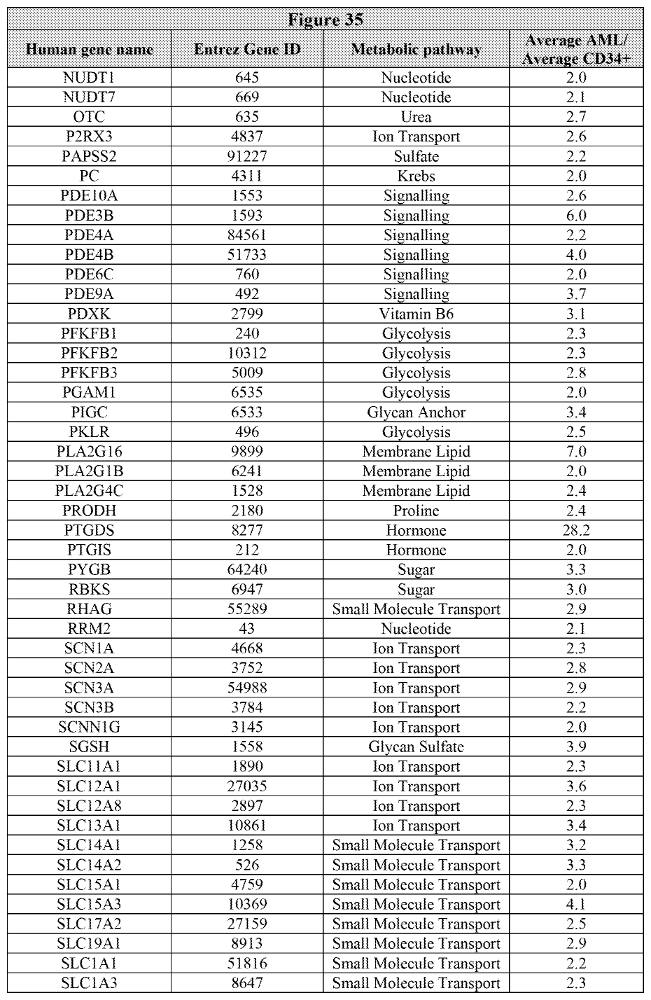

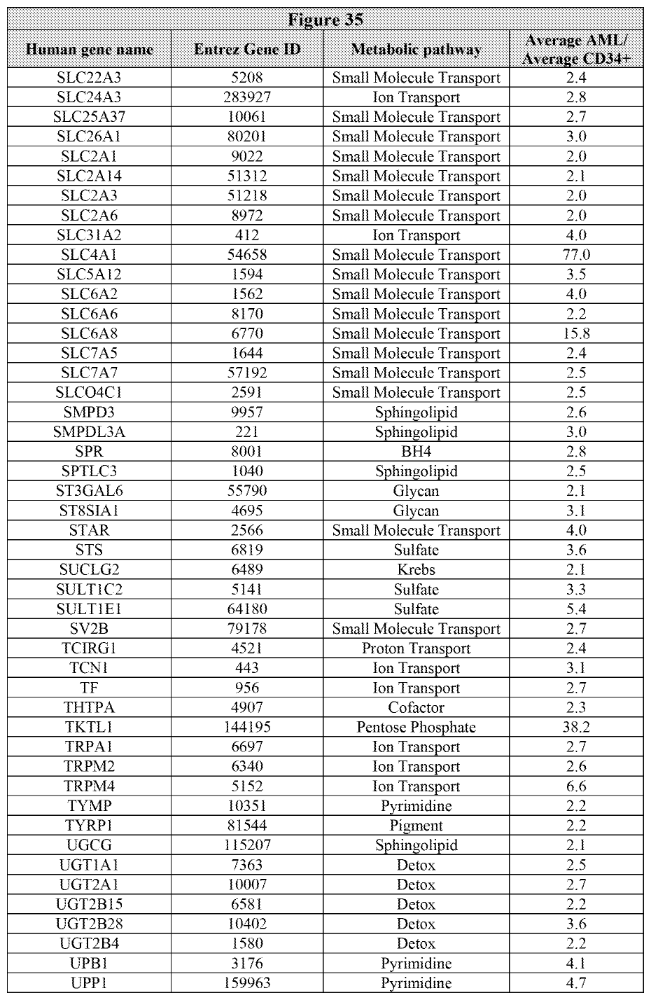

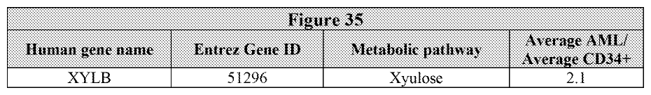

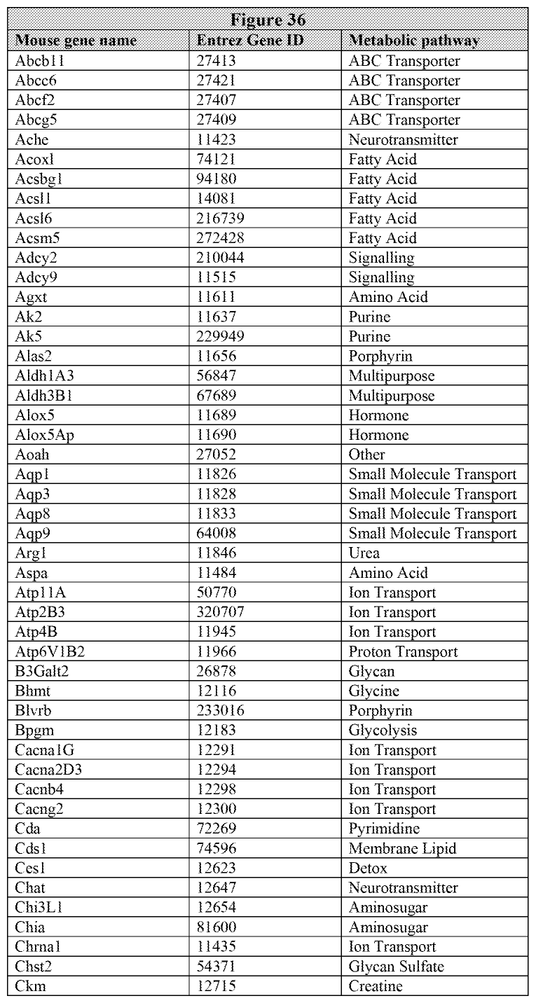

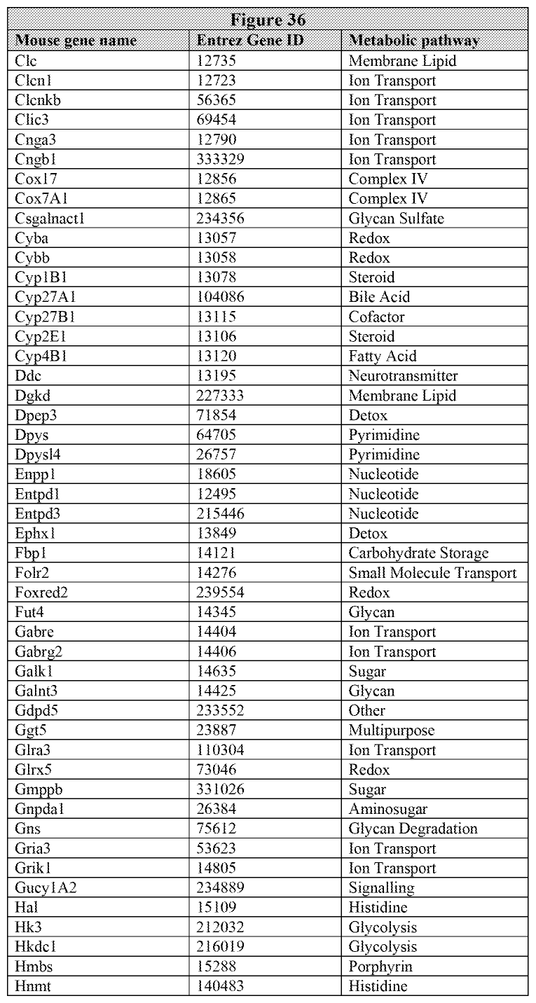

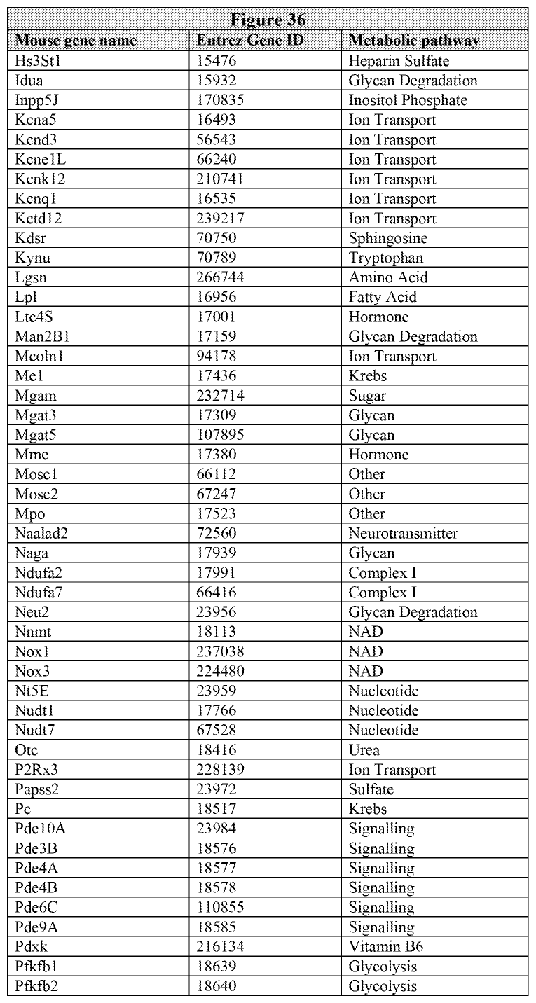

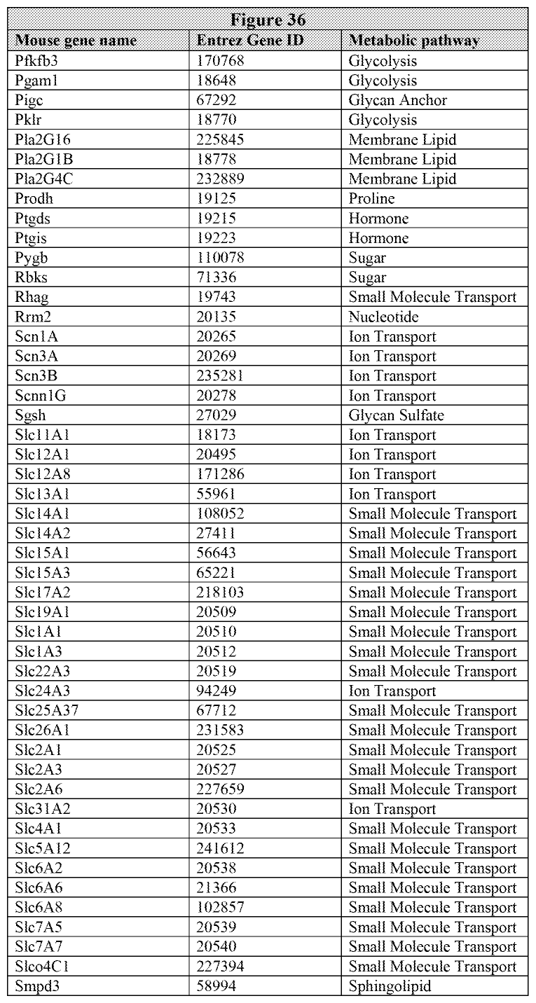

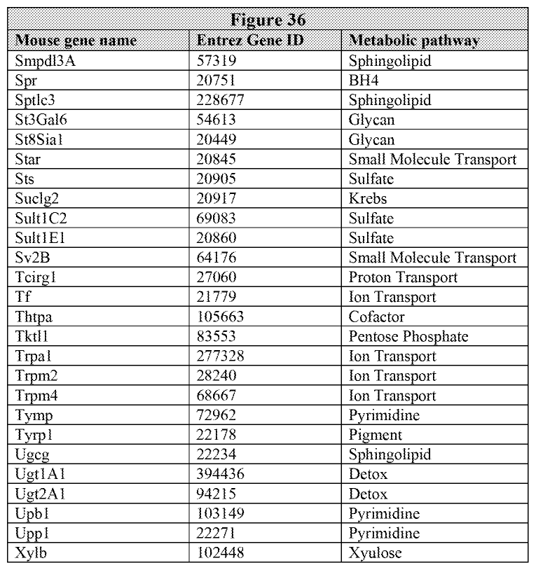

Further, FIGS. 33A and 34A-34G demonstrate that exposure to other pharmacological inhibitors of the vitamin B6 pathway including AOA and myriocin reduced the viability of leukemic cells in vitro. In contrast, human HL60 (Human promyelocytic leukemia) cells were less sensitive to treatment with myriocin (FIG. 33B). As shown in FIGS. 34H-341, administration of AOA significantly delayed leukemia disease maintenance in vivo and extended survival. Taken together, these results demonstrate that the vitamin B6 pathway regulates leukemia disease progression in vivo.

These results demonstrate that pharmacological inhibition of PDXK/vitamin B6 pathway can effectively inhibit leukemic cell proliferation, both in vitro and in vivo. Further, these results demonstrate that isoniazid may be used to treat AML in a subject without causing undesirable side effects, such as bone marrow suppression. Accordingly, the vitamin B6 pathway inhibitors disclosed herein are useful in methods for inhibiting leukemic cell proliferation and treating AML in a subject in need thereof.

### Example 5: Identification of PLP-Dependent Enzymes that are Essential for Leukemic Cell Proliferation

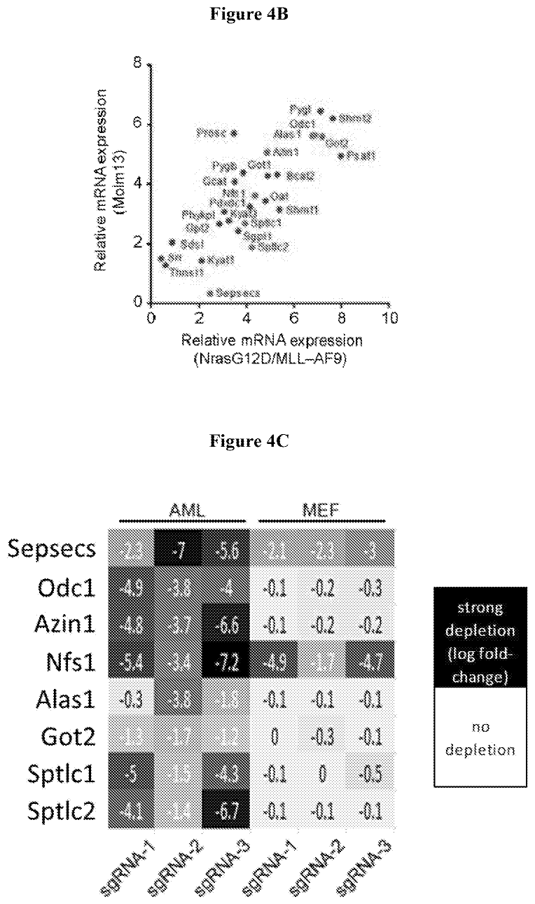

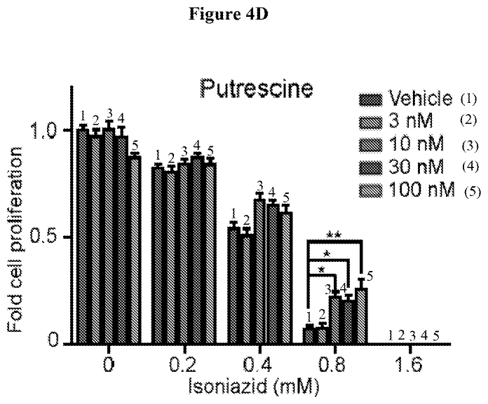

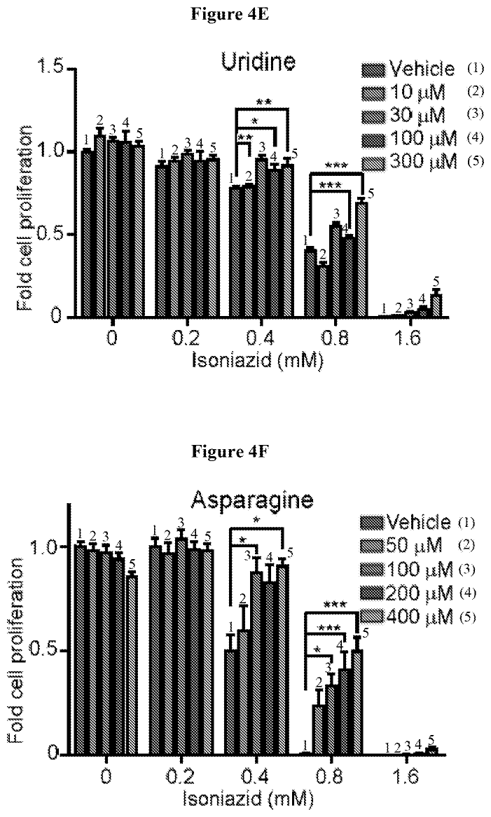

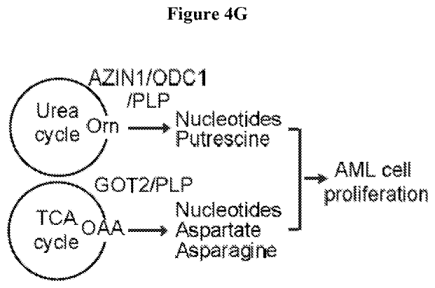

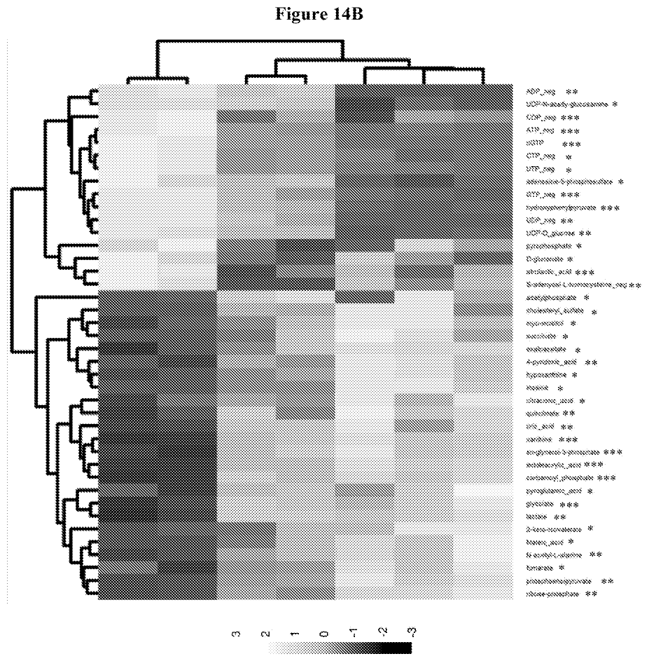

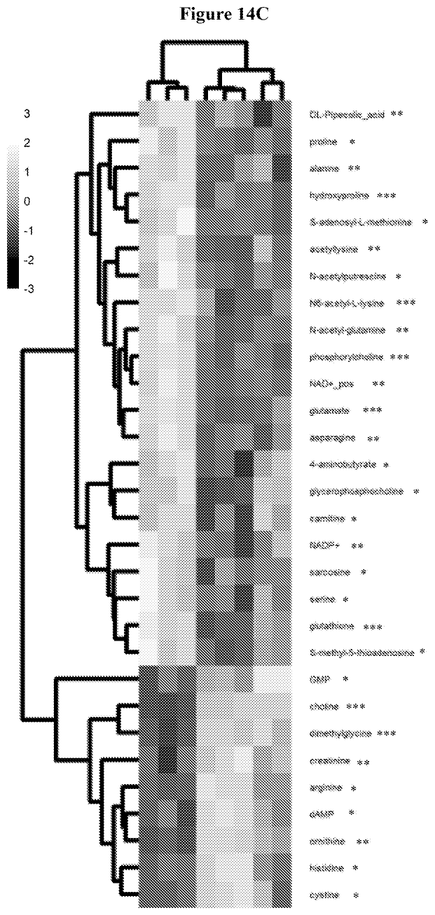

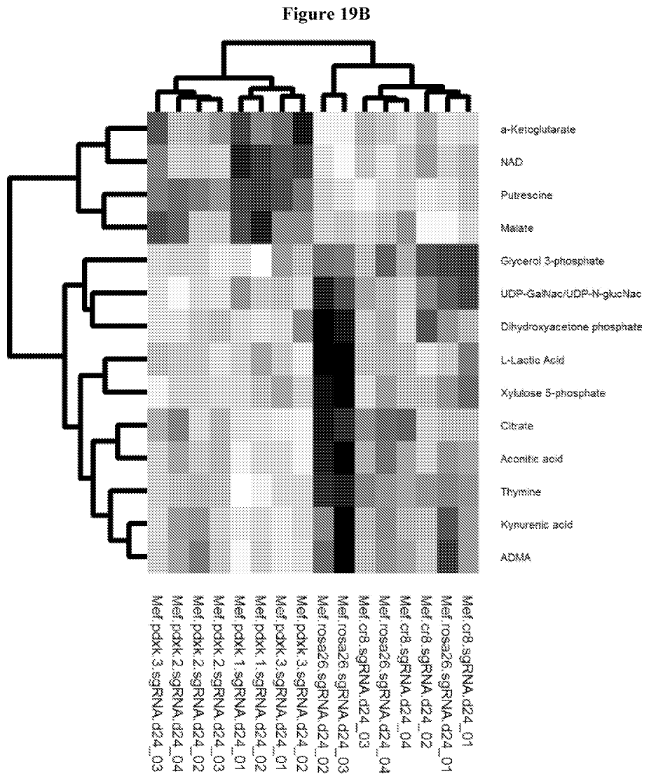

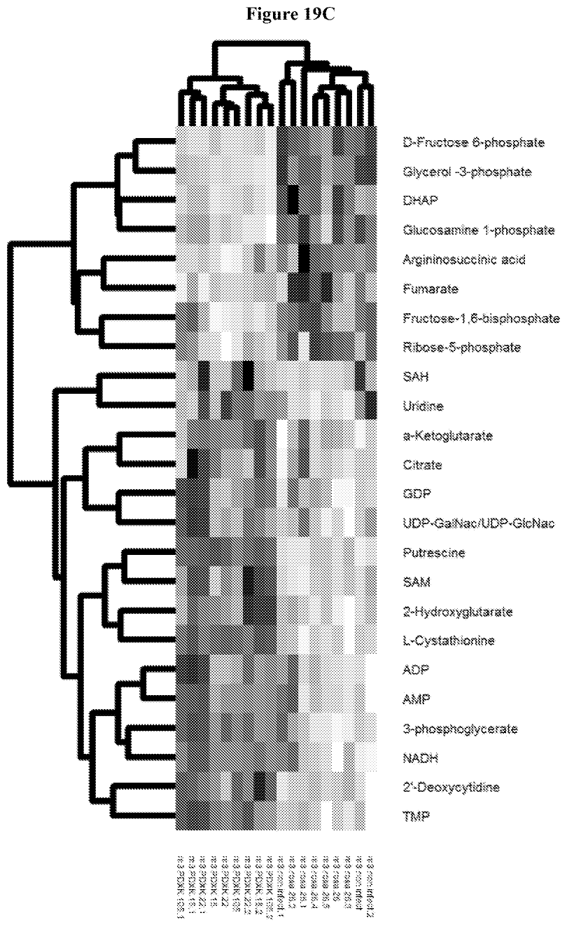

The downstream mechanism by which PLP contributes to leukemia maintenance was explored. Since PLP is a cofactor of nearly 60 metabolic enzymes, HPLC-MS metabolomics was used to determine how PDXK inhibition influenced the levels of a range of cellular metabolites. Genetic depletion of PDXK in leukemic cells led to changes in the levels of a wide range of metabolites, with putrescine, nucleosides, and several amino acids being the most dramatically decreased (FIG. 4A, FIGS. 14A-14C, and FIGS. 19A-19C). A decrease in mucleotide precursors (uridine-5′-diphosphoglucuronic acid (UDP), adenine, orotic acid, and N-carcarmoyl-DL-aspartic acid) and decreased levels of metabolites involved in nucleotide synthesis, including TMP, 2′-deoxycytidine, AMP, ADP, GDP, and uridine, were observed in PDXK-depleted leukemic cells.

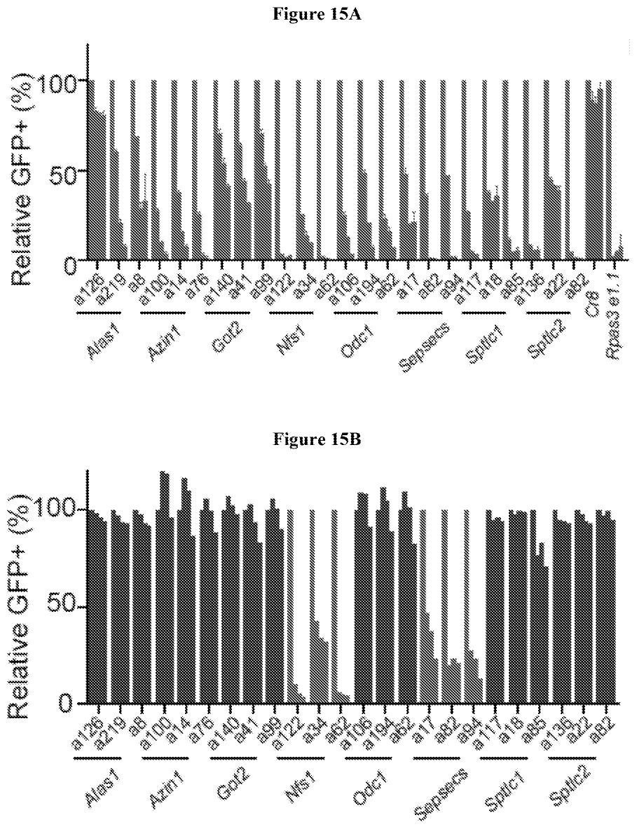

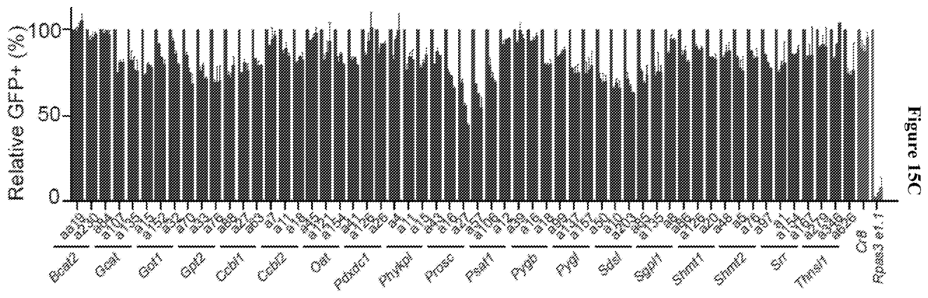

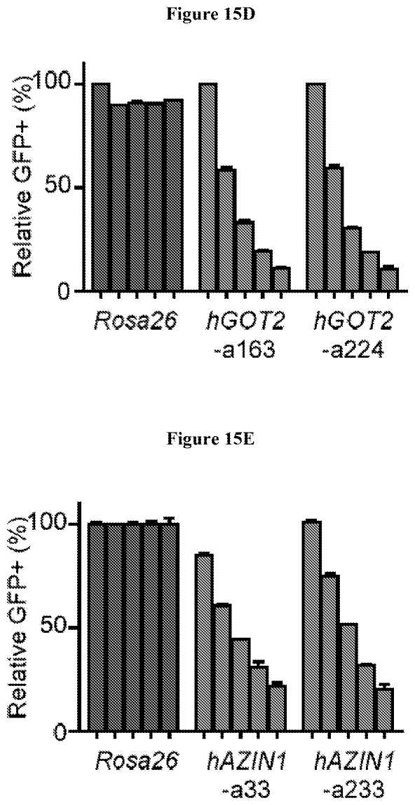

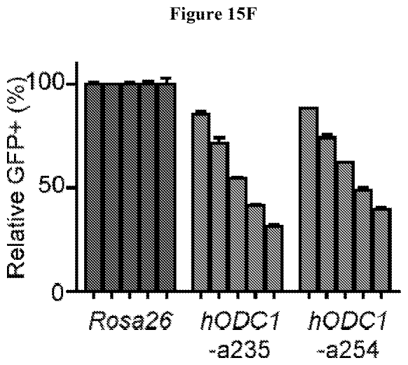

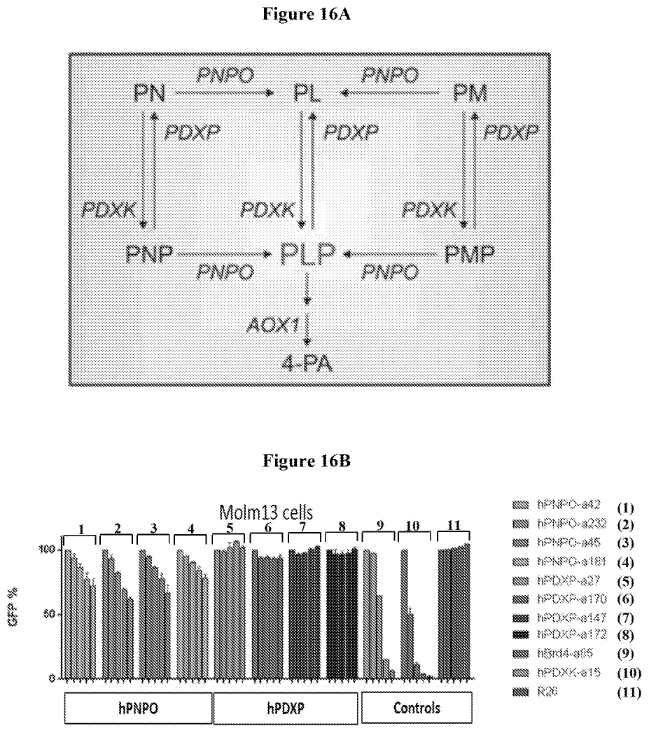

A CRISPR/Cas9 based gene knockout experiment was performed in parallel to functionally test whether any of the 27 PLP-dependent enzymes expressed in AML cells were important for leukemia cell proliferation (FIG. 4B and FIGS. 15A to 15C). Leukemia cells treated with sgRNAs targeting eight enzymes were reproducibly depleted, including glutamic-oxaloacetic transaminase 2 (GOT2), delta-aminolevulinate synthase (ALAS1), cysteine desulfurase (NFS1), serine palmitoyltransferase (SPTLC1 and SPTLC2), antizyme inhibitor 1 (AZIN1), ornithine decarboxylase (ODC1), O-phosphoseryl-tRNA selenium transferase (SEPSECS). Among these, sgRNAs targeting GOT2, ALAS1, SPTLC1, SPTLC2, AZIN1, and ODC1 affected proliferation in leukemic cells but not immortalized iMEFs (FIG. 4C and FIGS. 15D to 15F). Analysis of publically available datasets on CRISPR/Cas9 screens confirmed that these same enzymes are required for the proliferation of human AML cells in culture (FIGS. 6A and 6B) (See Wang et al., Cell 168:890-903 (2017)). Therefore, these enzymes are candidate PDXK effectors in leukemia maintenance.

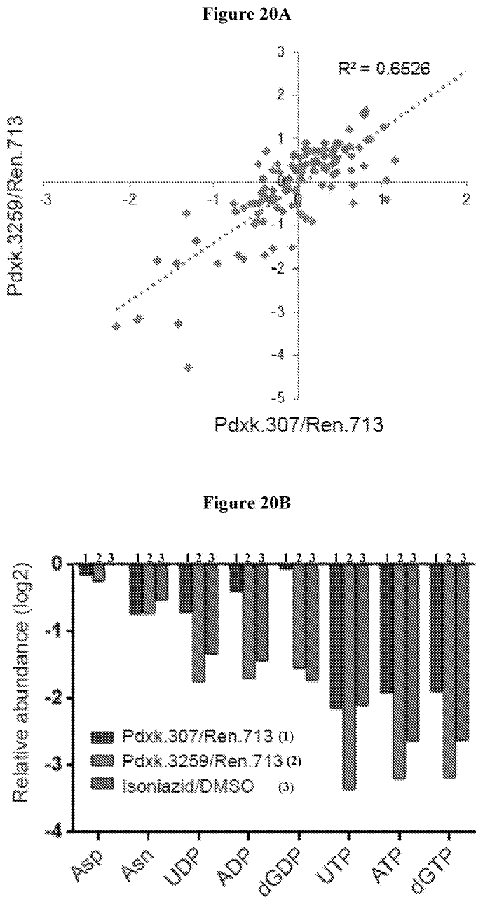

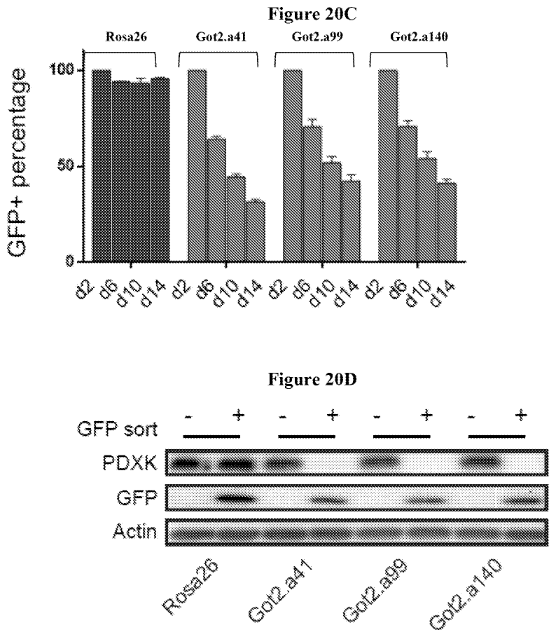

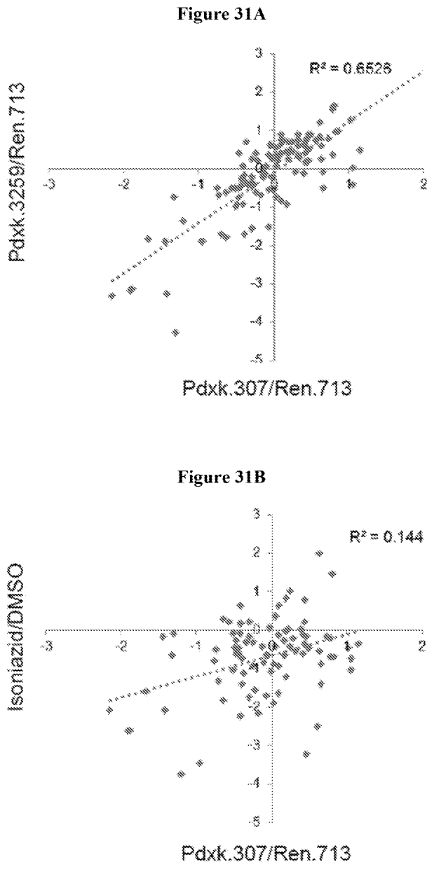

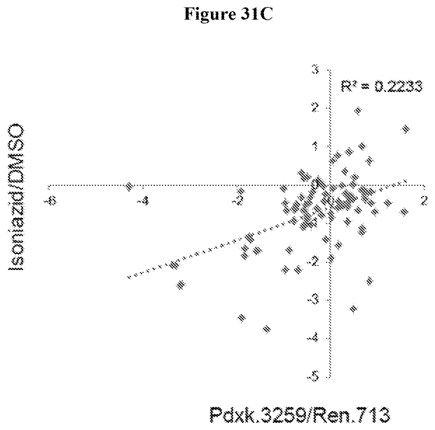

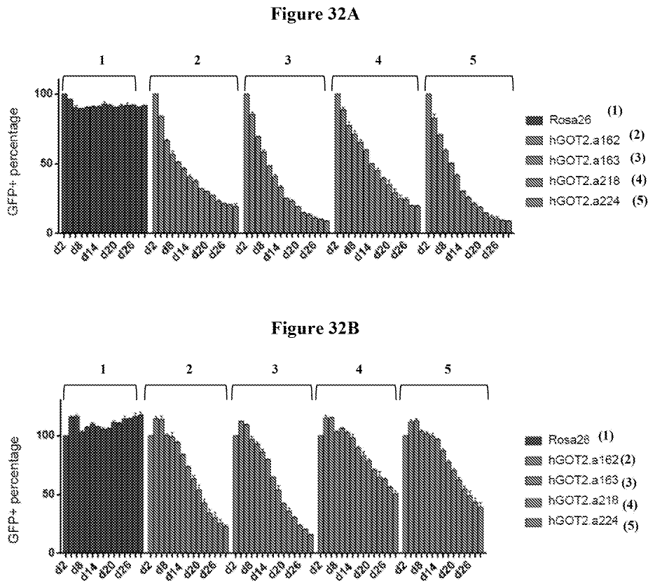

These functional results were intriguing in light of the changes in metabolite levels that were observed following PDXK inhibition. As shown in FIGS. 20A-20B and FIGS. 31A-31C, genetic inhibition of PDXK caused the depletion of asparagine and nucleotides, both of which are generated from aspartate that is a product of the transamination reaction catalyzed by GOT2. Pharmacological inhibition of the vitamin B6 pathway also recapitulated this metabolic change (FIG. 20B and FIGS. 31A-31C). Thus, FIGS. 20A-20B and FIGS. 31A-31C demonstrate that inhibition of PDXK decreased the level of GOT2-catalyzed transamination reaction products including asparagine and nucleotides. These results are consistent with a role of GOT2 in mediating the effect of PDXK and PLP in supporting leukemic cell proliferation (see FIGS. 20C-20D and FIGS. 32A-32B). ODC1 is a decarboxylase that catalyzes the formation of putrescine from ornithine, while AZIN1 binds to ornithine decarboxylase antizyme and stabilizes ODC1. Without wishing to be bound by theory it is believed that suppression of either or both of these enzymes may account for the decrease in putrescine observed following PDXK inhibition. Furthermore, GOT2 is a transaminase that regulates the formation of multiple nucleosides, aspartate, and asparagine, whose concentrations were also reduced upon PDXK depletion.

To further explore the functional relevance of these enzymes to leukemia cell maintenance, an experiment to determine whether exogenous addition of downstream metabolites could rescue, in whole or in part, the proliferative defect produced by PLP depletion following isoniazid treatment was performed. Indeed, the addition of putrescine, uridine, or asparagine partially rescued the proliferative block produced by isoniazid (FIGS. 4D-4F). These observations identify AZIN1, ODC1 and GOT2 as key PLP-dependent enzymes required for leukemia maintenance (FIG. 4G).

Taken together, these results demonstrate that AML cells are dependent on the vitamin B6 pathway for leukemic cell proliferation and that leukemic cell proliferation may be selectively inhibited by disrupting the gene function of PDXK, PNPO, AZIN1, ODC1, GOT2, ALAS1, SPTLC1, or SPTLC2. The elevated PDXK expression level observed in AML cells correlates with the increased demand of highly proliferative leukemic cells on biomass synthesis. PDXK contributes to biomass synthesis partially through the regulation of PLP dependent transaminase GOT2 and asparagine and nucleotides production. Accordingly, the vitamin B6 pathway inhibitors disclosed herein are useful in methods for inhibiting leukemic cell proliferation and treating AML in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating or preventing AML in a subject in need thereof, comprising administering to the subject
a therapeutically effective amount of at least one inhibitor of Vitamin B6 pathway selected from the group consisting of isoniazid, aftin-4, DFMO, gingkotoxin, aminooxyacetic acid, and myriocin, or
a therapeutically effective amount of at least one sgRNA or shRNA that targets one or more genes selected from the group consisting of PDXK, PNPO, AZIN1, ODC1, GOT2, ALAS1, SPTLC1, and SPTLC2.

2. (canceled)

3. The method of claim 1 wherein the at least one sgRNA or shRNA comprises a nucleic acid sequence selected from the group consisting of:
(SEQ ID NO: 1)
5′ TGGCTACGTGGGTAACAGAG 3′ (Pdxk sg-1),
(SEQ ID NO: 2)
5′ ATCCAGAGCCATGTTGTCCG 3′ (Pdxk sg-2),
(SEQ ID NO: 3)
5′ GTGCAGTTTTCAAACCACAC 3′ (Pdxk sg-3),
(SEQ ID NO: 4)
5′ GCTTGGGGTGCCTGCAGAGA 3′ (Odc1-a106),
(SEQ ID NO: 5)
5′ TGCTGTTGACAGTGAGCGCCAAGGTGAACGATGTCAATAATAGT
GAAGCCACAGATGTATTATTGACATCGTTCACCTTGATGCCTACTGC
CTCGGA 3′ (Pdxk.307),
(SEQ ID NO: 6)
TGCTGTTGACAGTGAGCGCCAGGTTCAATGTGAGGTTACATAGTGAA
GCCACAGATGTATGTAACCTCACATTGAACCTGATGCCTACTGCCTC
GGA 3′ (Pdxk.3259),
(SEQ ID NO: 7)
5′ ACGCCCAGGATGGGATCTGG 3′ (Got2.a41),
(SEQ ID NO: 8)
5′ AAAGAATACCTGCCCATTGG 3′ (Got2.a99),
(SEQ ID NO: 9)
5′ GACTGGAGCCTTAAGGGTCG 3′ (Got2.a140),
(SEQ ID NO: 10)
5′ ATACAGAGCCACGTCATCCG 3′ (hPdxk-aa15),
(SEQ ID NO: 11)
5′ CGGCTACGTGGGCAACCGGG 3′ (hPdxk-aa22),
(SEQ ID NO: 12)
5′ GCCTACCGTACACCAGCCTG 3′ (hPdxk-aa105),
(SEQ ID NO: 13)
5′ GTCCCCAGTGCCCACAAAGA 3′ (hPDXK-aa230),
(SEQ ID NO: 14)
5′ AATGGCTTTAGTGCAAGAAT 3′ (Azin1-a100),
(SEQ ID NO: 15)
5′ GAACTACTCCGTTGGCCTGT 3′ (Azin1-a14),
(SEQ ID NO: 16)
5′ GCCAAGATCTCAAGCACGGC 3′ (Azin1-a76),
(SEQ ID NO: 17)
5′ ATATTGACGTCATTGGTGTG 3′ (Odc1-a194),
(SEQ ID NO: 18)
5′ AGGCAGCAGCGTCTTCCGCA 3′ (ALAS1 sg-1),
(SEQ ID NO: 19)
5′ CACCGTTTTAAAAACTCGGT 3′ (ALAS2 sg-2),
(SEQ ID NO: 20)
5′ CTCGGGATAAGAATGGGCAT 3′ (ALAS1 sg-3),
(SEQ ID NO: 21)
5′ TGCGTAAAAGGGAGTGACGC 3′ (Odc1-a62),
(SEQ ID NO: 22)
5′ GCTGGCCAACCCTCGAGTTA 3′ (SPTLC1 sg-1),
(SEQ ID NO: 23)
5′ GATGGTGCAGGCGCTGTACG 3′ (SPTLC1 sg-2),
(SEQ ID NO: 24)
5′ TCAACTACAACATCGTGTCC 3′ (SPTLC1 sg-3),
(SEQ ID NO: 25)
5′ GCTCCAGGCACACTACAGAT 3′ (SPTLC2 sg-1),
(SEQ ID NO: 26)
5′ GAACGGCTGCGTCAAGAACG 3′ (SPTLC2 sg-2),
(SEQ ID NO: 27)
5′ AATCTCGAAGATATCCAAAG 3′ (SPTLC2 sg-3),
(SEQ ID NO: 28)
5′ GGTGTGTGGTTTCCCCAGGT 3′ (hGOT2.a162),
(SEQ ID NO: 29)
5′ GATGGGTGTGTGGTTTCCCC 3′ (hGOT2.a163),
(SEQ ID NO: 30)
5′ GGACGCGGGTCCACTCCCGT 3′ (hGOT2.a218),
(SEQ ID NO: 31)
5′ TGGACCCGCGTCCGGAACAG 3′ (hGOT2.a224),
(SEQ ID NO: 39)
5′ ACGATGAACATGTTAGACAT 3′ (hAZIN1-a233),
(SEQ ID NO: 40)
5′ CTATGTTTATGAACATACCC 3′ (hAZIN1-a33),
(SEQ ID NO: 41)
5′ TATCTGCTTGATATTGGCGG 3′ (hODC1-a235),
(SEQ ID NO: 42)
5′ CAACGCTGGGTTGATTACGC 3′ (hODC1-a254),
(SEQ ID NO: 982)
5′ GGAGGTCCTGGGGAACGTAC 3′ (Pdxk sg-4),
(SEQ ID NO: 983)
5′ CATGGCAGCGAAGAGGTCCC 3′ (Pdxk sg-5),
(SEQ ID NO: 984)
5′ AGCTGTCTTCGTGGGCACCG 3′ (Pdxk sg-6),
(SEQ ID NO: 985)
5′ TGTAACCTCACATTGAACCTGA 3′,
(SEQ ID NO: 986)
5′ TTATTGACATCGTTCACCTTGA 3′,
(SEQ ID NO: 987)
5′ CATGCGCAAGAGTTACCGCG 3′ (hPNPO-a42:);
(SEQ ID NO: 988)
5′ ATGACCGGATAGTCTTTCGG 3′ (hPNPO-a232);
(SEQ ID NO: 989)
5′ GAGTTACCGCGGGGACCGAG 3′ (hPNPO-a45);
and
(SEQ ID NO: 990)
5′ TTCTGTGATCCCTGATCGGG 3′ (hPNPO-a181).

4. The method of claim 1, wherein the subject displays elevated expression levels of PDXK protein in leukemic cells prior to treatment.

5. The method of claim 1, wherein treatment with the at least one sgRNA, shRNA, or inhibitor of Vitamin B6 pathway results in a decrease in PDXK and/or PLP levels in the subject compared to that observed prior to treatment.

6. The method of claim 1, wherein the subject has been diagnosed as having AML.

7. The method of claim 6, wherein the signs or symptoms of AML comprise one or more of leukemic cell proliferation, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, or loss of appetite.

8. The method of claim 1, wherein the subject harbors one or more point mutations in NRAS, DNMT3A, FLT3, KIT, IDH1, IDH2, CEBPA and NPM1.

9. The method of claim 1, wherein the subject harbors one or more gene fusions selected from the group consisting of CBFB-MYH11, DEK-NUP214, MLL-MLLT3, PML-RARA, RBM15-MKL1, RPN1-EVI1 and RUNX1-RUNX1T1.

10. The method of claim 1, wherein the subject is human.

11. The method of claim 1, wherein the at least one sgRNA, shRNA, or inhibitor of Vitamin B6 pathway is administered orally, topically, intranasally, systemically, intravenously, subcutaneously, intraperitoneally, intradermally, intraocularly, iontophoretically, transmucosally, or intramuscularly.

12. The method of claim 1, further comprising separately, sequentially or simultaneously administering one or more additional therapeutic agents to the subject.

13. The method of claim 12, wherein the one or more additional therapeutic agents are selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and combinations thereof.

14. The method of claim 1, wherein the at least one sgRNA, shRNA, or inhibitor of Vitamin B6 pathway is administered daily for 6 weeks 12 weeks or more.

15. (canceled)

16. A method for monitoring the therapeutic efficacy of a dosage of an inhibitor of Vitamin B6 pathway or an inhibitory RNA that targets a gene selected from the group consisting of PDXK, PNPO, AZIN1, ODC1, GOT2, ALAS1, SPTLC1, and SPTLC2 in a subject diagnosed with AML comprising:
(a) detecting PDXK protein levels or intracellular PLP levels in a test sample obtained from the subject after the subject has been administered the dosage of the inhibitor of Vitamin B6 pathway or the inhibitory RNA; and
(b) determining that the dosage of the inhibitor of Vitamin B6 pathway or the inhibitory RNA is effective when the PDXK protein levels or intracellular PLP levels in the test sample are reduced compared to that observed in a control sample obtained from the subject prior to administration of the inhibitor of Vitamin B6 pathway or the inhibitory RNA,
wherein the inhibitor of Vitamin B6 pathway is isoniazid, aftin-4, DFMO, gingkotoxin, aminooxyacetic acid, or myriocin.

17. (canceled)

18. The method of claim 16, wherein the inhibitory RNA is a shRNA or a sgRNA.

19. A method for inhibiting leukemic cell proliferation in a subject in need thereof, comprising administering to the subject
a therapeutically effective amount of at least one inhibitor of Vitamin B6 pathway selected from the group consisting of isoniazid, aftin-4, DFMO, gingkotoxin, aminooxyacetic acid, and myriocin, or
a therapeutically effective amount of at least one sgRNA or shRNA that targets one or more genes selected from the group consisting of PDXK, PNPO, AZIN1, ODC1, GOT2, ALAS1, SPTLC1, and SPTLC2,
wherein the subject suffers from a disease or condition characterized by elevated expression levels and/or increased activity of PDXK.

20. (canceled)

21. The method of claim 16, further comprising detecting intracellular levels of PLP in the subject.

22. The method of claim 19, wherein the at least one sgRNA or shRNA comprises a nucleic acid sequence selected from the group consisting of:
(SEQ ID NO: 1)
5′ TGGCTACGTGGGTAACAGAG 3′ (Pdxk sg-1),
(SEQ ID NO: 2)
5′ ATCCAGAGCCATGTTGTCCG 3′ (Pdxk sg-2),
(SEQ ID NO: 3)
5′ GTGCAGTTTTCAAACCACAC 3′ (Pdxk sg-3),
(SEQ ID NO: 4)
5′ GCTTGGGGTGCCTGCAGAGA 3′ (Odc1-a106),
(SEQ ID NO: 5)
5′ TGCTGTTGACAGTGAGCGCCAAGGTGAACGATGTCAATAATAGT
GAAGCCACAGATGTATTATTGACATCGTTCACCTTGATGCCTACTGC
CTCGGA 3′ (Pdxk.307),
(SEQ ID NO: 6)
TGCTGTTGACAGTGAGCGCCAGGTTCAATGTGAGGTTACATAGTGAA
GCCACAGATGTATGTAACCTCACATTGAACCTGATGCCTACTGCCTC
GGA 3′ (Pdxk.3259),
(SEQ ID NO: 7)
5′ ACGCCCAGGATGGGATCTGG 3′ (Got2.a41),
(SEQ ID NO: 8)
5′ AAAGAATACCTGCCCATTGG 3′ (Got2.a99),
(SEQ ID NO: 9)
5′ GACTGGAGCCTTAAGGGTCG 3′ (Got2.a140),
(SEQ ID NO: 10)
5′ ATACAGAGCCACGTCATCCG 3′ (hPdxk-aa15),
(SEQ ID NO: 11)
5′ CGGCTACGTGGGCAACCGGG 3′ (hPdxk-aa22),
(SEQ ID NO: 12)
5′ GCCTACCGTACACCAGCCTG 3′ (hPdxk-aa105),
(SEQ ID NO: 13)
5′ GTCCCCAGTGCCCACAAAGA 3′ (hPDXK-aa230),
(SEQ ID NO: 14)
5′ AATGGCTTTAGTGCAAGAAT 3′ (Azin1-a100),
(SEQ ID NO: 15)
5′ GAACTACTCCGTTGGCCTGT 3′ (Azin1-a14),
(SEQ ID NO: 16)
5′ GCCAAGATCTCAAGCACGGC 3′ (Azin1-a76),
(SEQ ID NO: 17)
5′ ATATTGACGTCATTGGTGTG 3′ (Odc1-a194),
(SEQ ID NO: 18)
5′ AGGCAGCAGCGTCTTCCGCA 3′ (ALAS1 sg-1),
(SEQ ID NO: 19)
5′ CACCGTTTTAAAAACTCGGT 3′ (ALAS2 sg-2),
(SEQ ID NO: 20)
5′ CTCGGGATAAGAATGGGCAT 3′ (ALAS1 sg-3),
(SEQ ID NO: 21)
5′ TGCGTAAAAGGGAGTGACGC 3′ (Odc1-a62),
(SEQ ID NO: 22)
5′ GCTGGCCAACCCTCGAGTTA 3′ (SPTLC1 sg-1),
(SEQ ID NO: 23)
5′ GATGGTGCAGGCGCTGTACG 3′ (SPTLC1 sg-2),
(SEQ ID NO: 24)
5′ TCAACTACAACATCGTGTCC 3′ (SPTLC1 sg-3),
(SEQ ID NO: 25)
5′ GCTCCAGGCACACTACAGAT 3′ (SPTLC2 sg-1),
(SEQ ID NO: 26)
5′ GAACGGCTGCGTCAAGAACG 3′ (SPTLC2 sg-2),
(SEQ ID NO: 27)
5′ AATCTCGAAGATATCCAAAG 3′ (SPTLC2 sg-3),
(SEQ ID NO: 28)
5′ GGTGTGTGGTTTCCCCAGGT 3′ (hGOT2.a162),
(SEQ ID NO: 29)
5′ GATGGGTGTGTGGTTTCCCC 3′ (hGOT2.a163),
(SEQ ID NO: 30)
5′ GGACGCGGGTCCACTCCCGT 3′ (hGOT2.a218),
(SEQ ID NO: 31)
5′ TGGACCCGCGTCCGGAACAG 3′ (hGOT2.a224),
(SEQ ID NO: 39)
5′ ACGATGAACATGTTAGACAT 3′ (hAZIN1-a233),
(SEQ ID NO: 40)
5′ CTATGTTTATGAACATACCC 3′ (hAZIN1-a33),
(SEQ ID NO: 41)
5′ TATCTGCTTGATATTGGCGG 3′ (hODC1-a235),
(SEQ ID NO: 42)
5′ CAACGCTGGGTTGATTACGC 3′ (hODC1-a254),
(SEQ ID NO: 982)
5′ GGAGGTCCTGGGGAACGTAC 3′ (Pdxk sg-4),
(SEQ ID NO: 983)
5′ CATGGCAGCGAAGAGGTCCC 3′ (Pdxk sg-5),
(SEQ ID NO: 984)
5′ AGCTGTCTTCGTGGGCACCG 3′ (Pdxk sg-6),
(SEQ ID NO: 985)
5′ TGTAACCTCACATTGAACCTGA 3′,
(SEQ ID NO: 986)
5′ TTATTGACATCGTTCACCTTGA 3′,
(SEQ ID NO: 987)
5′ CATGCGCAAGAGTTACCGCG 3′ (hPNPO-a42:);
(SEQ ID NO: 988)
5′ ATGACCGGATAGTCTTTCGG 3′ (hPNPO-a232);
(SEQ ID NO: 989)
5′ GAGTTACCGCGGGGACCGAG 3′ (hPNPO-a45);
and
(SEQ ID NO: 990)
5′ TTCTGTGATCCCTGATCGGG 3′ (hPNPO-a181).


SEQUENCE LISTING (996 sequences)

SEQ ID NO: 1 - DNA, 20 bp, Artificial Sequence
  Description of Artificial Sequence Synthetic oligonucleotide
tggctacgtgggtaacagag

SEQ ID NO: 2 - DNA, 20 bp, Artificial Sequence
  Description of Artificial Sequence Synthetic oligonucleotide
atccagagccatgttgtccg

SEQ ID NO: 3 - DNA, 20 bp, Artificial Sequence
  Description of Artificial Sequence Synthetic oligonucleotide
gtgcagttttcaaaccacac

SEQ ID NO: 4 - DNA, 20 bp, Artificial Sequence
  Description of Artificial Sequence Synthetic oligonucleotide
gcttggggtgcctgcagaga

SEQ ID NO: 5 - DNA, 97 bp, Artificial Sequence
  Description of Artificial Sequence Synthetic oligonucleotide
tgctgttgacagtgagcgccaaggtgaacgatgtcaataatagtgaagccacagatgtattattgacatcgttcaccttgatgcctactgcctcgga

SEQ ID NO: 6 - DNA, 97 bp, Artificial Sequence
  Description of Artificial Sequence Synthetic oligonucleotide
tgctgttgacagtgagcgccaggttcaatgtgaggttacatagtgaagccacagatgtatgtaacctcacattgaacctgatgcctactgcctcgga

SEQ ID NO: 7 - DNA, 20 bp, Artificial Sequence
  Description of Artificial Sequence Synthetic oligonucleotide
acgcccaggatgggatctgg

SEQ ID NO: 8 - DNA, 20 bp, Artificial Sequence
  Description of Artificial Sequence Synthetic oligonucleotide
aaagaatacctgcccattgg

SEQ ID NO: 9 - DNA, 20 bp, Artificial Sequence
  Description of Artificial Sequence Synthetic oligonucleotide
gactggagccttaagggtcg

SEQ ID NO: 10 - DNA, 20 bp, Artificial Sequence
  Description of Artificial Sequence Synthetic oligonucleotide
atacagagccacgtcatccg

SEQ ID NO: 11 - DNA, 20 bp, Artificial Sequence
  Description of Artificial Sequence Synthetic oligonucleotide
cggctacgtgggcaaccggg

SEQ ID NO: 12 - DNA, 20 bp, Artificial Sequence
  Description of Artificial Sequence Synthetic oligonucleotide
gcctaccgtacaccagcctg

SEQ ID NO: 13 - DNA, 20 bp, Artificial Sequence
  Description of Artificial Sequence Synthetic oligonucleotide
gtccccagtgcccacaaaga

SEQ ID NO: 14 - DNA, 20 bp, Artificial Sequence
  Description of Artificial Sequence Synthetic oligonucleotide
aatggctttagtgcaagaat

SEQ ID NO: 15 - DNA, 20 bp, Artificial Sequence
  Description of Artificial Sequence Synthetic oligonucleotide
gaactactccgttggcctgt

SEQ ID NO: 16 - DNA, 20 bp, Artificial Sequence
  Description of Artificial Sequence Synthetic oligonucleotide
gccaagatctcaagcacggc

SEQ ID NO: 17 - DNA, 20 bp, Artificial Sequence
  Description of Artificial Sequence Synthetic oligonucleotide
atattgacgtcattggtgtg

SEQ ID NO: 18 - DNA, 20 bp, Artificial Sequence
  Description of Artificial Sequence Synthetic oligonucleotide
aggcagcagcgtcttccgca

SEQ ID NO: 19 - DNA, 20 bp, Artificial Sequence
  Description of Artificial Sequence Synthetic oligonucleotide
caccgttttaaaaactcggt

SEQ ID NO: 20 - DNA, 20 bp, Artificial Sequence
  Description of Artificial Sequence Synthetic oligonucleotide
ctcgggataagaatgggcat

SEQ ID NO: 21 - DNA, 20 bp, Artificial Sequence
  Description of Artificial Sequence Synthetic oligonucleotide
tgcgtaaaagggagtgacgc

SEQ ID NO: 22 - DNA, 20 bp, Artificial Sequence
  Description of Artificial Sequence Synthetic oligonucleotide
gctggccaaccctcgagtta

SEQ ID NO: 23 - DNA, 20 bp, Artificial Sequence
  Description of Artificial Sequence Synthetic oligonucleotide
gatggtgcaggcgctgtacg

SEQ ID NO: 24 - DNA, 20 bp, Artificial Sequence
  Description of Artificial Sequence Synthetic oligonucleotide
tcaactacaacatcgtgtcc

SEQ ID NO: 25 - DNA, 20 bp, Artificial Sequence
  Description of Artificial Sequence Synthetic oligonucleotide
gctccaggcacactacagat

SEQ ID NO: 26 - DNA, 20 bp, Artificial Sequence
  Description of Artificial Sequence Synthetic oligonucleotide
gaacggctgcgtcaagaacg

SEQ ID NO: 27 - DNA, 20 bp, Artificial Sequence
  Description of Artificial Sequence Synthetic oligonucleotide
aatctcgaagatatccaaag

SEQ ID NO: 28 - DNA, 20 bp, Artificial Sequence
  Description of Artificial Sequence Synthetic oligonucleotide
ggtgtgtggtttccccaggt

SEQ ID NO: 29 - DNA, 20 bp, Artificial Sequence
  Description of Artificial Sequence Synthetic oligonucleotide
gatgggtgtgtggtttcccc

SEQ ID NO: 30 - DNA, 20 bp, Artificial Sequence
  Description of Artificial Sequence Synthetic oligonucleotide
ggacgcgggtccactcccgt

SEQ ID NO: 31 - DNA, 20 bp, Artificial Sequence
  Description of Artificial Sequence Synthetic oligonucleotide
tggacccgcgtccggaacag

SEQ ID NO: 32 - DNA, 20 bp, Artificial Sequence
  Description of Artificial Sequence Synthetic oligonucleotide
gtggaaaggacgaaacaccg

SEQ ID NO: 33 - DNA, 20 bp, Artificial Sequence
  Description of Artificial Sequence Synthetic oligonucleotide
gttttagagctagaaatagc

SEQ ID NO: 34 - DNA, 17 bp, Artificial Sequence
  Description of Artificial Sequence Synthetic oligonucleotide
ggccctgggggatcttt

SEQ ID NO: 35 - DNA, 17 bp, Artificial Sequence
  Description of Artificial Sequence Synthetic primer
aaagatcccccagggcc

SEQ ID NO: 36 - DNA, 30 bp, Artificial Sequence
  Description of Artificial Sequence Synthetic primer
ttatatatcttgtggaaaggacgaaacacc

SEQ ID NO: 37 - DNA, 37 bp, Artificial Sequence
  Description of Artificial Sequence Synthetic primer
tagccttattttaacttgctatttctagctctaaaac

SEQ ID NO: 38 - DNA, 31 bp, Artificial Sequence
  Description of Artificial Sequence Synthetic primer
atttcttggctttatatatcttgtggaaagg

SEQ ID NO: 39 - DNA, 20 bp, Artificial Sequence
  Description of Artificial Sequence Synthetic oligonucleotide
acgatgaacatgttagacat

SEQ ID NO: 40 - DNA, 20 bp, Artificial Sequence
  Description of Artificial Sequence Synthetic oligonucleotide
ctatgtttatgaacataccc

SEQ ID NO: 41 - DNA, 20 bp, Artificial Sequence
  Description of Artificial Sequence Synthetic oligonucleotide
tatctgcttgatattggcgg

SEQ ID NO: 42 - DNA, 20 bp, Artificial Sequence
  Description of Artificial Sequence Synthetic oligonucleotide
caacgctgggttgattacgc

SEQ ID NO: 43 - DNA, 20 bp, Artificial Sequence
  Description of Artificial Sequence Synthetic oligonucleotide
gatcttgggtttccgtatga

SEQ ID NO: 44 - DNA, 20 bp, Artificial Sequence
  Description of Artificial Sequence Synthetic oligonucleotide
gcggatgttttctgcgatag

SEQ ID NO: 45 - DNA, 20 bp, Artificial Sequence
  Description of Artificial Sequence Synthetic oligonucleotide
gagctgtcgacaggcgctga

SEQ ID NO: 46 - DNA, 20 bp, Artificial Sequence
  Description of Artificial Sequence Synthetic oligonucleotide
gagtctggagccgtaccaga

SEQ ID NO: 47 - DNA, 20 bp, Artificial Sequence
  Description of Artificial Sequence Synthetic oligonucleotide
gtgatgctattcccttcgcc

SEQ ID NO: 48 - DNA, 20 bp, Artificial Sequence
  Description of Artificial Sequence Synthetic oligonucleotide
ggcaggggacgtggtcaacc

SEQ ID NO: 49 - DNA, 20 bp, Artificial Sequence
  Description of Artificial Sequence Synthetic oligonucleotide
ggcaactcctcggcgtggac

SEQ ID NO: 50 - DNA, 20 bp, Artificial Sequence
  Description of Artificial Sequence Synthetic oligonucleotide
gaagggagtccggtcatccg

SEQ ID NO: 51 - DNA, 20 bp, Artificial Sequence
  Description of Artificial Sequence Synthetic oligonucleotide
gagaggcacggccacgttga

SEQ ID NO: 52 - DNA, 20 bp, Artificial Sequence
  Description of Artificial Sequence Synthetic oligonucleotide
ggctcagcactcacagcgcc

SEQ ID NO: 53 - DNA, 20 bp, Artificial Sequence
  Description of Artificial Sequence Synthetic oligonucleotide
gcacacaagcatcgaggtcc

SEQ ID NO: 54 - DNA, 20 bp, Artificial Sequence
  Description of Artificial Sequence Synthetic oligonucleotide
gccatgctgctctcggctat

SEQ ID NO: 55 - DNA, 20 bp, Artificial Sequence
  Description of Artificial Sequence Synthetic oligonucleotide
gatgaagggccgaataaaga

SEQ ID NO: 56 - DNA, 20 bp, Artificial Sequence
  Description of Artificial Sequence Synthetic oligonucleotide
gcgagctgtcaccggtgtcc

SEQ ID NO: 57 - DNA, 20 bp, Artificial Sequence
  Description of Artificial Sequence Synthetic oligonucleotide
gagggtgttttttcgctagg

SEQ ID NO: 58 - DNA, 20 bp, Artificial Sequence
  Description of Artificial Sequence Synthetic oligonucleotide
gttcttgttgtagaagtccg

SEQ ID NO: 59 - DNA, 20 bp, Artificial Sequence
  Description of Artificial Sequence Synthetic oligonucleotide
ggttccaccaaggcccgaca

SEQ ID NO: 60 - DNA, 20 bp, Artificial Sequence
  Description of Artificial Sequence Synthetic oligonucleotide
gcaccaactctccgtaagtc

SEQ ID NO: 61 - DNA, 20 bp, Artificial Sequence
  Description of Artificial Sequence Synthetic oligonucleotide
ggaatggctgcgagctgcgc

SEQ ID NO: 62 - DNA, 20 bp, Artificial Sequence
  Description of Artificial Sequence Synthetic oligonucleotide
gtctccttgtacatcgagag

SEQ ID NO: 63 - DNA, 20 bp, Artificial Sequence
  Description of Artificial Sequence Synthetic oligonucleotide
gcactgctggcccgccttgt

SEQ ID NO: 64 - DNA, 20 bp, Artificial Sequence
  Description of Artificial Sequence Synthetic oligonucleotide
gaatgtctgctaccagtacg

SEQ ID NO: 65 - DNA, 20 bp, Artificial Sequence
  Description of Artificial Sequence Synthetic oligonucleotide
gcttcactcaactctcggtt

SEQ ID NO: 66 - DNA, 20 bp, Artificial Sequence
  Description of Artificial Sequence Synthetic oligonucleotide
gtatctatgaactaccgagt

SEQ ID NO: 67 - DNA, 20 bp, Artificial Sequence
  Description of Artificial Sequence Synthetic oligonucleotide
gctgttttattgtccctatc

SEQ ID NO: 68 - DNA, 20 bp, Artificial Sequence
  Description of Artificial Sequence Synthetic oligonucleotide
ggcaagattcaatgcgatcc

SEQ ID NO: 69 - DNA, 20 bp, Artificial Sequence
  Description of Artificial Sequence Synthetic oligonucleotide
gagccatccgctacagccac

SEQ ID NO: 70 - DNA, 20 bp, Artificial Sequence
  Description of Artificial Sequence Synthetic oligonucleotide
gctctagccgaaggcaattt

SEQ ID NO: 71 - DNA, 20 bp, Artificial Sequence
  Description of Artificial Sequence Synthetic oligonucleotide
gccctctcacgttgctccca

SEQ ID NO: 72 - DNA, 20 bp, Artificial Sequence
  Description of Artificial Sequence Synthetic oligonucleotide
gaacatctggtgcacggtat

SEQ ID NO: 73 - DNA, 20 bp, Artificial Sequence
  Description of Artificial Sequence Synthetic oligonucleotide
gtatggacaacccgggcgga

SEQ ID NO: 74 - DNA, 20 bp, Artificial Sequence
  Description of Artificial Sequence Synthetic oligonucleotide
ggaggttcccgtacttatct

SEQ ID NO: 75 - DNA, 20 bp, Artificial Sequence
  Description of Artificial Sequence Synthetic oligonucleotide
gtgtccacaggtcgtagatc

SEQ ID NO: 76 - DNA, 20 bp, Artificial Sequence
  Description of Artificial Sequence Synthetic oligonucleotide
gggctactttgcgtgcaatc

SEQ ID NO: 77 - DNA, 20 bp, Artificial Sequence
  Description of Artificial Sequence Synthetic oligonucleotide
gatgtttgagaccgttgtag

SEQ ID NO: 78 - DNA, 20 bp, Artificial Sequence
  Description of Artificial Sequence Synthetic oligonucleotide
gtcgcaagatcttcgggttc

SEQ ID NO: 79 - DNA, 20 bp, Artificial Sequence
  Description of Artificial Sequence Synthetic oligonucleotide
gatgtcagaaccatgtacga

SEQ ID NO: 80 - DNA, 20 bp, Artificial Sequence
  Description of Artificial Sequence Synthetic oligonucleotide
gaacgtggtggccgatttct

SEQ ID NO: 81 - DNA, 20 bp, Artificial Sequence
  Description of Artificial Sequence Synthetic oligonucleotide
gaagcccacggcggaacacc

SEQ ID NO: 82 - DNA, 20 bp, Artificial Sequence
  Description of Artificial Sequence Synthetic oligonucleotide
ggagcccggcgatctgtgat

SEQ ID NO: 83 - DNA, 20 bp, Artificial Sequence
  Description of Artificial Sequence Synthetic oligonucleotide
gggcccagggtgtcgtagag

SEQ ID NO: 84 - DNA, 20 bp, Artificial Sequence
  Description of Artificial Sequence Synthetic oligonucleotide
ggatctcaagtatcggctgc

SEQ ID NO: 85 - DNA, 20 bp, Artificial Sequence
  Description of Artificial Sequence Synthetic oligonucleotide
gggcatccacttgcggagct

SEQ ID NO: 86 - DNA, 20 bp, Artificial Sequence
  Description of Artificial Sequence Synthetic oligonucleotide
gagttgaagtactcgggcac

SEQ ID NO: 87 - DNA, 20 bp, Artificial Sequence
  Description of Artificial Sequence Synthetic oligonucleotide
gtcactggtgacgatagact

SEQ ID NO: 88 - DNA, 20 bp, Artificial Sequence
  Description of Artificial Sequence Synthetic oligonucleotide
gaggggagcaatcgcttgca

SEQ ID NO: 89 - DNA, 20 bp, Artificial Sequence
  Description of Artificial Sequence Synthetic oligonucleotide
gtcgatatcaacatgcgcgt

SEQ ID NO: 90 - DNA, 20 bp, Artificial Sequence
  Description of Artificial Sequence Synthetic oligonucleotide
ggtccgcatgacccggtact

SEQ ID NO: 91 - DNA, 20 bp, Artificial Sequence
  Description of Artificial Sequence Synthetic oligonucleotide
gggggcaccatacctcacgt

SEQ ID NO: 92 - DNA, 20 bp, Artificial Sequence
  Description of Artificial Sequence Synthetic oligonucleotide
gaaggcatgcgcagatttgt

SEQ ID NO: 93 - DNA, 20 bp, Artificial Sequence
  Description of Artificial Sequence Synthetic oligonucleotide
gggagagttattgagcgcct

SEQ ID NO: 94 - DNA, 20 bp, Artificial Sequence
  Description of Artificial Sequence Synthetic oligonucleotide
gaggttcatggagtcgaatt

SEQ ID NO: 95 - DNA, 20 bp, Artificial Sequence
  Description of Artificial Sequence Synthetic oligonucleotide
gcgaggtccacgcataatgc

SEQ ID NO: 96 - DNA, 20 bp, Artificial Sequence
  Description of Artificial Sequence Synthetic oligonucleotide
gacttcccgccgccagcaca

SEQ ID NO: 97 - DNA, 20 bp, Artificial Sequence
  Description of Artificial Sequence Synthetic oligonucleotide
ggccagtggatgactccccg

SEQ ID NO: 98 - DNA, 20 bp, Artificial Sequence
  Description of Artificial Sequence Synthetic oligonucleotide
gcagagattgctgatcgcat

SEQ ID NO: 99 - DNA, 20 bp, Artificial Sequence
  Description of Artificial Sequence Synthetic oligonucleotide
gactggttgtcagcggttca

SEQ ID NO: 100 - DNA, 20 bp, Artificial Sequence
  Description of Artificial Sequence Synthetic oligonucleotide
gggagggccccggacccaac

SEQ ID NO: 101 - DNA, 20 bp, Artificial Sequence
  Description of Artificial Sequence Synthetic oligonucleotide
ggaaagccatttttgcacga

SEQ ID NO: 102 - DNA, 20 bp, Artificial Sequence
  Description of Artificial Sequence Synthetic oligonucleotide
ggtgagtgacgaaatggttg

SEQ ID NO: 103 - DNA, 20 bp, Artificial Sequence
  Description of Artificial Sequence Synthetic oligonucleotide
gagcctgcctcacagtccga

SEQ ID NO: 104 - DNA, 20 bp, Artificial Sequence
  Description of Artificial Sequence Synthetic oligonucleotide
gagactccttcgtgcaaaaa

SEQ ID NO: 105 - DNA, 20 bp, Artificial Sequence
  Description of Artificial Sequence Synthetic oligonucleotide
ggtctgtcatttggccaccg

SEQ ID NO: 106 - DNA, 20 bp, Artificial Sequence
  Description of Artificial Sequence Synthetic oligonucleotide
gttttgggccgaggtctagt

SEQ ID NO: 107 - DNA, 20 bp, Artificial Sequence
  Description of Artificial Sequence Synthetic oligonucleotide
gcaagaatgattttgggccg

SEQ ID NO: 108 - DNA, 20 bp, Artificial Sequence
  Description of Artificial Sequence Synthetic oligonucleotide
ggccggtcataccgccgata

SEQ ID NO: 109 - DNA, 20 bp, Artificial Sequence
  Description of Artificial Sequence Synthetic oligonucleotide
gagacgttaccgcccctaaa

SEQ ID NO: 110 - DNA, 20 bp, Artificial Sequence
  Description of Artificial Sequence Synthetic oligonucleotide
gggggcactcgcaatatctc

SEQ ID NO: 111 - DNA, 20 bp, Artificial Sequence
  Description of Artificial Sequence Synthetic oligonucleotide
ggttcaagactgtgaatcgt

SEQ ID NO: 112 - DNA, 20 bp, Artificial Sequence
  Description of Artificial Sequence Synthetic oligonucleotide
gagatgcctcggagaagtgt

SEQ ID NO: 113 - DNA, 20 bp, Artificial Sequence
  Description of Artificial Sequence Synthetic oligonucleotide
gtatggcctgcaagacacga

SEQ ID NO: 114 - DNA, 20 bp, Artificial Sequence
  Description of Artificial Sequence Synthetic oligonucleotide
gtggccccacacaccccgat

SEQ ID NO: 115 - DNA, 20 bp, Artificial Sequence
  Description of Artificial Sequence Synthetic oligonucleotide
gactggcacgaatccaagag

SEQ ID NO: 116 - DNA, 20 bp, Artificial Sequence
  Description of Artificial Sequence Synthetic oligonucleotide
gccttccaggtcgacgaaaa

SEQ ID NO: 117 - DNA, 20 bp, Artificial Sequence
  Description of Artificial Sequence Synthetic oligonucleotide
ggcggggatccccgtggcgc

SEQ ID NO: 118 - DNA, 20 bp, Artificial Sequence
  Description of Artificial Sequence Synthetic oligonucleotide
gggtgcttatcatcgtgccc

SEQ ID NO: 119 - DNA, 20 bp, Artificial Sequence
  Description of Artificial Sequence Synthetic oligonucleotide
gttacctggtccagatagcg

SEQ ID NO: 120 - DNA, 20 bp, Artificial Sequence
  Description of Artificial Sequence Synthetic oligonucleotide
gaacctgacactcgtgccgc

SEQ ID NO: 121 - DNA, 20 bp, Artificial Sequence
  Description of Artificial Sequence Synthetic oligonucleotide
gatccttcatccaagaccgc

SEQ ID NO: 122 - DNA, 20 bp, Artificial Sequence
  Description of Artificial Sequence Synthetic oligonucleotide
gaactgcgacccccagatag

SEQ ID NO: 123 - DNA, 20 bp, Artificial Sequence
  Description of Artificial Sequence Synthetic oligonucleotide
gatcgttgattacgagctac

SEQ ID NO: 124 - DNA, 20 bp, Artificial Sequence
  Description of Artificial Sequence Synthetic oligonucleotide
gcgagttcccatgttaccgc

SEQ ID NO: 125 - DNA, 20 bp, Artificial Sequence
  Description of Artificial Sequence Synthetic oligonucleotide
gtctacaatgacttcgaacg

SEQ ID NO: 126 - DNA, 20 bp, Artificial Sequence
  Description of Artificial Sequence Synthetic oligonucleotide
gtatcaaactggatatctcg

SEQ ID NO: 127 - DNA, 20 bp, Artificial Sequence
  Description of Artificial Sequence Synthetic oligonucleotide
gtcatccacggtgacatcgt

SEQ ID NO: 128 - DNA, 20 bp, Artificial Sequence
  Description of Artificial Sequence Synthetic oligonucleotide
gtacatcagtccggcgaagg

SEQ ID NO: 129 - DNA, 20 bp, Artificial Sequence
  Description of Artificial Sequence Synthetic oligonucleotide
gacaaacaggtacatcagtc

SEQ ID NO: 130 - DNA, 20 bp, Artificial Sequence
  Description of Artificial Sequence Synthetic oligonucleotide
gatgaaagcaaggcgcataa

SEQ ID NO: 131 - DNA, 20 bp, Artificial Sequence
  Description of Artificial Sequence Synthetic oligonucleotide
ggcagtgtagacccgctcaa

SEQ ID NO: 132 - DNA, 20 bp, Artificial Sequence
  Description of Artificial Sequence Synthetic oligonucleotide
gtgcagtccagagtaccaca

SEQ ID NO: 133 - DNA, 20 bp, Artificial Sequence
  Description of Artificial Sequence Synthetic oligonucleotide
ggcggggctaccactggcga

SEQ ID NO: 134 - DNA, 20 bp, Artificial Sequence
  Description of Artificial Sequence Synthetic oligonucleotide
gctctggaattttgcaagcg

SEQ ID NO: 135 - DNA, 20 bp, Artificial Sequence
  Description of Artificial Sequence Synthetic oligonucleotide
gacagtcacgccctcgccag

SEQ ID NO: 136 - DNA, 20 bp, Artificial Sequence
  Description of Artificial Sequence Synthetic oligonucleotide
gttcaatttcagacgctgcg

SEQ ID NO: 137 - DNA, 20 bp, Artificial Sequence
  Description of Artificial Sequence Synthetic oligonucleotide
gaaatgttacgaccacttct

SEQ ID NO: 138 - DNA, 20 bp, Artificial Sequence
  Description of Artificial Sequence Synthetic oligonucleotide
ggccaagtgaattgtcgact

SEQ ID NO: 139 - DNA, 20 bp, Artificial Sequence
  Description of Artificial Sequence Synthetic oligonucleotide
gagtgaattgtcgactaggg

SEQ ID NO: 140 - DNA, 20 bp, Artificial Sequence
  Description of Artificial Sequence Synthetic oligonucleotide
gccaagtgaattgtcgacta

SEQ ID NO: 141 - DNA, 20 bp, Artificial Sequence
  Description of Artificial Sequence Synthetic oligonucleotide
gtaccccactcacgtcattg

SEQ ID NO: 142 - DNA, 20 bp, Artificial Sequence
  Description of Artificial Sequence Synthetic oligonucleotide
ggttgtcctggaccagcgtc

SEQ ID NO: 143 - DNA, 20 bp, Artificial Sequence
  Description of Artificial Sequence Synthetic oligonucleotide
ggggggccttcttgggcgct

SEQ ID NO: 144 - DNA, 20 bp, Artificial Sequence
  Description of Artificial Sequence Synthetic oligonucleotide
gggtcacagcggggttcaag

SEQ ID NO: 145 - DNA, 20 bp, Artificial Sequence
  Description of Artificial Sequence Synthetic oligonucleotide
gacaatgagcttttcgtctc

SEQ ID NO: 146 - DNA, 20 bp, Artificial Sequence
  Description of Artificial Sequence Synthetic oligonucleotide
ggtcacagcggggttcaagt

SEQ ID NO: 147 - DNA, 20 bp, Artificial Sequence
  Description of Artificial Sequence Synthetic oligonucleotide
ggccaggaagcacattgcga

SEQ ID NO: 148 - DNA, 20 bp, Artificial Sequence
  Description of Artificial Sequence Synthetic oligonucleotide
gaccagccttttctcgactg

SEQ ID NO: 149 - DNA, 20 bp, Artificial Sequence
  Description of Artificial Sequence Synthetic oligonucleotide
gaaccatttgtatccgtgcc

SEQ ID NO: 150 - DNA, 20 bp, Artificial Sequence
  Description of Artificial Sequence Synthetic oligonucleotide
gacattgcaaaagacaccgc

SEQ ID NO: 151 - DNA, 20 bp, Artificial Sequence
  Description of Artificial Sequence Synthetic oligonucleotide
gtgctcatcacgggagaaaa

SEQ ID NO: 152 - DNA, 20 bp, Artificial Sequence
  Description of Artificial Sequence Synthetic oligonucleotide
ggtgaagaacccacggtctg

SEQ ID NO: 153 - DNA, 20 bp, Artificial Sequence
  Description of Artificial Sequence Synthetic oligonucleotide
gccaaagacatcgtgtacat

SEQ ID NO: 154 - DNA, 20 bp, Artificial Sequence
  Description of Artificial Sequence Synthetic oligonucleotide
gaataaacttactgttcccc

SEQ ID NO: 155 - DNA, 20 bp, Artificial Sequence
  Description of Artificial Sequence Synthetic oligonucleotide
gagtatgacgtgagagacca

SEQ ID NO: 156 - DNA, 20 bp, Artificial Sequence
  Description of Artificial Sequence Synthetic oligonucleotide
gtggctttccccacagaccg

SEQ ID NO: 157 - DNA, 20 bp, Artificial Sequence
  Description of Artificial Sequence Synthetic oligonucleotide
gccattgccaagtatcctgt

SEQ ID NO: 158 - DNA, 20 bp, Artificial Sequence
  Description of Artificial Sequence Synthetic oligonucleotide
gttctccaccgcccttggat

SEQ ID NO: 159 - DNA, 20 bp, Artificial Sequence
  Description of Artificial Sequence Synthetic oligonucleotide
gtacttggcaatggaacgag

SEQ ID NO: 160 - DNA, 20 bp, Artificial Sequence
  Description of Artificial Sequence Synthetic oligonucleotide
gtgcacttctccaccgccct

SEQ ID NO: 161 - DNA, 20 bp, Artificial Sequence
  Description of Artificial Sequence Synthetic oligonucleotide
gttcattaccaatccaaggg

SEQ ID NO: 162 - DNA, 20 bp, Artificial Sequence
  Description of Artificial Sequence Synthetic oligonucleotide
gatgaggttgcactggtcga

SEQ ID NO: 163 - DNA, 20 bp, Artificial Sequence
  Description of Artificial Sequence Synthetic oligonucleotide
gggctgttcacactcgatcg

SEQ ID NO: 164 - DNA, 20 bp, Artificial Sequence
  Description of Artificial Sequence Synthetic oligonucleotide
ggaggagacgattctattgt

SEQ ID NO: 165 - DNA, 20 bp, Artificial Sequence
  Description of Artificial Sequence Synthetic oligonucleotide
ggtatccatgtacgtcaccg

SEQ ID NO: 166 - DNA, 20 bp, Artificial Sequence
  Description of Artificial Sequence Synthetic oligonucleotide
ggaccgaatccgagcgccag

SEQ ID NO: 167 - DNA, 20 bp, Artificial Sequence
  Description of Artificial Sequence Synthetic oligonucleotide
ggaaaatcatgtcgtccttc

SEQ ID NO: 168 - DNA, 20 bp, Artificial Sequence
  Description of Artificial Sequence Synthetic oligonucleotide
gtttgttgcccacttggcgt

SEQ ID NO: 169 - DNA, 20 bp, Artificial Sequence
  Description of Artificial Sequence Synthetic oligonucleotide
gctggtatctgctcccgtac

SEQ ID NO: 170 - DNA, 20 bp, Artificial Sequence
  Description of Artificial Sequence Synthetic oligonucleotide
gcttcaaagccgtattgagc

SEQ ID NO: 171 - DNA, 20 bp, Artificial Sequence
  Description of Artificial Sequence Synthetic oligonucleotide
gaaagccaacgtacccaaga

SEQ ID NO: 172 - DNA, 20 bp, Artificial Sequence
  Description of Artificial Sequence Synthetic oligonucleotide
gatggtccgactcgccggtc

SEQ ID NO: 173 - DNA, 20 bp, Artificial Sequence
  Description of Artificial Sequence Synthetic oligonucleotide
gccatacacgtccggtctca

SEQ ID NO: 174 - DNA, 20 bp, Artificial Sequence
  Description of Artificial Sequence Synthetic oligonucleotide
gatagaaacctgcgtagtac

SEQ ID NO: 175 - DNA, 20 bp, Artificial Sequence
  Description of Artificial Sequence Synthetic oligonucleotide
gttgagaccggacgtgtatg

SEQ ID NO: 176 - DNA, 20 bp, Artificial Sequence
  Description of Artificial Sequence Synthetic oligonucleotide
gctctttccccatacacgtc

SEQ ID NO: 177 - DNA, 20 bp, Artificial Sequence
  Description of Artificial Sequence Synthetic oligonucleotide
gtggatagatccgacactga

SEQ ID NO: 178 - DNA, 20 bp, Artificial Sequence
  Description of Artificial Sequence Synthetic oligonucleotide
ggtcgagtattcaacggatc

SEQ ID NO: 179 - DNA, 20 bp, Artificial Sequence
  Description of Artificial Sequence Synthetic oligonucleotide
ggctggattaccacacaacg

SEQ ID NO: 180 - DNA, 20 bp, Artificial Sequence
  Description of Artificial Sequence Synthetic oligonucleotide
gggacaatctcagcgtatct

SEQ ID NO: 181 - DNA, 20 bp, Artificial Sequence
  Description of Artificial Sequence Synthetic oligonucleotide
ggtcctggtgatctcgatga

SEQ ID NO: 182 - DNA, 20 bp, Artificial Sequence
  Description of Artificial Sequence Synthetic oligonucleotide
ggagctatacggcaaacttg

SEQ ID NO: 183 - DNA, 20 bp, Artificial Sequence
  Description of Artificial Sequence Synthetic oligonucleotide
gaagagctatacggcaaact

SEQ ID NO: 184 - DNA, 20 bp, Artificial Sequence
  Description of Artificial Sequence Synthetic oligonucleotide
gtagctcccttcggaccatc

SEQ ID NO: 185 - DNA, 20 bp, Artificial Sequence
  Description of Artificial Sequence Synthetic oligonucleotide
gagagctatacggcaaactt

SEQ ID NO: 186 - DNA, 20 bp, Artificial Sequence
  Description of Artificial Sequence Synthetic oligonucleotide
gctgtgacatacactttccg

SEQ ID NO: 187 - DNA, 20 bp, Artificial Sequence
  Description of Artificial Sequence Synthetic oligonucleotide
gttcatggcggttacggaag

SEQ ID NO: 188 - DNA, 20 bp, Artificial Sequence
  Description of Artificial Sequence Synthetic oligonucleotide
gggcccagcggtggcaacga

SEQ ID NO: 189 - DNA, 20 bp, Artificial Sequence
  Description of Artificial Sequence Synthetic oligonucleotide
gccggtgcacagcgggcatt

SEQ ID NO: 190 - DNA, 20 bp, Artificial Sequence
  Description of Artificial Sequence Synthetic oligonucleotide
gaggcccagcggtggcaacg

SEQ ID NO: 191 - DNA, 20 bp, Artificial Sequence
  Description of Artificial Sequence Synthetic oligonucleotide
gcatctctgcgaagtcacga

SEQ ID NO: 192 - DNA, 20 bp, Artificial Sequence
  Description of Artificial Sequence Synthetic oligonucleotide
ggcccagcggtggcaacgag

SEQ ID NO: 193 - DNA, 20 bp, Artificial Sequence
  Description of Artificial Sequence Synthetic oligonucleotide
gccctcgggcccaatgcaca

SEQ ID NO: 194 - DNA, 20 bp, Artificial Sequence
  Description of Artificial Sequence Synthetic oligonucleotide
gaccatgtgcattgggcccg

SEQ ID NO: 195 - DNA, 20 bp, Artificial Sequence
  Description of Artificial Sequence Synthetic oligonucleotide
gccatgtgcattgggcccga

SEQ ID NO: 196 - DNA, 20 bp, Artificial Sequence
  Description of Artificial Sequence Synthetic oligonucleotide
gcgccgacaatggaggcgcc

SEQ ID NO: 197 - DNA, 20 bp, Artificial Sequence
  Description of Artificial Sequence Synthetic oligonucleotide
ggaagctgtcatcgtgctac

SEQ ID NO: 198 - DNA, 20 bp, Artificial Sequence
  Description of Artificial Sequence Synthetic oligonucleotide
ggtgggagatgttcggcagg

SEQ ID NO: 199 - DNA, 20 bp, Artificial Sequence
  Description of Artificial Sequence Synthetic oligonucleotide
gccactacagtaatgtccga

SEQ ID NO: 200 - DNA, 20 bp, Artificial Sequence
  Description of Artificial Sequence Synthetic oligonucleotide
ggttcggcaggcggccgatg

SEQ ID NO: 201 - DNA, 20 bp, Artificial Sequence
  Description of Artificial Sequence Synthetic oligonucleotide
gaagctgtcatcgtgctact

SEQ ID NO: 202 - DNA, 20 bp, Artificial Sequence
  Description of Artificial Sequence Synthetic oligonucleotide
gtgtgtgaatggacctgtta

SEQ ID NO: 203 - DNA, 20 bp, Artificial Sequence
  Description of Artificial Sequence Synthetic oligonucleotide
gagggatgagactcctctat

SEQ ID NO: 204 - DNA, 20 bp, Artificial Sequence
  Description of Artificial Sequence Synthetic oligonucleotide
gggaggaggctcggaactgt

SEQ ID NO: 205 - DNA, 20 bp, Artificial Sequence
  Description of Artificial Sequence Synthetic oligonucleotide
ggtctgaatgagcgtcacta

SEQ ID NO: 206 - DNA, 20 bp, Artificial Sequence
  Description of Artificial Sequence Synthetic oligonucleotide
ggccttgattggcctcaaca

SEQ ID NO: 207 - DNA, 20 bp, Artificial Sequence
  Description of Artificial Sequence Synthetic oligonucleotide
gatagcaaatactttggccg

SEQ ID NO: 208 - DNA, 20 bp, Artificial Sequence
  Description of Artificial Sequence Synthetic oligonucleotide
gcctcatagtccagaccgca

SEQ ID NO: 209 - DNA, 20 bp, Artificial Sequence
  Description of Artificial Sequence Synthetic oligonucleotide
gcacgagaatggcgacaagc

SEQ ID NO: 210 - DNA, 20 bp, Artificial Sequence
  Description of Artificial Sequence Synthetic oligonucleotide
gtcggccgtcgccgtctgtg

SEQ ID NO: 211 - DNA, 20 bp, Artificial Sequence
  Description of Artificial Sequence Synthetic oligonucleotide
ggtgccgtcaatgcgtcctg

SEQ ID NO: 212 - DNA, 20 bp, Artificial Sequence
  Description of Artificial Sequence Synthetic oligonucleotide
ggagaatattcggattcaac

SEQ ID NO: 213 - DNA, 20 bp, Artificial Sequence
  Description of Artificial Sequence Synthetic oligonucleotide
gatcccaagtccactgcgct

SEQ ID NO: 214 - DNA, 20 bp, Artificial Sequence
  Description of Artificial Sequence Synthetic oligonucleotide
gttttcaccgcaaaagctac

SEQ ID NO: 215 - DNA, 20 bp, Artificial Sequence
  Description of Artificial Sequence Synthetic oligonucleotide
gtctccaagcgcagtggact

SEQ ID NO: 216 - DNA, 20 bp, Artificial Sequence
  Description of Artificial Sequence Synthetic oligonucleotide
gtatgacccattccggatta

SEQ ID NO: 217 - DNA, 20 bp, Artificial Sequence
  Description of Artificial Sequence Synthetic oligonucleotide
ggttctccataatccggaat

SEQ ID NO: 218 - DNA, 20 bp, Artificial Sequence
  Description of Artificial Sequence Synthetic oligonucleotide
gatgggtggcctctgcatcg

SEQ ID NO: 219 - DNA, 20 bp, Artificial Sequence
  Description of Artificial Sequence Synthetic oligonucleotide
gggttctccataatccggaa

SEQ ID NO: 220 - DNA, 20 bp, Artificial Sequence
  Description of Artificial Sequence Synthetic oligonucleotide
gactaggtgtgtgtgccgag

SEQ ID NO: 221 - DNA, 20 bp, Artificial Sequence
  Description of Artificial Sequence Synthetic oligonucleotide
gctcggcacacacacctagt

SEQ ID NO: 222 - DNA, 20 bp, Artificial Sequence
  Description of Artificial Sequence Synthetic oligonucleotide
gggggctgccctgctcaccg

SEQ ID NO: 223 - DNA, 20 bp, Artificial Sequence
  Description of Artificial Sequence Synthetic oligonucleotide
gtccagaaggccatctccga

SEQ ID NO: 224 - DNA, 20 bp, Artificial Sequence
  Description of Artificial Sequence Synthetic oligonucleotide
ggatcatcaccatcgggtac

SEQ ID NO: 225 - DNA, 20 bp, Artificial Sequence
  Description of Artificial Sequence Synthetic oligonucleotide
gccttgaagatagtccggac

SEQ ID NO: 226 - DNA, 20 bp, Artificial Sequence
  Description of Artificial Sequence Synthetic oligonucleotide
ggggctattaacagttcgca

SEQ ID NO: 227 - DNA, 20 bp, Artificial Sequence
  Description of Artificial Sequence Synthetic oligonucleotide
gcttcgccacgtttgttcag

SEQ ID NO: 228 - DNA, 20 bp, Artificial Sequence
  Description of Artificial Sequence Synthetic oligonucleotide
gcttgaatggagcgaatcgt

SEQ ID NO: 229 - DNA, 20 bp, Artificial Sequence
  Description of Artificial Sequence Synthetic oligonucleotide
gcagcgctcgatcatgtcca

SEQ ID NO: 230 - DNA, 20 bp, Artificial Sequence
  Description of Artificial Sequence Synthetic oligonucleotide
ggccattgtgaagcggtttg

SEQ ID NO: 231 - DNA, 20 bp, Artificial Sequence
  Description of Artificial Sequence Synthetic oligonucleotide
gcagcaggccgattggaggc

SEQ ID NO: 232 - DNA, 20 bp, Artificial Sequence
  Description of Artificial Sequence Synthetic oligonucleotide
gcctgctgacgtcagacggg

SEQ ID NO: 233 - DNA, 20 bp, Artificial Sequence
  Description of Artificial Sequence Synthetic oligonucleotide
gactgcttgtctcttaagcg

SEQ ID NO: 234 - DNA, 20 bp, Artificial Sequence
  Description of Artificial Sequence Synthetic oligonucleotide
ggagacgcactgctttcgtc

SEQ ID NO: 235 - DNA, 20 bp, Artificial Sequence
  Description of Artificial Sequence Synthetic oligonucleotide
gcaggaacggggctaccgac

SEQ ID NO: 236 - DNA, 20 bp, Artificial Sequence
  Description of Artificial Sequence Synthetic oligonucleotide
gccataagaacgcaggaacg

SEQ ID NO: 237 - DNA, 20 bp, Artificial Sequence
  Description of Artificial Sequence Synthetic oligonucleotide
gcaaatttctgcgtcagtat

SEQ ID NO: 238 - DNA, 20 bp, Artificial Sequence
  Description of Artificial Sequence Synthetic oligonucleotide
gtcaaatttctgcgtcagta

SEQ ID NO: 239 - DNA, 20 bp, Artificial Sequence
  Description of Artificial Sequence Synthetic oligonucleotide
ggactgaggtaatgaaggtc

SEQ ID NO: 240 - DNA, 20 bp, Artificial Sequence
  Description of Artificial Sequence Synthetic oligonucleotide
gggaatacccaggctcacgt

SEQ ID NO: 241 - DNA, 20 bp, Artificial Sequence
  Description of Artificial Sequence Synthetic oligonucleotide
ggggaatacccaggctcacg

SEQ ID NO: 242 - DNA, 20 bp, Artificial Sequence
  Description of Artificial Sequence Synthetic oligonucleotide
gtgttatagagaacgacgtc

SEQ ID NO: 243 - DNA, 20 bp, Artificial Sequence
  Description of Artificial Sequence Synthetic oligonucleotide
ggacttgtacaatctcacgg

SEQ ID NO: 244 - DNA, 20 bp, Artificial Sequence
  Description of Artificial Sequence Synthetic oligonucleotide
gcgctgcatgacgaagtggt

SEQ ID NO: 245 - DNA, 20 bp, Artificial Sequence
  Description of Artificial Sequence Synthetic oligonucleotide
gacctgtttcagacgtacat

SEQ ID NO: 246 - DNA, 20 bp, Artificial Sequence
  Description of Artificial Sequence Synthetic oligonucleotide
gccaccacctaccgggttaa

SEQ ID NO: 247 - DNA, 20 bp, Artificial Sequence
  Description of Artificial Sequence Synthetic oligonucleotide
gctctaccgctgcgatcttt

SEQ ID NO: 248 - DNA, 20 bp, Artificial Sequence
  Description of Artificial Sequence Synthetic oligonucleotide
gcgggatgaaggtatttcgc

SEQ ID NO: 249 - DNA, 20 bp, Artificial Sequence
  Description of Artificial Sequence Synthetic oligonucleotide
ggtgttcaccacgtggcgct

SEQ ID NO: 250 - DNA, 20 bp, Artificial Sequence
  Description of Artificial Sequence Synthetic oligonucleotide
gtgcaaccccgacgggcctc

SEQ ID NO: 251 - DNA, 20 bp, Artificial Sequence
  Description of Artificial Sequence Synthetic oligonucleotide
ggcctctgggtgccgcggta

SEQ ID NO: 252 - DNA, 20 bp, Artificial Sequence
  Description of Artificial Sequence Synthetic oligonucleotide
gcactgcacggcgctctcca

SEQ ID NO: 253 - DNA, 20 bp, Artificial Sequence
  Description of Artificial Sequence Synthetic oligonucleotide
ggcctgacgggcgagttcaa

SEQ ID NO: 254 - DNA, 20 bp, Artificial Sequence
  Description of Artificial Sequence Synthetic oligonucleotide
ggcccttgaactcgcccgtc

SEQ ID NO: 255 - DNA, 20 bp, Artificial Sequence
  Description of Artificial Sequence Synthetic oligonucleotide
gtacaataagcttcgcgata

SEQ ID NO: 256 - DNA, 20 bp, Artificial Sequence
  Description of Artificial Sequence Synthetic oligonucleotide
gggctgtgtggccggcgatg

SEQ ID NO: 257 - DNA, 20 bp, Artificial Sequence
  Description of Artificial Sequence Synthetic oligonucleotide
gcatataaggcacaggacgg

SEQ ID NO: 258 - DNA, 20 bp, Artificial Sequence
  Description of Artificial Sequence Synthetic oligonucleotide
gaaacagcaaccgaccccca

SEQ ID NO: 259 - DNA, 20 bp, Artificial Sequence
  Description of Artificial Sequence Synthetic oligonucleotide
gtcggacaccgagcatgact

SEQ ID NO: 260 - DNA, 20 bp, Artificial Sequence
  Description of Artificial Sequence Synthetic oligonucleotide
gatgatgatcacgttgacct

SEQ ID NO: 261 - DNA, 20 bp, Artificial Sequence
  Description of Artificial Sequence Synthetic oligonucleotide
ggttccaaacggccaggaca

SEQ ID NO: 262 - DNA, 20 bp, Artificial Sequence
  Description of Artificial Sequence Synthetic oligonucleotide
gaaagagcggctcttccgtg

SEQ ID NO: 263 - DNA, 20 bp, Artificial Sequence
  Description of Artificial Sequence Synthetic oligonucleotide
gcttccatcacgtatccccc

SEQ ID NO: 264 - DNA, 20 bp, Artificial Sequence
  Description of Artificial Sequence Synthetic oligonucleotide
ggcgtgcgcaccaagaccgt

SEQ ID NO: 265 - DNA, 20 bp, Artificial Sequence
  Description of Artificial Sequence Synthetic oligonucleotide
gaggaccactccggtcaaga

SEQ ID NO: 266 - DNA, 20 bp, Artificial Sequence
  Description of Artificial Sequence Synthetic oligonucleotide
ggcccatcctgctgtacgat

SEQ ID NO: 267 - DNA, 20 bp, Artificial Sequence
  Description of Artificial Sequence Synthetic oligonucleotide
gctgcagatcgaggagtttc

SEQ ID NO: 268 - DNA, 20 bp, Artificial Sequence
  Description of Artificial Sequence Synthetic oligonucleotide
gcccatcctgctgtacgatg

SEQ ID NO: 269 - DNA, 20 bp, Artificial Sequence
  Description of Artificial Sequence Synthetic oligonucleotide
gtgctgaaggacttcgcacc

SEQ ID NO: 270 - DNA, 20 bp, Artificial Sequence
  Description of Artificial Sequence Synthetic oligonucleotide
gcatcatctcgattcgtgcg

SEQ ID NO: 271 - DNA, 20 bp, Artificial Sequence
  Description of Artificial Sequence Synthetic oligonucleotide
ggggagagtcgtgcatactc

SEQ ID NO: 272 - DNA, 20 bp, Artificial Sequence
  Description of Artificial Sequence Synthetic oligonucleotide
gaccccaacgtgttcaggat

SEQ ID NO: 273 - DNA, 20 bp, Artificial Sequence
  Description of Artificial Sequence Synthetic oligonucleotide
gaggttctggctccggttgt

SEQ ID NO: 274 - DNA, 20 bp, Artificial Sequence
  Description of Artificial Sequence Synthetic oligonucleotide
gtctggctccggttgtgggt

SEQ ID NO: 275 - DNA, 20 bp, Artificial Sequence
  Description of Artificial Sequence Synthetic oligonucleotide
gcctcttcaggtactggttc

SEQ ID NO: 276 - DNA, 20 bp, Artificial Sequence
  Description of Artificial Sequence Synthetic oligonucleotide
gcaagaaggcgcgtgatgcg

SEQ ID NO: 277 - DNA, 20 bp, Artificial Sequence
  Description of Artificial Sequence Synthetic oligonucleotide
gcagtacacttgaaaggcgg

SEQ ID NO: 278 - DNA, 20 bp, Artificial Sequence
  Description of Artificial Sequence Synthetic oligonucleotide
gattgataagcgtgttggcc

SEQ ID NO: 279 - DNA, 20 bp, Artificial Sequence
  Description of Artificial Sequence Synthetic oligonucleotide
gacaaggccgaggtacatgc

SEQ ID NO: 280 - DNA, 20 bp, Artificial Sequence
  Description of Artificial Sequence Synthetic oligonucleotide
ggcagtgagcagttccgcag

SEQ ID NO: 281 - DNA, 20 bp, Artificial Sequence
  Description of Artificial Sequence Synthetic oligonucleotide
ggggatttaccgaaccgaaa

SEQ ID NO: 282 - DNA, 20 bp, Artificial Sequence
  Description of Artificial Sequence Synthetic oligonucleotide
gaacctcaccgcagcgctgc

SEQ ID NO: 283 - DNA, 20 bp, Artificial Sequence
  Description of Artificial Sequence Synthetic oligonucleotide
gtgtctgctggagtatcccc

SEQ ID NO: 284 - DNA, 20 bp, Artificial Sequence
  Description of Artificial Sequence Synthetic oligonucleotide
gaccaggaattactacgtcc

SEQ ID NO: 285 - DNA, 20 bp, Artificial Sequence
  Description of Artificial Sequence Synthetic oligonucleotide
gcaccaggaattactacgtc

SEQ ID NO: 286 - DNA, 20 bp, Artificial Sequence
  Description of Artificial Sequence Synthetic oligonucleotide
gggggtccaccatggagcga

SEQ ID NO: 287 - DNA, 20 bp, Artificial Sequence
  Description of Artificial Sequence Synthetic oligonucleotide
gtatgtggccgtgactcggt

SEQ ID NO: 288 - DNA, 20 bp, Artificial Sequence
  Description of Artificial Sequence Synthetic oligonucleotide
gttaccagacgaattgtacg

SEQ ID NO: 289 - DNA, 20 bp, Artificial Sequence
  Description of Artificial Sequence Synthetic oligonucleotide
gccgagtcacggccacatac

SEQ ID NO: 290 - DNA, 20 bp, Artificial Sequence
  Description of Artificial Sequence Synthetic oligonucleotide
gattatactcgaaaactcct

SEQ ID NO: 291 - DNA, 20 bp, Artificial Sequence
  Description of Artificial Sequence Synthetic oligonucleotide
gtaggaactccgcatcgtcg

SEQ ID NO: 292 - DNA, 20 bp, Artificial Sequence
  Description of Artificial Sequence Synthetic oligonucleotide
gccactgatcggaaacgcgg

SEQ ID NO: 293 - DNA, 20 bp, Artificial Sequence
  Description of Artificial Sequence Synthetic oligonucleotide
gccacaatgaccggctgcgt

SEQ ID NO: 294 - DNA, 20 bp, Artificial Sequence
  Description of Artificial Sequence Synthetic oligonucleotide
gcttctcgagggccacgcgc

SEQ ID NO: 295 - DNA, 20 bp, Artificial Sequence
  Description of Artificial Sequence Synthetic oligonucleotide
gattgtagcgttccggtccc

SEQ ID NO: 296 - DNA, 20 bp, Artificial Sequence
  Description of Artificial Sequence Synthetic oligonucleotide
ggcagaggacgcgtccattt

SEQ ID NO: 297 - DNA, 20 bp, Artificial Sequence
  Description of Artificial Sequence Synthetic oligonucleotide
gaaggcatccgtgtagagcg

SEQ ID NO: 298 - DNA, 20 bp, Artificial Sequence
  Description of Artificial Sequence Synthetic oligonucleotide
gtacacggatgccttaaacg

SEQ ID NO: 299 - DNA, 20 bp, Artificial Sequence
  Description of Artificial Sequence Synthetic oligonucleotide
gccgcgcctgcggagtccgg

SEQ ID NO: 300 - DNA, 20 bp, Artificial Sequence
  Description of Artificial Sequence Synthetic oligonucleotide
gaagcagccgcgggctatgc

SEQ ID NO: 301 - DNA, 20 bp, Artificial Sequence
  Description of Artificial Sequence Synthetic oligonucleotide
ggggctacccggcctagttc

SEQ ID NO: 302 - DNA, 20 bp, Artificial Sequence
  Description of Artificial Sequence Synthetic oligonucleotide
gtgcatggcgctgcgcggta

SEQ ID NO: 303 - DNA, 20 bp, Artificial Sequence
  Description of Artificial Sequence Synthetic oligonucleotide
gctaagtatcctccgtgact

SEQ ID NO: 304 - DNA, 20 bp, Artificial Sequence
  Description of Artificial Sequence Synthetic oligonucleotide
gtgtagttggcaaagcgctt

SEQ ID NO: 305 - DNA, 20 bp, Artificial Sequence
  Description of Artificial Sequence Synthetic oligonucleotide
gcacatggaaggacgtgcgg

SEQ ID NO: 306 - DNA, 20 bp, Artificial Sequence
  Description of Artificial Sequence Synthetic oligonucleotide
gcacgatgcgcctctgaccc

SEQ ID NO: 307 - DNA, 20 bp, Artificial Sequence
  Description of Artificial Sequence Synthetic oligonucleotide
ggcaggcccgcaagaaacgg

SEQ ID NO: 308 - DNA, 20 bp, Artificial Sequence
  Description of Artificial Sequence Synthetic oligonucleotide
gaaagctgtatacagccgag

SEQ ID NO: 309 - DNA, 20 bp, Artificial Sequence
  Description of Artificial Sequence Synthetic oligonucleotide
gaagctgtatacagccgagg

SEQ ID NO: 310 - DNA, 20 bp, Artificial Sequence
  Description of Artificial Sequence Synthetic oligonucleotide
gctaaagctgtatacagccg

SEQ ID NO: 311 - DNA, 20 bp, Artificial Sequence
  Description of Artificial Sequence Synthetic oligonucleotide
gttatgtcctcgtatgtttc

SEQ ID NO: 312 - DNA, 20 bp, Artificial Sequence
  Description of Artificial Sequence Synthetic oligonucleotide
gggaaaagttgccatccgaa

SEQ ID NO: 313 - DNA, 20 bp, Artificial Sequence
  Description of Artificial Sequence Synthetic oligonucleotide
ggggaagtaagcgaagaaat

SEQ ID NO: 314 - DNA, 20 bp, Artificial Sequence
  Description of Artificial Sequence Synthetic oligonucleotide
gctgggtatgagctagccgt

SEQ ID NO: 315 - DNA, 20 bp, Artificial Sequence
  Description of Artificial Sequence Synthetic oligonucleotide
gcgctccagggcttcccgaa

SEQ ID NO: 316 - DNA, 20 bp, Artificial Sequence
  Description of Artificial Sequence Synthetic oligonucleotide
gacagtattgactccgatgg

SEQ ID NO: 317 - DNA, 20 bp, Artificial Sequence
  Description of Artificial Sequence Synthetic oligonucleotide
ggcctgagactcgtcatcca

SEQ ID NO: 318 - DNA, 20 bp, Artificial Sequence
  Description of Artificial Sequence Synthetic oligonucleotide
gctcgttccagcgatccaag

SEQ ID NO: 319 - DNA, 20 bp, Artificial Sequence
  Description of Artificial Sequence Synthetic oligonucleotide
gtgccggatctggcccgacg

SEQ ID NO: 320 - DNA, 20 bp, Artificial Sequence
  Description of Artificial Sequence Synthetic oligonucleotide
gacattggcctcaatcggga

SEQ ID NO: 321 - DNA, 20 bp, Artificial Sequence
  Description of Artificial Sequence Synthetic oligonucleotide
gggctaggcgtacggcatcc

SEQ ID NO: 322 - DNA, 20 bp, Artificial Sequence
  Description of Artificial Sequence Synthetic oligonucleotide
gctaatgacacggccacgtc

SEQ ID NO: 323 - DNA, 20 bp, Artificial Sequence
  Description of Artificial Sequence Synthetic oligonucleotide
gatctgcgcaatttcacccg

SEQ ID NO: 324 - DNA, 20 bp, Artificial Sequence
  Description of Artificial Sequence Synthetic oligonucleotide
gccaatgccacgaccgctcc

SEQ ID NO: 325 - DNA, 20 bp, Artificial Sequence
  Description of Artificial Sequence Synthetic oligonucleotide
gctgactgcgagcccatgaa

SEQ ID NO: 326 - DNA, 20 bp, Artificial Sequence
  Description of Artificial Sequence Synthetic oligonucleotide
ggagttaacgcctttatctc

SEQ ID NO: 327 - DNA, 20 bp, Artificial Sequence
  Description of Artificial Sequence Synthetic oligonucleotide
ggaaggtggtagccgatgcg

SEQ ID NO: 328 - DNA, 20 bp, Artificial Sequence
  Description of Artificial Sequence Synthetic oligonucleotide
gcacatgcacgacgtataaa

SEQ ID NO: 329 - DNA, 20 bp, Artificial Sequence
  Description of Artificial Sequence Synthetic oligonucleotide
gtcctgaggacgcatctcgg

SEQ ID NO: 330 - DNA, 20 bp, Artificial Sequence
  Description of Artificial Sequence Synthetic oligonucleotide
gcaccttggtgacgtatagt

SEQ ID NO: 331 - DNA, 20 bp, Artificial Sequence
  Description of Artificial Sequence Synthetic oligonucleotide
gcacctgaccgtcagtacac

SEQ ID NO: 332 - DNA, 20 bp, Artificial Sequence
  Description of Artificial Sequence Synthetic oligonucleotide
ggggagttgacgttcacacc

SEQ ID NO: 333 - DNA, 20 bp, Artificial Sequence
  Description of Artificial Sequence Synthetic oligonucleotide
gccgcctgctagagccgcct

SEQ ID NO: 334 - DNA, 20 bp, Artificial Sequence
  Description of Artificial Sequence Synthetic oligonucleotide
gtgggtgaccgctaatcatc

SEQ ID NO: 335 - DNA, 20 bp, Artificial Sequence
  Description of Artificial Sequence Synthetic oligonucleotide
gcatcagcatgccgaccagg

SEQ ID NO: 336 - DNA, 20 bp, Artificial Sequence
  Description of Artificial Sequence Synthetic oligonucleotide
gagaaggttgaccgcctggt

SEQ ID NO: 337 - DNA, 20 bp, Artificial Sequence
  Description of Artificial Sequence Synthetic oligonucleotide
gtgtgaaagcatcgataccc

SEQ ID NO: 338 - DNA, 20 bp, Artificial Sequence
  Description of Artificial Sequence Synthetic oligonucleotide
ggattgggaaacgtcttggt

SEQ ID NO: 339 - DNA, 20 bp, Artificial Sequence
  Description of Artificial Sequence Synthetic oligonucleotide
ggcgtacctggccgaatgca

SEQ ID NO: 340 - DNA, 20 bp, Artificial Sequence
  Description of Artificial Sequence Synthetic oligonucleotide
gtgggattgtgttggatgcg

SEQ ID NO: 341 - DNA, 20 bp, Artificial Sequence
  Description of Artificial Sequence Synthetic oligonucleotide
gcaatcggcagacgaggtcc

SEQ ID NO: 342 - DNA, 20 bp, Artificial Sequence
  Description of Artificial Sequence Synthetic oligonucleotide
gcaattccgtctctatggcg

SEQ ID NO: 343 - DNA, 20 bp, Artificial Sequence
  Description of Artificial Sequence Synthetic oligonucleotide
gagcgaacaatcggcagacg

SEQ ID NO: 344 - DNA, 20 bp, Artificial Sequence
  Description of Artificial Sequence Synthetic oligonucleotide
gcccagatatccttcgtggc

SEQ ID NO: 345 - DNA, 20 bp, Artificial Sequence
  Description of Artificial Sequence Synthetic oligonucleotide
ggcaccgtgtcgctggcgtt

SEQ ID NO: 346 - DNA, 20 bp, Artificial Sequence
  Description of Artificial Sequence Synthetic oligonucleotide
gatggcgtcgtgctcgatgc

SEQ ID NO: 347 - DNA, 20 bp, Artificial Sequence
  Description of Artificial Sequence Synthetic oligonucleotide
gtccacccagatatccttcg

SEQ ID NO: 348 - DNA, 20 bp, Artificial Sequence
  Description of Artificial Sequence Synthetic oligonucleotide
gacattcaaggcggcgactg

SEQ ID NO: 349 - DNA, 20 bp, Artificial Sequence
  Description of Artificial Sequence Synthetic oligonucleotide
gtgctcatcactcaggccgt

SEQ ID NO: 350 - DNA, 20 bp, Artificial Sequence
  Description of Artificial Sequence Synthetic oligonucleotide
ggagagcatccggcccttca

SEQ ID NO: 351 - DNA, 20 bp, Artificial Sequence
  Description of Artificial Sequence Synthetic oligonucleotide
gactcaggccgtgggtcttg

SEQ ID NO: 352 - DNA, 20 bp, Artificial Sequence
  Description of Artificial Sequence Synthetic oligonucleotide
gctacacagacgacatcaac

SEQ ID NO: 353 - DNA, 20 bp, Artificial Sequence
  Description of Artificial Sequence Synthetic oligonucleotide
gcctctagctatggtatcgc

SEQ ID NO: 354 - DNA, 20 bp, Artificial Sequence
  Description of Artificial Sequence Synthetic oligonucleotide
gttctcaggttcgattatga

SEQ ID NO: 355 - DNA, 20 bp, Artificial Sequence
  Description of Artificial Sequence Synthetic oligonucleotide
gctgccatagagcgcatacc

SEQ ID NO: 356 - DNA, 20 bp, Artificial Sequence
  Description of Artificial Sequence Synthetic oligonucleotide
gtggcgcaccgcagacgaga

SEQ ID NO: 357 - DNA, 20 bp, Artificial Sequence
  Description of Artificial Sequence Synthetic oligonucleotide
gcagccgggtatgcgctcta

SEQ ID NO: 358 - DNA, 20 bp, Artificial Sequence
  Description of Artificial Sequence Synthetic oligonucleotide
ggtagatgtctacgtgggca

SEQ ID NO: 359 - DNA, 20 bp, Artificial Sequence
  Description of Artificial Sequence Synthetic oligonucleotide
ggttgggggagcactcatac

SEQ ID NO: 360 - DNA, 20 bp, Artificial Sequence
  Description of Artificial Sequence Synthetic oligonucleotide
gggctttgtcttatggtgtt

SEQ ID NO: 361 - DNA, 20 bp, Artificial Sequence
  Description of Artificial Sequence Synthetic oligonucleotide
gtggcgtaaagagcgtttcc

SEQ ID NO: 362 - DNA, 20 bp, Artificial Sequence
  Description of Artificial Sequence Synthetic oligonucleotide
gcaggtaaaggaggtacgac

SEQ ID NO: 363 - DNA, 20 bp, Artificial Sequence
  Description of Artificial Sequence Synthetic oligonucleotide
gtcccaactagtgtagtcca

SEQ ID NO: 364 - DNA, 20 bp, Artificial Sequence
  Description of Artificial Sequence Synthetic oligonucleotide
gctacctgggtgacttcgcg

SEQ ID NO: 365 - DNA, 20 bp, Artificial Sequence
  Description of Artificial Sequence Synthetic oligonucleotide
gcaccatgtcgctggcgtcg

SEQ ID NO: 366 - DNA, 20 bp, Artificial Sequence
  Description of Artificial Sequence Synthetic oligonucleotide
gcggaggttgaactcggcgt

SEQ ID NO: 367 - DNA, 20 bp, Artificial Sequence
  Description of Artificial Sequence Synthetic oligonucleotide
ggtcgttgtcatcctcgggg

SEQ ID NO: 368 - DNA, 20 bp, Artificial Sequence
  Description of Artificial Sequence Synthetic oligonucleotide
gcttcctgcatcgagccggc

SEQ ID NO: 369 - DNA, 20 bp, Artificial Sequence
  Description of Artificial Sequence Synthetic oligonucleotide
gggtgaaggaactccacgac

SEQ ID NO: 370 - DNA, 20 bp, Artificial Sequence
  Description of Artificial Sequence Synthetic oligonucleotide
gaccggcgtcgcacacggtg

SEQ ID NO: 371 - DNA, 20 bp, Artificial Sequence
  Description of Artificial Sequence Synthetic oligonucleotide
gcagctccgcaacgcctggt

SEQ ID NO: 372 - DNA, 20 bp, Artificial Sequence
  Description of Artificial Sequence Synthetic oligonucleotide
ggcgtgttcgacgaccagga

SEQ ID NO: 373 - DNA, 20 bp, Artificial Sequence
  Description of Artificial Sequence Synthetic oligonucleotide
ggccgctgatgttgaagcgc

SEQ ID NO: 374 - DNA, 20 bp, Artificial Sequence
  Description of Artificial Sequence Synthetic oligonucleotide
gattggcagtcggcctatct

SEQ ID NO: 375 - DNA, 20 bp, Artificial Sequence
  Description of Artificial Sequence Synthetic oligonucleotide
gggggatggggctccggttc

SEQ ID NO: 376 - DNA, 20 bp, Artificial Sequence
  Description of Artificial Sequence Synthetic oligonucleotide
ggagattcaggcggagttag

SEQ ID NO: 377 - DNA, 20 bp, Artificial Sequence
  Description of Artificial Sequence Synthetic oligonucleotide
gttcaggctgaggttcgggt

SEQ ID NO: 378 - DNA, 20 bp, Artificial Sequence
  Description of Artificial Sequence Synthetic oligonucleotide
ggggctccggttccggctcc

SEQ ID NO: 379 - DNA, 20 bp, Artificial Sequence
  Description of Artificial Sequence Synthetic oligonucleotide
gcaggctgaggttcgggttg

SEQ ID NO: 380 - DNA, 20 bp, Artificial Sequence
  Description of Artificial Sequence Synthetic oligonucleotide
gcaccatattgctattcaac

SEQ ID NO: 381 - DNA, 20 bp, Artificial Sequence
  Description of Artificial Sequence Synthetic oligonucleotide
gaacttcgacctgacatcgg

SEQ ID NO: 382 - DNA, 20 bp, Artificial Sequence
  Description of Artificial Sequence Synthetic oligonucleotide
ggataaacaatttcttcacg

SEQ ID NO: 383 - DNA, 20 bp, Artificial Sequence
  Description of Artificial Sequence Synthetic oligonucleotide
ggttgtcatacccttccagc

SEQ ID NO: 384 - DNA, 20 bp, Artificial Sequence
  Description of Artificial Sequence Synthetic oligonucleotide
gacgtctgtcataccaggtt

SEQ ID NO: 385 - DNA, 20 bp, Artificial Sequence
  Description of Artificial Sequence Synthetic oligonucleotide
gttggcagcccccggacaga

SEQ ID NO: 386 - DNA, 20 bp, Artificial Sequence
  Description of Artificial Sequence Synthetic oligonucleotide
gctggagctcgtgaccgtga

SEQ ID NO: 387 - DNA, 20 bp, Artificial Sequence
  Description of Artificial Sequence Synthetic oligonucleotide
gccgcagcttccccgctcgt

SEQ ID NO: 388 - DNA, 20 bp, Artificial Sequence
  Description of Artificial Sequence Synthetic oligonucleotide
gcaaataggagcgcccatta

SEQ ID NO: 389 - DNA, 20 bp, Artificial Sequence
  Description of Artificial Sequence Synthetic oligonucleotide
ggatctatggatgcgatgtc

SEQ ID NO: 390 - DNA, 20 bp, Artificial Sequence
  Description of Artificial Sequence Synthetic oligonucleotide
ggctgcccctagcaaccgcg

SEQ ID NO: 391 - DNA, 20 bp, Artificial Sequence
  Description of Artificial Sequence Synthetic oligonucleotide
gatcaccgcgcggttgctag

SEQ ID NO: 392 - DNA, 20 bp, Artificial Sequence
  Description of Artificial Sequence Synthetic oligonucleotide
gtagcaaccgcgcggtgatc

SEQ ID NO: 393 - DNA, 20 bp, Artificial Sequence
  Description of Artificial Sequence Synthetic oligonucleotide
gatctccacgcctttggtgt

SEQ ID NO: 394 - DNA, 20 bp, Artificial Sequence
  Description of Artificial Sequence Synthetic oligonucleotide
ggtagcccatgcggttgtag

SEQ ID NO: 395 - DNA, 20 bp, Artificial Sequence
  Description of Artificial Sequence Synthetic oligonucleotide
gggtacctctacaaccgcat

SEQ ID NO: 396 - DNA, 20 bp, Artificial Sequence
  Description of Artificial Sequence Synthetic oligonucleotide
gaactgtccagcgacgatcc

SEQ ID NO: 397 - DNA, 20 bp, Artificial Sequence
  Description of Artificial Sequence Synthetic oligonucleotide
gatgtccttccggaaggcgc

SEQ ID NO: 398 - DNA, 20 bp, Artificial Sequence
  Description of Artificial Sequence Synthetic oligonucleotide
ggatctgctcggatattgga

SEQ ID NO: 399 - DNA, 20 bp, Artificial Sequence
  Description of Artificial Sequence Synthetic oligonucleotide
gggtgccttcacgcgtgctg

SEQ ID NO: 400 - DNA, 20 bp, Artificial Sequence
  Description of Artificial Sequence Synthetic oligonucleotide
gtgtaaaaacgtcctgccat

SEQ ID NO: 401 - DNA, 20 bp, Artificial Sequence
  Description of Artificial Sequence Synthetic oligonucleotide
ggctaccgcagcacgcgtga

SEQ ID NO: 402 - DNA, 20 bp, Artificial Sequence
  Description of Artificial Sequence Synthetic oligonucleotide
ggtaaaaacgtcctgccatt

SEQ ID NO: 403 - DNA, 20 bp, Artificial Sequence
  Description of Artificial Sequence Synthetic oligonucleotide
ggaccttgacccgtctatgt

SEQ ID NO: 404 - DNA, 20 bp, Artificial Sequence
  Description of Artificial Sequence Synthetic oligonucleotide
gcatttttcttcgtcagaag

SEQ ID NO: 405 - DNA, 20 bp, Artificial Sequence
  Description of Artificial Sequence Synthetic oligonucleotide
gatccaacatagacgggtca

SEQ ID NO: 406 - DNA, 20 bp, Artificial Sequence
  Description of Artificial Sequence Synthetic oligonucleotide
gattcactgtatgcaagacg

SEQ ID NO: 407 - DNA, 20 bp, Artificial Sequence
  Description of Artificial Sequence Synthetic oligonucleotide
gtccattgcagagacgacta

SEQ ID NO: 408 - DNA, 20 bp, Artificial Sequence
  Description of Artificial Sequence Synthetic oligonucleotide
gctgctcgggcgtccccttg

SEQ ID NO: 409 - DNA, 20 bp, Artificial Sequence
  Description of Artificial Sequence Synthetic oligonucleotide
gtacaacgtgctggacgacc

SEQ ID NO: 410 - DNA, 20 bp, Artificial Sequence
  Description of Artificial Sequence Synthetic oligonucleotide
ggggtcgtccagcacgttgt

SEQ ID NO: 411 - DNA, 20 bp, Artificial Sequence
  Description of Artificial Sequence Synthetic oligonucleotide
gcacggtgttcgcgactatg

SEQ ID NO: 412 - DNA, 20 bp, Artificial Sequence
  Description of Artificial Sequence Synthetic oligonucleotide
gtggcttctcaacaaaacgg

SEQ ID NO: 413 - DNA, 20 bp, Artificial Sequence
  Description of Artificial Sequence Synthetic oligonucleotide
gctcaacaaaacggtggatg

SEQ ID NO: 414 - DNA, 20 bp, Artificial Sequence
  Description of Artificial Sequence Synthetic oligonucleotide
gtgcagtttcatcggcacca

SEQ ID NO: 415 - DNA, 20 bp, Artificial Sequence
  Description of Artificial Sequence Synthetic oligonucleotide
gagagattcggattccaagc

SEQ ID NO: 416 - DNA, 20 bp, Artificial Sequence
  Description of Artificial Sequence Synthetic oligonucleotide
gtgtccaataagatcaacgc

SEQ ID NO: 417 - DNA, 20 bp, Artificial Sequence
  Description of Artificial Sequence Synthetic oligonucleotide
gctgacaagtacttcaccct

SEQ ID NO: 418 - DNA, 20 bp, Artificial Sequence
  Description of Artificial Sequence Synthetic oligonucleotide
ggccgagtgtgacgcctttg

SEQ ID NO: 419 - DNA, 20 bp, Artificial Sequence
  Description of Artificial Sequence Synthetic oligonucleotide
gatcgagtactataagaatg

SEQ ID NO: 420 - DNA, 20 bp, Artificial Sequence
  Description of Artificial Sequence Synthetic oligonucleotide
gctccagtccgcctgcgtct

SEQ ID NO: 421 - DNA, 20 bp, Artificial Sequence
  Description of Artificial Sequence Synthetic oligonucleotide
gagaggaacctaccgtcccg

SEQ ID NO: 422 - DNA, 20 bp, Artificial Sequence
  Description of Artificial Sequence Synthetic oligonucleotide
gcaggagccgtacaccttcc

SEQ ID NO: 423 - DNA, 20 bp, Artificial Sequence
  Description of Artificial Sequence Synthetic oligonucleotide
gctgctgctcaccgacgacc

SEQ ID NO: 424 - DNA, 20 bp, Artificial Sequence
  Description of Artificial Sequence Synthetic oligonucleotide
gagtgttgttaacgacgtgg

SEQ ID NO: 425 - DNA, 20 bp, Artificial Sequence
  Description of Artificial Sequence Synthetic oligonucleotide
gggaatccaagtggtctacg

SEQ ID NO: 426 - DNA, 20 bp, Artificial Sequence
  Description of Artificial Sequence Synthetic oligonucleotide
ggagtggatgtatcccgcag

SEQ ID NO: 427 - DNA, 20 bp, Artificial Sequence
  Description of Artificial Sequence Synthetic oligonucleotide
gctgacacacgacgcaatac

SEQ ID NO: 428 - DNA, 20 bp, Artificial Sequence
  Description of Artificial Sequence Synthetic oligonucleotide
gagaatggtagtcactacga

SEQ ID NO: 429 - DNA, 20 bp, Artificial Sequence
  Description of Artificial Sequence Synthetic oligonucleotide
gactgcacatcggcatcagt

SEQ ID NO: 430 - DNA, 20 bp, Artificial Sequence
  Description of Artificial Sequence Synthetic oligonucleotide
gttgacccttccgcggcgtt

SEQ ID NO: 431 - DNA, 20 bp, Artificial Sequence
  Description of Artificial Sequence Synthetic oligonucleotide
gcaaagcccaccgtatggtg

SEQ ID NO: 432 - DNA, 20 bp, Artificial Sequence
  Description of Artificial Sequence Synthetic oligonucleotide
gagagcccaacgccgcggaa

SEQ ID NO: 433 - DNA, 20 bp, Artificial Sequence
  Description of Artificial Sequence Synthetic oligonucleotide
gactcaccaagggcgccgtc

SEQ ID NO: 434 - DNA, 20 bp, Artificial Sequence
  Description of Artificial Sequence Synthetic oligonucleotide
ggctgggtaacggcatcaga

SEQ ID NO: 435 - DNA, 20 bp, Artificial Sequence
  Description of Artificial Sequence Synthetic oligonucleotide
gtctgcccagccactcttcg

SEQ ID NO: 436 - DNA, 20 bp, Artificial Sequence
  Description of Artificial Sequence Synthetic oligonucleotide
ggcccttgcaccgatgcaca

SEQ ID NO: 437 - DNA, 20 bp, Artificial Sequence
  Description of Artificial Sequence Synthetic oligonucleotide
ggctgcgacggaatgaaatc

SEQ ID NO: 438 - DNA, 20 bp, Artificial Sequence
  Description of Artificial Sequence Synthetic oligonucleotide
gatggcgctggctcgctcca

SEQ ID NO: 439 - DNA, 20 bp, Artificial Sequence
  Description of Artificial Sequence Synthetic oligonucleotide
ggtgaagtcgggctgattgt

SEQ ID NO: 440 - DNA, 20 bp, Artificial Sequence
  Description of Artificial Sequence Synthetic oligonucleotide
gctgcaggatggtgtggacg

SEQ ID NO: 441 - DNA, 20 bp, Artificial Sequence
  Description of Artificial Sequence Synthetic oligonucleotide
gatggaggaggcgcggcacg

SEQ ID NO: 442 - DNA, 20 bp, Artificial Sequence
  Description of Artificial Sequence Synthetic oligonucleotide
gttcctggatgcataccccg

SEQ ID NO: 443 - DNA, 20 bp, Artificial Sequence
  Description of Artificial Sequence Synthetic oligonucleotide
ggtgaagtcggcgttatcat

SEQ ID NO: 444 - DNA, 20 bp, Artificial Sequence
  Description of Artificial Sequence Synthetic oligonucleotide
gatgagcaacatcgacctcg

SEQ ID NO: 445 - DNA, 20 bp, Artificial Sequence
  Description of Artificial Sequence Synthetic oligonucleotide
gcctgggctcgctcaacccc

SEQ ID NO: 446 - DNA, 20 bp, Artificial Sequence
  Description of Artificial Sequence Synthetic oligonucleotide
gccaccttgtgtaggcgttt

SEQ ID NO: 447 - DNA, 20 bp, Artificial Sequence
  Description of Artificial Sequence Synthetic oligonucleotide
gaccttcaagacccgaaact

SEQ ID NO: 448 - DNA, 20 bp, Artificial Sequence
  Description of Artificial Sequence Synthetic oligonucleotide
gtatggtaggcacatccttc

SEQ ID NO: 449 - DNA, 20 bp, Artificial Sequence
  Description of Artificial Sequence Synthetic oligonucleotide
gtctctctgagcagtgccgt

SEQ ID NO: 450 - DNA, 20 bp, Artificial Sequence
  Description of Artificial Sequence Synthetic oligonucleotide
gcttcagggagtgaacgacc

SEQ ID NO: 451 - DNA, 20 bp, Artificial Sequence
  Description of Artificial Sequence Synthetic oligonucleotide
gaaggagctagaaaacgccc

SEQ ID NO: 452 - DNA, 20 bp, Artificial Sequence
  Description of Artificial Sequence Synthetic oligonucleotide
gtgcctaccatactacctcc

SEQ ID NO: 453 - DNA, 20 bp, Artificial Sequence
  Description of Artificial Sequence Synthetic oligonucleotide
ggaatatcaaaaacgtatgc

SEQ ID NO: 454 - DNA, 20 bp, Artificial Sequence
  Description of Artificial Sequence Synthetic oligonucleotide
ggcttccgggcataatagca

SEQ ID NO: 455 - DNA, 20 bp, Artificial Sequence
  Description of Artificial Sequence Synthetic oligonucleotide
gaagtccaggctcaataccc

SEQ ID NO: 456 - DNA, 20 bp, Artificial Sequence
  Description of Artificial Sequence Synthetic oligonucleotide
ggaagaagagccaccgaagt

SEQ ID NO: 457 - DNA, 20 bp, Artificial Sequence
  Description of Artificial Sequence Synthetic oligonucleotide
gggacagcacgcgctctgat

SEQ ID NO: 458 - DNA, 20 bp, Artificial Sequence
  Description of Artificial Sequence Synthetic oligonucleotide
gactttgggcgaagtgaaat

SEQ ID NO: 459 - DNA, 20 bp, Artificial Sequence
  Description of Artificial Sequence Synthetic oligonucleotide
gaagcagcaggcggatggtg

SEQ ID NO: 460 - DNA, 20 bp, Artificial Sequence
  Description of Artificial Sequence Synthetic oligonucleotide
gttgcgcaccccaatgatga

SEQ ID NO: 461 - DNA, 20 bp, Artificial Sequence
  Description of Artificial Sequence Synthetic oligonucleotide
gcggacagcacgcgctctga

SEQ ID NO: 462 - DNA, 20 bp, Artificial Sequence
  Description of Artificial Sequence Synthetic oligonucleotide
gggcgaggtgggcgtgcgtc

SEQ ID NO: 463 - DNA, 20 bp, Artificial Sequence
  Description of Artificial Sequence Synthetic oligonucleotide
ggaccagtacgaccttagtt

SEQ ID NO: 464 - DNA, 20 bp, Artificial Sequence
  Description of Artificial Sequence Synthetic oligonucleotide
ggtcccaactaaggtcgtac

SEQ ID NO: 465 - DNA, 20 bp, Artificial Sequence
  Description of Artificial Sequence Synthetic oligonucleotide
gtaggtgccgtacctcacag

SEQ ID NO: 466 - DNA, 20 bp, Artificial Sequence
  Description of Artificial Sequence Synthetic oligonucleotide
gtggagcctctggtctcgcg

SEQ ID NO: 467 - DNA, 20 bp, Artificial Sequence
  Description of Artificial Sequence Synthetic oligonucleotide
gagcgatggacagtggcgct

SEQ ID NO: 468 - DNA, 20 bp, Artificial Sequence
  Description of Artificial Sequence Synthetic oligonucleotide
gtggagttaggacggacccc

SEQ ID NO: 469 - DNA, 20 bp, Artificial Sequence
  Description of Artificial Sequence Synthetic oligonucleotide
gggtctcgcgcggaaagagg

SEQ ID NO: 470 - DNA, 20 bp, Artificial Sequence
  Description of Artificial Sequence Synthetic oligonucleotide
gggattcctggccgaaaggt

SEQ ID NO: 471 - DNA, 20 bp, Artificial Sequence
  Description of Artificial Sequence Synthetic oligonucleotide
gcatgaatcgtgatctttgt

SEQ ID NO: 472 - DNA, 20 bp, Artificial Sequence
  Description of Artificial Sequence Synthetic oligonucleotide
gatgaccaagaccgacacga

SEQ ID NO: 473 - DNA, 20 bp, Artificial Sequence
  Description of Artificial Sequence Synthetic oligonucleotide
gaggaggcgcgccacacacg

SEQ ID NO: 474 - DNA, 20 bp, Artificial Sequence
  Description of Artificial Sequence Synthetic oligonucleotide
gagtagcgcaggcgtttgac

SEQ ID NO: 475 - DNA, 20 bp, Artificial Sequence
  Description of Artificial Sequence Synthetic oligonucleotide
gggcgctggaactcattgcg

SEQ ID NO: 476 - DNA, 20 bp, Artificial Sequence
  Description of Artificial Sequence Synthetic oligonucleotide
gattgccatcgtgtcggtct

SEQ ID NO: 477 - DNA, 20 bp, Artificial Sequence
  Description of Artificial Sequence Synthetic oligonucleotide
gatgagcattatcgacgttg

SEQ ID NO: 478 - DNA, 20 bp, Artificial Sequence
  Description of Artificial Sequence Synthetic oligonucleotide
gtctgcctacgacgatgaac

SEQ ID NO: 479 - DNA, 20 bp, Artificial Sequence
  Description of Artificial Sequence Synthetic oligonucleotide
ggagtacaaagaccgcaagc

SEQ ID NO: 480 - DNA, 20 bp, Artificial Sequence
  Description of Artificial Sequence Synthetic oligonucleotide
gtgtggagagcgctattcgg

SEQ ID NO: 481 - DNA, 20 bp, Artificial Sequence
  Description of Artificial Sequence Synthetic oligonucleotide
gcgcctcgcggcccacgaaa

SEQ ID NO: 482 - DNA, 20 bp, Artificial Sequence
  Description of Artificial Sequence Synthetic oligonucleotide
gcttggcgctggtcgcctcg

SEQ ID NO: 483 - DNA, 20 bp, Artificial Sequence
  Description of Artificial Sequence Synthetic oligonucleotide
gagataagcgtcgttaccct

SEQ ID NO: 484 - DNA, 20 bp, Artificial Sequence
  Description of Artificial Sequence Synthetic oligonucleotide
ggcgtcgttacccttggcgc

SEQ ID NO: 485 - DNA, 20 bp, Artificial Sequence
  Description of Artificial Sequence Synthetic oligonucleotide
ggaacagtccgtaggcgatg

SEQ ID NO: 486 - DNA, 20 bp, Artificial Sequence
  Description of Artificial Sequence Synthetic oligonucleotide
gcagcgcggcctcgtagtgc

SEQ ID NO: 487 - DNA, 20 bp, Artificial Sequence
  Description of Artificial Sequence Synthetic oligonucleotide
ggcagcacgtggtacaccga

SEQ ID NO: 488 - DNA, 20 bp, Artificial Sequence
  Description of Artificial Sequence Synthetic oligonucleotide
gcggcaggcgcgcatgatga

SEQ ID NO: 489 - DNA, 20 bp, Artificial Sequence
  Description of Artificial Sequence Synthetic oligonucleotide
gttctctgcgctcgaaagcc

SEQ ID NO: 490 - DNA, 20 bp, Artificial Sequence
  Description of Artificial Sequence Synthetic oligonucleotide
ggaaacacttccaacccgtg

SEQ ID NO: 491 - DNA, 20 bp, Artificial Sequence
  Description of Artificial Sequence Synthetic oligonucleotide
gatgctgcatgtcgatcgcc

SEQ ID NO: 492 - DNA, 20 bp, Artificial Sequence
  Description of Artificial Sequence Synthetic oligonucleotide
ggacctcggcccacgtccgc

SEQ ID NO: 493 - DNA, 20 bp, Artificial Sequence
  Description of Artificial Sequence Synthetic oligonucleotide
gcgcgccaaccggagccccc

SEQ ID NO: 494 - DNA, 20 bp, Artificial Sequence
  Description of Artificial Sequence Synthetic oligonucleotide
gctgccaaactcgatgcggc

SEQ ID NO: 495 - DNA, 20 bp, Artificial Sequence
  Description of Artificial Sequence Synthetic oligonucleotide
gactctggttgcacgaaatg

SEQ ID NO: 496 - DNA, 20 bp, Artificial Sequence
  Description of Artificial Sequence Synthetic oligonucleotide
gaccatgaaggagcgacggg

SEQ ID NO: 497 - DNA, 20 bp, Artificial Sequence
  Description of Artificial Sequence Synthetic oligonucleotide
gttaatgagcataaattacc

SEQ ID NO: 498 - DNA, 20 bp, Artificial Sequence
  Description of Artificial Sequence Synthetic oligonucleotide
gtctcaagactataaccaag

SEQ ID NO: 499 - DNA, 20 bp, Artificial Sequence
  Description of Artificial Sequence Synthetic oligonucleotide
gccatgaaggagcgacgggt

SEQ ID NO: 500 - DNA, 20 bp, Artificial Sequence
  Description of Artificial Sequence Synthetic oligonucleotide
gaaggcaggtcccgcatttt

SEQ ID NO: 501 - DNA, 20 bp, Artificial Sequence
  Description of Artificial Sequence Synthetic oligonucleotide
gcttggccttcaaccgaaaa

SEQ ID NO: 502 - DNA, 20 bp, Artificial Sequence
  Description of Artificial Sequence Synthetic oligonucleotide
ggaagaattttgtgccgctt

SEQ ID NO: 503 - DNA, 20 bp, Artificial Sequence
  Description of Artificial Sequence Synthetic oligonucleotide
gtgatgtaggcaaacgccct

SEQ ID NO: 504 - DNA, 20 bp, Artificial Sequence
  Description of Artificial Sequence Synthetic oligonucleotide
gccttcaaccgaaaatggtt

SEQ ID NO: 505 - DNA, 20 bp, Artificial Sequence
  Description of Artificial Sequence Synthetic oligonucleotide
gttcaggagacgtcggaacc

SEQ ID NO: 506 - DNA, 20 bp, Artificial Sequence
  Description of Artificial Sequence Synthetic oligonucleotide
gcggagttttcatgccccgt

SEQ ID NO: 507 - DNA, 20 bp, Artificial Sequence
  Description of Artificial Sequence Synthetic oligonucleotide
gtctgagcattgtaccgggt

SEQ ID NO: 508 - DNA, 20 bp, Artificial Sequence
  Description of Artificial Sequence Synthetic oligonucleotide
ggcctaccacccgacaccag

SEQ ID NO: 509 - DNA, 20 bp, Artificial Sequence
  Description of Artificial Sequence Synthetic oligonucleotide
gcacttgaaaaaactgtgcg

SEQ ID NO: 510 - DNA, 20 bp, Artificial Sequence
  Description of Artificial Sequence Synthetic oligonucleotide
gggtcacctggtcgaagtat

SEQ ID NO: 511 - DNA, 20 bp, Artificial Sequence
  Description of Artificial Sequence Synthetic oligonucleotide
gcacagcagcaagaccttcg

SEQ ID NO: 512 - DNA, 20 bp, Artificial Sequence
  Description of Artificial Sequence Synthetic oligonucleotide
ggtagtagactggttgtatc

SEQ ID NO: 513 - DNA, 20 bp, Artificial Sequence
  Description of Artificial Sequence Synthetic oligonucleotide
gctacaaaatcagcgtcatc

SEQ ID NO: 514 - DNA, 20 bp, Artificial Sequence
  Description of Artificial Sequence Synthetic oligonucleotide
gagaggtggacgttgtctag

SEQ ID NO: 515 - DNA, 20 bp, Artificial Sequence
  Description of Artificial Sequence Synthetic oligonucleotide
ggacgcgctcgaactcggga

SEQ ID NO: 516 - DNA, 20 bp, Artificial Sequence
  Description of Artificial Sequence Synthetic oligonucleotide
gagacgcgctcgaactcggg

SEQ ID NO: 517 - DNA, 20 bp, Artificial Sequence
  Description of Artificial Sequence Synthetic oligonucleotide
gctcatacacacgatgatgt

SEQ ID NO: 518 - DNA, 20 bp, Artificial Sequence
  Description of Artificial Sequence Synthetic oligonucleotide
gtgttgtaaatgactacgcg

SEQ ID NO: 519 - DNA, 20 bp, Artificial Sequence
  Description of Artificial Sequence Synthetic oligonucleotide
gcagaggtaacacgtagggg

SEQ ID NO: 520 - DNA, 20 bp, Artificial Sequence
  Description of Artificial Sequence Synthetic oligonucleotide
ggtgggtcccgaagccaacg

SEQ ID NO: 521 - DNA, 20 bp, Artificial Sequence
  Description of Artificial Sequence Synthetic oligonucleotide
gctccagcgactggcggtat

SEQ ID NO: 522 - DNA, 20 bp, Artificial Sequence
  Description of Artificial Sequence Synthetic oligonucleotide
gggcgcttgccgatggcctc

SEQ ID NO: 523 - DNA, 20 bp, Artificial Sequence
  Description of Artificial Sequence Synthetic oligonucleotide
ggccatctcctcgggcggca

SEQ ID NO: 524 - DNA, 20 bp, Artificial Sequence
  Description of Artificial Sequence Synthetic oligonucleotide
gcgtcaccgcgtcggccgaa

SEQ ID NO: 525 - DNA, 20 bp, Artificial Sequence
  Description of Artificial Sequence Synthetic oligonucleotide
gggtatgcctatgtccgtgg

SEQ ID NO: 526 - DNA, 20 bp, Artificial Sequence
  Description of Artificial Sequence Synthetic oligonucleotide
ggggcgaatccccatccgcc

SEQ ID NO: 527 - DNA, 20 bp, Artificial Sequence
  Description of Artificial Sequence Synthetic oligonucleotide
ggggcttgcggcctttggcc

SEQ ID NO: 528 - DNA, 20 bp, Artificial Sequence
  Description of Artificial Sequence Synthetic oligonucleotide
gttaggaacagcgtctgttg

SEQ ID NO: 529 - DNA, 20 bp, Artificial Sequence
  Description of Artificial Sequence Synthetic oligonucleotide
gcctccacatgccgtgtaca

SEQ ID NO: 530 - DNA, 20 bp, Artificial Sequence
  Description of Artificial Sequence Synthetic oligonucleotide
gtgcggcaccggcgagtcag

SEQ ID NO: 531 - DNA, 20 bp, Artificial Sequence
  Description of Artificial Sequence Synthetic oligonucleotide
ggccgcagcccgatgtacag

SEQ ID NO: 532 - DNA, 20 bp, Artificial Sequence
  Description of Artificial Sequence Synthetic oligonucleotide
gcatattgtgtcggtactgc

SEQ ID NO: 533 - DNA, 20 bp, Artificial Sequence
  Description of Artificial Sequence Synthetic oligonucleotide
gacggtttcatagtcgtacc

SEQ ID NO: 534 - DNA, 20 bp, Artificial Sequence
  Description of Artificial Sequence Synthetic oligonucleotide
gcaaagtgtagcggatgtta

SEQ ID NO: 535 - DNA, 20 bp, Artificial Sequence
  Description of Artificial Sequence Synthetic oligonucleotide
gcaatcacataccgcaccac

SEQ ID NO: 536 - DNA, 20 bp, Artificial Sequence
  Description of Artificial Sequence Synthetic oligonucleotide
gaacctcttcggtatcccaa

SEQ ID NO: 537 - DNA, 20 bp, Artificial Sequence
  Description of Artificial Sequence Synthetic oligonucleotide
gaatctaatttcaccgccta

SEQ ID NO: 538 - DNA, 20 bp, Artificial Sequence
  Description of Artificial Sequence Synthetic oligonucleotide
gcggcaacacgaagtccacc

SEQ ID NO: 539 - DNA, 20 bp, Artificial Sequence
  Description of Artificial Sequence Synthetic oligonucleotide
gtggaatactgcaccaccgt

SEQ ID NO: 540 - DNA, 20 bp, Artificial Sequence
  Description of Artificial Sequence Synthetic oligonucleotide
ggtactcaacacgtgcctca

SEQ ID NO: 541 - DNA, 20 bp, Artificial Sequence
  Description of Artificial Sequence Synthetic oligonucleotide
gctgatttatatcgatattg

SEQ ID NO: 542 - DNA, 20 bp, Artificial Sequence
  Description of Artificial Sequence Synthetic oligonucleotide
gactccatagtctgcgtagc

SEQ ID NO: 543 - DNA, 20 bp, Artificial Sequence
  Description of Artificial Sequence Synthetic oligonucleotide
gtgctaggcggaaatccgta

SEQ ID NO: 544 - DNA, 20 bp, Artificial Sequence
  Description of Artificial Sequence Synthetic oligonucleotide
gctgtcatgacaccagcgta

SEQ ID NO: 545 - DNA, 20 bp, Artificial Sequence
  Description of Artificial Sequence Synthetic oligonucleotide
ggtggccagactgattcgtc

SEQ ID NO: 546 - DNA, 20 bp, Artificial Sequence
  Description of Artificial Sequence Synthetic oligonucleotide
gagtttgaccagcctcgact

SEQ ID NO: 547 - DNA, 20 bp, Artificial Sequence
  Description of Artificial Sequence Synthetic oligonucleotide
gtttatacacgatctgtaca

SEQ ID NO: 548 - DNA, 20 bp, Artificial Sequence
  Description of Artificial Sequence Synthetic oligonucleotide
gtggaagggaggccaagtcg

SEQ ID NO: 549 - DNA, 20 bp, Artificial Sequence
  Description of Artificial Sequence Synthetic oligonucleotide
gaaattactgtatcgggaac

SEQ ID NO: 550 - DNA, 20 bp, Artificial Sequence
  Description of Artificial Sequence Synthetic oligonucleotide
gtgtggatcttattcgaaga

SEQ ID NO: 551 - DNA, 20 bp, Artificial Sequence
  Description of Artificial Sequence Synthetic oligonucleotide
ggagaacaattacctgacgc

SEQ ID NO: 552 - DNA, 20 bp, Artificial Sequence
  Description of Artificial Sequence Synthetic oligonucleotide
ggaccgagtgcacggacatg

SEQ ID NO: 553 - DNA, 20 bp, Artificial Sequence
  Description of Artificial Sequence Synthetic oligonucleotide
gttggcgtcgcacgcgtccc

SEQ ID NO: 554 - DNA, 20 bp, Artificial Sequence
  Description of Artificial Sequence Synthetic oligonucleotide
ggggcacggtgtcgaaggtt

SEQ ID NO: 555 - DNA, 20 bp, Artificial Sequence
  Description of Artificial Sequence Synthetic oligonucleotide
ggcagctggtctcgcagttg

SEQ ID NO: 556 - DNA, 20 bp, Artificial Sequence
  Description of Artificial Sequence Synthetic oligonucleotide
ggcgatacttgtcattcggt

SEQ ID NO: 557 - DNA, 20 bp, Artificial Sequence
  Description of Artificial Sequence Synthetic oligonucleotide
gcgcctggagtcaatcgcaa

SEQ ID NO: 558 - DNA, 20 bp, Artificial Sequence
  Description of Artificial Sequence Synthetic oligonucleotide
gtggacgccagcggggtgta

SEQ ID NO: 559 - DNA, 20 bp, Artificial Sequence
  Description of Artificial Sequence Synthetic oligonucleotide
gtccttcgtggacgccagcg

SEQ ID NO: 560 - DNA, 20 bp, Artificial Sequence
  Description of Artificial Sequence Synthetic oligonucleotide
ggacgtagcgaccgcaacat

SEQ ID NO: 561 - DNA, 20 bp, Artificial Sequence
  Description of Artificial Sequence Synthetic oligonucleotide
gctgctgtcgttgagcgggt

SEQ ID NO: 562 - DNA, 20 bp, Artificial Sequence
  Description of Artificial Sequence Synthetic oligonucleotide
gaaggccgtggaatcggacg

SEQ ID NO: 563 - DNA, 20 bp, Artificial Sequence
  Description of Artificial Sequence Synthetic oligonucleotide
gatctccagctgatcggcgg

SEQ ID NO: 564 - DNA, 20 bp, Artificial Sequence
  Description of Artificial Sequence Synthetic oligonucleotide
gggagcagacgctgtgcccg

SEQ ID NO: 565 - DNA, 20 bp, Artificial Sequence
  Description of Artificial Sequence Synthetic oligonucleotide
gggaggtcatcgtgactcct

SEQ ID NO: 566 - DNA, 20 bp, Artificial Sequence
  Description of Artificial Sequence Synthetic oligonucleotide
gtggctgaaaacgcattgta

SEQ ID NO: 567 - DNA, 20 bp, Artificial Sequence
  Description of Artificial Sequence Synthetic oligonucleotide
ggattgttatcgcaagatat

SEQ ID NO: 568 - DNA, 20 bp, Artificial Sequence
  Description of Artificial Sequence Synthetic oligonucleotide
ggtcaactacgctcgtactg

SEQ ID NO: 569 - DNA, 20 bp, Artificial Sequence
  Description of Artificial Sequence Synthetic oligonucleotide
ggagaacatcgtaatgaacc

SEQ ID NO: 570 - DNA, 20 bp, Artificial Sequence
  Description of Artificial Sequence Synthetic oligonucleotide
gttggtgggcgtgacgcctc

SEQ ID NO: 571 - DNA, 20 bp, Artificial Sequence
  Description of Artificial Sequence Synthetic oligonucleotide
ggaaaccttcgcggagtgga

SEQ ID NO: 572 - DNA, 20 bp, Artificial Sequence
  Description of Artificial Sequence Synthetic oligonucleotide
ggctagtgaacggctcttta

SEQ ID NO: 573 - DNA, 20 bp, Artificial Sequence
  Description of Artificial Sequence Synthetic oligonucleotide
ggggacggcccgtggcgttc

SEQ ID NO: 574 - DNA, 20 bp, Artificial Sequence
  Description of Artificial Sequence Synthetic oligonucleotide
gctgcacctccgagcattcg

SEQ ID NO: 575 - DNA, 20 bp, Artificial Sequence
  Description of Artificial Sequence Synthetic oligonucleotide
gctgatccgcgaatgctcgg

SEQ ID NO: 576 - DNA, 20 bp, Artificial Sequence
  Description of Artificial Sequence Synthetic oligonucleotide
gcagggatttcatcgtgcaa

SEQ ID NO: 577 - DNA, 20 bp, Artificial Sequence
  Description of Artificial Sequence Synthetic oligonucleotide
gttcatcgtgcaacggtacg

SEQ ID NO: 578 - DNA, 20 bp, Artificial Sequence
  Description of Artificial Sequence Synthetic oligonucleotide
gggcccacggggagtttcgg

SEQ ID NO: 579 - DNA, 20 bp, Artificial Sequence
  Description of Artificial Sequence Synthetic oligonucleotide
ggggagtttcggaggtggct

SEQ ID NO: 580 - DNA, 20 bp, Artificial Sequence
  Description of Artificial Sequence Synthetic oligonucleotide
gtggggcccacggggagttt

SEQ ID NO: 581 - DNA, 20 bp, Artificial Sequence
  Description of Artificial Sequence Synthetic oligonucleotide
gggtacctgcttatgcctac

SEQ ID NO: 582 - DNA, 20 bp, Artificial Sequence
  Description of Artificial Sequence Synthetic oligonucleotide
ggatgttctgatacacgccc

SEQ ID NO: 583 - DNA, 20 bp, Artificial Sequence
  Description of Artificial Sequence Synthetic oligonucleotide
ggaagccaccaaccgtgtca

SEQ ID NO: 584 - DNA, 20 bp, Artificial Sequence
  Description of Artificial Sequence Synthetic oligonucleotide
gtttttaagatattctgcct

SEQ ID NO: 585 - DNA, 20 bp, Artificial Sequence
  Description of Artificial Sequence Synthetic oligonucleotide
gaatggctgaagaaggaacc

SEQ ID NO: 586 - DNA, 20 bp, Artificial Sequence
  Description of Artificial Sequence Synthetic oligonucleotide
gcacacacataggtgaccac

SEQ ID NO: 587 - DNA, 20 bp, Artificial Sequence
  Description of Artificial Sequence Synthetic oligonucleotide
gcagcatttaccagtattgc

SEQ ID NO: 588 - DNA, 20 bp, Artificial Sequence
  Description of Artificial Sequence Synthetic oligonucleotide
gcacgagtgggatgaccata

SEQ ID NO: 589 - DNA, 20 bp, Artificial Sequence
  Description of Artificial Sequence Synthetic oligonucleotide
gcaggcatcctcattttgcg

SEQ ID NO: 590 - DNA, 20 bp, Artificial Sequence
  Description of Artificial Sequence Synthetic oligonucleotide
ggatgatatagacgataaaa

SEQ ID NO: 591 - DNA, 20 bp, Artificial Sequence
  Description of Artificial Sequence Synthetic oligonucleotide
ggaattgacagctatcccgt

SEQ ID NO: 592 - DNA, 20 bp, Artificial Sequence
  Description of Artificial Sequence Synthetic oligonucleotide
gggatagtccgcattggcat

SEQ ID NO: 593 - DNA, 20 bp, Artificial Sequence
  Description of Artificial Sequence Synthetic oligonucleotide
gttctaagtcgcatcattcg

SEQ ID NO: 594 - DNA, 20 bp, Artificial Sequence
  Description of Artificial Sequence Synthetic oligonucleotide
gtttggggcatcgccaagtt

SEQ ID NO: 595 - DNA, 20 bp, Artificial Sequence
  Description of Artificial Sequence Synthetic oligonucleotide
gccacaaactcgttgcctac

SEQ ID NO: 596 - DNA, 20 bp, Artificial Sequence
  Description of Artificial Sequence Synthetic oligonucleotide
ggcgcaaacattaaggcacg

SEQ ID NO: 597 - DNA, 20 bp, Artificial Sequence
  Description of Artificial Sequence Synthetic oligonucleotide
gtaatgtttgcgctcagaat

SEQ ID NO: 598 - DNA, 20 bp, Artificial Sequence
  Description of Artificial Sequence Synthetic oligonucleotide
ggttcgaggtgtggacatcg

SEQ ID NO: 599 - DNA, 20 bp, Artificial Sequence
  Description of Artificial Sequence Synthetic oligonucleotide
ggtgaataagatcatcgccc

SEQ ID NO: 600 - DNA, 20 bp, Artificial Sequence
  Description of Artificial Sequence Synthetic oligonucleotide
gaggctggcgttgaggcact

SEQ ID NO: 601 - DNA, 20 bp, Artificial Sequence
  Description of Artificial Sequence Synthetic oligonucleotide
gcacatctcgtttgaatttg

SEQ ID NO: 602 - DNA, 20 bp, Artificial Sequence
  Description of Artificial Sequence Synthetic oligonucleotide
gtggaatggcttcgggggca

SEQ ID NO: 603 - DNA, 20 bp, Artificial Sequence
  Description of Artificial Sequence Synthetic oligonucleotide
ggaagaaagtggtctgagcg

SEQ ID NO: 604 - DNA, 20 bp, Artificial Sequence
  Description of Artificial Sequence Synthetic oligonucleotide
gtatcatcctatccagatcc

SEQ ID NO: 605 - DNA, 20 bp, Artificial Sequence
  Description of Artificial Sequence Synthetic oligonucleotide
ggatggatgcttctttagcc

SEQ ID NO: 606 - DNA, 20 bp, Artificial Sequence
  Description of Artificial Sequence Synthetic oligonucleotide
gaagtctcacagacaccgct

SEQ ID NO: 607 - DNA, 20 bp, Artificial Sequence
  Description of Artificial Sequence Synthetic oligonucleotide
gctgacagtccattgacaat

SEQ ID NO: 608 - DNA, 20 bp, Artificial Sequence
  Description of Artificial Sequence Synthetic oligonucleotide
ggagtgtaaggtaatcagcc

SEQ ID NO: 609 - DNA, 20 bp, Artificial Sequence
  Description of Artificial Sequence Synthetic oligonucleotide
gcgagaaggcctaccaagtg

SEQ ID NO: 610 - DNA, 20 bp, Artificial Sequence
  Description of Artificial Sequence Synthetic oligonucleotide
gctggatctcacaggtccga

SEQ ID NO: 611 - DNA, 20 bp, Artificial Sequence
  Description of Artificial Sequence Synthetic oligonucleotide
gccccacccgggaagcgctc

SEQ ID NO: 612 - DNA, 20 bp, Artificial Sequence
  Description of Artificial Sequence Synthetic oligonucleotide
gatctccacggtgtccacct

SEQ ID NO: 613 - DNA, 20 bp, Artificial Sequence
  Description of Artificial Sequence Synthetic oligonucleotide
gcctggccacctccgcgatc

SEQ ID NO: 614 - DNA, 20 bp, Artificial Sequence
  Description of Artificial Sequence Synthetic oligonucleotide
gccaggctctttgccgacgc

SEQ ID NO: 615 - DNA, 20 bp, Artificial Sequence
  Description of Artificial Sequence Synthetic oligonucleotide
gggccacctccgcgatccgg

SEQ ID NO: 616 - DNA, 20 bp, Artificial Sequence
  Description of Artificial Sequence Synthetic oligonucleotide
gctctaggcgtggttccccg

SEQ ID NO: 617 - DNA, 20 bp, Artificial Sequence
  Description of Artificial Sequence Synthetic oligonucleotide
gtacgtgcaaattcacccga

SEQ ID NO: 618 - DNA, 20 bp, Artificial Sequence
  Description of Artificial Sequence Synthetic oligonucleotide
gtgtgtaataaagatgccgt

SEQ ID NO: 619 - DNA, 20 bp, Artificial Sequence
  Description of Artificial Sequence Synthetic oligonucleotide
ggtgtaataaagatgccgtc

SEQ ID NO: 620 - DNA, 20 bp, Artificial Sequence
  Description of Artificial Sequence Synthetic oligonucleotide
gcatgggcgtgaacctcacc

SEQ ID NO: 621 - DNA, 20 bp, Artificial Sequence
  Description of Artificial Sequence Synthetic oligonucleotide
gaacattcccgatgcctacg

SEQ ID NO: 622 - DNA, 20 bp, Artificial Sequence
  Description of Artificial Sequence Synthetic oligonucleotide
gcgctgcctcggctcactat

SEQ ID NO: 623 - DNA, 20 bp, Artificial Sequence
  Description of Artificial Sequence Synthetic oligonucleotide
gttactttgatacgtcgagt

SEQ ID NO: 624 - DNA, 20 bp, Artificial Sequence
  Description of Artificial Sequence Synthetic oligonucleotide
ggcaaatccgtcggcttttg

SEQ ID NO: 625 - DNA, 20 bp, Artificial Sequence
  Description of Artificial Sequence Synthetic oligonucleotide
gttcctagacttggccacgt

SEQ ID NO: 626 - DNA, 20 bp, Artificial Sequence
  Description of Artificial Sequence Synthetic oligonucleotide
gctgctgtcgtgcctggtcg

SEQ ID NO: 627 - DNA, 20 bp, Artificial Sequence
  Description of Artificial Sequence Synthetic oligonucleotide
gtgctacctgggcgacttcg

SEQ ID NO: 628 - DNA, 20 bp, Artificial Sequence
  Description of Artificial Sequence Synthetic oligonucleotide
ggtgctgctcttcgccggcc

SEQ ID NO: 629 - DNA, 20 bp, Artificial Sequence
  Description of Artificial Sequence Synthetic oligonucleotide
gcgctcggcgagtaccgcgg

SEQ ID NO: 630 - DNA, 20 bp, Artificial Sequence
  Description of Artificial Sequence Synthetic oligonucleotide
gccggggcacgcctctcgct

SEQ ID NO: 631 - DNA, 20 bp, Artificial Sequence
  Description of Artificial Sequence Synthetic oligonucleotide
gacatacatgctgacgctgg

SEQ ID NO: 632 - DNA, 20 bp, Artificial Sequence
  Description of Artificial Sequence Synthetic oligonucleotide
gatcatgtatacgatattcc

SEQ ID NO: 633 - DNA, 20 bp, Artificial Sequence
  Description of Artificial Sequence Synthetic oligonucleotide
gacatcaatgtcccacgatt

SEQ ID NO: 634 - DNA, 20 bp, Artificial Sequence
  Description of Artificial Sequence Synthetic oligonucleotide
gaagggtctctgtcgcggcg

SEQ ID NO: 635 - DNA, 20 bp, Artificial Sequence
  Description of Artificial Sequence Synthetic oligonucleotide
ggaggagtcggccgtgtgcc

SEQ ID NO: 636 - DNA, 20 bp, Artificial Sequence
  Description of Artificial Sequence Synthetic oligonucleotide
gctgctagcagtcccgatcg

SEQ ID NO: 637 - DNA, 20 bp, Artificial Sequence
  Description of Artificial Sequence Synthetic oligonucleotide
gtagcagtcccgatcgtggc

SEQ ID NO: 638 - DNA, 20 bp, Artificial Sequence
  Description of Artificial Sequence Synthetic oligonucleotide
gtgtagctacgttttacaac

SEQ ID NO: 639 - DNA, 20 bp, Artificial Sequence
  Description of Artificial Sequence Synthetic oligonucleotide
gcagttcaggtacgtcctga

SEQ ID NO: 640 - DNA, 20 bp, Artificial Sequence
  Description of Artificial Sequence Synthetic oligonucleotide
gatcaaaggtgggcttccgt

SEQ ID NO: 641 - DNA, 20 bp, Artificial Sequence
  Description of Artificial Sequence Synthetic oligonucleotide
gggaaaattgttccgccacc

SEQ ID NO: 642 - DNA, 20 bp, Artificial Sequence
  Description of Artificial Sequence Synthetic oligonucleotide
gagagaccatcgaagcccta

SEQ ID NO: 643 - DNA, 20 bp, Artificial Sequence
  Description of Artificial Sequence Synthetic oligonucleotide
gccacctgcggagcgtgatt

SEQ ID NO: 644 - DNA, 20 bp, Artificial Sequence
  Description of Artificial Sequence Synthetic oligonucleotide
gtatcgccaccggcctgcct

SEQ ID NO: 645 - DNA, 20 bp, Artificial Sequence
  Description of Artificial Sequence Synthetic oligonucleotide
ggcaggctacgcccattgga

SEQ ID NO: 646 - DNA, 20 bp, Artificial Sequence
  Description of Artificial Sequence Synthetic oligonucleotide
gtgatgggcgacaagtggaa

SEQ ID NO: 647 - DNA, 20 bp, Artificial Sequence
  Description of Artificial Sequence Synthetic oligonucleotide
gaacttcatgagctgtacga

SEQ ID NO: 648 - DNA, 20 bp, Artificial Sequence
  Description of Artificial Sequence Synthetic oligonucleotide
ggacctcctccccgtgtaca

SEQ ID NO: 649 - DNA, 20 bp, Artificial Sequence
  Description of Artificial Sequence Synthetic oligonucleotide
ggaacttcatgagctgtacg

SEQ ID NO: 650 - DNA, 20 bp, Artificial Sequence
  Description of Artificial Sequence Synthetic oligonucleotide
gggctgtacggctttgaacg

SEQ ID NO: 651 - DNA, 20 bp, Artificial Sequence
  Description of Artificial Sequence Synthetic oligonucleotide
gggataggaacaccgactaa

SEQ ID NO: 652 - DNA, 20 bp, Artificial Sequence
  Description of Artificial Sequence Synthetic oligonucleotide
gttaggtcagtatcgacgag

SEQ ID NO: 653 - DNA, 20 bp, Artificial Sequence
  Description of Artificial Sequence Synthetic oligonucleotide
ggatgtgggcacacgctaca

SEQ ID NO: 654 - DNA, 20 bp, Artificial Sequence
  Description of Artificial Sequence Synthetic oligonucleotide
gtattcctttcggggtagtc

SEQ ID NO: 655 - DNA, 20 bp, Artificial Sequence
  Description of Artificial Sequence Synthetic oligonucleotide
gcaccaataccactcgggag

SEQ ID NO: 656 - DNA, 20 bp, Artificial Sequence
  Description of Artificial Sequence Synthetic oligonucleotide
gcaataccactcgggagagg

SEQ ID NO: 657 - DNA, 20 bp, Artificial Sequence
  Description of Artificial Sequence Synthetic oligonucleotide
gtgactaccccgaaaggaat

SEQ ID NO: 658 - DNA, 20 bp, Artificial Sequence
  Description of Artificial Sequence Synthetic oligonucleotide
gcataagttttaccccgggc

SEQ ID NO: 659 - DNA, 20 bp, Artificial Sequence
  Description of Artificial Sequence Synthetic oligonucleotide
ggcttactttcggactctca

SEQ ID NO: 660 - DNA, 20 bp, Artificial Sequence
  Description of Artificial Sequence Synthetic oligonucleotide
ggacctggcttacctttcgt

SEQ ID NO: 661 - DNA, 20 bp, Artificial Sequence
  Description of Artificial Sequence Synthetic oligonucleotide
gggataggtgttccaacgaa

SEQ ID NO: 662 - DNA, 20 bp, Artificial Sequence
  Description of Artificial Sequence Synthetic oligonucleotide
ggtgggagagtatcggcgtg

SEQ ID NO: 663 - DNA, 20 bp, Artificial Sequence
  Description of Artificial Sequence Synthetic oligonucleotide
ggtaagtcttaccccgggct

SEQ ID NO: 664 - DNA, 20 bp, Artificial Sequence
  Description of Artificial Sequence Synthetic oligonucleotide
gtgttgttgtttaggatcgc

SEQ ID NO: 665 - DNA, 20 bp, Artificial Sequence
  Description of Artificial Sequence Synthetic oligonucleotide
ggcggcgggcaggcgttgcg

SEQ ID NO: 666 - DNA, 20 bp, Artificial Sequence
  Description of Artificial Sequence Synthetic oligonucleotide
gtgtactgacagactccgtc

SEQ ID NO: 667 - DNA, 20 bp, Artificial Sequence
  Description of Artificial Sequence Synthetic oligonucleotide
gtattcgatcttatcgatgg

SEQ ID NO: 668 - DNA, 20 bp, Artificial Sequence
  Description of Artificial Sequence Synthetic oligonucleotide
gctcttcaggtcggcccacc

SEQ ID NO: 669 - DNA, 20 bp, Artificial Sequence
  Description of Artificial Sequence Synthetic oligonucleotide
gaagagtgggcggacccggt

SEQ ID NO: 670 - DNA, 20 bp, Artificial Sequence
  Description of Artificial Sequence Synthetic oligonucleotide
gttttattcgatcttatcga

SEQ ID NO: 671 - DNA, 20 bp, Artificial Sequence
  Description of Artificial Sequence Synthetic oligonucleotide
gagagtccatcgccaacatt

SEQ ID NO: 672 - DNA, 20 bp, Artificial Sequence
  Description of Artificial Sequence Synthetic oligonucleotide
gctttgcatcgccccttgtc

SEQ ID NO: 673 - DNA, 20 bp, Artificial Sequence
  Description of Artificial Sequence Synthetic oligonucleotide
ggccgcatctacattgacga

SEQ ID NO: 674 - DNA, 20 bp, Artificial Sequence
  Description of Artificial Sequence Synthetic oligonucleotide
gggatccgaagttccggaca

SEQ ID NO: 675 - DNA, 20 bp, Artificial Sequence
  Description of Artificial Sequence Synthetic oligonucleotide
gtcttttggatctgcgcttc

SEQ ID NO: 676 - DNA, 20 bp, Artificial Sequence
  Description of Artificial Sequence Synthetic oligonucleotide
ggtcatgtttgttattgacc

SEQ ID NO: 677 - DNA, 20 bp, Artificial Sequence
  Description of Artificial Sequence Synthetic oligonucleotide
gcaggtgaaatcgcaggagc

SEQ ID NO: 678 - DNA, 20 bp, Artificial Sequence
  Description of Artificial Sequence Synthetic oligonucleotide
gatagttgttgtaatccttc

SEQ ID NO: 679 - DNA, 20 bp, Artificial Sequence
  Description of Artificial Sequence Synthetic oligonucleotide
gcctgtctaagtcgtccact

SEQ ID NO: 680 - DNA, 20 bp, Artificial Sequence
  Description of Artificial Sequence Synthetic oligonucleotide
gcccagtggacgacttagac

SEQ ID NO: 681 - DNA, 20 bp, Artificial Sequence
  Description of Artificial Sequence Synthetic oligonucleotide
gacctgtctaagtcgtccac

SEQ ID NO: 682 - DNA, 20 bp, Artificial Sequence
  Description of Artificial Sequence Synthetic oligonucleotide
gctactgtggcttgggcggc

SEQ ID NO: 683 - DNA, 20 bp, Artificial Sequence
  Description of Artificial Sequence Synthetic oligonucleotide
gatgctgtcacatacctcgc

SEQ ID NO: 684 - DNA, 20 bp, Artificial Sequence
  Description of Artificial Sequence Synthetic oligonucleotide
ggctgtcacatacctcgcag

SEQ ID NO: 685 - DNA, 20 bp, Artificial Sequence
  Description of Artificial Sequence Synthetic oligonucleotide
gtgctgtcacatacctcgca

SEQ ID NO: 686 - DNA, 20 bp, Artificial Sequence
  Description of Artificial Sequence Synthetic oligonucleotide
gacaccaagacaagcgatgt

SEQ ID NO: 687 - DNA, 20 bp, Artificial Sequence
  Description of Artificial Sequence Synthetic oligonucleotide
ggggcccacatcgcttgtct

SEQ ID NO: 688 - DNA, 20 bp, Artificial Sequence
  Description of Artificial Sequence Synthetic oligonucleotide
gcacagaagcgccaatcact

SEQ ID NO: 689 - DNA, 20 bp, Artificial Sequence
  Description of Artificial Sequence Synthetic oligonucleotide
gctttgttgtgccggatcag

SEQ ID NO: 690 - DNA, 20 bp, Artificial Sequence
  Description of Artificial Sequence Synthetic oligonucleotide
gtatcgcagccggcggagtt

SEQ ID NO: 691 - DNA, 20 bp, Artificial Sequence
  Description of Artificial Sequence Synthetic oligonucleotide
ggcaggaggcgtatcgcagc

SEQ ID NO: 692 - DNA, 20 bp, Artificial Sequence
  Description of Artificial Sequence Synthetic oligonucleotide
gggaggcgtatcgcagccgg

SEQ ID NO: 693 - DNA, 20 bp, Artificial Sequence
  Description of Artificial Sequence Synthetic oligonucleotide
gggctgcagtaccatgatct

SEQ ID NO: 694 - DNA, 20 bp, Artificial Sequence
  Description of Artificial Sequence Synthetic oligonucleotide
gttgaggccgccttctgtgg

SEQ ID NO: 695 - DNA, 20 bp, Artificial Sequence
  Description of Artificial Sequence Synthetic oligonucleotide
gccagcttgagttggaggcg

SEQ ID NO: 696 - DNA, 20 bp, Artificial Sequence
  Description of Artificial Sequence Synthetic oligonucleotide
gctggggcgctggtacagcg

SEQ ID NO: 697 - DNA, 20 bp, Artificial Sequence
  Description of Artificial Sequence Synthetic oligonucleotide
gctctcatgggtgcgtggtg

SEQ ID NO: 698 - DNA, 20 bp, Artificial Sequence
  Description of Artificial Sequence Synthetic oligonucleotide
gtggtgcttacccgctgaca

SEQ ID NO: 699 - DNA, 20 bp, Artificial Sequence
  Description of Artificial Sequence Synthetic oligonucleotide
gacccagcaggaccgtgcga

SEQ ID NO: 700 - DNA, 20 bp, Artificial Sequence
  Description of Artificial Sequence Synthetic oligonucleotide
gggagcctcgagccagtttg

SEQ ID NO: 701 - DNA, 20 bp, Artificial Sequence
  Description of Artificial Sequence Synthetic oligonucleotide
gggtgtgggagctgcgtaca

SEQ ID NO: 702 - DNA, 20 bp, Artificial Sequence
  Description of Artificial Sequence Synthetic oligonucleotide
gcagccagaggaccccgtca

SEQ ID NO: 703 - DNA, 20 bp, Artificial Sequence
  Description of Artificial Sequence Synthetic oligonucleotide
gcagagcgaagaagtagtcg

SEQ ID NO: 704 - DNA, 20 bp, Artificial Sequence
  Description of Artificial Sequence Synthetic oligonucleotide
ggccggtgcatttctacggg

SEQ ID NO: 705 - DNA, 20 bp, Artificial Sequence
  Description of Artificial Sequence Synthetic oligonucleotide
ggcatatcatcactttggct

SEQ ID NO: 706 - DNA, 20 bp, Artificial Sequence
  Description of Artificial Sequence Synthetic oligonucleotide
gagctctgacaatggcccgc

SEQ ID NO: 707 - DNA, 20 bp, Artificial Sequence
  Description of Artificial Sequence Synthetic oligonucleotide
gtgctcccagtcgcgcagct

SEQ ID NO: 708 - DNA, 20 bp, Artificial Sequence
  Description of Artificial Sequence Synthetic oligonucleotide
gcactatgacgatgatattc

SEQ ID NO: 709 - DNA, 20 bp, Artificial Sequence
  Description of Artificial Sequence Synthetic oligonucleotide
gacttgaggtgttaccctgc

SEQ ID NO: 710 - DNA, 20 bp, Artificial Sequence
  Description of Artificial Sequence Synthetic oligonucleotide
gaatggggtacgattatgca

SEQ ID NO: 711 - DNA, 20 bp, Artificial Sequence
  Description of Artificial Sequence Synthetic oligonucleotide
gctgcataatcgtaccccat

SEQ ID NO: 712 - DNA, 20 bp, Artificial Sequence
  Description of Artificial Sequence Synthetic oligonucleotide
gcaggtgtgttatatcggcc

SEQ ID NO: 713 - DNA, 20 bp, Artificial Sequence
  Description of Artificial Sequence Synthetic oligonucleotide
gctgtttgttagcgatcttc

SEQ ID NO: 714 - DNA, 20 bp, Artificial Sequence
  Description of Artificial Sequence Synthetic oligonucleotide
gacctggagtaattcccggg

SEQ ID NO: 715 - DNA, 20 bp, Artificial Sequence
  Description of Artificial Sequence Synthetic oligonucleotide
gtcatgagcagtcggtaccg

SEQ ID NO: 716 - DNA, 20 bp, Artificial Sequence
  Description of Artificial Sequence Synthetic oligonucleotide
ggcacctggagtaattcccg

SEQ ID NO: 717 - DNA, 20 bp, Artificial Sequence
  Description of Artificial Sequence Synthetic oligonucleotide
gttcaatgtttagcttgacg

SEQ ID NO: 718 - DNA, 20 bp, Artificial Sequence
  Description of Artificial Sequence Synthetic oligonucleotide
gaaagacaacgaagcggcgg

SEQ ID NO: 719 - DNA, 20 bp, Artificial Sequence
  Description of Artificial Sequence Synthetic oligonucleotide
ggggaaagacaacgaagcgg

SEQ ID NO: 720 - DNA, 20 bp, Artificial Sequence
  Description of Artificial Sequence Synthetic oligonucleotide
ggaaagacaacgaagcggcg

SEQ ID NO: 721 - DNA, 20 bp, Artificial Sequence
  Description of Artificial Sequence Synthetic oligonucleotide
gggaaagacaacgaagcggc

SEQ ID NO: 722 - DNA, 20 bp, Artificial Sequence
  Description of Artificial Sequence Synthetic oligonucleotide
ggctggagtgaaggtcccgc

SEQ ID NO: 723 - DNA, 20 bp, Artificial Sequence
  Description of Artificial Sequence Synthetic oligonucleotide
ggttctccgttcatttcgac

SEQ ID NO: 724 - DNA, 20 bp, Artificial Sequence
  Description of Artificial Sequence Synthetic oligonucleotide
gagtctctgttaagcattcg

SEQ ID NO: 725 - DNA, 20 bp, Artificial Sequence
  Description of Artificial Sequence Synthetic oligonucleotide
gagctgtccaactggcgatg

SEQ ID NO: 726 - DNA, 20 bp, Artificial Sequence
  Description of Artificial Sequence Synthetic oligonucleotide
gtactttccttcgcgaccca

SEQ ID NO: 727 - DNA, 20 bp, Artificial Sequence
  Description of Artificial Sequence Synthetic oligonucleotide
gcaaaagcagaatcgcttgg

SEQ ID NO: 728 - DNA, 20 bp, Artificial Sequence
  Description of Artificial Sequence Synthetic oligonucleotide
ggcttcttgtcgccgctcag

SEQ ID NO: 729 - DNA, 20 bp, Artificial Sequence
  Description of Artificial Sequence Synthetic oligonucleotide
gctttgaagatagcgagagc

SEQ ID NO: 730 - DNA, 20 bp, Artificial Sequence
  Description of Artificial Sequence Synthetic oligonucleotide
gacaacctccagatcgcggt

SEQ ID NO: 731 - DNA, 20 bp, Artificial Sequence
  Description of Artificial Sequence Synthetic oligonucleotide
ggaggtcgggcgaaggacgt

SEQ ID NO: 732 - DNA, 20 bp, Artificial Sequence
  Description of Artificial Sequence Synthetic oligonucleotide
gagtgatggatacgtcctgc

SEQ ID NO: 733 - DNA, 20 bp, Artificial Sequence
  Description of Artificial Sequence Synthetic oligonucleotide
gactgcagacgaccttggaa

SEQ ID NO: 734 - DNA, 20 bp, Artificial Sequence
  Description of Artificial Sequence Synthetic oligonucleotide
gggaaatctttaccgccctc

SEQ ID NO: 735 - DNA, 20 bp, Artificial Sequence
  Description of Artificial Sequence Synthetic oligonucleotide
gagatatatgagtatcgaaa

SEQ ID NO: 736 - DNA, 20 bp, Artificial Sequence
  Description of Artificial Sequence Synthetic oligonucleotide
gcactgcagacgaccttgga

SEQ ID NO: 737 - DNA, 20 bp, Artificial Sequence
  Description of Artificial Sequence Synthetic oligonucleotide
ggccgctcccggggtctatg

SEQ ID NO: 738 - DNA, 20 bp, Artificial Sequence
  Description of Artificial Sequence Synthetic oligonucleotide
gcttcttcactggtcggaag

SEQ ID NO: 739 - DNA, 20 bp, Artificial Sequence
  Description of Artificial Sequence Synthetic oligonucleotide
gcagtacacctcggaaacgc

SEQ ID NO: 740 - DNA, 20 bp, Artificial Sequence
  Description of Artificial Sequence Synthetic oligonucleotide
ggttcatgagtcgaagaaac

SEQ ID NO: 741 - DNA, 20 bp, Artificial Sequence
  Description of Artificial Sequence Synthetic oligonucleotide
gcacggctgccggcgcatcg

SEQ ID NO: 742 - DNA, 20 bp, Artificial Sequence
  Description of Artificial Sequence Synthetic oligonucleotide
gccttggtccatccgcccga

SEQ ID NO: 743 - DNA, 20 bp, Artificial Sequence
  Description of Artificial Sequence Synthetic oligonucleotide
gggtgtgtccgggacaaagt

SEQ ID NO: 744 - DNA, 20 bp, Artificial Sequence
  Description of Artificial Sequence Synthetic oligonucleotide
gcctgagggtcgtagatctg

SEQ ID NO: 745 - DNA, 20 bp, Artificial Sequence
  Description of Artificial Sequence Synthetic oligonucleotide
ggacagacgtgaaggcgtta

SEQ ID NO: 746 - DNA, 20 bp, Artificial Sequence
  Description of Artificial Sequence Synthetic oligonucleotide
gcaaagggatacaccgtctc

SEQ ID NO: 747 - DNA, 20 bp, Artificial Sequence
  Description of Artificial Sequence Synthetic oligonucleotide
gggaagctgtgggcgttcac

SEQ ID NO: 748 - DNA, 20 bp, Artificial Sequence
  Description of Artificial Sequence Synthetic oligonucleotide
ggtagacagaacccgccggg

SEQ ID NO: 749 - DNA, 20 bp, Artificial Sequence
  Description of Artificial Sequence Synthetic oligonucleotide
gctagcatgataccgtccag

SEQ ID NO: 750 - DNA, 20 bp, Artificial Sequence
  Description of Artificial Sequence Synthetic oligonucleotide
ggcccacagcccggacaggt

SEQ ID NO: 751 - DNA, 20 bp, Artificial Sequence
  Description of Artificial Sequence Synthetic oligonucleotide
gcctgatttcccggagctta

SEQ ID NO: 752 - DNA, 20 bp, Artificial Sequence
  Description of Artificial Sequence Synthetic oligonucleotide
ggagtcgtaggcgtgaaacg

SEQ ID NO: 753 - DNA, 20 bp, Artificial Sequence
  Description of Artificial Sequence Synthetic oligonucleotide
gccgatgatgcggatgtcat

SEQ ID NO: 754 - DNA, 20 bp, Artificial Sequence
  Description of Artificial Sequence Synthetic oligonucleotide
gggactgcagttcttcgcaa

SEQ ID NO: 755 - DNA, 20 bp, Artificial Sequence
  Description of Artificial Sequence Synthetic oligonucleotide
gagtgaagaatgactccccg

SEQ ID NO: 756 - DNA, 20 bp, Artificial Sequence
  Description of Artificial Sequence Synthetic oligonucleotide
gccctcatcaccgtgctgtc

SEQ ID NO: 757 - DNA, 20 bp, Artificial Sequence
  Description of Artificial Sequence Synthetic oligonucleotide
gcacggtgatgagggccacg

SEQ ID NO: 758 - DNA, 20 bp, Artificial Sequence
  Description of Artificial Sequence Synthetic oligonucleotide
ggagatggactccgcaaagc

SEQ ID NO: 759 - DNA, 20 bp, Artificial Sequence
  Description of Artificial Sequence Synthetic oligonucleotide
gccatcaatgtccagtccgt

SEQ ID NO: 760 - DNA, 20 bp, Artificial Sequence
  Description of Artificial Sequence Synthetic oligonucleotide
gcttactcttcaaccattgg

SEQ ID NO: 761 - DNA, 20 bp, Artificial Sequence
  Description of Artificial Sequence Synthetic oligonucleotide
gaccatcaatgtccagtccg

SEQ ID NO: 762 - DNA, 20 bp, Artificial Sequence
  Description of Artificial Sequence Synthetic oligonucleotide
ggcgccacagcctccacgat

SEQ ID NO: 763 - DNA, 20 bp, Artificial Sequence
  Description of Artificial Sequence Synthetic oligonucleotide
ggcacaagaggatcgctctg

SEQ ID NO: 764 - DNA, 20 bp, Artificial Sequence
  Description of Artificial Sequence Synthetic oligonucleotide
gcatgaaggaattcgccaac

SEQ ID NO: 765 - DNA, 20 bp, Artificial Sequence
  Description of Artificial Sequence Synthetic oligonucleotide
gtgcagatcggcgatagcag

SEQ ID NO: 766 - DNA, 20 bp, Artificial Sequence
  Description of Artificial Sequence Synthetic oligonucleotide
gcaacgcgctcgagaaaacc

SEQ ID NO: 767 - DNA, 20 bp, Artificial Sequence
  Description of Artificial Sequence Synthetic oligonucleotide
gtcaacgcgctcgagaaaac

SEQ ID NO: 768 - DNA, 20 bp, Artificial Sequence
  Description of Artificial Sequence Synthetic oligonucleotide
gttgtagccatggagtccgg

SEQ ID NO: 769 - DNA, 20 bp, Artificial Sequence
  Description of Artificial Sequence Synthetic oligonucleotide
gcggactccatggctacaac

SEQ ID NO: 770 - DNA, 20 bp, Artificial Sequence
  Description of Artificial Sequence Synthetic oligonucleotide
ggacctggccgaccccaaca

SEQ ID NO: 771 - DNA, 20 bp, Artificial Sequence
  Description of Artificial Sequence Synthetic oligonucleotide
gccatggagtccggaggcga

SEQ ID NO: 772 - DNA, 20 bp, Artificial Sequence
  Description of Artificial Sequence Synthetic oligonucleotide
ggagtaccatcttcgccaag

SEQ ID NO: 773 - DNA, 20 bp, Artificial Sequence
  Description of Artificial Sequence Synthetic oligonucleotide
gttttatttggctatcaacg

SEQ ID NO: 774 - DNA, 20 bp, Artificial Sequence
  Description of Artificial Sequence Synthetic oligonucleotide
ggcaagcaacgaccatgaat

SEQ ID NO: 775 - DNA, 20 bp, Artificial Sequence
  Description of Artificial Sequence Synthetic oligonucleotide
gaacgtatggtaaatggccg

SEQ ID NO: 776 - DNA, 20 bp, Artificial Sequence
  Description of Artificial Sequence Synthetic oligonucleotide
gaggtttcggcaccgaagta

SEQ ID NO: 777 - DNA, 20 bp, Artificial Sequence
  Description of Artificial Sequence Synthetic oligonucleotide
gtgtccaatcgtgtagacga

SEQ ID NO: 778 - DNA, 20 bp, Artificial Sequence
  Description of Artificial Sequence Synthetic oligonucleotide
gtagagggtggtcgcgcagt

SEQ ID NO: 779 - DNA, 20 bp, Artificial Sequence
  Description of Artificial Sequence Synthetic oligonucleotide
ggcatcatcgtgggcctcgt

SEQ ID NO: 780 - DNA, 20 bp, Artificial Sequence
  Description of Artificial Sequence Synthetic oligonucleotide
gacctggtcagccccgaacg

SEQ ID NO: 781 - DNA, 20 bp, Artificial Sequence
  Description of Artificial Sequence Synthetic oligonucleotide
ggagggtggtcgcgcagtag

SEQ ID NO: 782 - DNA, 20 bp, Artificial Sequence
  Description of Artificial Sequence Synthetic oligonucleotide
ggtcagccccgaacgaggtg

SEQ ID NO: 783 - DNA, 20 bp, Artificial Sequence
  Description of Artificial Sequence Synthetic oligonucleotide
gcttgtattttgtccgccgg

SEQ ID NO: 784 - DNA, 20 bp, Artificial Sequence
  Description of Artificial Sequence Synthetic oligonucleotide
ggcttgctccgttcaagtgg

SEQ ID NO: 785 - DNA, 20 bp, Artificial Sequence
  Description of Artificial Sequence Synthetic oligonucleotide
gagctaatgcgtccacggag

SEQ ID NO: 786 - DNA, 20 bp, Artificial Sequence
  Description of Artificial Sequence Synthetic oligonucleotide
gcctccgtggacgcattagc

SEQ ID NO: 787 - DNA, 20 bp, Artificial Sequence
  Description of Artificial Sequence Synthetic oligonucleotide
gccagctaatgcgtccacgg

SEQ ID NO: 788 - DNA, 20 bp, Artificial Sequence
  Description of Artificial Sequence Synthetic oligonucleotide
ggtgggctccgccacttgaa

SEQ ID NO: 789 - DNA, 20 bp, Artificial Sequence
  Description of Artificial Sequence Synthetic oligonucleotide
gctttctaaagcgccctaag

SEQ ID NO: 790 - DNA, 20 bp, Artificial Sequence
  Description of Artificial Sequence Synthetic oligonucleotide
gcctgagcttcgtgtgcgtg

SEQ ID NO: 791 - DNA, 20 bp, Artificial Sequence
  Description of Artificial Sequence Synthetic oligonucleotide
gtcctacggtgaccagcgcc

SEQ ID NO: 792 - DNA, 20 bp, Artificial Sequence
  Description of Artificial Sequence Synthetic oligonucleotide
gattgatgccgtctgagtac

SEQ ID NO: 793 - DNA, 20 bp, Artificial Sequence
  Description of Artificial Sequence Synthetic oligonucleotide
gctttcaggtgtcgctgcac

SEQ ID NO: 794 - DNA, 20 bp, Artificial Sequence
  Description of Artificial Sequence Synthetic oligonucleotide
gcaaaggaatacaagatcgt

SEQ ID NO: 795 - DNA, 20 bp, Artificial Sequence
  Description of Artificial Sequence Synthetic oligonucleotide
gcctccgttgcgttcccccc

SEQ ID NO: 796 - DNA, 20 bp, Artificial Sequence
  Description of Artificial Sequence Synthetic oligonucleotide
gtttctagctacatgccgat

SEQ ID NO: 797 - DNA, 20 bp, Artificial Sequence
  Description of Artificial Sequence Synthetic oligonucleotide
gaacgaaggcattccgaaac

SEQ ID NO: 798 - DNA, 20 bp, Artificial Sequence
  Description of Artificial Sequence Synthetic oligonucleotide
ggctcattttatacggtcgg

SEQ ID NO: 799 - DNA, 20 bp, Artificial Sequence
  Description of Artificial Sequence Synthetic oligonucleotide
gtgtgcttcggtttcgtgat

SEQ ID NO: 800 - DNA, 20 bp, Artificial Sequence
  Description of Artificial Sequence Synthetic oligonucleotide
gcagccagcccgactactat

SEQ ID NO: 801 - DNA, 20 bp, Artificial Sequence
  Description of Artificial Sequence Synthetic oligonucleotide
gagcggtgactggcgctatg

SEQ ID NO: 802 - DNA, 20 bp, Artificial Sequence
  Description of Artificial Sequence Synthetic oligonucleotide
ggtgatgccaacgccgaaac

SEQ ID NO: 803 - DNA, 20 bp, Artificial Sequence
  Description of Artificial Sequence Synthetic oligonucleotide
gacaccaatagtagtcgggc

SEQ ID NO: 804 - DNA, 20 bp, Artificial Sequence
  Description of Artificial Sequence Synthetic oligonucleotide
ggtgtggaggatcgacgaca

SEQ ID NO: 805 - DNA, 20 bp, Artificial Sequence
  Description of Artificial Sequence Synthetic oligonucleotide
ggtgcatcattggcgtctgc

SEQ ID NO: 806 - DNA, 20 bp, Artificial Sequence
  Description of Artificial Sequence Synthetic oligonucleotide
ggcttgtgatcataattcga

SEQ ID NO: 807 - DNA, 20 bp, Artificial Sequence
  Description of Artificial Sequence Synthetic oligonucleotide
ggatcacggacgctttgcgg

SEQ ID NO: 808 - DNA, 20 bp, Artificial Sequence
  Description of Artificial Sequence Synthetic oligonucleotide
ggggaacatggtcaacggat

SEQ ID NO: 809 - DNA, 20 bp, Artificial Sequence
  Description of Artificial Sequence Synthetic oligonucleotide
gtgcttgtataccgggcttt

SEQ ID NO: 810 - DNA, 20 bp, Artificial Sequence
  Description of Artificial Sequence Synthetic oligonucleotide
gacccatcatcatcacgttc

SEQ ID NO: 811 - DNA, 20 bp, Artificial Sequence
  Description of Artificial Sequence Synthetic oligonucleotide
gggcctgaacgtgatgatga

SEQ ID NO: 812 - DNA, 20 bp, Artificial Sequence
  Description of Artificial Sequence Synthetic oligonucleotide
ggcaaaatacatggcgtgcg

SEQ ID NO: 813 - DNA, 20 bp, Artificial Sequence
  Description of Artificial Sequence Synthetic oligonucleotide
ggtattccttaaggcggtac

SEQ ID NO: 814 - DNA, 20 bp, Artificial Sequence
  Description of Artificial Sequence Synthetic oligonucleotide
gattccttaaggcggtactg

SEQ ID NO: 815 - DNA, 20 bp, Artificial Sequence
  Description of Artificial Sequence Synthetic oligonucleotide
gagggcagtggcaacccgaa

SEQ ID NO: 816 - DNA, 20 bp, Artificial Sequence
  Description of Artificial Sequence Synthetic oligonucleotide
gtggcaccttcagacggtgt

SEQ ID NO: 817 - DNA, 20 bp, Artificial Sequence
  Description of Artificial Sequence Synthetic oligonucleotide
gcatacacggtttgactcga

SEQ ID NO: 818 - DNA, 20 bp, Artificial Sequence
  Description of Artificial Sequence Synthetic oligonucleotide
gtggccatcttctctgtcgg

SEQ ID NO: 819 - DNA, 20 bp, Artificial Sequence
  Description of Artificial Sequence Synthetic oligonucleotide
gaccagctgggaatcgtcgt

SEQ ID NO: 820 - DNA, 20 bp, Artificial Sequence
  Description of Artificial Sequence Synthetic oligonucleotide
ggccaaagcgattaacaaag

SEQ ID NO: 821 - DNA, 20 bp, Artificial Sequence
  Description of Artificial Sequence Synthetic oligonucleotide
ggggctccacgtagagctgt

SEQ ID NO: 822 - DNA, 20 bp, Artificial Sequence
  Description of Artificial Sequence Synthetic oligonucleotide
gctgctcatcaatcgtaacg

SEQ ID NO: 823 - DNA, 20 bp, Artificial Sequence
  Description of Artificial Sequence Synthetic oligonucleotide
gtatcttggcgaatcccata

SEQ ID NO: 824 - DNA, 20 bp, Artificial Sequence
  Description of Artificial Sequence Synthetic oligonucleotide
gatgggattcgccaagatag

SEQ ID NO: 825 - DNA, 20 bp, Artificial Sequence
  Description of Artificial Sequence Synthetic oligonucleotide
gccagctgggcatcgttgtt

SEQ ID NO: 826 - DNA, 20 bp, Artificial Sequence
  Description of Artificial Sequence Synthetic oligonucleotide
gaggcacaaagcccgtgcac

SEQ ID NO: 827 - DNA, 20 bp, Artificial Sequence
  Description of Artificial Sequence Synthetic oligonucleotide
gtgcaccccctgatgttcgt

SEQ ID NO: 828 - DNA, 20 bp, Artificial Sequence
  Description of Artificial Sequence Synthetic oligonucleotide
ggatgctgacgggctttgcc

SEQ ID NO: 829 - DNA, 20 bp, Artificial Sequence
  Description of Artificial Sequence Synthetic oligonucleotide
gctaccttcgccgctgtgct

SEQ ID NO: 830 - DNA, 20 bp, Artificial Sequence
  Description of Artificial Sequence Synthetic oligonucleotide
ggggccccacgaacatcagg

SEQ ID NO: 831 - DNA, 20 bp, Artificial Sequence
  Description of Artificial Sequence Synthetic oligonucleotide
gttgcccagcacagcggcga

SEQ ID NO: 832 - DNA, 20 bp, Artificial Sequence
  Description of Artificial Sequence Synthetic oligonucleotide
gagagtcttgtggagcaatt

SEQ ID NO: 833 - DNA, 20 bp, Artificial Sequence
  Description of Artificial Sequence Synthetic oligonucleotide
ggtgctggtagtcttgctcc

SEQ ID NO: 834 - DNA, 20 bp, Artificial Sequence
  Description of Artificial Sequence Synthetic oligonucleotide
gcagatcaacttcagacaat

SEQ ID NO: 835 - DNA, 20 bp, Artificial Sequence
  Description of Artificial Sequence Synthetic oligonucleotide
gtctcggttatgcgccatgg

SEQ ID NO: 836 - DNA, 20 bp, Artificial Sequence
  Description of Artificial Sequence Synthetic oligonucleotide
gaggaggccgccgaaggtga

SEQ ID NO: 837 - DNA, 20 bp, Artificial Sequence
  Description of Artificial Sequence Synthetic oligonucleotide
gaacctggtaccggtgcaga

SEQ ID NO: 838 - DNA, 20 bp, Artificial Sequence
  Description of Artificial Sequence Synthetic oligonucleotide
ggaggatcagattcggcctc

SEQ ID NO: 839 - DNA, 20 bp, Artificial Sequence
  Description of Artificial Sequence Synthetic oligonucleotide
ggcacaggccggatctttgg

SEQ ID NO: 840 - DNA, 20 bp, Artificial Sequence
  Description of Artificial Sequence Synthetic oligonucleotide
gctgctgaagtctaccgctt

SEQ ID NO: 841 - DNA, 20 bp, Artificial Sequence
  Description of Artificial Sequence Synthetic oligonucleotide
gtggttgctgcatatcgaac

SEQ ID NO: 842 - DNA, 20 bp, Artificial Sequence
  Description of Artificial Sequence Synthetic oligonucleotide
ggtggtctacgcccctgctc

SEQ ID NO: 843 - DNA, 20 bp, Artificial Sequence
  Description of Artificial Sequence Synthetic oligonucleotide
gattctctatactggagtcg

SEQ ID NO: 844 - DNA, 20 bp, Artificial Sequence
  Description of Artificial Sequence Synthetic oligonucleotide
gattgacttctaccgcctca

SEQ ID NO: 845 - DNA, 20 bp, Artificial Sequence
  Description of Artificial Sequence Synthetic oligonucleotide
gtttggggtccgtacagttg

SEQ ID NO: 846 - DNA, 20 bp, Artificial Sequence
  Description of Artificial Sequence Synthetic oligonucleotide
gtgatgaggaacagcgtgta

SEQ ID NO: 847 - DNA, 20 bp, Artificial Sequence
  Description of Artificial Sequence Synthetic oligonucleotide
gctcctcaacgcctcggtgc

SEQ ID NO: 848 - DNA, 20 bp, Artificial Sequence
  Description of Artificial Sequence Synthetic oligonucleotide
gtggggcaatacaaccggga

SEQ ID NO: 849 - DNA, 20 bp, Artificial Sequence
  Description of Artificial Sequence Synthetic oligonucleotide
gcagaactcgatgacgggtg

SEQ ID NO: 850 - DNA, 20 bp, Artificial Sequence
  Description of Artificial Sequence Synthetic oligonucleotide
ggacgatgtagtacacgttc

SEQ ID NO: 851 - DNA, 20 bp, Artificial Sequence
  Description of Artificial Sequence Synthetic oligonucleotide
gctgcctgggttgtcgattc

SEQ ID NO: 852 - DNA, 20 bp, Artificial Sequence
  Description of Artificial Sequence Synthetic oligonucleotide
gttcaggtgtggatcgacgc

SEQ ID NO: 853 - DNA, 20 bp, Artificial Sequence
  Description of Artificial Sequence Synthetic oligonucleotide
gtgactctcgttcctacgca

SEQ ID NO: 854 - DNA, 20 bp, Artificial Sequence
  Description of Artificial Sequence Synthetic oligonucleotide
ggacttcatcatgtcgtgcg

SEQ ID NO: 855 - DNA, 20 bp, Artificial Sequence
  Description of Artificial Sequence Synthetic oligonucleotide
ggtgtccaagtctctcgcgg

SEQ ID NO: 856 - DNA, 20 bp, Artificial Sequence
  Description of Artificial Sequence Synthetic oligonucleotide
gagcagtgaagtacacgatc

SEQ ID NO: 857 - DNA, 20 bp, Artificial Sequence
  Description of Artificial Sequence Synthetic oligonucleotide
gtgggcttcgccgtgggcct

SEQ ID NO: 858 - DNA, 20 bp, Artificial Sequence
  Description of Artificial Sequence Synthetic oligonucleotide
gcgtgggcctcggtaacgtg

SEQ ID NO: 859 - DNA, 20 bp, Artificial Sequence
  Description of Artificial Sequence Synthetic oligonucleotide
gctcgggcaccggacaagtg

SEQ ID NO: 860 - DNA, 20 bp, Artificial Sequence
  Description of Artificial Sequence Synthetic oligonucleotide
gcccctacttacgcacgcag

SEQ ID NO: 861 - DNA, 20 bp, Artificial Sequence
  Description of Artificial Sequence Synthetic oligonucleotide
gcggactgtcgctcgtggtg

SEQ ID NO: 862 - DNA, 20 bp, Artificial Sequence
  Description of Artificial Sequence Synthetic oligonucleotide
ggcactctgctacgcggagc

SEQ ID NO: 863 - DNA, 20 bp, Artificial Sequence
  Description of Artificial Sequence Synthetic oligonucleotide
ggccctttgttacgctgaac

SEQ ID NO: 864 - DNA, 20 bp, Artificial Sequence
  Description of Artificial Sequence Synthetic oligonucleotide
gaacgcaaggaatcccccga

SEQ ID NO: 865 - DNA, 20 bp, Artificial Sequence
  Description of Artificial Sequence Synthetic oligonucleotide
ggggactccgctctacaccc

SEQ ID NO: 866 - DNA, 20 bp, Artificial Sequence
  Description of Artificial Sequence Synthetic oligonucleotide
gtgggggattcgaagaaggc

SEQ ID NO: 867 - DNA, 20 bp, Artificial Sequence
  Description of Artificial Sequence Synthetic oligonucleotide
gctggactttaccttgcgtt

SEQ ID NO: 868 - DNA, 20 bp, Artificial Sequence
  Description of Artificial Sequence Synthetic oligonucleotide
gttggggggtgttgcagcgc

SEQ ID NO: 869 - DNA, 20 bp, Artificial Sequence
  Description of Artificial Sequence Synthetic oligonucleotide
gcagctaagcgagcttgagg

SEQ ID NO: 870 - DNA, 20 bp, Artificial Sequence
  Description of Artificial Sequence Synthetic oligonucleotide
gcgttggcagcctcgtcgct

SEQ ID NO: 871 - DNA, 20 bp, Artificial Sequence
  Description of Artificial Sequence Synthetic oligonucleotide
gtgggcagagaatccgcaat

SEQ ID NO: 872 - DNA, 20 bp, Artificial Sequence
  Description of Artificial Sequence Synthetic oligonucleotide
ggacatctccgaaaggccca

SEQ ID NO: 873 - DNA, 20 bp, Artificial Sequence
  Description of Artificial Sequence Synthetic oligonucleotide
gggacatctccgaaaggccc

SEQ ID NO: 874 - DNA, 20 bp, Artificial Sequence
  Description of Artificial Sequence Synthetic oligonucleotide
gattacagatgaccgtacca

SEQ ID NO: 875 - DNA, 20 bp, Artificial Sequence
  Description of Artificial Sequence Synthetic oligonucleotide
gagaccttactggtgactat

SEQ ID NO: 876 - DNA, 20 bp, Artificial Sequence
  Description of Artificial Sequence Synthetic oligonucleotide
gtccacaggcaccgtgataa

SEQ ID NO: 877 - DNA, 20 bp, Artificial Sequence
  Description of Artificial Sequence Synthetic oligonucleotide
gtgctggcagccgccgatct

SEQ ID NO: 878 - DNA, 20 bp, Artificial Sequence
  Description of Artificial Sequence Synthetic oligonucleotide
gtcgatgctgcggcaactga

SEQ ID NO: 879 - DNA, 20 bp, Artificial Sequence
  Description of Artificial Sequence Synthetic oligonucleotide
gccagcctcggtgcccagat

SEQ ID NO: 880 - DNA, 20 bp, Artificial Sequence
  Description of Artificial Sequence Synthetic oligonucleotide
ggtgctggcagccgccgatc

SEQ ID NO: 881 - DNA, 20 bp, Artificial Sequence
  Description of Artificial Sequence Synthetic oligonucleotide
ggcctcggtgcccagatcgg

SEQ ID NO: 882 - DNA, 20 bp, Artificial Sequence
  Description of Artificial Sequence Synthetic oligonucleotide
gacagggatattcattgcgt

SEQ ID NO: 883 - DNA, 20 bp, Artificial Sequence
  Description of Artificial Sequence Synthetic oligonucleotide
ggggaaatatgatgattcga

SEQ ID NO: 884 - DNA, 20 bp, Artificial Sequence
  Description of Artificial Sequence Synthetic oligonucleotide
ggtctctgagatagccgaat

SEQ ID NO: 885 - DNA, 20 bp, Artificial Sequence
  Description of Artificial Sequence Synthetic oligonucleotide
ggactttgtgccgctgtatc

SEQ ID NO: 886 - DNA, 20 bp, Artificial Sequence
  Description of Artificial Sequence Synthetic oligonucleotide
gtgaacaacggtcctgtctt

SEQ ID NO: 887 - DNA, 20 bp, Artificial Sequence
  Description of Artificial Sequence Synthetic oligonucleotide
gataattttccgttgcccta

SEQ ID NO: 888 - DNA, 20 bp, Artificial Sequence
  Description of Artificial Sequence Synthetic oligonucleotide
gtagggcaacggaaaattat

SEQ ID NO: 889 - DNA, 20 bp, Artificial Sequence
  Description of Artificial Sequence Synthetic oligonucleotide
gaacggaaaattattggtac

SEQ ID NO: 890 - DNA, 20 bp, Artificial Sequence
  Description of Artificial Sequence Synthetic oligonucleotide
ggagtacgtggaattatcga

SEQ ID NO: 891 - DNA, 20 bp, Artificial Sequence
  Description of Artificial Sequence Synthetic oligonucleotide
gctccttctcgttgggcagc

SEQ ID NO: 892 - DNA, 20 bp, Artificial Sequence
  Description of Artificial Sequence Synthetic oligonucleotide
ggagctccccatcgtaccac

SEQ ID NO: 893 - DNA, 20 bp, Artificial Sequence
  Description of Artificial Sequence Synthetic oligonucleotide
gcgttgggcagccggtagac

SEQ ID NO: 894 - DNA, 20 bp, Artificial Sequence
  Description of Artificial Sequence Synthetic oligonucleotide
ggttgggcagccggtagaca

SEQ ID NO: 895 - DNA, 20 bp, Artificial Sequence
  Description of Artificial Sequence Synthetic oligonucleotide
ggaacttgtggaccgcatgg

SEQ ID NO: 896 - DNA, 20 bp, Artificial Sequence
  Description of Artificial Sequence Synthetic oligonucleotide
ggatgggtcaagttcgacgt

SEQ ID NO: 897 - DNA, 20 bp, Artificial Sequence
  Description of Artificial Sequence Synthetic oligonucleotide
gaacaacttactcaatctct

SEQ ID NO: 898 - DNA, 20 bp, Artificial Sequence
  Description of Artificial Sequence Synthetic oligonucleotide
gcctcctagccctaaagtag

SEQ ID NO: 899 - DNA, 20 bp, Artificial Sequence
  Description of Artificial Sequence Synthetic oligonucleotide
gaggcagattcagaccttcg

SEQ ID NO: 900 - DNA, 20 bp, Artificial Sequence
  Description of Artificial Sequence Synthetic oligonucleotide
gcacggccaccccaaccatc

SEQ ID NO: 901 - DNA, 20 bp, Artificial Sequence
  Description of Artificial Sequence Synthetic oligonucleotide
gtaccaggttacctgattta

SEQ ID NO: 902 - DNA, 20 bp, Artificial Sequence
  Description of Artificial Sequence Synthetic oligonucleotide
gcctttcacatggtcaaacc

SEQ ID NO: 903 - DNA, 20 bp, Artificial Sequence
  Description of Artificial Sequence Synthetic oligonucleotide
gttcctactaccacttctac

SEQ ID NO: 904 - DNA, 20 bp, Artificial Sequence
  Description of Artificial Sequence Synthetic oligonucleotide
gctagatgatctatctttgc

SEQ ID NO: 905 - DNA, 20 bp, Artificial Sequence
  Description of Artificial Sequence Synthetic oligonucleotide
gttgtggagaaatttatgca

SEQ ID NO: 906 - DNA, 20 bp, Artificial Sequence
  Description of Artificial Sequence Synthetic oligonucleotide
gttcagttccgtatggttcc

SEQ ID NO: 907 - DNA, 20 bp, Artificial Sequence
  Description of Artificial Sequence Synthetic oligonucleotide
gggcgacatctttggcgttc

SEQ ID NO: 908 - DNA, 20 bp, Artificial Sequence
  Description of Artificial Sequence Synthetic oligonucleotide
gttgctacatatcctaaatc

SEQ ID NO: 909 - DNA, 20 bp, Artificial Sequence
  Description of Artificial Sequence Synthetic oligonucleotide
gggtccactggaagcgccca

SEQ ID NO: 910 - DNA, 20 bp, Artificial Sequence
  Description of Artificial Sequence Synthetic oligonucleotide
gaccgtgaacaccttttccg

SEQ ID NO: 911 - DNA, 20 bp, Artificial Sequence
  Description of Artificial Sequence Synthetic oligonucleotide
gtcttatcacgagagaaacg

SEQ ID NO: 912 - DNA, 20 bp, Artificial Sequence
  Description of Artificial Sequence Synthetic oligonucleotide
ggccaggccgcccaagatga

SEQ ID NO: 913 - DNA, 20 bp, Artificial Sequence
  Description of Artificial Sequence Synthetic oligonucleotide
gtagggctgatagtctacct

SEQ ID NO: 914 - DNA, 20 bp, Artificial Sequence
  Description of Artificial Sequence Synthetic oligonucleotide
gacaccagacctccgcgatg

SEQ ID NO: 915 - DNA, 20 bp, Artificial Sequence
  Description of Artificial Sequence Synthetic oligonucleotide
gcaggaggaaacggatcgct

SEQ ID NO: 916 - DNA, 20 bp, Artificial Sequence
  Description of Artificial Sequence Synthetic oligonucleotide
gaggagcttcgggatgttcg

SEQ ID NO: 917 - DNA, 20 bp, Artificial Sequence
  Description of Artificial Sequence Synthetic oligonucleotide
gacgcttcgtggtagacgtc

SEQ ID NO: 918 - DNA, 20 bp, Artificial Sequence
  Description of Artificial Sequence Synthetic oligonucleotide
gggaggtctggtgtgccgcg

SEQ ID NO: 919 - DNA, 20 bp, Artificial Sequence
  Description of Artificial Sequence Synthetic oligonucleotide
ggatgggagctcaaatgtcc

SEQ ID NO: 920 - DNA, 20 bp, Artificial Sequence
  Description of Artificial Sequence Synthetic oligonucleotide
gacatcggaagctgcgattg

SEQ ID NO: 921 - DNA, 20 bp, Artificial Sequence
  Description of Artificial Sequence Synthetic oligonucleotide
gggtatcatagtatgtgtct

SEQ ID NO: 922 - DNA, 20 bp, Artificial Sequence
  Description of Artificial Sequence Synthetic oligonucleotide
gtgtccatgggctccagata

SEQ ID NO: 923 - DNA, 20 bp, Artificial Sequence
  Description of Artificial Sequence Synthetic oligonucleotide
gtgcgacaacgagaaggtag

SEQ ID NO: 924 - DNA, 20 bp, Artificial Sequence
  Description of Artificial Sequence Synthetic oligonucleotide
gcacaaattcctcgaccact

SEQ ID NO: 925 - DNA, 20 bp, Artificial Sequence
  Description of Artificial Sequence Synthetic oligonucleotide
gtctttgatgtgaaccgcat

SEQ ID NO: 926 - DNA, 20 bp, Artificial Sequence
  Description of Artificial Sequence Synthetic oligonucleotide
ggtcatcagctacgtaaaca

SEQ ID NO: 927 - DNA, 20 bp, Artificial Sequence
  Description of Artificial Sequence Synthetic oligonucleotide
gtttgaggcgagtcctcaat

SEQ ID NO: 928 - DNA, 20 bp, Artificial Sequence
  Description of Artificial Sequence Synthetic oligonucleotide
ggaggcgagtcctcaatagg

SEQ ID NO: 929 - DNA, 20 bp, Artificial Sequence
  Description of Artificial Sequence Synthetic oligonucleotide
gaagtcacagcacatgacgg

SEQ ID NO: 930 - DNA, 20 bp, Artificial Sequence
  Description of Artificial Sequence Synthetic oligonucleotide
gaccatgagcgctgctccga

SEQ ID NO: 931 - DNA, 20 bp, Artificial Sequence
  Description of Artificial Sequence Synthetic oligonucleotide
gcggagcagcgctcatggtg

SEQ ID NO: 932 - DNA, 20 bp, Artificial Sequence
  Description of Artificial Sequence Synthetic oligonucleotide
gagcgctgctccgatggtga

SEQ ID NO: 933 - DNA, 20 bp, Artificial Sequence
  Description of Artificial Sequence Synthetic oligonucleotide
gatatcactacttccggtat

SEQ ID NO: 934 - DNA, 20 bp, Artificial Sequence
  Description of Artificial Sequence Synthetic oligonucleotide
ggcaagacataagtgatacg

SEQ ID NO: 935 - DNA, 20 bp, Artificial Sequence
  Description of Artificial Sequence Synthetic oligonucleotide
gctttgcatcacgcctccat

SEQ ID NO: 936 - DNA, 20 bp, Artificial Sequence
  Description of Artificial Sequence Synthetic oligonucleotide
gtcatggtgatcaaataacg

SEQ ID NO: 937 - DNA, 20 bp, Artificial Sequence
  Description of Artificial Sequence Synthetic oligonucleotide
ggccttcccgtgctgcaaag

SEQ ID NO: 938 - DNA, 20 bp, Artificial Sequence
  Description of Artificial Sequence Synthetic oligonucleotide
gagtccctcgcggttgtgga

SEQ ID NO: 939 - DNA, 20 bp, Artificial Sequence
  Description of Artificial Sequence Synthetic oligonucleotide
gcctgtcagtgtaccggggc

SEQ ID NO: 940 - DNA, 20 bp, Artificial Sequence
  Description of Artificial Sequence Synthetic oligonucleotide
gttcttggccccaccggtca

SEQ ID NO: 941 - DNA, 20 bp, Artificial Sequence
  Description of Artificial Sequence Synthetic oligonucleotide
ggtcaatggtgaaggtaacg

SEQ ID NO: 942 - DNA, 20 bp, Artificial Sequence
  Description of Artificial Sequence Synthetic oligonucleotide
gggcagccgtagttaccgag

SEQ ID NO: 943 - DNA, 20 bp, Artificial Sequence
  Description of Artificial Sequence Synthetic oligonucleotide
gcaccagcccgcgacgcaga

SEQ ID NO: 944 - DNA, 20 bp, Artificial Sequence
  Description of Artificial Sequence Synthetic oligonucleotide
gttcctgacccccgtgcgtc

SEQ ID NO: 945 - DNA, 20 bp, Artificial Sequence
  Description of Artificial Sequence Synthetic oligonucleotide
gcacgccaccggtaccttgc

SEQ ID NO: 946 - DNA, 20 bp, Artificial Sequence
  Description of Artificial Sequence Synthetic oligonucleotide
gacttaggcgaggcgaagcc

SEQ ID NO: 947 - DNA, 20 bp, Artificial Sequence
  Description of Artificial Sequence Synthetic oligonucleotide
gtaagctggaatcgattcct

SEQ ID NO: 948 - DNA, 20 bp, Artificial Sequence
  Description of Artificial Sequence Synthetic oligonucleotide
gatcctcagtaagaaggccg

SEQ ID NO: 949 - DNA, 20 bp, Artificial Sequence
  Description of Artificial Sequence Synthetic oligonucleotide
gtctgggtgacaccgaaccc

SEQ ID NO: 950 - DNA, 20 bp, Artificial Sequence
  Description of Artificial Sequence Synthetic oligonucleotide
gataagctggaatcgattcc

SEQ ID NO: 951 - DNA, 20 bp, Artificial Sequence
  Description of Artificial Sequence Synthetic oligonucleotide
ggactgcggccgctgatcat

SEQ ID NO: 952 - DNA, 20 bp, Artificial Sequence
  Description of Artificial Sequence Synthetic oligonucleotide
gatgtggatttctctcacga

SEQ ID NO: 953 - DNA, 20 bp, Artificial Sequence
  Description of Artificial Sequence Synthetic oligonucleotide
gcggtcatctttaccatcgt

SEQ ID NO: 954 - DNA, 20 bp, Artificial Sequence
  Description of Artificial Sequence Synthetic oligonucleotide
gctgtgggacttgccgtcct

SEQ ID NO: 955 - DNA, 20 bp, Artificial Sequence
  Description of Artificial Sequence Synthetic oligonucleotide
ggccgccattatccccacga

SEQ ID NO: 956 - DNA, 20 bp, Artificial Sequence
  Description of Artificial Sequence Synthetic oligonucleotide
gcagtccccggtccagagga

SEQ ID NO: 957 - DNA, 20 bp, Artificial Sequence
  Description of Artificial Sequence Synthetic oligonucleotide
ggaaagtggggttggtccac

SEQ ID NO: 958 - DNA, 20 bp, Artificial Sequence
  Description of Artificial Sequence Synthetic oligonucleotide
gcgtatgtagccgacagaca

SEQ ID NO: 959 - DNA, 20 bp, Artificial Sequence
  Description of Artificial Sequence Synthetic oligonucleotide
gtcatatggatttgtgatag

SEQ ID NO: 960 - DNA, 20 bp, Artificial Sequence
  Description of Artificial Sequence Synthetic oligonucleotide
gcagaaggggcacgaagttg

SEQ ID NO: 961 - DNA, 20 bp, Artificial Sequence
  Description of Artificial Sequence Synthetic oligonucleotide
gctgcacaatgccgagttta

SEQ ID NO: 962 - DNA, 20 bp, Artificial Sequence
  Description of Artificial Sequence Synthetic oligonucleotide
gcttgggcacgtaggacaat

SEQ ID NO: 963 - DNA, 20 bp, Artificial Sequence
  Description of Artificial Sequence Synthetic oligonucleotide
gcacgttcttcactcgctgt

SEQ ID NO: 964 - DNA, 20 bp, Artificial Sequence
  Description of Artificial Sequence Synthetic oligonucleotide
ggagtggtttattcccccta

SEQ ID NO: 965 - DNA, 20 bp, Artificial Sequence
  Description of Artificial Sequence Synthetic oligonucleotide
gagaaagtccagatagttga

SEQ ID NO: 966 - DNA, 20 bp, Artificial Sequence
  Description of Artificial Sequence Synthetic oligonucleotide
ggccatctccttcaactatc

SEQ ID NO: 967 - DNA, 20 bp, Artificial Sequence
  Description of Artificial Sequence Synthetic oligonucleotide
gtttcttactcggtcagtga

SEQ ID NO: 968 - DNA, 20 bp, Artificial Sequence
  Description of Artificial Sequence Synthetic oligonucleotide
gactatctggactttctata

SEQ ID NO: 969 - DNA, 20 bp, Artificial Sequence
  Description of Artificial Sequence Synthetic oligonucleotide
gctgaagggatatgccttcg

SEQ ID NO: 970 - DNA, 20 bp, Artificial Sequence
  Description of Artificial Sequence Synthetic oligonucleotide
gttgctgagtcggccgagga

SEQ ID NO: 971 - DNA, 20 bp, Artificial Sequence
  Description of Artificial Sequence Synthetic oligonucleotide
gctgaggtggccgcaatgtg

SEQ ID NO: 972 - DNA, 20 bp, Artificial Sequence
  Description of Artificial Sequence Synthetic oligonucleotide
gagccccacgcgcacgatct

SEQ ID NO: 973 - DNA, 20 bp, Artificial Sequence
  Description of Artificial Sequence Synthetic oligonucleotide
ggcccccagatcgtgcgcgt

SEQ ID NO: 974 - DNA, 20 bp, Artificial Sequence
  Description of Artificial Sequence Synthetic oligonucleotide
gacatcaccatcatccgcat

SEQ ID NO: 975 - DNA, 20 bp, Artificial Sequence
  Description of Artificial Sequence Synthetic oligonucleotide
gttggcacttcaggcgggat

SEQ ID NO: 976 - DNA, 20 bp, Artificial Sequence
  Description of Artificial Sequence Synthetic oligonucleotide
gaacaccttcatcaagtatg

SEQ ID NO: 977 - DNA, 20 bp, Artificial Sequence
  Description of Artificial Sequence Synthetic oligonucleotide
gtcacgtctccaaacatagc

SEQ ID NO: 978 - DNA, 20 bp, Artificial Sequence
  Description of Artificial Sequence Synthetic oligonucleotide
gctggaaaagcttcgctatc

SEQ ID NO: 979 - DNA, 20 bp, Artificial Sequence
  Description of Artificial Sequence Synthetic oligonucleotide
gtattcccaagcgtttcacg

SEQ ID NO: 980 - DNA, 20 bp, Artificial Sequence
  Description of Artificial Sequence Synthetic oligonucleotide
gtaccatcactgtagtcgat

SEQ ID NO: 981 - DNA, 20 bp, Artificial Sequence
  Description of Artificial Sequence Synthetic oligonucleotide
gtacctacccaaattccgga

SEQ ID NO: 982 - DNA, 20 bp, Artificial Sequence
  Description of Artificial Sequence Synthetic oligonucleotide
ggaggtcctggggaacgtac

SEQ ID NO: 983 - DNA, 20 bp, Artificial Sequence
  Description of Artificial Sequence Synthetic oligonucleotide
catggcagcgaagaggtccc

SEQ ID NO: 984 - DNA, 20 bp, Artificial Sequence
  Description of Artificial Sequence Synthetic oligonucleotide
agctgtcttcgtgggcaccg

SEQ ID NO: 985 - DNA, 22 bp, Artificial Sequence
  Description of Artificial Sequence Synthetic oligonucleotide
tgtaacctcacattgaacctga

SEQ ID NO: 986 - DNA, 22 bp, Artificial Sequence
  Description of Artificial Sequence Synthetic oligonucleotide
ttattgacatcgttcaccttga

SEQ ID NO: 987 - DNA, 20 bp, Artificial Sequence
  Description of Artificial Sequence Synthetic oligonucleotide
catgcgcaagagttaccgcg

SEQ ID NO: 988 - DNA, 20 bp, Artificial Sequence
  Description of Artificial Sequence Synthetic oligonucleotide
atgaccggatagtctttcgg

SEQ ID NO: 989 - DNA, 20 bp, Artificial Sequence
  Description of Artificial Sequence Synthetic oligonucleotide
gagttaccgcggggaccgag

SEQ ID NO: 990 - DNA, 20 bp, Artificial Sequence
  Description of Artificial Sequence Synthetic oligonucleotide
ttctgtgatccctgatcggg

SEQ ID NO: 991 - DNA, 137 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 992 - DNA, 123 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 993 - DNA, 110 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 994 - DNA, 102 bp, Artificial Sequence
  Description of Artificial Sequence Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 995 - DNA, 88 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtgttttttt

SEQ ID NO: 996 - DNA, 76 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt